SEQUENCE LISTING (2046 sequences)

SEQ ID NO: 1 - DNA, 10 bp, Artificial Sequence
  primer
agacgtcgtt

SEQ ID NO: 2 - DNA, 10 bp, Artificial Sequence
  primer
gaggcgatat

SEQ ID NO: 3 - DNA, 10 bp, Artificial Sequence
  primer
gtgcgaacgt

SEQ ID NO: 4 - DNA, 10 bp, Artificial Sequence
  primer
ttatactgcc

SEQ ID NO: 5 - DNA, 10 bp, Artificial Sequence
  primer
caagttcgca

SEQ ID NO: 6 - DNA, 10 bp, Artificial Sequence
  primer
acaaggtagt

SEQ ID NO: 7 - DNA, 10 bp, Artificial Sequence
  primer
acacagcgac

SEQ ID NO: 8 - DNA, 10 bp, Artificial Sequence
  primer
ttaccgatgt

SEQ ID NO: 9 - DNA, 10 bp, Artificial Sequence
  primer
cacagagtcg

SEQ ID NO: 10 - DNA, 10 bp, Artificial Sequence
  primer
ttcagcgcgt

SEQ ID NO: 11 - DNA, 10 bp, Artificial Sequence
  primer
aggaccgtga

SEQ ID NO: 12 - DNA, 10 bp, Artificial Sequence
  primer
gtctgttcgc

SEQ ID NO: 13 - DNA, 10 bp, Artificial Sequence
  primer
acctgtccac

SEQ ID NO: 14 - DNA, 10 bp, Artificial Sequence
  primer
ccgcaatgac

SEQ ID NO: 15 - DNA, 10 bp, Artificial Sequence
  primer
ctgccgatca

SEQ ID NO: 16 - DNA, 10 bp, Artificial Sequence
  primer
tacacggagc

SEQ ID NO: 17 - DNA, 10 bp, Artificial Sequence
  primer
ccgcattcat

SEQ ID NO: 18 - DNA, 10 bp, Artificial Sequence
  primer
gactctagac

SEQ ID NO: 19 - DNA, 10 bp, Artificial Sequence
  primer
ggagaactta

SEQ ID NO: 20 - DNA, 10 bp, Artificial Sequence
  primer
tccggtatgc

SEQ ID NO: 21 - DNA, 10 bp, Artificial Sequence
  primer
ggtcaggagt

SEQ ID NO: 22 - DNA, 10 bp, Artificial Sequence
  primer
acattggcag

SEQ ID NO: 23 - DNA, 10 bp, Artificial Sequence
  primer
cgtagactgc

SEQ ID NO: 24 - DNA, 10 bp, Artificial Sequence
  primer
agactgtact

SEQ ID NO: 25 - DNA, 10 bp, Artificial Sequence
  primer
tagacgcagt

SEQ ID NO: 26 - DNA, 10 bp, Artificial Sequence
  primer
ccgataatct

SEQ ID NO: 27 - DNA, 10 bp, Artificial Sequence
  primer
gagagctagt

SEQ ID NO: 28 - DNA, 10 bp, Artificial Sequence
  primer
gtaccgcgtt

SEQ ID NO: 29 - DNA, 10 bp, Artificial Sequence
  primer
gacttgcgca

SEQ ID NO: 30 - DNA, 10 bp, Artificial Sequence
  primer
cgtgattgcg

SEQ ID NO: 31 - DNA, 10 bp, Artificial Sequence
  primer
atcgtctctg

SEQ ID NO: 32 - DNA, 10 bp, Artificial Sequence
  primer
cgtagctacg

SEQ ID NO: 33 - DNA, 10 bp, Artificial Sequence
  primer
gccgaatagt

SEQ ID NO: 34 - DNA, 10 bp, Artificial Sequence
  primer
gtacctaggc

SEQ ID NO: 35 - DNA, 10 bp, Artificial Sequence
  primer
gcttacatga

SEQ ID NO: 36 - DNA, 10 bp, Artificial Sequence
  primer
tccacgtagt

SEQ ID NO: 37 - DNA, 10 bp, Artificial Sequence
  primer
agaggccatc

SEQ ID NO: 38 - DNA, 10 bp, Artificial Sequence
  primer
cggtgatgct

SEQ ID NO: 39 - DNA, 10 bp, Artificial Sequence
  primer
cactgtgctt

SEQ ID NO: 40 - DNA, 10 bp, Artificial Sequence
  primer
catgatggct

SEQ ID NO: 41 - DNA, 10 bp, Artificial Sequence
  primer
gccacacatg

SEQ ID NO: 42 - DNA, 10 bp, Artificial Sequence
  primer
cacacactgt

SEQ ID NO: 43 - DNA, 10 bp, Artificial Sequence
  primer
cagaatcata

SEQ ID NO: 44 - DNA, 10 bp, Artificial Sequence
  primer
atcgtctacg

SEQ ID NO: 45 - DNA, 10 bp, Artificial Sequence
  primer
cgagcaatac

SEQ ID NO: 46 - DNA, 10 bp, Artificial Sequence
  primer
acaagcgcac

SEQ ID NO: 47 - DNA, 10 bp, Artificial Sequence
  primer
gcttagatgt

SEQ ID NO: 48 - DNA, 10 bp, Artificial Sequence
  primer
tgcattctgg

SEQ ID NO: 49 - DNA, 10 bp, Artificial Sequence
  primer
tgtcggacca

SEQ ID NO: 50 - DNA, 10 bp, Artificial Sequence
  primer
aggcactcgt

SEQ ID NO: 51 - DNA, 10 bp, Artificial Sequence
  primer
ctgcatgtga

SEQ ID NO: 52 - DNA, 10 bp, Artificial Sequence
  primer
accacgccta

SEQ ID NO: 53 - DNA, 10 bp, Artificial Sequence
  primer
gaggtcgtac

SEQ ID NO: 54 - DNA, 10 bp, Artificial Sequence
  primer
aatactctgt

SEQ ID NO: 55 - DNA, 10 bp, Artificial Sequence
  primer
tgccaactga

SEQ ID NO: 56 - DNA, 10 bp, Artificial Sequence
  primer
cctgttcggt

SEQ ID NO: 57 - DNA, 10 bp, Artificial Sequence
  primer
gtagagagtt

SEQ ID NO: 58 - DNA, 10 bp, Artificial Sequence
  primer
tacagcgtaa

SEQ ID NO: 59 - DNA, 10 bp, Artificial Sequence
  primer
tgacgtgatg

SEQ ID NO: 60 - DNA, 10 bp, Artificial Sequence
  primer
agacgtcggt

SEQ ID NO: 61 - DNA, 10 bp, Artificial Sequence
  primer
cgctaggttc

SEQ ID NO: 62 - DNA, 10 bp, Artificial Sequence
  primer
gccttatagc

SEQ ID NO: 63 - DNA, 10 bp, Artificial Sequence
  primer
ccttcgatct

SEQ ID NO: 64 - DNA, 10 bp, Artificial Sequence
  primer
aggcaacgtg

SEQ ID NO: 65 - DNA, 10 bp, Artificial Sequence
  primer
tgagcggtgt

SEQ ID NO: 66 - DNA, 10 bp, Artificial Sequence
  primer
gtgtcgaacg

SEQ ID NO: 67 - DNA, 10 bp, Artificial Sequence
  primer
cgatgttgcg

SEQ ID NO: 68 - DNA, 10 bp, Artificial Sequence
  primer
aacaagacac

SEQ ID NO: 69 - DNA, 10 bp, Artificial Sequence
  primer
gatgctggtt

SEQ ID NO: 70 - DNA, 10 bp, Artificial Sequence
  primer
accggtagtc

SEQ ID NO: 71 - DNA, 10 bp, Artificial Sequence
  primer
gtgactagca

SEQ ID NO: 72 - DNA, 10 bp, Artificial Sequence
  primer
agcctatatt

SEQ ID NO: 73 - DNA, 10 bp, Artificial Sequence
  primer
tcgtgagctt

SEQ ID NO: 74 - DNA, 10 bp, Artificial Sequence
  primer
acactatggc

SEQ ID NO: 75 - DNA, 10 bp, Artificial Sequence
  primer
gactctgtcg

SEQ ID NO: 76 - DNA, 10 bp, Artificial Sequence
  primer
tcgatgatgc

SEQ ID NO: 77 - DNA, 10 bp, Artificial Sequence
  primer
cttggacact

SEQ ID NO: 78 - DNA, 10 bp, Artificial Sequence
  primer
ggctgatcgt

SEQ ID NO: 79 - DNA, 10 bp, Artificial Sequence
  primer
actcacaggc

SEQ ID NO: 80 - DNA, 10 bp, Artificial Sequence
  primer
atgtgcgtac

SEQ ID NO: 81 - DNA, 10 bp, Artificial Sequence
  primer
caccatcgat

SEQ ID NO: 82 - DNA, 10 bp, Artificial Sequence
  primer
agccattaac

SEQ ID NO: 83 - DNA, 10 bp, Artificial Sequence
  primer
aatcgactgt

SEQ ID NO: 84 - DNA, 10 bp, Artificial Sequence
  primer
aatactagcg

SEQ ID NO: 85 - DNA, 10 bp, Artificial Sequence
  primer
tcgtcactga

SEQ ID NO: 86 - DNA, 10 bp, Artificial Sequence
  primer
caggctctta

SEQ ID NO: 87 - DNA, 10 bp, Artificial Sequence
  primer
ggtcggtgat

SEQ ID NO: 88 - DNA, 10 bp, Artificial Sequence
  primer
cattaggcgt

SEQ ID NO: 89 - DNA, 10 bp, Artificial Sequence
  primer
actcgcgagt

SEQ ID NO: 90 - DNA, 10 bp, Artificial Sequence
  primer
ttccgaataa

SEQ ID NO: 91 - DNA, 10 bp, Artificial Sequence
  primer
tgagcatcgt

SEQ ID NO: 92 - DNA, 10 bp, Artificial Sequence
  primer
gccacgtaac

SEQ ID NO: 93 - DNA, 10 bp, Artificial Sequence
  primer
gaactacatg

SEQ ID NO: 94 - DNA, 10 bp, Artificial Sequence
  primer
tcgtgaggac

SEQ ID NO: 95 - DNA, 10 bp, Artificial Sequence
  primer
gcggccttaa

SEQ ID NO: 96 - DNA, 10 bp, Artificial Sequence
  primer
gctaaggacc

SEQ ID NO: 97 - DNA, 10 bp, Artificial Sequence
  primer
atagccatta

SEQ ID NO: 98 - DNA, 10 bp, Artificial Sequence
  primer
cagtaatcat

SEQ ID NO: 99 - DNA, 10 bp, Artificial Sequence
  primer
actccttaat

SEQ ID NO: 100 - DNA, 10 bp, Artificial Sequence
  primer
tcgaacatta

SEQ ID NO: 101 - DNA, 10 bp, Artificial Sequence
  primer
attatgaggt

SEQ ID NO: 102 - DNA, 10 bp, Artificial Sequence
  primer
aatcttagag

SEQ ID NO: 103 - DNA, 10 bp, Artificial Sequence
  primer
ttagatgatg

SEQ ID NO: 104 - DNA, 10 bp, Artificial Sequence
  primer
tacatatctg

SEQ ID NO: 105 - DNA, 10 bp, Artificial Sequence
  primer
tccttaatca

SEQ ID NO: 106 - DNA, 10 bp, Artificial Sequence
  primer
gttgagatta

SEQ ID NO: 107 - DNA, 10 bp, Artificial Sequence
  primer
tgttaacgta

SEQ ID NO: 108 - DNA, 10 bp, Artificial Sequence
  primer
catacagtaa

SEQ ID NO: 109 - DNA, 10 bp, Artificial Sequence
  primer
cttatacgaa

SEQ ID NO: 110 - DNA, 10 bp, Artificial Sequence
  primer
agatctatgt

SEQ ID NO: 111 - DNA, 10 bp, Artificial Sequence
  primer
aagacttagt

SEQ ID NO: 112 - DNA, 10 bp, Artificial Sequence
  primer
tgcgcaataa

SEQ ID NO: 113 - DNA, 10 bp, Artificial Sequence
  primer
ttggccatat

SEQ ID NO: 114 - DNA, 10 bp, Artificial Sequence
  primer
tattacgagg

SEQ ID NO: 115 - DNA, 10 bp, Artificial Sequence
  primer
ttatgatcgc

SEQ ID NO: 116 - DNA, 10 bp, Artificial Sequence
  primer
aacttaggag

SEQ ID NO: 117 - DNA, 10 bp, Artificial Sequence
  primer
tcacaatcgt

SEQ ID NO: 118 - DNA, 10 bp, Artificial Sequence
  primer
gagtatatgg

SEQ ID NO: 119 - DNA, 10 bp, Artificial Sequence
  primer
atcaggacaa

SEQ ID NO: 120 - DNA, 10 bp, Artificial Sequence
  primer
gtactgatag

SEQ ID NO: 121 - DNA, 10 bp, Artificial Sequence
  primer
cttatactcg

SEQ ID NO: 122 - DNA, 10 bp, Artificial Sequence
  primer
taacggacta

SEQ ID NO: 123 - DNA, 10 bp, Artificial Sequence
  primer
gcgttgtata

SEQ ID NO: 124 - DNA, 10 bp, Artificial Sequence
  primer
cttaagtgct

SEQ ID NO: 125 - DNA, 10 bp, Artificial Sequence
  primer
atacgactgt

SEQ ID NO: 126 - DNA, 10 bp, Artificial Sequence
  primer
actgttatcg

SEQ ID NO: 127 - DNA, 10 bp, Artificial Sequence
  primer
aatcttgacg

SEQ ID NO: 128 - DNA, 10 bp, Artificial Sequence
  primer
acatcacctt

SEQ ID NO: 129 - DNA, 10 bp, Artificial Sequence
  primer
ggtatagtac

SEQ ID NO: 130 - DNA, 10 bp, Artificial Sequence
  primer
ctaatccaca

SEQ ID NO: 131 - DNA, 10 bp, Artificial Sequence
  primer
gcaccttatt

SEQ ID NO: 132 - DNA, 10 bp, Artificial Sequence
  primer
attgacggta

SEQ ID NO: 133 - DNA, 10 bp, Artificial Sequence
  primer
gacatatggt

SEQ ID NO: 134 - DNA, 10 bp, Artificial Sequence
  primer
gatagtcgta

SEQ ID NO: 135 - DNA, 10 bp, Artificial Sequence
  primer
caattatcgc

SEQ ID NO: 136 - DNA, 10 bp, Artificial Sequence
  primer
cttaggtgat

SEQ ID NO: 137 - DNA, 10 bp, Artificial Sequence
  primer
catactactg

SEQ ID NO: 138 - DNA, 10 bp, Artificial Sequence
  primer
taacgcgaat

SEQ ID NO: 139 - DNA, 10 bp, Artificial Sequence
  primer
caagttacga

SEQ ID NO: 140 - DNA, 10 bp, Artificial Sequence
  primer
aatctcaagg

SEQ ID NO: 141 - DNA, 10 bp, Artificial Sequence
  primer
gcaatcatca

SEQ ID NO: 142 - DNA, 10 bp, Artificial Sequence
  primer
tgtaacgttc

SEQ ID NO: 143 - DNA, 10 bp, Artificial Sequence
  primer
tatcgttggt

SEQ ID NO: 144 - DNA, 10 bp, Artificial Sequence
  primer
cgcttaagat

SEQ ID NO: 145 - DNA, 10 bp, Artificial Sequence
  primer
ttagaactgg

SEQ ID NO: 146 - DNA, 10 bp, Artificial Sequence
  primer
gtcataacgt

SEQ ID NO: 147 - DNA, 10 bp, Artificial Sequence
  primer
agagcagtat

SEQ ID NO: 148 - DNA, 10 bp, Artificial Sequence
  primer
caacatcact

SEQ ID NO: 149 - DNA, 10 bp, Artificial Sequence
  primer
cagaagctta

SEQ ID NO: 150 - DNA, 10 bp, Artificial Sequence
  primer
aactaacgtg

SEQ ID NO: 151 - DNA, 10 bp, Artificial Sequence
  primer
ttataccgct

SEQ ID NO: 152 - DNA, 10 bp, Artificial Sequence
  primer
gaattcgaga

SEQ ID NO: 153 - DNA, 10 bp, Artificial Sequence
  primer
ttacgtaacc

SEQ ID NO: 154 - DNA, 10 bp, Artificial Sequence
  primer
gcatggttaa

SEQ ID NO: 155 - DNA, 10 bp, Artificial Sequence
  primer
gcacctaatt

SEQ ID NO: 156 - DNA, 10 bp, Artificial Sequence
  primer
tgtaggttgt

SEQ ID NO: 157 - DNA, 10 bp, Artificial Sequence
  primer
ccatctggaa

SEQ ID NO: 158 - DNA, 10 bp, Artificial Sequence
  primer
ttcgcgttga

SEQ ID NO: 159 - DNA, 10 bp, Artificial Sequence
  primer
aaccgaggtt

SEQ ID NO: 160 - DNA, 10 bp, Artificial Sequence
  primer
gtacgctgtt

SEQ ID NO: 161 - DNA, 10 bp, Artificial Sequence
  primer
agtatcctgg

SEQ ID NO: 162 - DNA, 10 bp, Artificial Sequence
  primer
ggttgtacag

SEQ ID NO: 163 - DNA, 10 bp, Artificial Sequence
  primer
acgtacacca

SEQ ID NO: 164 - DNA, 10 bp, Artificial Sequence
  primer
tgtcgagcaa

SEQ ID NO: 165 - DNA, 10 bp, Artificial Sequence
  primer
gtcgtgttac

SEQ ID NO: 166 - DNA, 10 bp, Artificial Sequence
  primer
gtgcaatagg

SEQ ID NO: 167 - DNA, 10 bp, Artificial Sequence
  primer
actcgatgct

SEQ ID NO: 168 - DNA, 10 bp, Artificial Sequence
  primer
gaatcgcgta

SEQ ID NO: 169 - DNA, 10 bp, Artificial Sequence
  primer
cggtcattgt

SEQ ID NO: 170 - DNA, 10 bp, Artificial Sequence
  primer
atcaggcgat

SEQ ID NO: 171 - DNA, 10 bp, Artificial Sequence
  primer
gtaagatgcg

SEQ ID NO: 172 - DNA, 10 bp, Artificial Sequence
  primer
ggtctcttga

SEQ ID NO: 173 - DNA, 10 bp, Artificial Sequence
  primer
tcctcgctaa

SEQ ID NO: 174 - DNA, 10 bp, Artificial Sequence
  primer
ctgcgtgata

SEQ ID NO: 175 - DNA, 10 bp, Artificial Sequence
  primer
catactcgtc

SEQ ID NO: 176 - DNA, 10 bp, Artificial Sequence
  primer
atctgagctc

SEQ ID NO: 177 - DNA, 10 bp, Artificial Sequence
  primer
acggatagtg

SEQ ID NO: 178 - DNA, 10 bp, Artificial Sequence
  primer
actgcaatgc

SEQ ID NO: 179 - DNA, 10 bp, Artificial Sequence
  primer
taacgacgtg

SEQ ID NO: 180 - DNA, 10 bp, Artificial Sequence
  primer
tagactgtcg

SEQ ID NO: 181 - DNA, 10 bp, Artificial Sequence
  primer
cagcacttca

SEQ ID NO: 182 - DNA, 10 bp, Artificial Sequence
  primer
aacattcgcc

SEQ ID NO: 183 - DNA, 10 bp, Artificial Sequence
  primer
actagtgcgt

SEQ ID NO: 184 - DNA, 10 bp, Artificial Sequence
  primer
acgctgttct

SEQ ID NO: 185 - DNA, 10 bp, Artificial Sequence
  primer
cgtcgaatgc

SEQ ID NO: 186 - DNA, 10 bp, Artificial Sequence
  primer
ctctgacggt

SEQ ID NO: 187 - DNA, 10 bp, Artificial Sequence
  primer
gtcgccatgt

SEQ ID NO: 188 - DNA, 10 bp, Artificial Sequence
  primer
ggtccacgtt

SEQ ID NO: 189 - DNA, 10 bp, Artificial Sequence
  primer
cgagcgactt

SEQ ID NO: 190 - DNA, 10 bp, Artificial Sequence
  primer
ttgacgcgtg

SEQ ID NO: 191 - DNA, 10 bp, Artificial Sequence
  primer
ctgagagcct

SEQ ID NO: 192 - DNA, 10 bp, Artificial Sequence
  primer
cgcgctaact

SEQ ID NO: 193 - DNA, 10 bp, Artificial Sequence
  primer
ggtcgtcaag

SEQ ID NO: 194 - DNA, 10 bp, Artificial Sequence
  primer
aggttgacca

SEQ ID NO: 195 - DNA, 10 bp, Artificial Sequence
  primer
taacggcaac

SEQ ID NO: 196 - DNA, 10 bp, Artificial Sequence
  primer
gaggctggat

SEQ ID NO: 197 - DNA, 10 bp, Artificial Sequence
  primer
gtgcacacct

SEQ ID NO: 198 - DNA, 10 bp, Artificial Sequence
  primer
tgaggaccag

SEQ ID NO: 199 - DNA, 10 bp, Artificial Sequence
  primer
tacttgcgag

SEQ ID NO: 200 - DNA, 10 bp, Artificial Sequence
  primer
aactgtgaga

SEQ ID NO: 201 - DNA, 10 bp, Artificial Sequence
  primer
ctccatcaac

SEQ ID NO: 202 - DNA, 10 bp, Artificial Sequence
  primer
cggactgtta

SEQ ID NO: 203 - DNA, 10 bp, Artificial Sequence
  primer
taggacagtc

SEQ ID NO: 204 - DNA, 10 bp, Artificial Sequence
  primer
agaggacaca

SEQ ID NO: 205 - DNA, 10 bp, Artificial Sequence
  primer
acattcgcgg

SEQ ID NO: 206 - DNA, 10 bp, Artificial Sequence
  primer
gcttactgca

SEQ ID NO: 207 - DNA, 10 bp, Artificial Sequence
  primer
caatacgtaa

SEQ ID NO: 208 - DNA, 10 bp, Artificial Sequence
  primer
agacttgcgc

SEQ ID NO: 209 - DNA, 10 bp, Artificial Sequence
  primer
gagcggtgtt

SEQ ID NO: 210 - DNA, 10 bp, Artificial Sequence
  primer
cgtgagaggt

SEQ ID NO: 211 - DNA, 10 bp, Artificial Sequence
  primer
aatccgtcag

SEQ ID NO: 212 - DNA, 10 bp, Artificial Sequence
  primer
atacgtaccg

SEQ ID NO: 213 - DNA, 10 bp, Artificial Sequence
  primer
aactgattcc

SEQ ID NO: 214 - DNA, 10 bp, Artificial Sequence
  primer
ctgagcgtac

SEQ ID NO: 215 - DNA, 10 bp, Artificial Sequence
  primer
gtcggattcg

SEQ ID NO: 216 - DNA, 10 bp, Artificial Sequence
  primer
gccgaccata

SEQ ID NO: 217 - DNA, 10 bp, Artificial Sequence
  primer
gcagaactaa

SEQ ID NO: 218 - DNA, 10 bp, Artificial Sequence
  primer
ctaacgaccg

SEQ ID NO: 219 - DNA, 10 bp, Artificial Sequence
  primer
gctggaccat

SEQ ID NO: 220 - DNA, 10 bp, Artificial Sequence
  primer
gacgcggtta

SEQ ID NO: 221 - DNA, 10 bp, Artificial Sequence
  primer
agtggtgagc

SEQ ID NO: 222 - DNA, 10 bp, Artificial Sequence
  primer
caggcagtca

SEQ ID NO: 223 - DNA, 10 bp, Artificial Sequence
  primer
tctgacgtca

SEQ ID NO: 224 - DNA, 10 bp, Artificial Sequence
  primer
tacatgacgt

SEQ ID NO: 225 - DNA, 10 bp, Artificial Sequence
  primer
tgaggcaacc

SEQ ID NO: 226 - DNA, 10 bp, Artificial Sequence
  primer
caactgcagt

SEQ ID NO: 227 - DNA, 10 bp, Artificial Sequence
  primer
cggagatacg

SEQ ID NO: 228 - DNA, 10 bp, Artificial Sequence
  primer
cttcgcaagt

SEQ ID NO: 229 - DNA, 10 bp, Artificial Sequence
  primer
ctggcatacg

SEQ ID NO: 230 - DNA, 10 bp, Artificial Sequence
  primer
taacgttcgc

SEQ ID NO: 231 - DNA, 10 bp, Artificial Sequence
  primer
ccggcgttaa

SEQ ID NO: 232 - DNA, 10 bp, Artificial Sequence
  primer
acaagacgcc

SEQ ID NO: 233 - DNA, 10 bp, Artificial Sequence
  primer
ccattagact

SEQ ID NO: 234 - DNA, 10 bp, Artificial Sequence
  primer
gtctgtgaca

SEQ ID NO: 235 - DNA, 10 bp, Artificial Sequence
  primer
ggcattggac

SEQ ID NO: 236 - DNA, 10 bp, Artificial Sequence
  primer
tcttcgcacg

SEQ ID NO: 237 - DNA, 10 bp, Artificial Sequence
  primer
tagcctgtgc

SEQ ID NO: 238 - DNA, 10 bp, Artificial Sequence
  primer
cactgaccta

SEQ ID NO: 239 - DNA, 10 bp, Artificial Sequence
  primer
ccgcacgatt

SEQ ID NO: 240 - DNA, 10 bp, Artificial Sequence
  primer
atagcacacg

SEQ ID NO: 241 - DNA, 10 bp, Artificial Sequence
  primer
gcacgtcata

SEQ ID NO: 242 - DNA, 10 bp, Artificial Sequence
  primer
aagccgttgg

SEQ ID NO: 243 - DNA, 10 bp, Artificial Sequence
  primer
cggaccgtta

SEQ ID NO: 244 - DNA, 10 bp, Artificial Sequence
  primer
tacacagcgt

SEQ ID NO: 245 - DNA, 10 bp, Artificial Sequence
  primer
cggacttcag

SEQ ID NO: 246 - DNA, 10 bp, Artificial Sequence
  primer
tagaacgtca

SEQ ID NO: 247 - DNA, 10 bp, Artificial Sequence
  primer
ggcattggag

SEQ ID NO: 248 - DNA, 10 bp, Artificial Sequence
  primer
ggcactcgtt

SEQ ID NO: 249 - DNA, 10 bp, Artificial Sequence
  primer
gtaccgttaa

SEQ ID NO: 250 - DNA, 10 bp, Artificial Sequence
  primer
aatacgtgtc

SEQ ID NO: 251 - DNA, 10 bp, Artificial Sequence
  primer
ccattgacgt

SEQ ID NO: 252 - DNA, 10 bp, Artificial Sequence
  primer
cgtgaatcgc

SEQ ID NO: 253 - DNA, 10 bp, Artificial Sequence
  primer
atcaacgcgg

SEQ ID NO: 254 - DNA, 10 bp, Artificial Sequence
  primer
cgccaaggta

SEQ ID NO: 255 - DNA, 10 bp, Artificial Sequence
  primer
agaagacgcc

SEQ ID NO: 256 - DNA, 10 bp, Artificial Sequence
  primer
ccgcatagtc

SEQ ID NO: 257 - DNA, 10 bp, Artificial Sequence
  primer
cttatatgtg

SEQ ID NO: 258 - DNA, 10 bp, Artificial Sequence
  primer
ggtctcatcg

SEQ ID NO: 259 - DNA, 10 bp, Artificial Sequence
  primer
ccaccatgtc

SEQ ID NO: 260 - DNA, 10 bp, Artificial Sequence
  primer
acgaatgtgt

SEQ ID NO: 261 - DNA, 10 bp, Artificial Sequence
  primer
ggtagtaaca

SEQ ID NO: 262 - DNA, 10 bp, Artificial Sequence
  primer
gccacttaat

SEQ ID NO: 263 - DNA, 10 bp, Artificial Sequence
  primer
atattgcgcc

SEQ ID NO: 264 - DNA, 10 bp, Artificial Sequence
  primer
gaccaatagt

SEQ ID NO: 265 - DNA, 10 bp, Artificial Sequence
  primer
aacaacacgg

SEQ ID NO: 266 - DNA, 10 bp, Artificial Sequence
  primer
atagccgatg

SEQ ID NO: 267 - DNA, 10 bp, Artificial Sequence
  primer
cgagagcata

SEQ ID NO: 268 - DNA, 10 bp, Artificial Sequence
  primer
cgagacatga

SEQ ID NO: 269 - DNA, 10 bp, Artificial Sequence
  primer
cgccaagtta

SEQ ID NO: 270 - DNA, 10 bp, Artificial Sequence
  primer
ttataatcgc

SEQ ID NO: 271 - DNA, 10 bp, Artificial Sequence
  primer
tagaagtgca

SEQ ID NO: 272 - DNA, 10 bp, Artificial Sequence
  primer
ggaggcatgt

SEQ ID NO: 273 - DNA, 10 bp, Artificial Sequence
  primer
gccacttcga

SEQ ID NO: 274 - DNA, 10 bp, Artificial Sequence
  primer
tccacggtac

SEQ ID NO: 275 - DNA, 10 bp, Artificial Sequence
  primer
caactatgca

SEQ ID NO: 276 - DNA, 10 bp, Artificial Sequence
  primer
caaggaggac

SEQ ID NO: 277 - DNA, 10 bp, Artificial Sequence
  primer
gaggtaccta

SEQ ID NO: 278 - DNA, 10 bp, Artificial Sequence
  primer
gagcgcataa

SEQ ID NO: 279 - DNA, 10 bp, Artificial Sequence
  primer
tcgtcacgtg

SEQ ID NO: 280 - DNA, 10 bp, Artificial Sequence
  primer
aactgtgaca

SEQ ID NO: 281 - DNA, 10 bp, Artificial Sequence
  primer
tccacgtgag

SEQ ID NO: 282 - DNA, 10 bp, Artificial Sequence
  primer
acactgctct

SEQ ID NO: 283 - DNA, 10 bp, Artificial Sequence
  primer
tacggtgagc

SEQ ID NO: 284 - DNA, 10 bp, Artificial Sequence
  primer
cggactaagt

SEQ ID NO: 285 - DNA, 10 bp, Artificial Sequence
  primer
aagccacgtt

SEQ ID NO: 286 - DNA, 10 bp, Artificial Sequence
  primer
caattactcg

SEQ ID NO: 287 - DNA, 10 bp, Artificial Sequence
  primer
tctggccata

SEQ ID NO: 288 - DNA, 10 bp, Artificial Sequence
  primer
tcaggctagt

SEQ ID NO: 289 - DNA, 10 bp, Artificial Sequence
  primer
ttgacccgga

SEQ ID NO: 290 - DNA, 10 bp, Artificial Sequence
  primer
tttttatggt

SEQ ID NO: 291 - DNA, 10 bp, Artificial Sequence
  primer
atgtggtgcg

SEQ ID NO: 292 - DNA, 10 bp, Artificial Sequence
  primer
aaggcgctag

SEQ ID NO: 293 - DNA, 10 bp, Artificial Sequence
  primer
tccaactttg

SEQ ID NO: 294 - DNA, 10 bp, Artificial Sequence
  primer
ccatcccatc

SEQ ID NO: 295 - DNA, 10 bp, Artificial Sequence
  primer
caatacgagg

SEQ ID NO: 296 - DNA, 10 bp, Artificial Sequence
  primer
gagtgttacc

SEQ ID NO: 297 - DNA, 10 bp, Artificial Sequence
  primer
gcctcctgta

SEQ ID NO: 298 - DNA, 10 bp, Artificial Sequence
  primer
cgaaggttgc

SEQ ID NO: 299 - DNA, 10 bp, Artificial Sequence
  primer
gaggtgctat

SEQ ID NO: 300 - DNA, 10 bp, Artificial Sequence
  primer
taggataatt

SEQ ID NO: 301 - DNA, 10 bp, Artificial Sequence
  primer
cgttgtcctc

SEQ ID NO: 302 - DNA, 10 bp, Artificial Sequence
  primer
tgagaccagc

SEQ ID NO: 303 - DNA, 10 bp, Artificial Sequence
  primer
tgcccaagct

SEQ ID NO: 304 - DNA, 10 bp, Artificial Sequence
  primer
tactgaatcg

SEQ ID NO: 305 - DNA, 10 bp, Artificial Sequence
  primer
ttacatagtc

SEQ ID NO: 306 - DNA, 10 bp, Artificial Sequence
  primer
acaaaggaaa

SEQ ID NO: 307 - DNA, 10 bp, Artificial Sequence
  primer
ctcgcttggg

SEQ ID NO: 308 - DNA, 10 bp, Artificial Sequence
  primer
ccttgcgtca

SEQ ID NO: 309 - DNA, 10 bp, Artificial Sequence
  primer
taattccgaa

SEQ ID NO: 310 - DNA, 10 bp, Artificial Sequence
  primer
gtgagcttga

SEQ ID NO: 311 - DNA, 10 bp, Artificial Sequence
  primer
atgccgattc

SEQ ID NO: 312 - DNA, 10 bp, Artificial Sequence
  primer
gcttgggctt

SEQ ID NO: 313 - DNA, 10 bp, Artificial Sequence
  primer
acaaagcgcc

SEQ ID NO: 314 - DNA, 10 bp, Artificial Sequence
  primer
gaaagctcta

SEQ ID NO: 315 - DNA, 10 bp, Artificial Sequence
  primer
taccgaccgt

SEQ ID NO: 316 - DNA, 10 bp, Artificial Sequence
  primer
tcgaagagac

SEQ ID NO: 317 - DNA, 10 bp, Artificial Sequence
  primer
gtcgcttacg

SEQ ID NO: 318 - DNA, 10 bp, Artificial Sequence
  primer
gggctctcca

SEQ ID NO: 319 - DNA, 10 bp, Artificial Sequence
  primer
gcgcccttgt

SEQ ID NO: 320 - DNA, 10 bp, Artificial Sequence
  primer
ggcaataggc

SEQ ID NO: 321 - DNA, 10 bp, Artificial Sequence
  primer
caagtcagga

SEQ ID NO: 322 - DNA, 10 bp, Artificial Sequence
  primer
gggtcgcaat

SEQ ID NO: 323 - DNA, 10 bp, Artificial Sequence
  primer
cagcaaccta

SEQ ID NO: 324 - DNA, 10 bp, Artificial Sequence
  primer
ttcccgccac

SEQ ID NO: 325 - DNA, 10 bp, Artificial Sequence
  primer
tgtgcatttt

SEQ ID NO: 326 - DNA, 10 bp, Artificial Sequence
  primer
atcaacgacg

SEQ ID NO: 327 - DNA, 10 bp, Artificial Sequence
  primer
gtgacgtcca

SEQ ID NO: 328 - DNA, 10 bp, Artificial Sequence
  primer
cgatctagtc

SEQ ID NO: 329 - DNA, 10 bp, Artificial Sequence
  primer
ttacatcctg

SEQ ID NO: 330 - DNA, 10 bp, Artificial Sequence
  primer
agccttcaat

SEQ ID NO: 331 - DNA, 10 bp, Artificial Sequence
  primer
tccatccgat

SEQ ID NO: 332 - DNA, 10 bp, Artificial Sequence
  primer
gactgggtct

SEQ ID NO: 333 - DNA, 10 bp, Artificial Sequence
  primer
ttcggtggag

SEQ ID NO: 334 - DNA, 10 bp, Artificial Sequence
  primer
gaccagcaca

SEQ ID NO: 335 - DNA, 10 bp, Artificial Sequence
  primer
cattaacgga

SEQ ID NO: 336 - DNA, 10 bp, Artificial Sequence
  primer
tttttcttga

SEQ ID NO: 337 - DNA, 10 bp, Artificial Sequence
  primer
cattgcactg

SEQ ID NO: 338 - DNA, 10 bp, Artificial Sequence
  primer
tgcggcgatc

SEQ ID NO: 339 - DNA, 10 bp, Artificial Sequence
  primer
atattgcggt

SEQ ID NO: 340 - DNA, 10 bp, Artificial Sequence
  primer
gacgtcgctc

SEQ ID NO: 341 - DNA, 10 bp, Artificial Sequence
  primer
tcgcttatcg

SEQ ID NO: 342 - DNA, 10 bp, Artificial Sequence
  primer
gcgcagacac

SEQ ID NO: 343 - DNA, 10 bp, Artificial Sequence
  primer
catgtattgt

SEQ ID NO: 344 - DNA, 10 bp, Artificial Sequence
  primer
tctataacct

SEQ ID NO: 345 - DNA, 10 bp, Artificial Sequence
  primer
gtggagacaa

SEQ ID NO: 346 - DNA, 10 bp, Artificial Sequence
  primer
cgaagattat

SEQ ID NO: 347 - DNA, 10 bp, Artificial Sequence
  primer
tagcaactgc

SEQ ID NO: 348 - DNA, 10 bp, Artificial Sequence
  primer
ataatcggta

SEQ ID NO: 349 - DNA, 10 bp, Artificial Sequence
  primer
caggatgggt

SEQ ID NO: 350 - DNA, 10 bp, Artificial Sequence
  primer
gacgattccc

SEQ ID NO: 351 - DNA, 10 bp, Artificial Sequence
  primer
cacgccttac

SEQ ID NO: 352 - DNA, 10 bp, Artificial Sequence
  primer
agttggttcc

SEQ ID NO: 353 - DNA, 10 bp, Artificial Sequence
  primer
tcttatcagg

SEQ ID NO: 354 - DNA, 10 bp, Artificial Sequence
  primer
cgagaagttc

SEQ ID NO: 355 - DNA, 10 bp, Artificial Sequence
  primer
gtggtagaat

SEQ ID NO: 356 - DNA, 10 bp, Artificial Sequence
  primer
taggcttgtg

SEQ ID NO: 357 - DNA, 10 bp, Artificial Sequence
  primer
atgcgttacg

SEQ ID NO: 358 - DNA, 10 bp, Artificial Sequence
  primer
actaccgagg

SEQ ID NO: 359 - DNA, 10 bp, Artificial Sequence
  primer
cgagttggtg

SEQ ID NO: 360 - DNA, 10 bp, Artificial Sequence
  primer
ggacgatcaa

SEQ ID NO: 361 - DNA, 10 bp, Artificial Sequence
  primer
aacagtatgc

SEQ ID NO: 362 - DNA, 10 bp, Artificial Sequence
  primer
ttggctgatc

SEQ ID NO: 363 - DNA, 10 bp, Artificial Sequence
  primer
aggattggaa

SEQ ID NO: 364 - DNA, 10 bp, Artificial Sequence
  primer
catatggaga

SEQ ID NO: 365 - DNA, 10 bp, Artificial Sequence
  primer
ctgcaggttt

SEQ ID NO: 366 - DNA, 10 bp, Artificial Sequence
  primer
ctctcttttt

SEQ ID NO: 367 - DNA, 10 bp, Artificial Sequence
  primer
agtaggggtc

SEQ ID NO: 368 - DNA, 10 bp, Artificial Sequence
  primer
acaccgcaag

SEQ ID NO: 369 - DNA, 10 bp, Artificial Sequence
  primer
gaagcgggag

SEQ ID NO: 370 - DNA, 10 bp, Artificial Sequence
  primer
gatacggact

SEQ ID NO: 371 - DNA, 10 bp, Artificial Sequence
  primer
tacgacgtgt

SEQ ID NO: 372 - DNA, 10 bp, Artificial Sequence
  primer
gtgcctcctt

SEQ ID NO: 373 - DNA, 10 bp, Artificial Sequence
  primer
ggtgactgat

SEQ ID NO: 374 - DNA, 10 bp, Artificial Sequence
  primer
atatcttacg

SEQ ID NO: 375 - DNA, 10 bp, Artificial Sequence
  primer
aatcatacgg

SEQ ID NO: 376 - DNA, 10 bp, Artificial Sequence
  primer
ctcttgggac

SEQ ID NO: 377 - DNA, 10 bp, Artificial Sequence
  primer
gacgacaaat

SEQ ID NO: 378 - DNA, 10 bp, Artificial Sequence
  primer
gttgcgaggt

SEQ ID NO: 379 - DNA, 10 bp, Artificial Sequence
  primer
aaaccgcacc

SEQ ID NO: 380 - DNA, 10 bp, Artificial Sequence
  primer
gctaacacgt

SEQ ID NO: 381 - DNA, 10 bp, Artificial Sequence
  primer
atcatgaggg

SEQ ID NO: 382 - DNA, 10 bp, Artificial Sequence
  primer
gattcacgta

SEQ ID NO: 383 - DNA, 10 bp, Artificial Sequence
  primer
tctcgaaaag

SEQ ID NO: 384 - DNA, 10 bp, Artificial Sequence
  primer
ctcgtaacca

SEQ ID NO: 385 - DNA, 10 bp, Artificial Sequence
  primer
gttacacacg

SEQ ID NO: 386 - DNA, 10 bp, Artificial Sequence
  primer
cgtgaagggt

SEQ ID NO: 387 - DNA, 10 bp, Artificial Sequence
  primer
acgagcatct

SEQ ID NO: 388 - DNA, 10 bp, Artificial Sequence
  primer
acgagggatt

SEQ ID NO: 389 - DNA, 10 bp, Artificial Sequence
  primer
gcaacgtcgg

SEQ ID NO: 390 - DNA, 10 bp, Artificial Sequence
  primer
cacggctagg

SEQ ID NO: 391 - DNA, 10 bp, Artificial Sequence
  primer
cgtgactctc

SEQ ID NO: 392 - DNA, 10 bp, Artificial Sequence
  primer
tctagacgca

SEQ ID NO: 393 - DNA, 10 bp, Artificial Sequence
  primer
ctgcgcacat

SEQ ID NO: 394 - DNA, 10 bp, Artificial Sequence
  primer
atgcttgaca

SEQ ID NO: 395 - DNA, 10 bp, Artificial Sequence
  primer
tttgtcgaca

SEQ ID NO: 396 - DNA, 10 bp, Artificial Sequence
  primer
acgtgtcagc

SEQ ID NO: 397 - DNA, 10 bp, Artificial Sequence
  primer
gaaaacatta

SEQ ID NO: 398 - DNA, 10 bp, Artificial Sequence
  primer
acattaacgg

SEQ ID NO: 399 - DNA, 10 bp, Artificial Sequence
  primer
gtacaggtcc

SEQ ID NO: 400 - DNA, 10 bp, Artificial Sequence
  primer
ctatgtgtac

SEQ ID NO: 401 - DNA, 10 bp, Artificial Sequence
  primer
gcgtacatta

SEQ ID NO: 402 - DNA, 10 bp, Artificial Sequence
  primer
gatttgtggc

SEQ ID NO: 403 - DNA, 10 bp, Artificial Sequence
  primer
tcgcgcgcta

SEQ ID NO: 404 - DNA, 10 bp, Artificial Sequence
  primer
acaagggcga

SEQ ID NO: 405 - DNA, 10 bp, Artificial Sequence
  primer
aacgcgcgat

SEQ ID NO: 406 - DNA, 10 bp, Artificial Sequence
  primer
cgtaaatgcg

SEQ ID NO: 407 - DNA, 10 bp, Artificial Sequence
  primer
taggcactac

SEQ ID NO: 408 - DNA, 10 bp, Artificial Sequence
  primer
gcgaggatcg

SEQ ID NO: 409 - DNA, 10 bp, Artificial Sequence
  primer
cacgtttact

SEQ ID NO: 410 - DNA, 10 bp, Artificial Sequence
  primer
taccaccacg

SEQ ID NO: 411 - DNA, 10 bp, Artificial Sequence
  primer
ttaacaggac

SEQ ID NO: 412 - DNA, 10 bp, Artificial Sequence
  primer
gctgtataac

SEQ ID NO: 413 - DNA, 10 bp, Artificial Sequence
  primer
gttgctggca

SEQ ID NO: 414 - DNA, 10 bp, Artificial Sequence
  primer
agtgtggcca

SEQ ID NO: 415 - DNA, 10 bp, Artificial Sequence
  primer
ctgcggttgt

SEQ ID NO: 416 - DNA, 10 bp, Artificial Sequence
  primer
tagatcagcg

SEQ ID NO: 417 - DNA, 10 bp, Artificial Sequence
  primer
ttccggttat

SEQ ID NO: 418 - DNA, 10 bp, Artificial Sequence
  primer
gataaactgt

SEQ ID NO: 419 - DNA, 10 bp, Artificial Sequence
  primer
tacagttgcc

SEQ ID NO: 420 - DNA, 10 bp, Artificial Sequence
  primer
cgatggcgaa

SEQ ID NO: 421 - DNA, 10 bp, Artificial Sequence
  primer
ccgacgtcag

SEQ ID NO: 422 - DNA, 10 bp, Artificial Sequence
  primer
tatggtgcaa

SEQ ID NO: 423 - DNA, 10 bp, Artificial Sequence
  primer
gacgacagtc

SEQ ID NO: 424 - DNA, 10 bp, Artificial Sequence
  primer
gtcaccgtcc

SEQ ID NO: 425 - DNA, 10 bp, Artificial Sequence
  primer
ggttttaaca

SEQ ID NO: 426 - DNA, 10 bp, Artificial Sequence
  primer
gaggacagta

SEQ ID NO: 427 - DNA, 10 bp, Artificial Sequence
  primer
gttacctaag

SEQ ID NO: 428 - DNA, 10 bp, Artificial Sequence
  primer
atcacgtgtt

SEQ ID NO: 429 - DNA, 10 bp, Artificial Sequence
  primer
taaggcctgg

SEQ ID NO: 430 - DNA, 10 bp, Artificial Sequence
  primer
tgttcgtagc

SEQ ID NO: 431 - DNA, 10 bp, Artificial Sequence
  primer
tgaggacgtg

SEQ ID NO: 432 - DNA, 10 bp, Artificial Sequence
  primer
gtgctgtgta

SEQ ID NO: 433 - DNA, 10 bp, Artificial Sequence
  primer
gagggtacgc

SEQ ID NO: 434 - DNA, 10 bp, Artificial Sequence
  primer
ccgtgattgt

SEQ ID NO: 435 - DNA, 10 bp, Artificial Sequence
  primer
aaaatcgcct

SEQ ID NO: 436 - DNA, 10 bp, Artificial Sequence
  primer
cgatcgcagt

SEQ ID NO: 437 - DNA, 10 bp, Artificial Sequence
  primer
acgcaataag

SEQ ID NO: 438 - DNA, 10 bp, Artificial Sequence
  primer
aaggtgcatc

SEQ ID NO: 439 - DNA, 10 bp, Artificial Sequence
  primer
cgcgtagata

SEQ ID NO: 440 - DNA, 10 bp, Artificial Sequence
  primer
cgagcagtgc

SEQ ID NO: 441 - DNA, 10 bp, Artificial Sequence
  primer
atacgtgacg

SEQ ID NO: 442 - DNA, 10 bp, Artificial Sequence
  primer
agattgcgcg

SEQ ID NO: 443 - DNA, 10 bp, Artificial Sequence
  primer
acgtgatgcc

SEQ ID NO: 444 - DNA, 10 bp, Artificial Sequence
  primer
gtacgcatcg

SEQ ID NO: 445 - DNA, 10 bp, Artificial Sequence
  primer
tcccgactta

SEQ ID NO: 446 - DNA, 10 bp, Artificial Sequence
  primer
gtttttacac

SEQ ID NO: 447 - DNA, 10 bp, Artificial Sequence
  primer
cctgagcgtg

SEQ ID NO: 448 - DNA, 10 bp, Artificial Sequence
  primer
cggcattgta

SEQ ID NO: 449 - DNA, 10 bp, Artificial Sequence
  primer
tagagtgcgt

SEQ ID NO: 450 - DNA, 10 bp, Artificial Sequence
  primer
atggccagac

SEQ ID NO: 451 - DNA, 10 bp, Artificial Sequence
  primer
cttagcatgc

SEQ ID NO: 452 - DNA, 10 bp, Artificial Sequence
  primer
acaacacctg

SEQ ID NO: 453 - DNA, 10 bp, Artificial Sequence
  primer
agtgactatc

SEQ ID NO: 454 - DNA, 10 bp, Artificial Sequence
  primer
catgctacac

SEQ ID NO: 455 - DNA, 10 bp, Artificial Sequence
  primer
aaagcgggcg

SEQ ID NO: 456 - DNA, 10 bp, Artificial Sequence
  primer
agatcgccgt

SEQ ID NO: 457 - DNA, 10 bp, Artificial Sequence
  primer
cgtagatatt

SEQ ID NO: 458 - DNA, 10 bp, Artificial Sequence
  primer
aatggcagac

SEQ ID NO: 459 - DNA, 10 bp, Artificial Sequence
  primer
gtataacgtg

SEQ ID NO: 460 - DNA, 10 bp, Artificial Sequence
  primer
atgtgcgtca

SEQ ID NO: 461 - DNA, 10 bp, Artificial Sequence
  primer
cctgccaact

SEQ ID NO: 462 - DNA, 10 bp, Artificial Sequence
  primer
tttataactc

SEQ ID NO: 463 - DNA, 10 bp, Artificial Sequence
  primer
acggttacgc

SEQ ID NO: 464 - DNA, 10 bp, Artificial Sequence
  primer
tagcctcttg

SEQ ID NO: 465 - DNA, 10 bp, Artificial Sequence
  primer
tcgcgaagtt

SEQ ID NO: 466 - DNA, 10 bp, Artificial Sequence
  primer
gtctacaacc

SEQ ID NO: 467 - DNA, 10 bp, Artificial Sequence
  primer
gtctactgcg

SEQ ID NO: 468 - DNA, 10 bp, Artificial Sequence
  primer
gttgcgtctc

SEQ ID NO: 469 - DNA, 10 bp, Artificial Sequence
  primer
gggccgctaa

SEQ ID NO: 470 - DNA, 10 bp, Artificial Sequence
  primer
gtacgtcgga

SEQ ID NO: 471 - DNA, 10 bp, Artificial Sequence
  primer
agcgagagac

SEQ ID NO: 472 - DNA, 10 bp, Artificial Sequence
  primer
tggctacggt

SEQ ID NO: 473 - DNA, 10 bp, Artificial Sequence
  primer
aggcatcacg

SEQ ID NO: 474 - DNA, 10 bp, Artificial Sequence
  primer
tagctcctcg

SEQ ID NO: 475 - DNA, 10 bp, Artificial Sequence
  primer
ggctagtcag

SEQ ID NO: 476 - DNA, 10 bp, Artificial Sequence
  primer
ctcactttat

SEQ ID NO: 477 - DNA, 10 bp, Artificial Sequence
  primer
acggccacgt

SEQ ID NO: 478 - DNA, 10 bp, Artificial Sequence
  primer
agcgtatatc

SEQ ID NO: 479 - DNA, 10 bp, Artificial Sequence
  primer
gacacgtcta

SEQ ID NO: 480 - DNA, 10 bp, Artificial Sequence
  primer
gccagcgtac

SEQ ID NO: 481 - DNA, 10 bp, Artificial Sequence
  primer
aacattagcg

SEQ ID NO: 482 - DNA, 10 bp, Artificial Sequence
  primer
agtgtgctat

SEQ ID NO: 483 - DNA, 10 bp, Artificial Sequence
  primer
cacgagcgtt

SEQ ID NO: 484 - DNA, 10 bp, Artificial Sequence
  primer
gtaacgccta

SEQ ID NO: 485 - DNA, 10 bp, Artificial Sequence
  primer
cacatagtac

SEQ ID NO: 486 - DNA, 10 bp, Artificial Sequence
  primer
cgcgatatcg

SEQ ID NO: 487 - DNA, 10 bp, Artificial Sequence
  primer
cgttctgtgc

SEQ ID NO: 488 - DNA, 10 bp, Artificial Sequence
  primer
ctgatcgcat

SEQ ID NO: 489 - DNA, 10 bp, Artificial Sequence
  primer
tggcgtgaga

SEQ ID NO: 490 - DNA, 10 bp, Artificial Sequence
  primer
ttgccaggct

SEQ ID NO: 491 - DNA, 10 bp, Artificial Sequence
  primer
gttatacaca

SEQ ID NO: 492 - DNA, 10 bp, Artificial Sequence
  primer
agtgccaact

SEQ ID NO: 493 - DNA, 10 bp, Artificial Sequence
  primer
tcacgtagca

SEQ ID NO: 494 - DNA, 10 bp, Artificial Sequence
  primer
taattcagcg

SEQ ID NO: 495 - DNA, 10 bp, Artificial Sequence
  primer
aagtatcgtc

SEQ ID NO: 496 - DNA, 10 bp, Artificial Sequence
  primer
cacagttact

SEQ ID NO: 497 - DNA, 10 bp, Artificial Sequence
  primer
ccttaccgtg

SEQ ID NO: 498 - DNA, 10 bp, Artificial Sequence
  primer
acggtgtcgt

SEQ ID NO: 499 - DNA, 10 bp, Artificial Sequence
  primer
cgcgtaagac

SEQ ID NO: 500 - DNA, 10 bp, Artificial Sequence
  primer
ttcgcaccag

SEQ ID NO: 501 - DNA, 10 bp, Artificial Sequence
  primer
cacgaacaga

SEQ ID NO: 502 - DNA, 10 bp, Artificial Sequence
  primer
gttggacatt

SEQ ID NO: 503 - DNA, 10 bp, Artificial Sequence
  primer
ggtgcttaag

SEQ ID NO: 504 - DNA, 10 bp, Artificial Sequence
  primer
tcggtctcgt

SEQ ID NO: 505 - DNA, 10 bp, Artificial Sequence
  primer
tctagtacgc

SEQ ID NO: 506 - DNA, 10 bp, Artificial Sequence
  primer
ttaggccgag

SEQ ID NO: 507 - DNA, 10 bp, Artificial Sequence
  primer
cgtcaagagc

SEQ ID NO: 508 - DNA, 10 bp, Artificial Sequence
  primer
acatgtctac

SEQ ID NO: 509 - DNA, 10 bp, Artificial Sequence
  primer
atcgttacgt

SEQ ID NO: 510 - DNA, 10 bp, Artificial Sequence
  primer
acggatcgtt

SEQ ID NO: 511 - DNA, 10 bp, Artificial Sequence
  primer
aatcttggcg

SEQ ID NO: 512 - DNA, 10 bp, Artificial Sequence
  primer
agtatctggt

SEQ ID NO: 513 - DNA, 10 bp, Artificial Sequence
  primer
caaccgacgt

SEQ ID NO: 514 - DNA, 10 bp, Artificial Sequence
  primer
tggtaacgcg

SEQ ID NO: 515 - DNA, 10 bp, Artificial Sequence
  primer
gtgcagacat

SEQ ID NO: 516 - DNA, 10 bp, Artificial Sequence
  primer
gtctagttgc

SEQ ID NO: 517 - DNA, 10 bp, Artificial Sequence
  primer
caattcgacg

SEQ ID NO: 518 - DNA, 10 bp, Artificial Sequence
  primer
cttagcacct

SEQ ID NO: 519 - DNA, 10 bp, Artificial Sequence
  primer
taatgtcgca

SEQ ID NO: 520 - DNA, 10 bp, Artificial Sequence
  primer
caatcggtac

SEQ ID NO: 521 - DNA, 10 bp, Artificial Sequence
  primer
agcacgcatt

SEQ ID NO: 522 - DNA, 10 bp, Artificial Sequence
  primer
aggtcctcgt

SEQ ID NO: 523 - DNA, 10 bp, Artificial Sequence
  primer
ttgtgcctgc

SEQ ID NO: 524 - DNA, 10 bp, Artificial Sequence
  primer
accgcctgta

SEQ ID NO: 525 - DNA, 10 bp, Artificial Sequence
  primer
gtacgtcagg

SEQ ID NO: 526 - DNA, 10 bp, Artificial Sequence
  primer
gcacacaact

SEQ ID NO: 527 - DNA, 10 bp, Artificial Sequence
  primer
tgagcactta

SEQ ID NO: 528 - DNA, 10 bp, Artificial Sequence
  primer
gtgccgcata

SEQ ID NO: 529 - DNA, 10 bp, Artificial Sequence
  primer
atgttttcgc

SEQ ID NO: 530 - DNA, 10 bp, Artificial Sequence
  primer
acacttaggt

SEQ ID NO: 531 - DNA, 10 bp, Artificial Sequence
  primer
cgtgccgtga

SEQ ID NO: 532 - DNA, 10 bp, Artificial Sequence
  primer
ttactaatca

SEQ ID NO: 533 - DNA, 10 bp, Artificial Sequence
  primer
gtggcaggta

SEQ ID NO: 534 - DNA, 10 bp, Artificial Sequence
  primer
gcgcgatatg

SEQ ID NO: 535 - DNA, 10 bp, Artificial Sequence
  primer
gaacgacgtt

SEQ ID NO: 536 - DNA, 10 bp, Artificial Sequence
  primer
atcaggagtg

SEQ ID NO: 537 - DNA, 10 bp, Artificial Sequence
  primer
gccagtaagt

SEQ ID NO: 538 - DNA, 10 bp, Artificial Sequence
  primer
gcaagaagca

SEQ ID NO: 539 - DNA, 10 bp, Artificial Sequence
  primer
aactccgcca

SEQ ID NO: 540 - DNA, 10 bp, Artificial Sequence
  primer
acttgagcct

SEQ ID NO: 541 - DNA, 10 bp, Artificial Sequence
  primer
cgtgatcgtg

SEQ ID NO: 542 - DNA, 10 bp, Artificial Sequence
  primer
aattagcgaa

SEQ ID NO: 543 - DNA, 10 bp, Artificial Sequence
  primer
acttccttag

SEQ ID NO: 544 - DNA, 10 bp, Artificial Sequence
  primer
tgtgctgata

SEQ ID NO: 545 - DNA, 10 bp, Artificial Sequence
  primer
aggcggctga

SEQ ID NO: 546 - DNA, 10 bp, Artificial Sequence
  primer
cgtttagagc

SEQ ID NO: 547 - DNA, 10 bp, Artificial Sequence
  primer
acgcgtctaa

SEQ ID NO: 548 - DNA, 10 bp, Artificial Sequence
  primer
gcgaatgtac

SEQ ID NO: 549 - DNA, 10 bp, Artificial Sequence
  primer
cgtgatccaa

SEQ ID NO: 550 - DNA, 10 bp, Artificial Sequence
  primer
caaccagatg

SEQ ID NO: 551 - DNA, 10 bp, Artificial Sequence
  primer
accattaacc

SEQ ID NO: 552 - DNA, 10 bp, Artificial Sequence
  primer
cgattcacgt

SEQ ID NO: 553 - DNA, 10 bp, Artificial Sequence
  primer
ctagaacctg

SEQ ID NO: 554 - DNA, 10 bp, Artificial Sequence
  primer
cctaacgaca

SEQ ID NO: 555 - DNA, 10 bp, Artificial Sequence
  primer
gacgtgcatg

SEQ ID NO: 556 - DNA, 10 bp, Artificial Sequence
  primer
atgtaacctt

SEQ ID NO: 557 - DNA, 10 bp, Artificial Sequence
  primer
gatacagtcg

SEQ ID NO: 558 - DNA, 10 bp, Artificial Sequence
  primer
cgtatgtctc

SEQ ID NO: 559 - DNA, 10 bp, Artificial Sequence
  primer
agattatcga

SEQ ID NO: 560 - DNA, 10 bp, Artificial Sequence
  primer
atactggtaa

SEQ ID NO: 561 - DNA, 10 bp, Artificial Sequence
  primer
gttgagtagc

SEQ ID NO: 562 - DNA, 10 bp, Artificial Sequence
  primer
accattatca

SEQ ID NO: 563 - DNA, 10 bp, Artificial Sequence
  primer
cacacttcag

SEQ ID NO: 564 - DNA, 10 bp, Artificial Sequence
  primer
gactagcggt

SEQ ID NO: 565 - DNA, 10 bp, Artificial Sequence
  primer
aattgtcgag

SEQ ID NO: 566 - DNA, 10 bp, Artificial Sequence
  primer
ctaaggacgt

SEQ ID NO: 567 - DNA, 10 bp, Artificial Sequence
  primer
attacgatga

SEQ ID NO: 568 - DNA, 10 bp, Artificial Sequence
  primer
attgaagact

SEQ ID NO: 569 - DNA, 10 bp, Artificial Sequence
  primer
gcttgtacgt

SEQ ID NO: 570 - DNA, 10 bp, Artificial Sequence
  primer
cctacgtcac

SEQ ID NO: 571 - DNA, 10 bp, Artificial Sequence
  primer
cacaacttag

SEQ ID NO: 572 - DNA, 10 bp, Artificial Sequence
  primer
gcggttcatc

SEQ ID NO: 573 - DNA, 10 bp, Artificial Sequence
  primer
gtactcatct

SEQ ID NO: 574 - DNA, 10 bp, Artificial Sequence
  primer
gtgcatcagt

SEQ ID NO: 575 - DNA, 10 bp, Artificial Sequence
  primer
tcacatccta

SEQ ID NO: 576 - DNA, 10 bp, Artificial Sequence
  primer
cacgcgctat

SEQ ID NO: 577 - DNA, 8 bp, Artificial Sequence
  primer
ctatcttg

SEQ ID NO: 578 - DNA, 8 bp, Artificial Sequence
  primer
aagtgcgt

SEQ ID NO: 579 - DNA, 8 bp, Artificial Sequence
  primer
acatgcga

SEQ ID NO: 580 - DNA, 8 bp, Artificial Sequence
  primer
accaatgg

SEQ ID NO: 581 - DNA, 8 bp, Artificial Sequence
  primer
tgcgttga

SEQ ID NO: 582 - DNA, 8 bp, Artificial Sequence
  primer
gacatgtc

SEQ ID NO: 583 - DNA, 8 bp, Artificial Sequence
  primer
ttgtgcgt

SEQ ID NO: 584 - DNA, 8 bp, Artificial Sequence
  primer
acatcgca

SEQ ID NO: 585 - DNA, 8 bp, Artificial Sequence
  primer
gaagacga

SEQ ID NO: 586 - DNA, 8 bp, Artificial Sequence
  primer
tcgataga

SEQ ID NO: 587 - DNA, 8 bp, Artificial Sequence
  primer
tcttgcaa

SEQ ID NO: 588 - DNA, 8 bp, Artificial Sequence
  primer
agcaagtt

SEQ ID NO: 589 - DNA, 8 bp, Artificial Sequence
  primer
ttcatgga

SEQ ID NO: 590 - DNA, 8 bp, Artificial Sequence
  primer
tcaattcg

SEQ ID NO: 591 - DNA, 8 bp, Artificial Sequence
  primer
cggtatgt

SEQ ID NO: 592 - DNA, 8 bp, Artificial Sequence
  primer
accactac

SEQ ID NO: 593 - DNA, 8 bp, Artificial Sequence
  primer
tcgcttat

SEQ ID NO: 594 - DNA, 8 bp, Artificial Sequence
  primer
tctcgact

SEQ ID NO: 595 - DNA, 8 bp, Artificial Sequence
  primer
gaatcggt

SEQ ID NO: 596 - DNA, 8 bp, Artificial Sequence
  primer
gttacaag

SEQ ID NO: 597 - DNA, 8 bp, Artificial Sequence
  primer
ctgtgtag

SEQ ID NO: 598 - DNA, 8 bp, Artificial Sequence
  primer
tggtagaa

SEQ ID NO: 599 - DNA, 8 bp, Artificial Sequence
  primer
atactgcg

SEQ ID NO: 600 - DNA, 8 bp, Artificial Sequence
  primer
aactcgtc

SEQ ID NO: 601 - DNA, 8 bp, Artificial Sequence
  primer
atatgtgc

SEQ ID NO: 602 - DNA, 8 bp, Artificial Sequence
  primer
aagttgcg

SEQ ID NO: 603 - DNA, 8 bp, Artificial Sequence
  primer
gatcatgt

SEQ ID NO: 604 - DNA, 8 bp, Artificial Sequence
  primer
ttgttgct

SEQ ID NO: 605 - DNA, 8 bp, Artificial Sequence
  primer
cctcttag

SEQ ID NO: 606 - DNA, 8 bp, Artificial Sequence
  primer
tcacagct

SEQ ID NO: 607 - DNA, 8 bp, Artificial Sequence
  primer
agattgac

SEQ ID NO: 608 - DNA, 8 bp, Artificial Sequence
  primer
agcctgat

SEQ ID NO: 609 - DNA, 8 bp, Artificial Sequence
  primer
cgtcaagt

SEQ ID NO: 610 - DNA, 8 bp, Artificial Sequence
  primer
aagtagac

SEQ ID NO: 611 - DNA, 8 bp, Artificial Sequence
  primer
tcagacaa

SEQ ID NO: 612 - DNA, 8 bp, Artificial Sequence
  primer
tccttgac

SEQ ID NO: 613 - DNA, 8 bp, Artificial Sequence
  primer
gtagctgt

SEQ ID NO: 614 - DNA, 8 bp, Artificial Sequence
  primer
cgtcgtaa

SEQ ID NO: 615 - DNA, 8 bp, Artificial Sequence
  primer
ccaatgga

SEQ ID NO: 616 - DNA, 8 bp, Artificial Sequence
  primer
ttgagaga

SEQ ID NO: 617 - DNA, 8 bp, Artificial Sequence
  primer
acaacacc

SEQ ID NO: 618 - DNA, 8 bp, Artificial Sequence
  primer
tctagtac

SEQ ID NO: 619 - DNA, 8 bp, Artificial Sequence
  primer
gaggaagt

SEQ ID NO: 620 - DNA, 8 bp, Artificial Sequence
  primer
gcgtattg

SEQ ID NO: 621 - DNA, 8 bp, Artificial Sequence
  primer
aagtagct

SEQ ID NO: 622 - DNA, 8 bp, Artificial Sequence
  primer
tgaacctt

SEQ ID NO: 623 - DNA, 8 bp, Artificial Sequence
  primer
tgtgttac

SEQ ID NO: 624 - DNA, 8 bp, Artificial Sequence
  primer
taacctga

SEQ ID NO: 625 - DNA, 8 bp, Artificial Sequence
  primer
gctattcc

SEQ ID NO: 626 - DNA, 8 bp, Artificial Sequence
  primer
gttagatg

SEQ ID NO: 627 - DNA, 8 bp, Artificial Sequence
  primer
caggataa

SEQ ID NO: 628 - DNA, 8 bp, Artificial Sequence
  primer
accgtagt

SEQ ID NO: 629 - DNA, 8 bp, Artificial Sequence
  primer
ccgtgtat

SEQ ID NO: 630 - DNA, 8 bp, Artificial Sequence
  primer
tccactct

SEQ ID NO: 631 - DNA, 8 bp, Artificial Sequence
  primer
tagctcat

SEQ ID NO: 632 - DNA, 8 bp, Artificial Sequence
  primer
cgctaata

SEQ ID NO: 633 - DNA, 8 bp, Artificial Sequence
  primer
tacctctg

SEQ ID NO: 634 - DNA, 8 bp, Artificial Sequence
  primer
tgcactac

SEQ ID NO: 635 - DNA, 8 bp, Artificial Sequence
  primer
cttggaag

SEQ ID NO: 636 - DNA, 8 bp, Artificial Sequence
  primer
aatgcacg

SEQ ID NO: 637 - DNA, 8 bp, Artificial Sequence
  primer
cactgtta

SEQ ID NO: 638 - DNA, 8 bp, Artificial Sequence
  primer
tcgactag

SEQ ID NO: 639 - DNA, 8 bp, Artificial Sequence
  primer
ctaggtta

SEQ ID NO: 640 - DNA, 8 bp, Artificial Sequence
  primer
gcagatgt

SEQ ID NO: 641 - DNA, 8 bp, Artificial Sequence
  primer
agttcaga

SEQ ID NO: 642 - DNA, 8 bp, Artificial Sequence
  primer
ctccatca

SEQ ID NO: 643 - DNA, 8 bp, Artificial Sequence
  primer
tggttacg

SEQ ID NO: 644 - DNA, 8 bp, Artificial Sequence
  primer
acgtagca

SEQ ID NO: 645 - DNA, 8 bp, Artificial Sequence
  primer
ctcttcca

SEQ ID NO: 646 - DNA, 8 bp, Artificial Sequence
  primer
cgtcagat

SEQ ID NO: 647 - DNA, 8 bp, Artificial Sequence
  primer
tggatcat

SEQ ID NO: 648 - DNA, 8 bp, Artificial Sequence
  primer
atatcgac

SEQ ID NO: 649 - DNA, 8 bp, Artificial Sequence
  primer
ttgtggag

SEQ ID NO: 650 - DNA, 8 bp, Artificial Sequence
  primer
ttagagca

SEQ ID NO: 651 - DNA, 8 bp, Artificial Sequence
  primer
taactacc

SEQ ID NO: 652 - DNA, 8 bp, Artificial Sequence
  primer
ctatgagg

SEQ ID NO: 653 - DNA, 8 bp, Artificial Sequence
  primer
cttctcac

SEQ ID NO: 654 - DNA, 8 bp, Artificial Sequence
  primer
cgttctct

SEQ ID NO: 655 - DNA, 8 bp, Artificial Sequence
  primer
gtcactat

SEQ ID NO: 656 - DNA, 8 bp, Artificial Sequence
  primer
tcgttagc

SEQ ID NO: 657 - DNA, 8 bp, Artificial Sequence
  primer
atcgtgta

SEQ ID NO: 658 - DNA, 8 bp, Artificial Sequence
  primer
gagagcaa

SEQ ID NO: 659 - DNA, 8 bp, Artificial Sequence
  primer
agacgcaa

SEQ ID NO: 660 - DNA, 8 bp, Artificial Sequence
  primer
tccagtta

SEQ ID NO: 661 - DNA, 8 bp, Artificial Sequence
  primer
aatgccac

SEQ ID NO: 662 - DNA, 8 bp, Artificial Sequence
  primer
atcacgtg

SEQ ID NO: 663 - DNA, 8 bp, Artificial Sequence
  primer
actgtgca

SEQ ID NO: 664 - DNA, 8 bp, Artificial Sequence
  primer
tcactgca

SEQ ID NO: 665 - DNA, 8 bp, Artificial Sequence
  primer
gcatccaa

SEQ ID NO: 666 - DNA, 8 bp, Artificial Sequence
  primer
agcactat

SEQ ID NO: 667 - DNA, 8 bp, Artificial Sequence
  primer
cgaaggat

SEQ ID NO: 668 - DNA, 8 bp, Artificial Sequence
  primer
ccttgtgt

SEQ ID NO: 669 - DNA, 8 bp, Artificial Sequence
  primer
tgcggata

SEQ ID NO: 670 - DNA, 8 bp, Artificial Sequence
  primer
aggaatgg

SEQ ID NO: 671 - DNA, 8 bp, Artificial Sequence
  primer
atcgtaac

SEQ ID NO: 672 - DNA, 8 bp, Artificial Sequence
  primer
gaatgtct

SEQ ID NO: 673 - DNA, 9 bp, Artificial Sequence
  primer
ttgctacat

SEQ ID NO: 674 - DNA, 9 bp, Artificial Sequence
  primer
taacgtatg

SEQ ID NO: 675 - DNA, 9 bp, Artificial Sequence
  primer
cagtatgta

SEQ ID NO: 676 - DNA, 9 bp, Artificial Sequence
  primer
tcaataacg

SEQ ID NO: 677 - DNA, 9 bp, Artificial Sequence
  primer
cacacttat

SEQ ID NO: 678 - DNA, 9 bp, Artificial Sequence
  primer
gactgtaat

SEQ ID NO: 679 - DNA, 9 bp, Artificial Sequence
  primer
tatacactg

SEQ ID NO: 680 - DNA, 9 bp, Artificial Sequence
  primer
actgcatta

SEQ ID NO: 681 - DNA, 9 bp, Artificial Sequence
  primer
acattaagc

SEQ ID NO: 682 - DNA, 9 bp, Artificial Sequence
  primer
catattacg

SEQ ID NO: 683 - DNA, 9 bp, Artificial Sequence
  primer
atatctacg

SEQ ID NO: 684 - DNA, 9 bp, Artificial Sequence
  primer
agtaactgt

SEQ ID NO: 685 - DNA, 9 bp, Artificial Sequence
  primer
atgacgtta

SEQ ID NO: 686 - DNA, 9 bp, Artificial Sequence
  primer
attatgcga

SEQ ID NO: 687 - DNA, 9 bp, Artificial Sequence
  primer
agtatacac

SEQ ID NO: 688 - DNA, 9 bp, Artificial Sequence
  primer
ttagcgtta

SEQ ID NO: 689 - DNA, 9 bp, Artificial Sequence
  primer
tatgacact

SEQ ID NO: 690 - DNA, 9 bp, Artificial Sequence
  primer
attaacgct

SEQ ID NO: 691 - DNA, 9 bp, Artificial Sequence
  primer
taggacaat

SEQ ID NO: 692 - DNA, 9 bp, Artificial Sequence
  primer
aagacgtta

SEQ ID NO: 693 - DNA, 9 bp, Artificial Sequence
  primer
tataagcgt

SEQ ID NO: 694 - DNA, 9 bp, Artificial Sequence
  primer
atacctggc

SEQ ID NO: 695 - DNA, 9 bp, Artificial Sequence
  primer
ctcgagatc

SEQ ID NO: 696 - DNA, 9 bp, Artificial Sequence
  primer
atggtgagg

SEQ ID NO: 697 - DNA, 9 bp, Artificial Sequence
  primer
atgtcgacg

SEQ ID NO: 698 - DNA, 9 bp, Artificial Sequence
  primer
gacgtctga

SEQ ID NO: 699 - DNA, 9 bp, Artificial Sequence
  primer
tacactgcg

SEQ ID NO: 700 - DNA, 9 bp, Artificial Sequence
  primer
atcgtcagg

SEQ ID NO: 701 - DNA, 9 bp, Artificial Sequence
  primer
tgcacgtac

SEQ ID NO: 702 - DNA, 9 bp, Artificial Sequence
  primer
gtcgtgcat

SEQ ID NO: 703 - DNA, 9 bp, Artificial Sequence
  primer
gagtgttac

SEQ ID NO: 704 - DNA, 9 bp, Artificial Sequence
  primer
agactgtac

SEQ ID NO: 705 - DNA, 9 bp, Artificial Sequence
  primer
tgcgactta

SEQ ID NO: 706 - DNA, 9 bp, Artificial Sequence
  primer
tgtccgtaa

SEQ ID NO: 707 - DNA, 9 bp, Artificial Sequence
  primer
gtaatcgag

SEQ ID NO: 708 - DNA, 9 bp, Artificial Sequence
  primer
gtaccttag

SEQ ID NO: 709 - DNA, 9 bp, Artificial Sequence
  primer
atcacgtgt

SEQ ID NO: 710 - DNA, 9 bp, Artificial Sequence
  primer
acttagcgt

SEQ ID NO: 711 - DNA, 9 bp, Artificial Sequence
  primer
gtaatcgtg

SEQ ID NO: 712 - DNA, 9 bp, Artificial Sequence
  primer
atgccgtta

SEQ ID NO: 713 - DNA, 9 bp, Artificial Sequence
  primer
ataacgtgc

SEQ ID NO: 714 - DNA, 9 bp, Artificial Sequence
  primer
ctacgttgt

SEQ ID NO: 715 - DNA, 9 bp, Artificial Sequence
  primer
tatgacgca

SEQ ID NO: 716 - DNA, 9 bp, Artificial Sequence
  primer
ccgataaca

SEQ ID NO: 717 - DNA, 9 bp, Artificial Sequence
  primer
atgcgcata

SEQ ID NO: 718 - DNA, 9 bp, Artificial Sequence
  primer
gataagcgt

SEQ ID NO: 719 - DNA, 9 bp, Artificial Sequence
  primer
atatctgcg

SEQ ID NO: 720 - DNA, 9 bp, Artificial Sequence
  primer
acttagacg

SEQ ID NO: 721 - DNA, 9 bp, Artificial Sequence
  primer
atcaccgta

SEQ ID NO: 722 - DNA, 9 bp, Artificial Sequence
  primer
taagacacg

SEQ ID NO: 723 - DNA, 9 bp, Artificial Sequence
  primer
aatgccgta

SEQ ID NO: 724 - DNA, 9 bp, Artificial Sequence
  primer
aatcacgtg

SEQ ID NO: 725 - DNA, 9 bp, Artificial Sequence
  primer
tcgttagtc

SEQ ID NO: 726 - DNA, 9 bp, Artificial Sequence
  primer
catcatgtc

SEQ ID NO: 727 - DNA, 9 bp, Artificial Sequence
  primer
taagacggt

SEQ ID NO: 728 - DNA, 9 bp, Artificial Sequence
  primer
tgcatagtg

SEQ ID NO: 729 - DNA, 9 bp, Artificial Sequence
  primer
gagcgttat

SEQ ID NO: 730 - DNA, 9 bp, Artificial Sequence
  primer
tgccttaca

SEQ ID NO: 731 - DNA, 9 bp, Artificial Sequence
  primer
ttcgcgtta

SEQ ID NO: 732 - DNA, 9 bp, Artificial Sequence
  primer
gtgttaacg

SEQ ID NO: 733 - DNA, 9 bp, Artificial Sequence
  primer
gacactgaa

SEQ ID NO: 734 - DNA, 9 bp, Artificial Sequence
  primer
ctgttatcg

SEQ ID NO: 735 - DNA, 9 bp, Artificial Sequence
  primer
ggtcgttat

SEQ ID NO: 736 - DNA, 9 bp, Artificial Sequence
  primer
cgagagtat

SEQ ID NO: 737 - DNA, 9 bp, Artificial Sequence
  primer
atacagtcc

SEQ ID NO: 738 - DNA, 9 bp, Artificial Sequence
  primer
aattcacgc

SEQ ID NO: 739 - DNA, 9 bp, Artificial Sequence
  primer
tatgtgcac

SEQ ID NO: 740 - DNA, 9 bp, Artificial Sequence
  primer
gatgacgta

SEQ ID NO: 741 - DNA, 9 bp, Artificial Sequence
  primer
gatgcgata

SEQ ID NO: 742 - DNA, 9 bp, Artificial Sequence
  primer
gagcgatta

SEQ ID NO: 743 - DNA, 9 bp, Artificial Sequence
  primer
tgtcacaga

SEQ ID NO: 744 - DNA, 9 bp, Artificial Sequence
  primer
tactaaccg

SEQ ID NO: 745 - DNA, 9 bp, Artificial Sequence
  primer
cataacgag

SEQ ID NO: 746 - DNA, 9 bp, Artificial Sequence
  primer
cgtatacct

SEQ ID NO: 747 - DNA, 9 bp, Artificial Sequence
  primer
tatcacgtg

SEQ ID NO: 748 - DNA, 9 bp, Artificial Sequence
  primer
gaacgttac

SEQ ID NO: 749 - DNA, 9 bp, Artificial Sequence
  primer
gtcgtatac

SEQ ID NO: 750 - DNA, 9 bp, Artificial Sequence
  primer
atgtcgaca

SEQ ID NO: 751 - DNA, 9 bp, Artificial Sequence
  primer
atacagcac

SEQ ID NO: 752 - DNA, 9 bp, Artificial Sequence
  primer
tacttacgc

SEQ ID NO: 753 - DNA, 9 bp, Artificial Sequence
  primer
aactacggt

SEQ ID NO: 754 - DNA, 9 bp, Artificial Sequence
  primer
tagaacggt

SEQ ID NO: 755 - DNA, 9 bp, Artificial Sequence
  primer
gaatgtcac

SEQ ID NO: 756 - DNA, 9 bp, Artificial Sequence
  primer
tgtacgtct

SEQ ID NO: 757 - DNA, 9 bp, Artificial Sequence
  primer
aacattgcg

SEQ ID NO: 758 - DNA, 9 bp, Artificial Sequence
  primer
ttgaacgct

SEQ ID NO: 759 - DNA, 9 bp, Artificial Sequence
  primer
aatcaggac

SEQ ID NO: 760 - DNA, 9 bp, Artificial Sequence
  primer
attcgcaca

SEQ ID NO: 761 - DNA, 9 bp, Artificial Sequence
  primer
ccatgtact

SEQ ID NO: 762 - DNA, 9 bp, Artificial Sequence
  primer
tgtcctgtt

SEQ ID NO: 763 - DNA, 9 bp, Artificial Sequence
  primer
taattgcgc

SEQ ID NO: 764 - DNA, 9 bp, Artificial Sequence
  primer
gatagtgtg

SEQ ID NO: 765 - DNA, 9 bp, Artificial Sequence
  primer
atagacgca

SEQ ID NO: 766 - DNA, 9 bp, Artificial Sequence
  primer
tgtaccgtt

SEQ ID NO: 767 - DNA, 9 bp, Artificial Sequence
  primer
attgtcgca

SEQ ID NO: 768 - DNA, 9 bp, Artificial Sequence
  primer
gtcacgtaa

SEQ ID NO: 769 - DNA, 11 bp, Artificial Sequence
  primer
ttacactatgc

SEQ ID NO: 770 - DNA, 11 bp, Artificial Sequence
  primer
gcgatagtcgt

SEQ ID NO: 771 - DNA, 11 bp, Artificial Sequence
  primer
ctattcacagt

SEQ ID NO: 772 - DNA, 11 bp, Artificial Sequence
  primer
agagtcactgt

SEQ ID NO: 773 - DNA, 11 bp, Artificial Sequence
  primer
agagtcgaagc

SEQ ID NO: 774 - DNA, 11 bp, Artificial Sequence
  primer
ctgaatatgtg

SEQ ID NO: 775 - DNA, 11 bp, Artificial Sequence
  primer
actccacagga

SEQ ID NO: 776 - DNA, 11 bp, Artificial Sequence
  primer
atcctcgtaag

SEQ ID NO: 777 - DNA, 11 bp, Artificial Sequence
  primer
taccatcgcct

SEQ ID NO: 778 - DNA, 11 bp, Artificial Sequence
  primer
aacgcctataa

SEQ ID NO: 779 - DNA, 11 bp, Artificial Sequence
  primer
ctgtcgaactt

SEQ ID NO: 780 - DNA, 11 bp, Artificial Sequence
  primer
tcagatgtccg

SEQ ID NO: 781 - DNA, 11 bp, Artificial Sequence
  primer
ctgcttatcgt

SEQ ID NO: 782 - DNA, 11 bp, Artificial Sequence
  primer
acattcgcaca

SEQ ID NO: 783 - DNA, 11 bp, Artificial Sequence
  primer
ccttaatgcat

SEQ ID NO: 784 - DNA, 11 bp, Artificial Sequence
  primer
ggctagctact

SEQ ID NO: 785 - DNA, 11 bp, Artificial Sequence
  primer
ttccagttggc

SEQ ID NO: 786 - DNA, 11 bp, Artificial Sequence
  primer
gagtcacaagg

SEQ ID NO: 787 - DNA, 11 bp, Artificial Sequence
  primer
cagaaggttca

SEQ ID NO: 788 - DNA, 11 bp, Artificial Sequence
  primer
tcaacgtgcag

SEQ ID NO: 789 - DNA, 11 bp, Artificial Sequence
  primer
caagcttacta

SEQ ID NO: 790 - DNA, 11 bp, Artificial Sequence
  primer
agaactcgttg

SEQ ID NO: 791 - DNA, 11 bp, Artificial Sequence
  primer
ccgatacagag

SEQ ID NO: 792 - DNA, 11 bp, Artificial Sequence
  primer
gtacgctgatc

SEQ ID NO: 793 - DNA, 11 bp, Artificial Sequence
  primer
tcctcagtgaa

SEQ ID NO: 794 - DNA, 11 bp, Artificial Sequence
  primer
gagccaacatt

SEQ ID NO: 795 - DNA, 11 bp, Artificial Sequence
  primer
gagatcgatgg

SEQ ID NO: 796 - DNA, 11 bp, Artificial Sequence
  primer
atcgtcagctg

SEQ ID NO: 797 - DNA, 11 bp, Artificial Sequence
  primer
gaagcacacgt

SEQ ID NO: 798 - DNA, 11 bp, Artificial Sequence
  primer
atcacgcaacc

SEQ ID NO: 799 - DNA, 11 bp, Artificial Sequence
  primer
tcgaatagtcg

SEQ ID NO: 800 - DNA, 11 bp, Artificial Sequence
  primer
tattaccgtct

SEQ ID NO: 801 - DNA, 11 bp, Artificial Sequence
  primer
cagtcacgaca

SEQ ID NO: 802 - DNA, 11 bp, Artificial Sequence
  primer
ttactcgacgt

SEQ ID NO: 803 - DNA, 11 bp, Artificial Sequence
  primer
gcaatgttgaa

SEQ ID NO: 804 - DNA, 11 bp, Artificial Sequence
  primer
gacacgagcaa

SEQ ID NO: 805 - DNA, 11 bp, Artificial Sequence
  primer
cgagattacaa

SEQ ID NO: 806 - DNA, 11 bp, Artificial Sequence
  primer
taccgactaca

SEQ ID NO: 807 - DNA, 11 bp, Artificial Sequence
  primer
accgttgccat

SEQ ID NO: 808 - DNA, 11 bp, Artificial Sequence
  primer
atgtaatcgcc

SEQ ID NO: 809 - DNA, 11 bp, Artificial Sequence
  primer
aagcctgatgt

SEQ ID NO: 810 - DNA, 11 bp, Artificial Sequence
  primer
aagtaacgtgg

SEQ ID NO: 811 - DNA, 11 bp, Artificial Sequence
  primer
gtagaggttgg

SEQ ID NO: 812 - DNA, 11 bp, Artificial Sequence
  primer
ctcttgcctca

SEQ ID NO: 813 - DNA, 11 bp, Artificial Sequence
  primer
atcgtgaagtg

SEQ ID NO: 814 - DNA, 11 bp, Artificial Sequence
  primer
accagcactat

SEQ ID NO: 815 - DNA, 11 bp, Artificial Sequence
  primer
caccagaatgt

SEQ ID NO: 816 - DNA, 11 bp, Artificial Sequence
  primer
gagtgaacaac

SEQ ID NO: 817 - DNA, 11 bp, Artificial Sequence
  primer
taacgttacgc

SEQ ID NO: 818 - DNA, 11 bp, Artificial Sequence
  primer
cttggatcttg

SEQ ID NO: 819 - DNA, 11 bp, Artificial Sequence
  primer
gttccaacgtt

SEQ ID NO: 820 - DNA, 11 bp, Artificial Sequence
  primer
caaggaccgta

SEQ ID NO: 821 - DNA, 11 bp, Artificial Sequence
  primer
gacttcacgca

SEQ ID NO: 822 - DNA, 11 bp, Artificial Sequence
  primer
cacactactgg

SEQ ID NO: 823 - DNA, 11 bp, Artificial Sequence
  primer
tcagatgaatc

SEQ ID NO: 824 - DNA, 11 bp, Artificial Sequence
  primer
tatggatctgg

SEQ ID NO: 825 - DNA, 11 bp, Artificial Sequence
  primer
tcttaggtgtg

SEQ ID NO: 826 - DNA, 11 bp, Artificial Sequence
  primer
tgtcagcgtca

SEQ ID NO: 827 - DNA, 11 bp, Artificial Sequence
  primer
gtctaggacag

SEQ ID NO: 828 - DNA, 11 bp, Artificial Sequence
  primer
gcctcttcata

SEQ ID NO: 829 - DNA, 11 bp, Artificial Sequence
  primer
agaagtgttac

SEQ ID NO: 830 - DNA, 11 bp, Artificial Sequence
  primer
catgaggcttg

SEQ ID NO: 831 - DNA, 11 bp, Artificial Sequence
  primer
tggattgctca

SEQ ID NO: 832 - DNA, 11 bp, Artificial Sequence
  primer
atctacctaag

SEQ ID NO: 833 - DNA, 11 bp, Artificial Sequence
  primer
atgagcagtga

SEQ ID NO: 834 - DNA, 11 bp, Artificial Sequence
  primer
ccaggagatac

SEQ ID NO: 835 - DNA, 11 bp, Artificial Sequence
  primer
ccgttatactt

SEQ ID NO: 836 - DNA, 11 bp, Artificial Sequence
  primer
ctcagtacaag

SEQ ID NO: 837 - DNA, 11 bp, Artificial Sequence
  primer
ggtgatcgtag

SEQ ID NO: 838 - DNA, 11 bp, Artificial Sequence
  primer
cgaacgagaca

SEQ ID NO: 839 - DNA, 11 bp, Artificial Sequence
  primer
actacgagctt

SEQ ID NO: 840 - DNA, 11 bp, Artificial Sequence
  primer
ttgccacagca

SEQ ID NO: 841 - DNA, 11 bp, Artificial Sequence
  primer
gtcaactctac

SEQ ID NO: 842 - DNA, 11 bp, Artificial Sequence
  primer
tggactgtgtc

SEQ ID NO: 843 - DNA, 11 bp, Artificial Sequence
  primer
ggaatggactt

SEQ ID NO: 844 - DNA, 11 bp, Artificial Sequence
  primer
cgagaacataa

SEQ ID NO: 845 - DNA, 11 bp, Artificial Sequence
  primer
acctggtcagt

SEQ ID NO: 846 - DNA, 11 bp, Artificial Sequence
  primer
cgaacgacaca

SEQ ID NO: 847 - DNA, 11 bp, Artificial Sequence
  primer
agtctagccat

SEQ ID NO: 848 - DNA, 11 bp, Artificial Sequence
  primer
aggcctagatg

SEQ ID NO: 849 - DNA, 11 bp, Artificial Sequence
  primer
ggtgcgttagt

SEQ ID NO: 850 - DNA, 11 bp, Artificial Sequence
  primer
attgtgtccga

SEQ ID NO: 851 - DNA, 11 bp, Artificial Sequence
  primer
gcagacattaa

SEQ ID NO: 852 - DNA, 11 bp, Artificial Sequence
  primer
attggctcatg

SEQ ID NO: 853 - DNA, 11 bp, Artificial Sequence
  primer
gaggttacatg

SEQ ID NO: 854 - DNA, 11 bp, Artificial Sequence
  primer
cctataggacc

SEQ ID NO: 855 - DNA, 11 bp, Artificial Sequence
  primer
ttagacggtct

SEQ ID NO: 856 - DNA, 11 bp, Artificial Sequence
  primer
gattgacgcac

SEQ ID NO: 857 - DNA, 11 bp, Artificial Sequence
  primer
aagacacctcg

SEQ ID NO: 858 - DNA, 11 bp, Artificial Sequence
  primer
tcgaataatcg

SEQ ID NO: 859 - DNA, 11 bp, Artificial Sequence
  primer
tctatgtcgga

SEQ ID NO: 860 - DNA, 11 bp, Artificial Sequence
  primer
tcgcatgaacc

SEQ ID NO: 861 - DNA, 11 bp, Artificial Sequence
  primer
tgttatgtctc

SEQ ID NO: 862 - DNA, 11 bp, Artificial Sequence
  primer
tggatcctaca

SEQ ID NO: 863 - DNA, 11 bp, Artificial Sequence
  primer
atcgttcagcc

SEQ ID NO: 864 - DNA, 11 bp, Artificial Sequence
  primer
taccgcaagca

SEQ ID NO: 865 - DNA, 12 bp, Artificial Sequence
  primer
gctgttgaaccg

SEQ ID NO: 866 - DNA, 12 bp, Artificial Sequence
  primer
atactccgagat

SEQ ID NO: 867 - DNA, 12 bp, Artificial Sequence
  primer
cttaaggagcgc

SEQ ID NO: 868 - DNA, 12 bp, Artificial Sequence
  primer
tatactacaagc

SEQ ID NO: 869 - DNA, 12 bp, Artificial Sequence
  primer
tagtggtcgtca

SEQ ID NO: 870 - DNA, 12 bp, Artificial Sequence
  primer
gtgcttcaggag

SEQ ID NO: 871 - DNA, 12 bp, Artificial Sequence
  primer
gacgcatacctc

SEQ ID NO: 872 - DNA, 12 bp, Artificial Sequence
  primer
cctacctgtgga

SEQ ID NO: 873 - DNA, 12 bp, Artificial Sequence
  primer
gcggtcacatat

SEQ ID NO: 874 - DNA, 12 bp, Artificial Sequence
  primer
ctgcattcacga

SEQ ID NO: 875 - DNA, 12 bp, Artificial Sequence
  primer
tggatccttcat

SEQ ID NO: 876 - DNA, 12 bp, Artificial Sequence
  primer
ttgtgctggact

SEQ ID NO: 877 - DNA, 12 bp, Artificial Sequence
  primer
attgagagctat

SEQ ID NO: 878 - DNA, 12 bp, Artificial Sequence
  primer
tcgctaatgtag

SEQ ID NO: 879 - DNA, 12 bp, Artificial Sequence
  primer
ctactggcacaa

SEQ ID NO: 880 - DNA, 12 bp, Artificial Sequence
  primer
agagccagtcgt

SEQ ID NO: 881 - DNA, 12 bp, Artificial Sequence
  primer
aatactggctaa

SEQ ID NO: 882 - DNA, 12 bp, Artificial Sequence
  primer
ctgcatgcataa

SEQ ID NO: 883 - DNA, 12 bp, Artificial Sequence
  primer
ttgtcacaactc

SEQ ID NO: 884 - DNA, 12 bp, Artificial Sequence
  primer
tgctaactctcc

SEQ ID NO: 885 - DNA, 12 bp, Artificial Sequence
  primer
tctctagttcgg

SEQ ID NO: 886 - DNA, 12 bp, Artificial Sequence
  primer
ttacgtccgcaa

SEQ ID NO: 887 - DNA, 12 bp, Artificial Sequence
  primer
gtgttgctacca

SEQ ID NO: 888 - DNA, 12 bp, Artificial Sequence
  primer
cgcatgtatgcc

SEQ ID NO: 889 - DNA, 12 bp, Artificial Sequence
  primer
cctgttctgatt

SEQ ID NO: 890 - DNA, 12 bp, Artificial Sequence
  primer
taagatgcttga

SEQ ID NO: 891 - DNA, 12 bp, Artificial Sequence
  primer
atatatctcagc

SEQ ID NO: 892 - DNA, 12 bp, Artificial Sequence
  primer
ttcctcgtggtt

SEQ ID NO: 893 - DNA, 12 bp, Artificial Sequence
  primer
atgtcgatctag

SEQ ID NO: 894 - DNA, 12 bp, Artificial Sequence
  primer
catccactaatc

SEQ ID NO: 895 - DNA, 12 bp, Artificial Sequence
  primer
gcctctggtaac

SEQ ID NO: 896 - DNA, 12 bp, Artificial Sequence
  primer
agtcaagagatt

SEQ ID NO: 897 - DNA, 12 bp, Artificial Sequence
  primer
actgaggcgttc

SEQ ID NO: 898 - DNA, 12 bp, Artificial Sequence
  primer
taaggctgacat

SEQ ID NO: 899 - DNA, 12 bp, Artificial Sequence
  primer
agttcgcataca

SEQ ID NO: 900 - DNA, 12 bp, Artificial Sequence
  primer
gcagaattgcga

SEQ ID NO: 901 - DNA, 12 bp, Artificial Sequence
  primer
ggttatgaagaa

SEQ ID NO: 902 - DNA, 12 bp, Artificial Sequence
  primer
agaagtcgcctc

SEQ ID NO: 903 - DNA, 12 bp, Artificial Sequence
  primer
ttcgcgttattg

SEQ ID NO: 904 - DNA, 12 bp, Artificial Sequence
  primer
tacctggtcggt

SEQ ID NO: 905 - DNA, 12 bp, Artificial Sequence
  primer
ggttaccgagga

SEQ ID NO: 906 - DNA, 12 bp, Artificial Sequence
  primer
acacacttctag

SEQ ID NO: 907 - DNA, 12 bp, Artificial Sequence
  primer
ggaagtgattaa

SEQ ID NO: 908 - DNA, 12 bp, Artificial Sequence
  primer
tccatcagataa

SEQ ID NO: 909 - DNA, 12 bp, Artificial Sequence
  primer
tgtctgtatcat

SEQ ID NO: 910 - DNA, 12 bp, Artificial Sequence
  primer
aattggctatag

SEQ ID NO: 911 - DNA, 12 bp, Artificial Sequence
  primer
acgtcggaaggt

SEQ ID NO: 912 - DNA, 12 bp, Artificial Sequence
  primer
aggcatccgttg

SEQ ID NO: 913 - DNA, 12 bp, Artificial Sequence
  primer
accgtcgcttga

SEQ ID NO: 914 - DNA, 12 bp, Artificial Sequence
  primer
taccgtcaagtg

SEQ ID NO: 915 - DNA, 12 bp, Artificial Sequence
  primer
ctcgatatagtt

SEQ ID NO: 916 - DNA, 12 bp, Artificial Sequence
  primer
cgtcaacgtggt

SEQ ID NO: 917 - DNA, 12 bp, Artificial Sequence
  primer
tagtcaacgtag

SEQ ID NO: 918 - DNA, 12 bp, Artificial Sequence
  primer
tgagtaggtcag

SEQ ID NO: 919 - DNA, 12 bp, Artificial Sequence
  primer
cttggcatgtac

SEQ ID NO: 920 - DNA, 12 bp, Artificial Sequence
  primer
tgccgagacttc

SEQ ID NO: 921 - DNA, 12 bp, Artificial Sequence
  primer
ctaagacttaag

SEQ ID NO: 922 - DNA, 12 bp, Artificial Sequence
  primer
ttctcgtgtgcg

SEQ ID NO: 923 - DNA, 12 bp, Artificial Sequence
  primer
cacctgcacgat

SEQ ID NO: 924 - DNA, 12 bp, Artificial Sequence
  primer
attaagcctaag

SEQ ID NO: 925 - DNA, 12 bp, Artificial Sequence
  primer
ggtggaaccatg

SEQ ID NO: 926 - DNA, 12 bp, Artificial Sequence
  primer
actaacgcgact

SEQ ID NO: 927 - DNA, 12 bp, Artificial Sequence
  primer
cagttgtgctat

SEQ ID NO: 928 - DNA, 12 bp, Artificial Sequence
  primer
acgctgttagca

SEQ ID NO: 929 - DNA, 12 bp, Artificial Sequence
  primer
gtcaacgctaag

SEQ ID NO: 930 - DNA, 12 bp, Artificial Sequence
  primer
agcttaggtatg

SEQ ID NO: 931 - DNA, 12 bp, Artificial Sequence
  primer
cgcaggacgatt

SEQ ID NO: 932 - DNA, 12 bp, Artificial Sequence
  primer
aaccggctgtct

SEQ ID NO: 933 - DNA, 12 bp, Artificial Sequence
  primer
gttgctcacgtg

SEQ ID NO: 934 - DNA, 12 bp, Artificial Sequence
  primer
gaatcttccgcg

SEQ ID NO: 935 - DNA, 12 bp, Artificial Sequence
  primer
agagcgtacacg

SEQ ID NO: 936 - DNA, 12 bp, Artificial Sequence
  primer
aaggctaatgtc

SEQ ID NO: 937 - DNA, 12 bp, Artificial Sequence
  primer
tctatgtagacg

SEQ ID NO: 938 - DNA, 12 bp, Artificial Sequence
  primer
agacggtctagt

SEQ ID NO: 939 - DNA, 12 bp, Artificial Sequence
  primer
ttggtcacacgc

SEQ ID NO: 940 - DNA, 12 bp, Artificial Sequence
  primer
gtcgatatatgg

SEQ ID NO: 941 - DNA, 12 bp, Artificial Sequence
  primer
aacatggatacg

SEQ ID NO: 942 - DNA, 12 bp, Artificial Sequence
  primer
ttcgcagttcct

SEQ ID NO: 943 - DNA, 12 bp, Artificial Sequence
  primer
cgcatgttgtgc

SEQ ID NO: 944 - DNA, 12 bp, Artificial Sequence
  primer
tgttaagttgga

SEQ ID NO: 945 - DNA, 12 bp, Artificial Sequence
  primer
caagtgtgatga

SEQ ID NO: 946 - DNA, 12 bp, Artificial Sequence
  primer
ctggtaccacgt

SEQ ID NO: 947 - DNA, 12 bp, Artificial Sequence
  primer
cgctaggatcac

SEQ ID NO: 948 - DNA, 12 bp, Artificial Sequence
  primer
tgctcattacgg

SEQ ID NO: 949 - DNA, 12 bp, Artificial Sequence
  primer
tgctcagtaaca

SEQ ID NO: 950 - DNA, 12 bp, Artificial Sequence
  primer
acgatcatagcc

SEQ ID NO: 951 - DNA, 12 bp, Artificial Sequence
  primer
acgatacgtgga

SEQ ID NO: 952 - DNA, 12 bp, Artificial Sequence
  primer
gttcgatgatgg

SEQ ID NO: 953 - DNA, 12 bp, Artificial Sequence
  primer
aagagctgtgcc

SEQ ID NO: 954 - DNA, 12 bp, Artificial Sequence
  primer
ggttggatcaac

SEQ ID NO: 955 - DNA, 12 bp, Artificial Sequence
  primer
gcgcgcttatga

SEQ ID NO: 956 - DNA, 12 bp, Artificial Sequence
  primer
cgtcgatcatca

SEQ ID NO: 957 - DNA, 12 bp, Artificial Sequence
  primer
gagactgcactc

SEQ ID NO: 958 - DNA, 12 bp, Artificial Sequence
  primer
gatagatcgcat

SEQ ID NO: 959 - DNA, 12 bp, Artificial Sequence
  primer
ggccatcatcag

SEQ ID NO: 960 - DNA, 12 bp, Artificial Sequence
  primer
ggtgttccactg

SEQ ID NO: 961 - DNA, 14 bp, Artificial Sequence
  primer
agctatacagaggt

SEQ ID NO: 962 - DNA, 14 bp, Artificial Sequence
  primer
aggccgttctgtct

SEQ ID NO: 963 - DNA, 14 bp, Artificial Sequence
  primer
cattggtctgctat

SEQ ID NO: 964 - DNA, 14 bp, Artificial Sequence
  primer
ctacatacgcgcca

SEQ ID NO: 965 - DNA, 14 bp, Artificial Sequence
  primer
gcttaacggcgctt

SEQ ID NO: 966 - DNA, 14 bp, Artificial Sequence
  primer
tacgatactccacc

SEQ ID NO: 967 - DNA, 14 bp, Artificial Sequence
  primer
accggcataagaag

SEQ ID NO: 968 - DNA, 14 bp, Artificial Sequence
  primer
ggatgcttcgataa

SEQ ID NO: 969 - DNA, 14 bp, Artificial Sequence
  primer
gtgtacctgaatgt

SEQ ID NO: 970 - DNA, 14 bp, Artificial Sequence
  primer
cgcggatacacaga

SEQ ID NO: 971 - DNA, 14 bp, Artificial Sequence
  primer
ttccacggcactgt

SEQ ID NO: 972 - DNA, 14 bp, Artificial Sequence
  primer
tagccaggcaacaa

SEQ ID NO: 973 - DNA, 14 bp, Artificial Sequence
  primer
agcgtcaacacgta

SEQ ID NO: 974 - DNA, 14 bp, Artificial Sequence
  primer
taacgctactcgcg

SEQ ID NO: 975 - DNA, 14 bp, Artificial Sequence
  primer
tagatagacgatct

SEQ ID NO: 976 - DNA, 14 bp, Artificial Sequence
  primer
actcttgcaatgct

SEQ ID NO: 977 - DNA, 14 bp, Artificial Sequence
  primer
actcggttaggtcg

SEQ ID NO: 978 - DNA, 14 bp, Artificial Sequence
  primer
cattatctacgcat

SEQ ID NO: 979 - DNA, 14 bp, Artificial Sequence
  primer
cacaccggcgatta

SEQ ID NO: 980 - DNA, 14 bp, Artificial Sequence
  primer
tacgcagtactgtg

SEQ ID NO: 981 - DNA, 14 bp, Artificial Sequence
  primer
caagcgcgtgaatg

SEQ ID NO: 982 - DNA, 14 bp, Artificial Sequence
  primer
gaatggactgacga

SEQ ID NO: 983 - DNA, 14 bp, Artificial Sequence
  primer
ctagcgctgaagtt

SEQ ID NO: 984 - DNA, 14 bp, Artificial Sequence
  primer
tgcggcagaccaat

SEQ ID NO: 985 - DNA, 14 bp, Artificial Sequence
  primer
aaggcatagagatt

SEQ ID NO: 986 - DNA, 14 bp, Artificial Sequence
  primer
ttctcctcgccatg

SEQ ID NO: 987 - DNA, 14 bp, Artificial Sequence
  primer
tcattggtcgtgaa

SEQ ID NO: 988 - DNA, 14 bp, Artificial Sequence
  primer
attacgctatacga

SEQ ID NO: 989 - DNA, 14 bp, Artificial Sequence
  primer
atgatcctccacgg

SEQ ID NO: 990 - DNA, 14 bp, Artificial Sequence
  primer
cgtcgttagtaatc

SEQ ID NO: 991 - DNA, 14 bp, Artificial Sequence
  primer
tgcacatagtctca

SEQ ID NO: 992 - DNA, 14 bp, Artificial Sequence
  primer
gtcaaggagtcacg

SEQ ID NO: 993 - DNA, 14 bp, Artificial Sequence
  primer
ggttggaatcttgc

SEQ ID NO: 994 - DNA, 14 bp, Artificial Sequence
  primer
catcggtgcactca

SEQ ID NO: 995 - DNA, 14 bp, Artificial Sequence
  primer
aatgcactagacgt

SEQ ID NO: 996 - DNA, 14 bp, Artificial Sequence
  primer
tacagtcaggctcg

SEQ ID NO: 997 - DNA, 14 bp, Artificial Sequence
  primer
agagaagcttagcc

SEQ ID NO: 998 - DNA, 14 bp, Artificial Sequence
  primer
ccataggatcgtat

SEQ ID NO: 999 - DNA, 14 bp, Artificial Sequence
  primer
ttgtgctacacctg

SEQ ID NO: 1000 - DNA, 14 bp, Artificial Sequence
  primer
ctccagtaatacta

SEQ ID NO: 1001 - DNA, 14 bp, Artificial Sequence
  primer
tgatgccgatgtgg

SEQ ID NO: 1002 - DNA, 14 bp, Artificial Sequence
  primer
gtcataccgcttaa

SEQ ID NO: 1003 - DNA, 14 bp, Artificial Sequence
  primer
acgttctcttgaga

SEQ ID NO: 1004 - DNA, 14 bp, Artificial Sequence
  primer
cagccatatcgtgt

SEQ ID NO: 1005 - DNA, 14 bp, Artificial Sequence
  primer
ttgaacgtagcaat

SEQ ID NO: 1006 - DNA, 14 bp, Artificial Sequence
  primer
acaatcgcggtaat

SEQ ID NO: 1007 - DNA, 14 bp, Artificial Sequence
  primer
gttcctgtagatcc

SEQ ID NO: 1008 - DNA, 14 bp, Artificial Sequence
  primer
agagccttacggca

SEQ ID NO: 1009 - DNA, 14 bp, Artificial Sequence
  primer
aatatggcgccacc

SEQ ID NO: 1010 - DNA, 14 bp, Artificial Sequence
  primer
accatataggttcg

SEQ ID NO: 1011 - DNA, 14 bp, Artificial Sequence
  primer
atgcaccacagctg

SEQ ID NO: 1012 - DNA, 14 bp, Artificial Sequence
  primer
ctactattgaacag

SEQ ID NO: 1013 - DNA, 14 bp, Artificial Sequence
  primer
tgccatcactctag

SEQ ID NO: 1014 - DNA, 14 bp, Artificial Sequence
  primer
gcgaacgagaatcg

SEQ ID NO: 1015 - DNA, 14 bp, Artificial Sequence
  primer
gaatcaaggagacc

SEQ ID NO: 1016 - DNA, 14 bp, Artificial Sequence
  primer
caacatctatgcag

SEQ ID NO: 1017 - DNA, 14 bp, Artificial Sequence
  primer
caatccgtcatgga

SEQ ID NO: 1018 - DNA, 14 bp, Artificial Sequence
  primer
agctcttagccata

SEQ ID NO: 1019 - DNA, 14 bp, Artificial Sequence
  primer
aacaaggcaactgg

SEQ ID NO: 1020 - DNA, 14 bp, Artificial Sequence
  primer
gtcgtcgctcctat

SEQ ID NO: 1021 - DNA, 14 bp, Artificial Sequence
  primer
gtcatcattagatg

SEQ ID NO: 1022 - DNA, 14 bp, Artificial Sequence
  primer
gcactaagtagcag

SEQ ID NO: 1023 - DNA, 14 bp, Artificial Sequence
  primer
accttaccggacct

SEQ ID NO: 1024 - DNA, 14 bp, Artificial Sequence
  primer
gctcaggtatgtca

SEQ ID NO: 1025 - DNA, 14 bp, Artificial Sequence
  primer
tgtcacgagttagt

SEQ ID NO: 1026 - DNA, 14 bp, Artificial Sequence
  primer
cagatgacttacgt

SEQ ID NO: 1027 - DNA, 14 bp, Artificial Sequence
  primer
gaagtagcgattga

SEQ ID NO: 1028 - DNA, 14 bp, Artificial Sequence
  primer
gcaggcaatctgta

SEQ ID NO: 1029 - DNA, 14 bp, Artificial Sequence
  primer
ccttatacaacaag

SEQ ID NO: 1030 - DNA, 14 bp, Artificial Sequence
  primer
ccttagattgattg

SEQ ID NO: 1031 - DNA, 14 bp, Artificial Sequence
  primer
agccacgagtgata

SEQ ID NO: 1032 - DNA, 14 bp, Artificial Sequence
  primer
ggatgactcgtgac

SEQ ID NO: 1033 - DNA, 14 bp, Artificial Sequence
  primer
cttcgttcgccatt

SEQ ID NO: 1034 - DNA, 14 bp, Artificial Sequence
  primer
tcttgcgtattgat

SEQ ID NO: 1035 - DNA, 14 bp, Artificial Sequence
  primer
cttaacgtggtggc

SEQ ID NO: 1036 - DNA, 14 bp, Artificial Sequence
  primer
tgctgttacggaag

SEQ ID NO: 1037 - DNA, 14 bp, Artificial Sequence
  primer
ctgaattagttctc

SEQ ID NO: 1038 - DNA, 14 bp, Artificial Sequence
  primer
cctccaagtacaga

SEQ ID NO: 1039 - DNA, 14 bp, Artificial Sequence
  primer
ctggtaattcgcgg

SEQ ID NO: 1040 - DNA, 14 bp, Artificial Sequence
  primer
cgactgcaatctgg

SEQ ID NO: 1041 - DNA, 14 bp, Artificial Sequence
  primer
tggatcgcgattgg

SEQ ID NO: 1042 - DNA, 14 bp, Artificial Sequence
  primer
cgactattcctgcg

SEQ ID NO: 1043 - DNA, 14 bp, Artificial Sequence
  primer
caagtaggtccgtc

SEQ ID NO: 1044 - DNA, 14 bp, Artificial Sequence
  primer
agtaatcagtgttc

SEQ ID NO: 1045 - DNA, 14 bp, Artificial Sequence
  primer
ttattctcactacg

SEQ ID NO: 1046 - DNA, 14 bp, Artificial Sequence
  primer
catgtcttcttcgt

SEQ ID NO: 1047 - DNA, 14 bp, Artificial Sequence
  primer
aggcacataccatc

SEQ ID NO: 1048 - DNA, 14 bp, Artificial Sequence
  primer
aggttagaggatgt

SEQ ID NO: 1049 - DNA, 14 bp, Artificial Sequence
  primer
caactggcaagtgc

SEQ ID NO: 1050 - DNA, 14 bp, Artificial Sequence
  primer
cgctcacatagagg

SEQ ID NO: 1051 - DNA, 14 bp, Artificial Sequence
  primer
gcaatgtcgagatc

SEQ ID NO: 1052 - DNA, 14 bp, Artificial Sequence
  primer
gttctgtggtgctc

SEQ ID NO: 1053 - DNA, 14 bp, Artificial Sequence
  primer
aagtgatcagacta

SEQ ID NO: 1054 - DNA, 14 bp, Artificial Sequence
  primer
attgaaggattcca

SEQ ID NO: 1055 - DNA, 14 bp, Artificial Sequence
  primer
acgccatgctacta

SEQ ID NO: 1056 - DNA, 14 bp, Artificial Sequence
  primer
ctgaagatgtctgc

SEQ ID NO: 1057 - DNA, 16 bp, Artificial Sequence
  primer
gacaatctctgccgat

SEQ ID NO: 1058 - DNA, 16 bp, Artificial Sequence
  primer
ggtccgcctaatgtaa

SEQ ID NO: 1059 - DNA, 16 bp, Artificial Sequence
  primer
agccacaggcaattcc

SEQ ID NO: 1060 - DNA, 16 bp, Artificial Sequence
  primer
atctcaagttctcaac

SEQ ID NO: 1061 - DNA, 16 bp, Artificial Sequence
  primer
tgtaacgcatacgacg

SEQ ID NO: 1062 - DNA, 16 bp, Artificial Sequence
  primer
tatctcgaataccagc

SEQ ID NO: 1063 - DNA, 16 bp, Artificial Sequence
  primer
accgcaacacaggcaa

SEQ ID NO: 1064 - DNA, 16 bp, Artificial Sequence
  primer
ggccagtaacatgact

SEQ ID NO: 1065 - DNA, 16 bp, Artificial Sequence
  primer
gtgaacagttaaggtg

SEQ ID NO: 1066 - DNA, 16 bp, Artificial Sequence
  primer
ccaggatccgtattgc

SEQ ID NO: 1067 - DNA, 16 bp, Artificial Sequence
  primer
gacctagcactagacc

SEQ ID NO: 1068 - DNA, 16 bp, Artificial Sequence
  primer
cgccatcctattcacg

SEQ ID NO: 1069 - DNA, 16 bp, Artificial Sequence
  primer
aagtgcagtaatggaa

SEQ ID NO: 1070 - DNA, 16 bp, Artificial Sequence
  primer
tcaacgcgttcgtcta

SEQ ID NO: 1071 - DNA, 16 bp, Artificial Sequence
  primer
agcggccactatctaa

SEQ ID NO: 1072 - DNA, 16 bp, Artificial Sequence
  primer
ctcggcgccatataga

SEQ ID NO: 1073 - DNA, 16 bp, Artificial Sequence
  primer
cgataacttagaagaa

SEQ ID NO: 1074 - DNA, 16 bp, Artificial Sequence
  primer
cataggatgtgacgcc

SEQ ID NO: 1075 - DNA, 16 bp, Artificial Sequence
  primer
ggcttgtcgtcgtatc

SEQ ID NO: 1076 - DNA, 16 bp, Artificial Sequence
  primer
cttgtctgaatattag

SEQ ID NO: 1077 - DNA, 16 bp, Artificial Sequence
  primer
acagttcgagtgtcgg

SEQ ID NO: 1078 - DNA, 16 bp, Artificial Sequence
  primer
ctctaacctgtgacgt

SEQ ID NO: 1079 - DNA, 16 bp, Artificial Sequence
  primer
cgcgctaattcaacaa

SEQ ID NO: 1080 - DNA, 16 bp, Artificial Sequence
  primer
actcacgaatgcggca

SEQ ID NO: 1081 - DNA, 16 bp, Artificial Sequence
  primer
aatcttcggcattcat

SEQ ID NO: 1082 - DNA, 16 bp, Artificial Sequence
  primer
aagtatcaggatcgcg

SEQ ID NO: 1083 - DNA, 16 bp, Artificial Sequence
  primer
agtaactctgcagaca

SEQ ID NO: 1084 - DNA, 16 bp, Artificial Sequence
  primer
ggattgaacattgtgc

SEQ ID NO: 1085 - DNA, 16 bp, Artificial Sequence
  primer
gtgatgctcacgcatc

SEQ ID NO: 1086 - DNA, 16 bp, Artificial Sequence
  primer
cgtagcgtaacggata

SEQ ID NO: 1087 - DNA, 16 bp, Artificial Sequence
  primer
tgcgatgcaccgttag

SEQ ID NO: 1088 - DNA, 16 bp, Artificial Sequence
  primer
ccagtatgctctcagg

SEQ ID NO: 1089 - DNA, 16 bp, Artificial Sequence
  primer
aatgacgttgaagcct

SEQ ID NO: 1090 - DNA, 16 bp, Artificial Sequence
  primer
tcgattctataggagt

SEQ ID NO: 1091 - DNA, 16 bp, Artificial Sequence
  primer
cgataggttcagctat

SEQ ID NO: 1092 - DNA, 16 bp, Artificial Sequence
  primer
ccatgttgatagaata

SEQ ID NO: 1093 - DNA, 16 bp, Artificial Sequence
  primer
gagccacttctacagg

SEQ ID NO: 1094 - DNA, 16 bp, Artificial Sequence
  primer
gcgaactctcggtaat

SEQ ID NO: 1095 - DNA, 16 bp, Artificial Sequence
  primer
gacctgagtagctggt

SEQ ID NO: 1096 - DNA, 16 bp, Artificial Sequence
  primer
cgagtctattagcctg

SEQ ID NO: 1097 - DNA, 16 bp, Artificial Sequence
  primer
gtagtgccatacacct

SEQ ID NO: 1098 - DNA, 16 bp, Artificial Sequence
  primer
ccagtggtctatagca

SEQ ID NO: 1099 - DNA, 16 bp, Artificial Sequence
  primer
gtcagtgcgttattgc

SEQ ID NO: 1100 - DNA, 16 bp, Artificial Sequence
  primer
agtgtcggagtgacga

SEQ ID NO: 1101 - DNA, 16 bp, Artificial Sequence
  primer
aatctccgctatagtt

SEQ ID NO: 1102 - DNA, 16 bp, Artificial Sequence
  primer
cgagtaggtctgactt

SEQ ID NO: 1103 - DNA, 16 bp, Artificial Sequence
  primer
ctgtcgctctaataac

SEQ ID NO: 1104 - DNA, 16 bp, Artificial Sequence
  primer
gctgtcaatataactg

SEQ ID NO: 1105 - DNA, 16 bp, Artificial Sequence
  primer
agctcaagttgaatcc

SEQ ID NO: 1106 - DNA, 16 bp, Artificial Sequence
  primer
aattcatgctcctaac

SEQ ID NO: 1107 - DNA, 16 bp, Artificial Sequence
  primer
ccaaggtctggtgata

SEQ ID NO: 1108 - DNA, 16 bp, Artificial Sequence
  primer
ctccacgtatcttgaa

SEQ ID NO: 1109 - DNA, 16 bp, Artificial Sequence
  primer
tagccgaacaacactt

SEQ ID NO: 1110 - DNA, 16 bp, Artificial Sequence
  primer
agtacacgacatatgc

SEQ ID NO: 1111 - DNA, 16 bp, Artificial Sequence
  primer
acgttctagactcctg

SEQ ID NO: 1112 - DNA, 16 bp, Artificial Sequence
  primer
cgactcaagcactgct

SEQ ID NO: 1113 - DNA, 16 bp, Artificial Sequence
  primer
tgaagctcacgattaa

SEQ ID NO: 1114 - DNA, 16 bp, Artificial Sequence
  primer
tatctaacgtatggta

SEQ ID NO: 1115 - DNA, 16 bp, Artificial Sequence
  primer
tataccatgttccttg

SEQ ID NO: 1116 - DNA, 16 bp, Artificial Sequence
  primer
ttcctacgatgacttc

SEQ ID NO: 1117 - DNA, 16 bp, Artificial Sequence
  primer
ctctccaatatgtgcc

SEQ ID NO: 1118 - DNA, 16 bp, Artificial Sequence
  primer
gagtagagtcttgcca

SEQ ID NO: 1119 - DNA, 16 bp, Artificial Sequence
  primer
gcgagatgtggtccta

SEQ ID NO: 1120 - DNA, 16 bp, Artificial Sequence
  primer
aagctacacggaccac

SEQ ID NO: 1121 - DNA, 16 bp, Artificial Sequence
  primer
atacaactggcaaccg

SEQ ID NO: 1122 - DNA, 16 bp, Artificial Sequence
  primer
cggtagatgctatgct

SEQ ID NO: 1123 - DNA, 16 bp, Artificial Sequence
  primer
tcttgaccggtcatca

SEQ ID NO: 1124 - DNA, 16 bp, Artificial Sequence
  primer
agatcgtgcatgcgat

SEQ ID NO: 1125 - DNA, 16 bp, Artificial Sequence
  primer
tcctcgagacagcctt

SEQ ID NO: 1126 - DNA, 16 bp, Artificial Sequence
  primer
tagccggtaccactta

SEQ ID NO: 1127 - DNA, 16 bp, Artificial Sequence
  primer
gtaaggcagcgtgcaa

SEQ ID NO: 1128 - DNA, 16 bp, Artificial Sequence
  primer
tagtctgctcctggtc

SEQ ID NO: 1129 - DNA, 16 bp, Artificial Sequence
  primer
tggattatagcagcag

SEQ ID NO: 1130 - DNA, 16 bp, Artificial Sequence
  primer
aagaatgatcagacat

SEQ ID NO: 1131 - DNA, 16 bp, Artificial Sequence
  primer
cagcgctatatacctc

SEQ ID NO: 1132 - DNA, 16 bp, Artificial Sequence
  primer
gagtagtacctccacc

SEQ ID NO: 1133 - DNA, 16 bp, Artificial Sequence
  primer
gacgtgatcctctaga

SEQ ID NO: 1134 - DNA, 16 bp, Artificial Sequence
  primer
gttccgttcactacga

SEQ ID NO: 1135 - DNA, 16 bp, Artificial Sequence
  primer
tgcaagcaccaggatg

SEQ ID NO: 1136 - DNA, 16 bp, Artificial Sequence
  primer
ttagttggcggctgag

SEQ ID NO: 1137 - DNA, 16 bp, Artificial Sequence
  primer
cagatgcagacatacg

SEQ ID NO: 1138 - DNA, 16 bp, Artificial Sequence
  primer
gacgcttgatgattat

SEQ ID NO: 1139 - DNA, 16 bp, Artificial Sequence
  primer
tggatcacgactagga

SEQ ID NO: 1140 - DNA, 16 bp, Artificial Sequence
  primer
ctcgtcggtataacgc

SEQ ID NO: 1141 - DNA, 16 bp, Artificial Sequence
  primer
aagcacggatgcgatt

SEQ ID NO: 1142 - DNA, 16 bp, Artificial Sequence
  primer
agatcttccggtgaac

SEQ ID NO: 1143 - DNA, 16 bp, Artificial Sequence
  primer
ggacaatagcaacctg

SEQ ID NO: 1144 - DNA, 16 bp, Artificial Sequence
  primer
gataatcggttccaat

SEQ ID NO: 1145 - DNA, 16 bp, Artificial Sequence
  primer
ctcaagctacagttgt

SEQ ID NO: 1146 - DNA, 16 bp, Artificial Sequence
  primer
gttggcatgatgtaga

SEQ ID NO: 1147 - DNA, 16 bp, Artificial Sequence
  primer
cagcatgaggtaagtg

SEQ ID NO: 1148 - DNA, 16 bp, Artificial Sequence
  primer
gcctcatcacacgtca

SEQ ID NO: 1149 - DNA, 16 bp, Artificial Sequence
  primer
tcgatactacacatcg

SEQ ID NO: 1150 - DNA, 16 bp, Artificial Sequence
  primer
tacacgaggcttgatc

SEQ ID NO: 1151 - DNA, 16 bp, Artificial Sequence
  primer
ttctcgtgtccgcatt

SEQ ID NO: 1152 - DNA, 16 bp, Artificial Sequence
  primer
ggtgaagcaacagcat

SEQ ID NO: 1153 - DNA, 18 bp, Artificial Sequence
  primer
cgaaccgactgtacagtt

SEQ ID NO: 1154 - DNA, 18 bp, Artificial Sequence
  primer
ccgactgcggataagtta

SEQ ID NO: 1155 - DNA, 18 bp, Artificial Sequence
  primer
cgacaggtaggtaagcag

SEQ ID NO: 1156 - DNA, 18 bp, Artificial Sequence
  primer
tgatacgttggtatacag

SEQ ID NO: 1157 - DNA, 18 bp, Artificial Sequence
  primer
ctactatagaatacgtag

SEQ ID NO: 1158 - DNA, 18 bp, Artificial Sequence
  primer
agactgtggcaatggcat

SEQ ID NO: 1159 - DNA, 18 bp, Artificial Sequence
  primer
ggaagactgatacaacga

SEQ ID NO: 1160 - DNA, 18 bp, Artificial Sequence
  primer
tatgcacatatagcgctt

SEQ ID NO: 1161 - DNA, 18 bp, Artificial Sequence
  primer
catggtaatcgaccgagg

SEQ ID NO: 1162 - DNA, 18 bp, Artificial Sequence
  primer
gtcattgccgtcattgcc

SEQ ID NO: 1163 - DNA, 18 bp, Artificial Sequence
  primer
cctaagaactccgaagct

SEQ ID NO: 1164 - DNA, 18 bp, Artificial Sequence
  primer
tcgctcaccgtactagga

SEQ ID NO: 1165 - DNA, 18 bp, Artificial Sequence
  primer
tattactgtcacagcagg

SEQ ID NO: 1166 - DNA, 18 bp, Artificial Sequence
  primer
tgagacaggctacgagtc

SEQ ID NO: 1167 - DNA, 18 bp, Artificial Sequence
  primer
aagctatgcgaacacgtt

SEQ ID NO: 1168 - DNA, 18 bp, Artificial Sequence
  primer
aacggaggagtgagccaa

SEQ ID NO: 1169 - DNA, 18 bp, Artificial Sequence
  primer
ccactatggacatcatgg

SEQ ID NO: 1170 - DNA, 18 bp, Artificial Sequence
  primer
atggtggtggatagctcg

SEQ ID NO: 1171 - DNA, 18 bp, Artificial Sequence
  primer
tcaccggttacacatcgc

SEQ ID NO: 1172 - DNA, 18 bp, Artificial Sequence
  primer
aagatactgagatatgga

SEQ ID NO: 1173 - DNA, 18 bp, Artificial Sequence
  primer
gacctgttcttgaactag

SEQ ID NO: 1174 - DNA, 18 bp, Artificial Sequence
  primer
aagtagagctctcggtta

SEQ ID NO: 1175 - DNA, 18 bp, Artificial Sequence
  primer
ctatgttcttactctctt

SEQ ID NO: 1176 - DNA, 18 bp, Artificial Sequence
  primer
caaggctataagcggtta

SEQ ID NO: 1177 - DNA, 18 bp, Artificial Sequence
  primer
gaagctaattaaccgata

SEQ ID NO: 1178 - DNA, 18 bp, Artificial Sequence
  primer
ttcacgtctgccaagcac

SEQ ID NO: 1179 - DNA, 18 bp, Artificial Sequence
  primer
atcgtatagatcgagaca

SEQ ID NO: 1180 - DNA, 18 bp, Artificial Sequence
  primer
gtcacagattcacatcat

SEQ ID NO: 1181 - DNA, 18 bp, Artificial Sequence
  primer
gtgcctgtgaactatcag

SEQ ID NO: 1182 - DNA, 18 bp, Artificial Sequence
  primer
cagcgtacaagatagtcg

SEQ ID NO: 1183 - DNA, 18 bp, Artificial Sequence
  primer
gcatggcatggtagacct

SEQ ID NO: 1184 - DNA, 18 bp, Artificial Sequence
  primer
ggtatgctactcttcgca

SEQ ID NO: 1185 - DNA, 18 bp, Artificial Sequence
  primer
atgttcagtcacaagcga

SEQ ID NO: 1186 - DNA, 18 bp, Artificial Sequence
  primer
taggaagtgtgtaatagc

SEQ ID NO: 1187 - DNA, 18 bp, Artificial Sequence
  primer
aatccatgtagctgtacg

SEQ ID NO: 1188 - DNA, 18 bp, Artificial Sequence
  primer
ccagattcactggcatag

SEQ ID NO: 1189 - DNA, 18 bp, Artificial Sequence
  primer
ttgtctctacgtaatatc

SEQ ID NO: 1190 - DNA, 18 bp, Artificial Sequence
  primer
gtggtgcttgtgacaatt

SEQ ID NO: 1191 - DNA, 18 bp, Artificial Sequence
  primer
cagcctacttggctgaga

SEQ ID NO: 1192 - DNA, 18 bp, Artificial Sequence
  primer
tactcaatgcatctgtgt

SEQ ID NO: 1193 - DNA, 18 bp, Artificial Sequence
  primer
tgtagagagacgaatata

SEQ ID NO: 1194 - DNA, 18 bp, Artificial Sequence
  primer
gcctacaaccatcctact

SEQ ID NO: 1195 - DNA, 18 bp, Artificial Sequence
  primer
gcgtggcattgagattca

SEQ ID NO: 1196 - DNA, 18 bp, Artificial Sequence
  primer
gcatgccagctaactgag

SEQ ID NO: 1197 - DNA, 18 bp, Artificial Sequence
  primer
gcgagtaatccggttgga

SEQ ID NO: 1198 - DNA, 18 bp, Artificial Sequence
  primer
gcctctaccagaacgtca

SEQ ID NO: 1199 - DNA, 18 bp, Artificial Sequence
  primer
gtcagcagaagactgacc

SEQ ID NO: 1200 - DNA, 18 bp, Artificial Sequence
  primer
gataacagacgtagcagg

SEQ ID NO: 1201 - DNA, 18 bp, Artificial Sequence
  primer
caggagatcgcatgtcgt

SEQ ID NO: 1202 - DNA, 18 bp, Artificial Sequence
  primer
ctggaaggaatggagcca

SEQ ID NO: 1203 - DNA, 18 bp, Artificial Sequence
  primer
attggttctctaccacaa

SEQ ID NO: 1204 - DNA, 18 bp, Artificial Sequence
  primer
ctcattgttgacggctca

SEQ ID NO: 1205 - DNA, 18 bp, Artificial Sequence
  primer
ttcaggactgtagttcat

SEQ ID NO: 1206 - DNA, 18 bp, Artificial Sequence
  primer
agaccgcactaactcaag

SEQ ID NO: 1207 - DNA, 18 bp, Artificial Sequence
  primer
ggaatattgtgcagaccg

SEQ ID NO: 1208 - DNA, 18 bp, Artificial Sequence
  primer
cctattactaatagctca

SEQ ID NO: 1209 - DNA, 18 bp, Artificial Sequence
  primer
atggcatgagtacttcgg

SEQ ID NO: 1210 - DNA, 18 bp, Artificial Sequence
  primer
gacacgtatgcgtctagc

SEQ ID NO: 1211 - DNA, 18 bp, Artificial Sequence
  primer
gaaggtacggaatctgtt

SEQ ID NO: 1212 - DNA, 18 bp, Artificial Sequence
  primer
tataacgtccgacactgt

SEQ ID NO: 1213 - DNA, 18 bp, Artificial Sequence
  primer
gctaatacattaccgccg

SEQ ID NO: 1214 - DNA, 18 bp, Artificial Sequence
  primer
gaagccaacactcctgac

SEQ ID NO: 1215 - DNA, 18 bp, Artificial Sequence
  primer
cgaataacgagctgtgat

SEQ ID NO: 1216 - DNA, 18 bp, Artificial Sequence
  primer
gcctaccgatcgcactta

SEQ ID NO: 1217 - DNA, 18 bp, Artificial Sequence
  primer
ctgaggagaatagcctgc

SEQ ID NO: 1218 - DNA, 18 bp, Artificial Sequence
  primer
cagcatggacagtacttc

SEQ ID NO: 1219 - DNA, 18 bp, Artificial Sequence
  primer
ggtatagagccttcctta

SEQ ID NO: 1220 - DNA, 18 bp, Artificial Sequence
  primer
cgctctgcatatatagca

SEQ ID NO: 1221 - DNA, 18 bp, Artificial Sequence
  primer
cggctctactatgctcgt

SEQ ID NO: 1222 - DNA, 18 bp, Artificial Sequence
  primer
cctaatgcgaagctcacc

SEQ ID NO: 1223 - DNA, 18 bp, Artificial Sequence
  primer
acaaccggtgaggcagta

SEQ ID NO: 1224 - DNA, 18 bp, Artificial Sequence
  primer
ttggttcgaaccaaccgc

SEQ ID NO: 1225 - DNA, 18 bp, Artificial Sequence
  primer
atactaggttgaactaag

SEQ ID NO: 1226 - DNA, 18 bp, Artificial Sequence
  primer
gcgttgagagtaacatat

SEQ ID NO: 1227 - DNA, 18 bp, Artificial Sequence
  primer
agttgtataataagcgtc

SEQ ID NO: 1228 - DNA, 18 bp, Artificial Sequence
  primer
gtatgatgccgtccaatt

SEQ ID NO: 1229 - DNA, 18 bp, Artificial Sequence
  primer
ggactctctgaagagtct

SEQ ID NO: 1230 - DNA, 18 bp, Artificial Sequence
  primer
ggactctcttgacttgaa

SEQ ID NO: 1231 - DNA, 18 bp, Artificial Sequence
  primer
gataacagtgcttcgtcc

SEQ ID NO: 1232 - DNA, 18 bp, Artificial Sequence
  primer
ggccattatagatgaact

SEQ ID NO: 1233 - DNA, 18 bp, Artificial Sequence
  primer
atagagagcacagagcag

SEQ ID NO: 1234 - DNA, 18 bp, Artificial Sequence
  primer
gtgtgagtgtatcataac

SEQ ID NO: 1235 - DNA, 18 bp, Artificial Sequence
  primer
ataaccttagtgcgcgtc

SEQ ID NO: 1236 - DNA, 18 bp, Artificial Sequence
  primer
ccgactgatatgcatgga

SEQ ID NO: 1237 - DNA, 18 bp, Artificial Sequence
  primer
ggatatctgatcgcatca

SEQ ID NO: 1238 - DNA, 18 bp, Artificial Sequence
  primer
cagcattaacgaggcgaa

SEQ ID NO: 1239 - DNA, 18 bp, Artificial Sequence
  primer
gcgaggcctacatattcg

SEQ ID NO: 1240 - DNA, 18 bp, Artificial Sequence
  primer
cgataagtggtaaggtct

SEQ ID NO: 1241 - DNA, 18 bp, Artificial Sequence
  primer
agatcctgagtcgagcaa

SEQ ID NO: 1242 - DNA, 18 bp, Artificial Sequence
  primer
aagatataacgagaccga

SEQ ID NO: 1243 - DNA, 18 bp, Artificial Sequence
  primer
ccgactgattgagaacgt

SEQ ID NO: 1244 - DNA, 18 bp, Artificial Sequence
  primer
tcggcttatatgacacgt

SEQ ID NO: 1245 - DNA, 18 bp, Artificial Sequence
  primer
aataacgtacgccggagg

SEQ ID NO: 1246 - DNA, 18 bp, Artificial Sequence
  primer
aacacagcattgcgcacg

SEQ ID NO: 1247 - DNA, 18 bp, Artificial Sequence
  primer
gtagtctgacagcaacaa

SEQ ID NO: 1248 - DNA, 18 bp, Artificial Sequence
  primer
agaatgacttgagctgct

SEQ ID NO: 1249 - DNA, 20 bp, Artificial Sequence
  primer
actggtagtaacgtccacct

SEQ ID NO: 1250 - DNA, 20 bp, Artificial Sequence
  primer
agactggttgttattcgcct

SEQ ID NO: 1251 - DNA, 20 bp, Artificial Sequence
  primer
tatcattgacagcgagctca

SEQ ID NO: 1252 - DNA, 20 bp, Artificial Sequence
  primer
tggagtctgaagaaggactc

SEQ ID NO: 1253 - DNA, 20 bp, Artificial Sequence
  primer
catctggactacggcaacga

SEQ ID NO: 1254 - DNA, 20 bp, Artificial Sequence
  primer
aactgtcataagacagacaa

SEQ ID NO: 1255 - DNA, 20 bp, Artificial Sequence
  primer
cctcaacatgacatacaccg

SEQ ID NO: 1256 - DNA, 20 bp, Artificial Sequence
  primer
caataccgttcgcgattcta

SEQ ID NO: 1257 - DNA, 20 bp, Artificial Sequence
  primer
gcgtctacgttgattcggcc

SEQ ID NO: 1258 - DNA, 20 bp, Artificial Sequence
  primer
tgaacagaggcacttgcagg

SEQ ID NO: 1259 - DNA, 20 bp, Artificial Sequence
  primer
cgactagaacctactactgc

SEQ ID NO: 1260 - DNA, 20 bp, Artificial Sequence
  primer
gcaccgcacgtggagagata

SEQ ID NO: 1261 - DNA, 20 bp, Artificial Sequence
  primer
ctgagagaccgactgatgcg

SEQ ID NO: 1262 - DNA, 20 bp, Artificial Sequence
  primer
tcgtccttctacttaatgat

SEQ ID NO: 1263 - DNA, 20 bp, Artificial Sequence
  primer
caagctataccatccgaatt

SEQ ID NO: 1264 - DNA, 20 bp, Artificial Sequence
  primer
caatacgtatagtcttagat

SEQ ID NO: 1265 - DNA, 20 bp, Artificial Sequence
  primer
ccatccacagtgacctatgt

SEQ ID NO: 1266 - DNA, 20 bp, Artificial Sequence
  primer
tatccgttggagaaggttca

SEQ ID NO: 1267 - DNA, 20 bp, Artificial Sequence
  primer
cgcctaggtacctgagtacg

SEQ ID NO: 1268 - DNA, 20 bp, Artificial Sequence
  primer
cagagtgctcgtgttcgcga

SEQ ID NO: 1269 - DNA, 20 bp, Artificial Sequence
  primer
cgcttggacatccttaagaa

SEQ ID NO: 1270 - DNA, 20 bp, Artificial Sequence
  primer
gaccgcatgattagtcttac

SEQ ID NO: 1271 - DNA, 20 bp, Artificial Sequence
  primer
cttggccgtagtcactcagt

SEQ ID NO: 1272 - DNA, 20 bp, Artificial Sequence
  primer
gatagcgatattcagttcgc

SEQ ID NO: 1273 - DNA, 20 bp, Artificial Sequence
  primer
atccaacactaagacaacca

SEQ ID NO: 1274 - DNA, 20 bp, Artificial Sequence
  primer
ccattctgttgcgtgtcctc

SEQ ID NO: 1275 - DNA, 20 bp, Artificial Sequence
  primer
acattctgtacgcttgcagc

SEQ ID NO: 1276 - DNA, 20 bp, Artificial Sequence
  primer
tgctgaacgccaatcgctta

SEQ ID NO: 1277 - DNA, 20 bp, Artificial Sequence
  primer
tcctctacaagaatattgcg

SEQ ID NO: 1278 - DNA, 20 bp, Artificial Sequence
  primer
cgaccaacgcagcctgattc

SEQ ID NO: 1279 - DNA, 20 bp, Artificial Sequence
  primer
attgcgagcttgagtagcgc

SEQ ID NO: 1280 - DNA, 20 bp, Artificial Sequence
  primer
aaggtgcgagcataggaatc

SEQ ID NO: 1281 - DNA, 20 bp, Artificial Sequence
  primer
cacttaagtgtgatatagat

SEQ ID NO: 1282 - DNA, 20 bp, Artificial Sequence
  primer
atcggtatgctgacctagac

SEQ ID NO: 1283 - DNA, 20 bp, Artificial Sequence
  primer
tacaatctcgaatgcaggat

SEQ ID NO: 1284 - DNA, 20 bp, Artificial Sequence
  primer
ccatatgaagcgcagccgtc

SEQ ID NO: 1285 - DNA, 20 bp, Artificial Sequence
  primer
cgtctcgtggacattcgagg

SEQ ID NO: 1286 - DNA, 20 bp, Artificial Sequence
  primer
ccgagtacagaagcgtggaa

SEQ ID NO: 1287 - DNA, 20 bp, Artificial Sequence
  primer
ttacgtggtcgacaggcagt

SEQ ID NO: 1288 - DNA, 20 bp, Artificial Sequence
  primer
agctgcaatctgcatgatta

SEQ ID NO: 1289 - DNA, 20 bp, Artificial Sequence
  primer
acctgccgaagcagcctaca

SEQ ID NO: 1290 - DNA, 20 bp, Artificial Sequence
  primer
aacatgataaccacatggtt

SEQ ID NO: 1291 - DNA, 20 bp, Artificial Sequence
  primer
atccgactgattgaattacc

SEQ ID NO: 1292 - DNA, 20 bp, Artificial Sequence
  primer
tcacgctgactcttatcagg

SEQ ID NO: 1293 - DNA, 20 bp, Artificial Sequence
  primer
gcgcgctcgaagtacaacat

SEQ ID NO: 1294 - DNA, 20 bp, Artificial Sequence
  primer
acagccagatgcgttgttcc

SEQ ID NO: 1295 - DNA, 20 bp, Artificial Sequence
  primer
ggagctctgacctgcaagaa

SEQ ID NO: 1296 - DNA, 20 bp, Artificial Sequence
  primer
aacattagcctcaagtaaga

SEQ ID NO: 1297 - DNA, 20 bp, Artificial Sequence
  primer
tgtgattatgccgaatgagg

SEQ ID NO: 1298 - DNA, 20 bp, Artificial Sequence
  primer
gagtaataatccaatcagta

SEQ ID NO: 1299 - DNA, 20 bp, Artificial Sequence
  primer
ctccttggcgacagctgaac

SEQ ID NO: 1300 - DNA, 20 bp, Artificial Sequence
  primer
ttacgcacacatacacagac

SEQ ID NO: 1301 - DNA, 20 bp, Artificial Sequence
  primer
acgccgtatggcgacttagg

SEQ ID NO: 1302 - DNA, 20 bp, Artificial Sequence
  primer
agaacgacaattacgatggc

SEQ ID NO: 1303 - DNA, 20 bp, Artificial Sequence
  primer
tgctaacgtaccactgccac

SEQ ID NO: 1304 - DNA, 20 bp, Artificial Sequence
  primer
catccagaatgtctatcata

SEQ ID NO: 1305 - DNA, 20 bp, Artificial Sequence
  primer
ggagaacgcctatagcactc

SEQ ID NO: 1306 - DNA, 20 bp, Artificial Sequence
  primer
acctcttgtgacggccagtc

SEQ ID NO: 1307 - DNA, 20 bp, Artificial Sequence
  primer
tgccataacttggcataaga

SEQ ID NO: 1308 - DNA, 20 bp, Artificial Sequence
  primer
acaattgtctgaccacgctc

SEQ ID NO: 1309 - DNA, 20 bp, Artificial Sequence
  primer
tcgtcaccttcacagaacga

SEQ ID NO: 1310 - DNA, 20 bp, Artificial Sequence
  primer
agcagcagatgatgatccaa

SEQ ID NO: 1311 - DNA, 20 bp, Artificial Sequence
  primer
tcgtgccttggattccagga

SEQ ID NO: 1312 - DNA, 20 bp, Artificial Sequence
  primer
tgttatagccacgatactat

SEQ ID NO: 1313 - DNA, 20 bp, Artificial Sequence
  primer
aatctcacctgtaccttccg

SEQ ID NO: 1314 - DNA, 20 bp, Artificial Sequence
  primer
gagtagcggaagcgttagcg

SEQ ID NO: 1315 - DNA, 20 bp, Artificial Sequence
  primer
aatactccggcgaggtatac

SEQ ID NO: 1316 - DNA, 20 bp, Artificial Sequence
  primer
ttcgcatccttgcacgaaca

SEQ ID NO: 1317 - DNA, 20 bp, Artificial Sequence
  primer
aaccggctaatactactggc

SEQ ID NO: 1318 - DNA, 20 bp, Artificial Sequence
  primer
ctagcatcttagacaccaga

SEQ ID NO: 1319 - DNA, 20 bp, Artificial Sequence
  primer
tagttgcgtgatacaagata

SEQ ID NO: 1320 - DNA, 20 bp, Artificial Sequence
  primer
tcgtctcgacacagttggtc

SEQ ID NO: 1321 - DNA, 20 bp, Artificial Sequence
  primer
tccgttcgcgtgcgaactga

SEQ ID NO: 1322 - DNA, 20 bp, Artificial Sequence
  primer
tctgactctggtgtacagtc

SEQ ID NO: 1323 - DNA, 20 bp, Artificial Sequence
  primer
acagcgcaattatatcctgt

SEQ ID NO: 1324 - DNA, 20 bp, Artificial Sequence
  primer
agatccgtacgtgagactag

SEQ ID NO: 1325 - DNA, 20 bp, Artificial Sequence
  primer
tacattgaagcatccgaaca

SEQ ID NO: 1326 - DNA, 20 bp, Artificial Sequence
  primer
ctcctgagagatcaacgcca

SEQ ID NO: 1327 - DNA, 20 bp, Artificial Sequence
  primer
tcacctcgaatgagttcgtt

SEQ ID NO: 1328 - DNA, 20 bp, Artificial Sequence
  primer
tagcgacttaaggtccaagc

SEQ ID NO: 1329 - DNA, 20 bp, Artificial Sequence
  primer
agtacgtattgccgtgcaag

SEQ ID NO: 1330 - DNA, 20 bp, Artificial Sequence
  primer
agccacgaaccgacgtcata

SEQ ID NO: 1331 - DNA, 20 bp, Artificial Sequence
  primer
tgatgtgtacgctactacta

SEQ ID NO: 1332 - DNA, 20 bp, Artificial Sequence
  primer
ccactgtgtgcagcagacga

SEQ ID NO: 1333 - DNA, 20 bp, Artificial Sequence
  primer
ctattgtacagcgaacgctg

SEQ ID NO: 1334 - DNA, 20 bp, Artificial Sequence
  primer
ctccgatatcgcacggatcg

SEQ ID NO: 1335 - DNA, 20 bp, Artificial Sequence
  primer
aacttatcgtcggacgcatg

SEQ ID NO: 1336 - DNA, 20 bp, Artificial Sequence
  primer
tatcctaattcgtgccggtc

SEQ ID NO: 1337 - DNA, 20 bp, Artificial Sequence
  primer
acagccttcctgtgtggact

SEQ ID NO: 1338 - DNA, 20 bp, Artificial Sequence
  primer
cctccgtgaggatcgtacca

SEQ ID NO: 1339 - DNA, 20 bp, Artificial Sequence
  primer
gctctaagtaacagaactaa

SEQ ID NO: 1340 - DNA, 20 bp, Artificial Sequence
  primer
gacttaccgcgcgttctggt

SEQ ID NO: 1341 - DNA, 20 bp, Artificial Sequence
  primer
tctgaggatacacatgtgga

SEQ ID NO: 1342 - DNA, 20 bp, Artificial Sequence
  primer
tgtaatcacactggtgtcgg

SEQ ID NO: 1343 - DNA, 20 bp, Artificial Sequence
  primer
cactaggcggcagacataca

SEQ ID NO: 1344 - DNA, 20 bp, Artificial Sequence
  primer
ctagagcacagtaccacgtt

SEQ ID NO: 1345 - DNA, 22 bp, Artificial Sequence
  primer
ttcagaggtctacgcttccggt

SEQ ID NO: 1346 - DNA, 22 bp, Artificial Sequence
  primer
aacacagactgcgttatgccaa

SEQ ID NO: 1347 - DNA, 22 bp, Artificial Sequence
  primer
tgctgagttctatacagcagtg

SEQ ID NO: 1348 - DNA, 22 bp, Artificial Sequence
  primer
acctattatatgatagcgtcat

SEQ ID NO: 1349 - DNA, 22 bp, Artificial Sequence
  primer
atcgtgagctacagtgaatgca

SEQ ID NO: 1350 - DNA, 22 bp, Artificial Sequence
  primer
cgtgatgtatccggccttgcag

SEQ ID NO: 1351 - DNA, 22 bp, Artificial Sequence
  primer
tcttctggtcctagagttgtgc

SEQ ID NO: 1352 - DNA, 22 bp, Artificial Sequence
  primer
tgatgtcggcggcggatcagat

SEQ ID NO: 1353 - DNA, 22 bp, Artificial Sequence
  primer
tcggccttagcgttcagcatcc

SEQ ID NO: 1354 - DNA, 22 bp, Artificial Sequence
  primer
ttaagtaggtcagccactgcac

SEQ ID NO: 1355 - DNA, 22 bp, Artificial Sequence
  primer
ccaggtgagttgatctgacacc

SEQ ID NO: 1356 - DNA, 22 bp, Artificial Sequence
  primer
tatactattactgtgttcgatc

SEQ ID NO: 1357 - DNA, 22 bp, Artificial Sequence
  primer
ccgcagtatgtctagtgttgtc

SEQ ID NO: 1358 - DNA, 22 bp, Artificial Sequence
  primer
gtctaccgcgtacgaagctctc

SEQ ID NO: 1359 - DNA, 22 bp, Artificial Sequence
  primer
atgcgagtccgtggtcgatcct

SEQ ID NO: 1360 - DNA, 22 bp, Artificial Sequence
  primer
tggtagattggtgtgagaacta

SEQ ID NO: 1361 - DNA, 22 bp, Artificial Sequence
  primer
aggttcgtcgatcaactgctaa

SEQ ID NO: 1362 - DNA, 22 bp, Artificial Sequence
  primer
acgacaagcatcctgcgatatc

SEQ ID NO: 1363 - DNA, 22 bp, Artificial Sequence
  primer
ttgaatcacagagagcgtgatt

SEQ ID NO: 1364 - DNA, 22 bp, Artificial Sequence
  primer
gtacttagtgcttacgtcagct

SEQ ID NO: 1365 - DNA, 22 bp, Artificial Sequence
  primer
gattattaaggccaagctcata

SEQ ID NO: 1366 - DNA, 22 bp, Artificial Sequence
  primer
gcatgcagagacgtactcatcg

SEQ ID NO: 1367 - DNA, 22 bp, Artificial Sequence
  primer
tagcggatggtgtcctggcact

SEQ ID NO: 1368 - DNA, 22 bp, Artificial Sequence
  primer
tacggctgccaacttaataact

SEQ ID NO: 1369 - DNA, 22 bp, Artificial Sequence
  primer
ctcatatgacaacttctatagt

SEQ ID NO: 1370 - DNA, 22 bp, Artificial Sequence
  primer
caagcaatagttgtcggccacc

SEQ ID NO: 1371 - DNA, 22 bp, Artificial Sequence
  primer
ttcagcaatccgtactgctaga

SEQ ID NO: 1372 - DNA, 22 bp, Artificial Sequence
  primer
tgagacgttgctgacattctcc

SEQ ID NO: 1373 - DNA, 22 bp, Artificial Sequence
  primer
gttccgatgagttagatgtata

SEQ ID NO: 1374 - DNA, 22 bp, Artificial Sequence
  primer
ttgacgcttggaggagtacaag

SEQ ID NO: 1375 - DNA, 22 bp, Artificial Sequence
  primer
ttcatgttacctccacattgtg

SEQ ID NO: 1376 - DNA, 22 bp, Artificial Sequence
  primer
gagcacgtgccagattgcaacc

SEQ ID NO: 1377 - DNA, 22 bp, Artificial Sequence
  primer
ggtcgacaagcacaagccttct

SEQ ID NO: 1378 - DNA, 22 bp, Artificial Sequence
  primer
taggcaggtaagatgaccgact

SEQ ID NO: 1379 - DNA, 22 bp, Artificial Sequence
  primer
cgaggcatgccaagtcgccaat

SEQ ID NO: 1380 - DNA, 22 bp, Artificial Sequence
  primer
agtgttgataggcggatgagag

SEQ ID NO: 1381 - DNA, 22 bp, Artificial Sequence
  primer
ttcggtctagacctctcacaat

SEQ ID NO: 1382 - DNA, 22 bp, Artificial Sequence
  primer
gtgacgctcatatcttgccacc

SEQ ID NO: 1383 - DNA, 22 bp, Artificial Sequence
  primer
gatgtaattctacgcgcggact

SEQ ID NO: 1384 - DNA, 22 bp, Artificial Sequence
  primer
gatggcgatgttgcattacatg

SEQ ID NO: 1385 - DNA, 22 bp, Artificial Sequence
  primer
tatgctctgaattaacgtagaa

SEQ ID NO: 1386 - DNA, 22 bp, Artificial Sequence
  primer
aggcaatatggtgatccgtagc

SEQ ID NO: 1387 - DNA, 22 bp, Artificial Sequence
  primer
tgacagcgatgcatacagtagt

SEQ ID NO: 1388 - DNA, 22 bp, Artificial Sequence
  primer
ttctgctaacggtatccaatac

SEQ ID NO: 1389 - DNA, 22 bp, Artificial Sequence
  primer
gagtcgtccatacgatctagga

SEQ ID NO: 1390 - DNA, 22 bp, Artificial Sequence
  primer
agacggactcaacgccaattcc

SEQ ID NO: 1391 - DNA, 22 bp, Artificial Sequence
  primer
gtagtgttgagcggaccgagct

SEQ ID NO: 1392 - DNA, 22 bp, Artificial Sequence
  primer
aatataactagatcatagccag

SEQ ID NO: 1393 - DNA, 22 bp, Artificial Sequence
  primer
tcaatcggagaatacagaacgt

SEQ ID NO: 1394 - DNA, 22 bp, Artificial Sequence
  primer
atctccgtcgtccgaaccaaca

SEQ ID NO: 1395 - DNA, 22 bp, Artificial Sequence
  primer
taggcgttcagcggtatgctta

SEQ ID NO: 1396 - DNA, 22 bp, Artificial Sequence
  primer
tgcgtgctatacaacctatacg

SEQ ID NO: 1397 - DNA, 22 bp, Artificial Sequence
  primer
atggccggcatacatctgtatg

SEQ ID NO: 1398 - DNA, 22 bp, Artificial Sequence
  primer
tgatgctgacataacactgaat

SEQ ID NO: 1399 - DNA, 22 bp, Artificial Sequence
  primer
atccaaggtacctgaacatcct

SEQ ID NO: 1400 - DNA, 22 bp, Artificial Sequence
  primer
tagtgacgaccaggtgagcctc

SEQ ID NO: 1401 - DNA, 22 bp, Artificial Sequence
  primer
aggaggatccgtcaagtcgacc

SEQ ID NO: 1402 - DNA, 22 bp, Artificial Sequence
  primer
agagtatgccagatcgtgaggc

SEQ ID NO: 1403 - DNA, 22 bp, Artificial Sequence
  primer
ccactcactaggatggctgcgt

SEQ ID NO: 1404 - DNA, 22 bp, Artificial Sequence
  primer
tatccaacctgttatagcgatt

SEQ ID NO: 1405 - DNA, 22 bp, Artificial Sequence
  primer
tcttgcagtgagttgagtctgc

SEQ ID NO: 1406 - DNA, 22 bp, Artificial Sequence
  primer
ccactgttgtacatacacctgg

SEQ ID NO: 1407 - DNA, 22 bp, Artificial Sequence
  primer
atgcgcgtaggccactaagtcc

SEQ ID NO: 1408 - DNA, 22 bp, Artificial Sequence
  primer
acagcggtctacaaccgactgc

SEQ ID NO: 1409 - DNA, 22 bp, Artificial Sequence
  primer
tcgcgctccagacaattgcagc

SEQ ID NO: 1410 - DNA, 22 bp, Artificial Sequence
  primer
ccggtagaccaggagtggtcat

SEQ ID NO: 1411 - DNA, 22 bp, Artificial Sequence
  primer
atctcctaacctagagccatct

SEQ ID NO: 1412 - DNA, 22 bp, Artificial Sequence
  primer
ccacatcgaatctaacaactac

SEQ ID NO: 1413 - DNA, 22 bp, Artificial Sequence
  primer
tagtcttattgaatacgtccta

SEQ ID NO: 1414 - DNA, 22 bp, Artificial Sequence
  primer
tccttaagccttggaactggcg

SEQ ID NO: 1415 - DNA, 22 bp, Artificial Sequence
  primer
ccgtgatggattgacgtagagg

SEQ ID NO: 1416 - DNA, 22 bp, Artificial Sequence
  primer
gcctggataacagatgtcttag

SEQ ID NO: 1417 - DNA, 22 bp, Artificial Sequence
  primer
ctcgacctataatcttctgcca

SEQ ID NO: 1418 - DNA, 22 bp, Artificial Sequence
  primer
agctacttctccttcctaatca

SEQ ID NO: 1419 - DNA, 22 bp, Artificial Sequence
  primer
acacgctattgccttccagtta

SEQ ID NO: 1420 - DNA, 22 bp, Artificial Sequence
  primer
aagcctgtgcatgcaatgagaa

SEQ ID NO: 1421 - DNA, 22 bp, Artificial Sequence
  primer
tcgttggttatagcacaacttc

SEQ ID NO: 1422 - DNA, 22 bp, Artificial Sequence
  primer
gcgatgccttccaacataccaa

SEQ ID NO: 1423 - DNA, 22 bp, Artificial Sequence
  primer
ccaccgttagcacgtgctacgt

SEQ ID NO: 1424 - DNA, 22 bp, Artificial Sequence
  primer
gttaccacaatgccgccatcaa

SEQ ID NO: 1425 - DNA, 22 bp, Artificial Sequence
  primer
ggtgcattaagaacgaactacc

SEQ ID NO: 1426 - DNA, 22 bp, Artificial Sequence
  primer
tccttccggataatgccgattc

SEQ ID NO: 1427 - DNA, 22 bp, Artificial Sequence
  primer
aaccgcaacttctagcggaaga

SEQ ID NO: 1428 - DNA, 22 bp, Artificial Sequence
  primer
tccttaagcagttgaacctagg

SEQ ID NO: 1429 - DNA, 22 bp, Artificial Sequence
  primer
tactaagtcagataagatcaga

SEQ ID NO: 1430 - DNA, 22 bp, Artificial Sequence
  primer
ttcgccataactagatgaatgc

SEQ ID NO: 1431 - DNA, 22 bp, Artificial Sequence
  primer
aagaagttagacgcggtggctg

SEQ ID NO: 1432 - DNA, 22 bp, Artificial Sequence
  primer
gtatctgatcgaagagcggtgg

SEQ ID NO: 1433 - DNA, 22 bp, Artificial Sequence
  primer
tcaagagctacgaagtaagtcc

SEQ ID NO: 1434 - DNA, 22 bp, Artificial Sequence
  primer
cgagtacacagcagcataccta

SEQ ID NO: 1435 - DNA, 22 bp, Artificial Sequence
  primer
ctcgataagttactctgctaga

SEQ ID NO: 1436 - DNA, 22 bp, Artificial Sequence
  primer
atggtgctggttctccgtctgt

SEQ ID NO: 1437 - DNA, 22 bp, Artificial Sequence
  primer
tcaagcggtccaaggctgagac

SEQ ID NO: 1438 - DNA, 22 bp, Artificial Sequence
  primer
tgtcctgctctgttgctaccgt

SEQ ID NO: 1439 - DNA, 22 bp, Artificial Sequence
  primer
agtcatatcgcgtcacacgttg

SEQ ID NO: 1440 - DNA, 22 bp, Artificial Sequence
  primer
ggtgaataaggacatgagaagc

SEQ ID NO: 1441 - DNA, 24 bp, Artificial Sequence
  primer
cctgatcttatctagtagagactc

SEQ ID NO: 1442 - DNA, 24 bp, Artificial Sequence
  primer
ttctgtgtaggtgtgccaatcacc

SEQ ID NO: 1443 - DNA, 24 bp, Artificial Sequence
  primer
gacttccagatgcttaagacgaca

SEQ ID NO: 1444 - DNA, 24 bp, Artificial Sequence
  primer
gtccttcgacggagaacatccgag

SEQ ID NO: 1445 - DNA, 24 bp, Artificial Sequence
  primer
cttggttagtgtaccgtcaacgtc

SEQ ID NO: 1446 - DNA, 24 bp, Artificial Sequence
  primer
aagcggcatgtgcctaatcgacgt

SEQ ID NO: 1447 - DNA, 24 bp, Artificial Sequence
  primer
cgaccgtcgttacacggaatccga

SEQ ID NO: 1448 - DNA, 24 bp, Artificial Sequence
  primer
tcgcaagtgtgccgttctgttcat

SEQ ID NO: 1449 - DNA, 24 bp, Artificial Sequence
  primer
cgtactgaagttcggagtcgccgt

SEQ ID NO: 1450 - DNA, 24 bp, Artificial Sequence
  primer
ccactacagaatggtagcagatca

SEQ ID NO: 1451 - DNA, 24 bp, Artificial Sequence
  primer
agtaggagagaggcctacacaaca

SEQ ID NO: 1452 - DNA, 24 bp, Artificial Sequence
  primer
agccaagatactcgttcggtatgg

SEQ ID NO: 1453 - DNA, 24 bp, Artificial Sequence
  primer
gttccgagtacattgaatcctggc

SEQ ID NO: 1454 - DNA, 24 bp, Artificial Sequence
  primer
aggcgtacgagttattgccagagg

SEQ ID NO: 1455 - DNA, 24 bp, Artificial Sequence
  primer
gtggcatcacacatatctcagcat

SEQ ID NO: 1456 - DNA, 24 bp, Artificial Sequence
  primer
gagaccgatatgttgatgccagaa

SEQ ID NO: 1457 - DNA, 24 bp, Artificial Sequence
  primer
caactgtagccagtcgattgctat

SEQ ID NO: 1458 - DNA, 24 bp, Artificial Sequence
  primer
tatcaatgcaatgagaggatgcag

SEQ ID NO: 1459 - DNA, 24 bp, Artificial Sequence
  primer
gtatgctcggctccaagtactgtt

SEQ ID NO: 1460 - DNA, 24 bp, Artificial Sequence
  primer
agagactcttataggcttgacgga

SEQ ID NO: 1461 - DNA, 24 bp, Artificial Sequence
  primer
acttaacagatatggatcatcgcc

SEQ ID NO: 1462 - DNA, 24 bp, Artificial Sequence
  primer
aatcagagcgagtctcgcttcagg

SEQ ID NO: 1463 - DNA, 24 bp, Artificial Sequence
  primer
accaccgaggaacaggtgcgacaa

SEQ ID NO: 1464 - DNA, 24 bp, Artificial Sequence
  primer
tggtacatgtcaaccgtaagcctg

SEQ ID NO: 1465 - DNA, 24 bp, Artificial Sequence
  primer
cgtgccgcggtgttcttgtatatg

SEQ ID NO: 1466 - DNA, 24 bp, Artificial Sequence
  primer
gacaagcgcgcgtgagacatatca

SEQ ID NO: 1467 - DNA, 24 bp, Artificial Sequence
  primer
agtgcactccgaacaagagttagt

SEQ ID NO: 1468 - DNA, 24 bp, Artificial Sequence
  primer
cctcattaccgcgttaggagtccg

SEQ ID NO: 1469 - DNA, 24 bp, Artificial Sequence
  primer
tgcttattgcttagttgctatctc

SEQ ID NO: 1470 - DNA, 24 bp, Artificial Sequence
  primer
gcgtgatcctgttctattcgttag

SEQ ID NO: 1471 - DNA, 24 bp, Artificial Sequence
  primer
ggccagaactatgacgagtataag

SEQ ID NO: 1472 - DNA, 24 bp, Artificial Sequence
  primer
gatggcgactatctaattgcaatg

SEQ ID NO: 1473 - DNA, 24 bp, Artificial Sequence
  primer
tagtaaccatagctctgtacaact

SEQ ID NO: 1474 - DNA, 24 bp, Artificial Sequence
  primer
cgtgatcgccaatacacatgtcgc

SEQ ID NO: 1475 - DNA, 24 bp, Artificial Sequence
  primer
taataacggatcgatatgcacgcg

SEQ ID NO: 1476 - DNA, 24 bp, Artificial Sequence
  primer
atcatcgcgctaatactatctgaa

SEQ ID NO: 1477 - DNA, 24 bp, Artificial Sequence
  primer
cacgtgcgtgcaggtcactagtat

SEQ ID NO: 1478 - DNA, 24 bp, Artificial Sequence
  primer
aggtccaatgccgagcgatcagaa

SEQ ID NO: 1479 - DNA, 24 bp, Artificial Sequence
  primer
cagcataacaacgagccaggtcag

SEQ ID NO: 1480 - DNA, 24 bp, Artificial Sequence
  primer
atggcgtccaatactccgacctat

SEQ ID NO: 1481 - DNA, 24 bp, Artificial Sequence
  primer
aggaacatcgtgaataatgaagac

SEQ ID NO: 1482 - DNA, 24 bp, Artificial Sequence
  primer
tctcgacgttcatgtaattaagga

SEQ ID NO: 1483 - DNA, 24 bp, Artificial Sequence
  primer
tcgcggttaaccttacttagacga

SEQ ID NO: 1484 - DNA, 24 bp, Artificial Sequence
  primer
atcatatctacggctctggcgccg

SEQ ID NO: 1485 - DNA, 24 bp, Artificial Sequence
  primer
gcagatggagaccagaggtacagg

SEQ ID NO: 1486 - DNA, 24 bp, Artificial Sequence
  primer
agacagaagattaccacgtgctat

SEQ ID NO: 1487 - DNA, 24 bp, Artificial Sequence
  primer
ccacggacaacatgccgcttaact

SEQ ID NO: 1488 - DNA, 24 bp, Artificial Sequence
  primer
cttgaagtctcaagctatgagaga

SEQ ID NO: 1489 - DNA, 24 bp, Artificial Sequence
  primer
acagcagtcgtgcttaggtcactg

SEQ ID NO: 1490 - DNA, 24 bp, Artificial Sequence
  primer
aggtgttaatgaacgtaggtgaga

SEQ ID NO: 1491 - DNA, 24 bp, Artificial Sequence
  primer
agccactatgttcaaggctgagcc

SEQ ID NO: 1492 - DNA, 24 bp, Artificial Sequence
  primer
gcaggcggtgtcgtgtgacaatga

SEQ ID NO: 1493 - DNA, 24 bp, Artificial Sequence
  primer
agccattgctacagaggttactta

SEQ ID NO: 1494 - DNA, 24 bp, Artificial Sequence
  primer
acaatcgaacctacactgagtccg

SEQ ID NO: 1495 - DNA, 24 bp, Artificial Sequence
  primer
ccgatctcaataggtaccacgaac

SEQ ID NO: 1496 - DNA, 24 bp, Artificial Sequence
  primer
gatacgtggcgctatgctaattaa

SEQ ID NO: 1497 - DNA, 24 bp, Artificial Sequence
  primer
agagagatggcacacattgacgtc

SEQ ID NO: 1498 - DNA, 24 bp, Artificial Sequence
  primer
ctcaactcatccttgtagccgatg

SEQ ID NO: 1499 - DNA, 24 bp, Artificial Sequence
  primer
gtggaataacgcgatacgactctt

SEQ ID NO: 1500 - DNA, 24 bp, Artificial Sequence
  primer
atctaccatgcgaatgctctctag

SEQ ID NO: 1501 - DNA, 24 bp, Artificial Sequence
  primer
atacgcacgcctgacacaaggacc

SEQ ID NO: 1502 - DNA, 24 bp, Artificial Sequence
  primer
gtccactctcagtgtgtagagtcc

SEQ ID NO: 1503 - DNA, 24 bp, Artificial Sequence
  primer
aatatatccagattctctgtgcag

SEQ ID NO: 1504 - DNA, 24 bp, Artificial Sequence
  primer
ccttccgccacatgttcgacaagg

SEQ ID NO: 1505 - DNA, 24 bp, Artificial Sequence
  primer
actgtgccatcatccgaggagcca

SEQ ID NO: 1506 - DNA, 24 bp, Artificial Sequence
  primer
tctatgccgctatggcgtcgtgta

SEQ ID NO: 1507 - DNA, 24 bp, Artificial Sequence
  primer
cgtaacctaaggtaatatgtctgc

SEQ ID NO: 1508 - DNA, 24 bp, Artificial Sequence
  primer
tactgaccgtatcaagattactaa

SEQ ID NO: 1509 - DNA, 24 bp, Artificial Sequence
  primer
tcatcggagcgccatacggtacgt

SEQ ID NO: 1510 - DNA, 24 bp, Artificial Sequence
  primer
gcaagaggaatgaacgaagtgatt

SEQ ID NO: 1511 - DNA, 24 bp, Artificial Sequence
  primer
ggctgattgacatcctgacttagt

SEQ ID NO: 1512 - DNA, 24 bp, Artificial Sequence
  primer
aaggcgctagattggattaacgta

SEQ ID NO: 1513 - DNA, 24 bp, Artificial Sequence
  primer
gctagctagaagaataggattcgt

SEQ ID NO: 1514 - DNA, 24 bp, Artificial Sequence
  primer
caggtgacggcctctataactcat

SEQ ID NO: 1515 - DNA, 24 bp, Artificial Sequence
  primer
caggttacacataccactatcttc

SEQ ID NO: 1516 - DNA, 24 bp, Artificial Sequence
  primer
ttgctacgtaccgtcttaatccgt

SEQ ID NO: 1517 - DNA, 24 bp, Artificial Sequence
  primer
ctcaacatgtcttgcaagcttcga

SEQ ID NO: 1518 - DNA, 24 bp, Artificial Sequence
  primer
ggtgcggtacgtagaaccagatca

SEQ ID NO: 1519 - DNA, 24 bp, Artificial Sequence
  primer
aatgctctccaagatcctgaccta

SEQ ID NO: 1520 - DNA, 24 bp, Artificial Sequence
  primer
gcttcgcaggtctggatgatggag

SEQ ID NO: 1521 - DNA, 24 bp, Artificial Sequence
  primer
acattgaccagacagcaccttgcg

SEQ ID NO: 1522 - DNA, 24 bp, Artificial Sequence
  primer
aggtatcaatgtgcttaataggcg

SEQ ID NO: 1523 - DNA, 24 bp, Artificial Sequence
  primer
tccggacacacgattagtaacgga

SEQ ID NO: 1524 - DNA, 24 bp, Artificial Sequence
  primer
tacgaagtactacagatcggtcag

SEQ ID NO: 1525 - DNA, 24 bp, Artificial Sequence
  primer
aattgtcagacgaatactgctgga

SEQ ID NO: 1526 - DNA, 24 bp, Artificial Sequence
  primer
tgaatcatgagccagaggttatgc

SEQ ID NO: 1527 - DNA, 24 bp, Artificial Sequence
  primer
cacaagacacgtcattaacatcaa

SEQ ID NO: 1528 - DNA, 24 bp, Artificial Sequence
  primer
gaatgactacattactccgccagg

SEQ ID NO: 1529 - DNA, 24 bp, Artificial Sequence
  primer
agccagagatactggaacttgact

SEQ ID NO: 1530 - DNA, 24 bp, Artificial Sequence
  primer
tatcagacacatcacaatggatac

SEQ ID NO: 1531 - DNA, 24 bp, Artificial Sequence
  primer
ctaggacaccgctagtcggttgaa

SEQ ID NO: 1532 - DNA, 24 bp, Artificial Sequence
  primer
gtataactgcgtgtcctggtgtat

SEQ ID NO: 1533 - DNA, 24 bp, Artificial Sequence
  primer
atgcaatactaaggtggacctccg

SEQ ID NO: 1534 - DNA, 24 bp, Artificial Sequence
  primer
atgcagacgcttgcgataagtcat

SEQ ID NO: 1535 - DNA, 24 bp, Artificial Sequence
  primer
ttgctcgatacacgtagaccagtg

SEQ ID NO: 1536 - DNA, 24 bp, Artificial Sequence
  primer
tactggaggacgattgtctatcat

SEQ ID NO: 1537 - DNA, 26 bp, Artificial Sequence
  primer
actaaggcacgctgattcgagcatta

SEQ ID NO: 1538 - DNA, 26 bp, Artificial Sequence
  primer
cggattctggcacgtacaagtagcag

SEQ ID NO: 1539 - DNA, 26 bp, Artificial Sequence
  primer
ttatggctccagatctagtcaccagc

SEQ ID NO: 1540 - DNA, 26 bp, Artificial Sequence
  primer
catacactccaggcatgtatgatagg

SEQ ID NO: 1541 - DNA, 26 bp, Artificial Sequence
  primer
agttgtaagccaacgagtgtagcgta

SEQ ID NO: 1542 - DNA, 26 bp, Artificial Sequence
  primer
gtatcagctccttcctctgattccgg

SEQ ID NO: 1543 - DNA, 26 bp, Artificial Sequence
  primer
aacatacagaatgtctatggtcagct

SEQ ID NO: 1544 - DNA, 26 bp, Artificial Sequence
  primer
gactcatattcatgttcagtatagag

SEQ ID NO: 1545 - DNA, 26 bp, Artificial Sequence
  primer
agagtgaacgaacgtgaccgacgctc

SEQ ID NO: 1546 - DNA, 26 bp, Artificial Sequence
  primer
aattggcgtccttgccacaacatctt

SEQ ID NO: 1547 - DNA, 26 bp, Artificial Sequence
  primer
tcgtagacgcctcgtacatccgagat

SEQ ID NO: 1548 - DNA, 26 bp, Artificial Sequence
  primer
ccggctcgtgaggcgataatcatata

SEQ ID NO: 1549 - DNA, 26 bp, Artificial Sequence
  primer
agtcctgatcacgaccacgactcacg

SEQ ID NO: 1550 - DNA, 26 bp, Artificial Sequence
  primer
ggcactcaatcctccatggagaagct

SEQ ID NO: 1551 - DNA, 26 bp, Artificial Sequence
  primer
tcatcattcctcacgttcaccggtga

SEQ ID NO: 1552 - DNA, 26 bp, Artificial Sequence
  primer
tcaactctgtgctaaccggtcgtaca

SEQ ID NO: 1553 - DNA, 26 bp, Artificial Sequence
  primer
tgttcttatgcattaatgccaggctt

SEQ ID NO: 1554 - DNA, 26 bp, Artificial Sequence
  primer
gattcacgacctcaacagcatcactc

SEQ ID NO: 1555 - DNA, 26 bp, Artificial Sequence
  primer
ggcgagttcgaccagaatgctggaca

SEQ ID NO: 1556 - DNA, 26 bp, Artificial Sequence
  primer
ttccgtatacaatgcgattaagatct

SEQ ID NO: 1557 - DNA, 26 bp, Artificial Sequence
  primer
gagtaatccgtaaccggccaacgttg

SEQ ID NO: 1558 - DNA, 26 bp, Artificial Sequence
  primer
cgcttccatcatggtacggtacgtat

SEQ ID NO: 1559 - DNA, 26 bp, Artificial Sequence
  primer
ccgtcgtggtgtgttgactggtcaac

SEQ ID NO: 1560 - DNA, 26 bp, Artificial Sequence
  primer
tattcgcatctccgtattagttgtag

SEQ ID NO: 1561 - DNA, 26 bp, Artificial Sequence
  primer
tattattgtattctaggcggtgcaac

SEQ ID NO: 1562 - DNA, 26 bp, Artificial Sequence
  primer
aggctgcctacttcctcgtcatctcg

SEQ ID NO: 1563 - DNA, 26 bp, Artificial Sequence
  primer
gtaacatacggctcatcgaatgcatc

SEQ ID NO: 1564 - DNA, 26 bp, Artificial Sequence
  primer
ttatggcacggatattaccgtacgcc

SEQ ID NO: 1565 - DNA, 26 bp, Artificial Sequence
  primer
atagcacttcctctaatgctctgctg

SEQ ID NO: 1566 - DNA, 26 bp, Artificial Sequence
  primer
tcacaggcaatagcctaatattatat

SEQ ID NO: 1567 - DNA, 26 bp, Artificial Sequence
  primer
ggcggatgttcgttaatattataagg

SEQ ID NO: 1568 - DNA, 26 bp, Artificial Sequence
  primer
tgcaatagccgttgtctctgccagcg

SEQ ID NO: 1569 - DNA, 26 bp, Artificial Sequence
  primer
tacagcgcgttggcgagtactgatag

SEQ ID NO: 1570 - DNA, 26 bp, Artificial Sequence
  primer
tgcagttagtaccttctcacgccaac

SEQ ID NO: 1571 - DNA, 26 bp, Artificial Sequence
  primer
ccattggctacctagcagactctacc

SEQ ID NO: 1572 - DNA, 26 bp, Artificial Sequence
  primer
aacagtagctcgcgtcttgctctcgt

SEQ ID NO: 1573 - DNA, 26 bp, Artificial Sequence
  primer
gcagtccatcagctctcgcttataga

SEQ ID NO: 1574 - DNA, 26 bp, Artificial Sequence
  primer
tatctctctgtcgccagcttgaccaa

SEQ ID NO: 1575 - DNA, 26 bp, Artificial Sequence
  primer
cagactgttcaagcttgctgtaggag

SEQ ID NO: 1576 - DNA, 26 bp, Artificial Sequence
  primer
taaccggaactcgttcagcaacattc

SEQ ID NO: 1577 - DNA, 26 bp, Artificial Sequence
  primer
tcaattatgcatgtcgtccgatctct

SEQ ID NO: 1578 - DNA, 26 bp, Artificial Sequence
  primer
ttgtctaagtcaacctgtggataatc

SEQ ID NO: 1579 - DNA, 26 bp, Artificial Sequence
  primer
tctaagagtggtatgaccaggagtcc

SEQ ID NO: 1580 - DNA, 26 bp, Artificial Sequence
  primer
tcgtagtactactggaacaggtaatc

SEQ ID NO: 1581 - DNA, 26 bp, Artificial Sequence
  primer
atgtcaacattctaatcatctctcgg

SEQ ID NO: 1582 - DNA, 26 bp, Artificial Sequence
  primer
agcgcgcaactgttacggtgatccga

SEQ ID NO: 1583 - DNA, 26 bp, Artificial Sequence
  primer
gcgatagaataatggtgtcacacacg

SEQ ID NO: 1584 - DNA, 26 bp, Artificial Sequence
  primer
aaggctgcgatgagaggcgtacatcg

SEQ ID NO: 1585 - DNA, 26 bp, Artificial Sequence
  primer
ggttcatggtctcagtcgtgatcgcg

SEQ ID NO: 1586 - DNA, 26 bp, Artificial Sequence
  primer
tagtgactctatgtcacctcggagcc

SEQ ID NO: 1587 - DNA, 26 bp, Artificial Sequence
  primer
atgtgatagcaatggcacctctagtc

SEQ ID NO: 1588 - DNA, 26 bp, Artificial Sequence
  primer
tcgcgaagtgtaatgcatcatccgct

SEQ ID NO: 1589 - DNA, 26 bp, Artificial Sequence
  primer
atgtggcgacgatccaagttcaacgc

SEQ ID NO: 1590 - DNA, 26 bp, Artificial Sequence
  primer
accttgtatgagtcggagtgtccggc

SEQ ID NO: 1591 - DNA, 26 bp, Artificial Sequence
  primer
acctcaagagagtagacagttgagtt

SEQ ID NO: 1592 - DNA, 26 bp, Artificial Sequence
  primer
ggtgtaatcctgtgtgcgaagctggt

SEQ ID NO: 1593 - DNA, 26 bp, Artificial Sequence
  primer
atagcggaactgtacgacgctccagt

SEQ ID NO: 1594 - DNA, 26 bp, Artificial Sequence
  primer
aagcacgagtcgaccattagcctgga

SEQ ID NO: 1595 - DNA, 26 bp, Artificial Sequence
  primer
attccggtaacatcagaaggtacaat

SEQ ID NO: 1596 - DNA, 26 bp, Artificial Sequence
  primer
gtgcaacggcagtccagtatcctggt

SEQ ID NO: 1597 - DNA, 26 bp, Artificial Sequence
  primer
ccatcttatacacggtgaccgaagat

SEQ ID NO: 1598 - DNA, 26 bp, Artificial Sequence
  primer
gcacttaatcaagcttgagtgatgct

SEQ ID NO: 1599 - DNA, 26 bp, Artificial Sequence
  primer
agtattacgtgagtacgaagatagca

SEQ ID NO: 1600 - DNA, 26 bp, Artificial Sequence
  primer
ttcttaggttaagttccttctggacc

SEQ ID NO: 1601 - DNA, 26 bp, Artificial Sequence
  primer
gtccttgctagacactgaccgttgct

SEQ ID NO: 1602 - DNA, 26 bp, Artificial Sequence
  primer
gccgctatgtgtgctgcatcctaagc

SEQ ID NO: 1603 - DNA, 26 bp, Artificial Sequence
  primer
ccatcaataacagacttatgttgtga

SEQ ID NO: 1604 - DNA, 26 bp, Artificial Sequence
  primer
cgcgtgtgcttacaagtgctaacaag

SEQ ID NO: 1605 - DNA, 26 bp, Artificial Sequence
  primer
cgatatgtgttcgcaataagagagcc

SEQ ID NO: 1606 - DNA, 26 bp, Artificial Sequence
  primer
cgcggatgtgagcggctcaattagca

SEQ ID NO: 1607 - DNA, 26 bp, Artificial Sequence
  primer
gctgcatgactatcggatggaggcat

SEQ ID NO: 1608 - DNA, 26 bp, Artificial Sequence
  primer
ctatgccgtgtatggtacgagtggcg

SEQ ID NO: 1609 - DNA, 26 bp, Artificial Sequence
  primer
ccggctggagttcattacgtaggctg

SEQ ID NO: 1610 - DNA, 26 bp, Artificial Sequence
  primer
tgtaggcctactgagctagtattaga

SEQ ID NO: 1611 - DNA, 26 bp, Artificial Sequence
  primer
ccgtcaagtgactattcttctaatct

SEQ ID NO: 1612 - DNA, 26 bp, Artificial Sequence
  primer
ggtcttacgccagagactgcgcttct

SEQ ID NO: 1613 - DNA, 26 bp, Artificial Sequence
  primer
cgaagtgtgattattaactgtaatct

SEQ ID NO: 1614 - DNA, 26 bp, Artificial Sequence
  primer
gcacgcgtggccgtaagcatcgatta

SEQ ID NO: 1615 - DNA, 26 bp, Artificial Sequence
  primer
atcctgcgtcggaacgtactatagct

SEQ ID NO: 1616 - DNA, 26 bp, Artificial Sequence
  primer
agtatcatcatatccattcgcagtac

SEQ ID NO: 1617 - DNA, 26 bp, Artificial Sequence
  primer
agtcctgacgttcatatatagactcc

SEQ ID NO: 1618 - DNA, 26 bp, Artificial Sequence
  primer
cttgcagtaatctgaatctgaaggtt

SEQ ID NO: 1619 - DNA, 26 bp, Artificial Sequence
  primer
ataacttggttccagtaacgcatagt

SEQ ID NO: 1620 - DNA, 26 bp, Artificial Sequence
  primer
gataaggatatggctgtagcgaagtg

SEQ ID NO: 1621 - DNA, 26 bp, Artificial Sequence
  primer
gtggagcgttacagacatgctgaaca

SEQ ID NO: 1622 - DNA, 26 bp, Artificial Sequence
  primer
cgcttccggcaggcgtcatataagtc

SEQ ID NO: 1623 - DNA, 26 bp, Artificial Sequence
  primer
ataacattctaacctctataagccga

SEQ ID NO: 1624 - DNA, 26 bp, Artificial Sequence
  primer
acgatctatgatccatatggacttcc

SEQ ID NO: 1625 - DNA, 26 bp, Artificial Sequence
  primer
tgaagctcagatatcatgcctcgagc

SEQ ID NO: 1626 - DNA, 26 bp, Artificial Sequence
  primer
agacttcaccgcaataactcgtagat

SEQ ID NO: 1627 - DNA, 26 bp, Artificial Sequence
  primer
agactaagacatacgccatcaccgct

SEQ ID NO: 1628 - DNA, 26 bp, Artificial Sequence
  primer
tgtagcgtgatgtatcgtaattctgt

SEQ ID NO: 1629 - DNA, 26 bp, Artificial Sequence
  primer
tgtgctattggcacctcacgctgacc

SEQ ID NO: 1630 - DNA, 26 bp, Artificial Sequence
  primer
tgtagataagtatccagcgactctct

SEQ ID NO: 1631 - DNA, 26 bp, Artificial Sequence
  primer
aattcgccaattgtgtgtaggcgcaa

SEQ ID NO: 1632 - DNA, 26 bp, Artificial Sequence
  primer
cgattatgagtacttgtagaccagct

SEQ ID NO: 1633 - DNA, 28 bp, Artificial Sequence
  primer
ttgcaagaacaacgtatctcatatgaac

SEQ ID NO: 1634 - DNA, 28 bp, Artificial Sequence
  primer
caccgtgctgttattacttggtattcgg

SEQ ID NO: 1635 - DNA, 28 bp, Artificial Sequence
  primer
cacgtgtattgttgcaccagaacgacaa

SEQ ID NO: 1636 - DNA, 28 bp, Artificial Sequence
  primer
atgcacgtaattacttccggagaagacg

SEQ ID NO: 1637 - DNA, 28 bp, Artificial Sequence
  primer
tatgttgtctgatatggttcatgtggca

SEQ ID NO: 1638 - DNA, 28 bp, Artificial Sequence
  primer
agcgcgactagttgatgccaacattgta

SEQ ID NO: 1639 - DNA, 28 bp, Artificial Sequence
  primer
ataggcaggtccaggctcggaacaagtc

SEQ ID NO: 1640 - DNA, 28 bp, Artificial Sequence
  primer
gcggtagtcggtcaagaactagaaccgt

SEQ ID NO: 1641 - DNA, 28 bp, Artificial Sequence
  primer
actatacactctagctattaggaagcat

SEQ ID NO: 1642 - DNA, 28 bp, Artificial Sequence
  primer
gatcatcttgcttctcctgtggagataa

SEQ ID NO: 1643 - DNA, 28 bp, Artificial Sequence
  primer
ctactacgagtccataactgatagcctc

SEQ ID NO: 1644 - DNA, 28 bp, Artificial Sequence
  primer
gcacagacacctgtcctatctagcagga

SEQ ID NO: 1645 - DNA, 28 bp, Artificial Sequence
  primer
aagcgaggcgcgaaggagatggaaggat

SEQ ID NO: 1646 - DNA, 28 bp, Artificial Sequence
  primer
ctgaagacgccagtctggataggtgcct

SEQ ID NO: 1647 - DNA, 28 bp, Artificial Sequence
  primer
gtaagctctgtccttcgagattgataag

SEQ ID NO: 1648 - DNA, 28 bp, Artificial Sequence
  primer
ggttagagagattattgtgcgcatccat

SEQ ID NO: 1649 - DNA, 28 bp, Artificial Sequence
  primer
ccaggaggacctatgatcttgccgccat

SEQ ID NO: 1650 - DNA, 28 bp, Artificial Sequence
  primer
actattcgagctactgtatgtgtatccg

SEQ ID NO: 1651 - DNA, 28 bp, Artificial Sequence
  primer
gacatcgcgatacgtaactccggagtgt

SEQ ID NO: 1652 - DNA, 28 bp, Artificial Sequence
  primer
ccgcaattcgtctatatattctagcata

SEQ ID NO: 1653 - DNA, 28 bp, Artificial Sequence
  primer
ctacacttgaggttgatgctcaagatca

SEQ ID NO: 1654 - DNA, 28 bp, Artificial Sequence
  primer
cgatcagttctagttcaccgcggacaat

SEQ ID NO: 1655 - DNA, 28 bp, Artificial Sequence
  primer
aagaatgatgattggccgcgaaccaagc

SEQ ID NO: 1656 - DNA, 28 bp, Artificial Sequence
  primer
cacgaccggaactagactcctaccaatt

SEQ ID NO: 1657 - DNA, 28 bp, Artificial Sequence
  primer
agttgcctgtgagtgaggctactatctc

SEQ ID NO: 1658 - DNA, 28 bp, Artificial Sequence
  primer
gattcttccgatgatcatgccactacaa

SEQ ID NO: 1659 - DNA, 28 bp, Artificial Sequence
  primer
cgctgaagtgaactatgcaagcaccgca

SEQ ID NO: 1660 - DNA, 28 bp, Artificial Sequence
  primer
attatcgtgatggtgagactgagctcgt

SEQ ID NO: 1661 - DNA, 28 bp, Artificial Sequence
  primer
cgaggccactctgagccaggtaagtatc

SEQ ID NO: 1662 - DNA, 28 bp, Artificial Sequence
  primer
tgccgaggacagccgatcacatcttcgt

SEQ ID NO: 1663 - DNA, 28 bp, Artificial Sequence
  primer
gttgacatgaaggttatcgtcgatattc

SEQ ID NO: 1664 - DNA, 28 bp, Artificial Sequence
  primer
gtggtccaggtcaagctctgatcgaatg

SEQ ID NO: 1665 - DNA, 28 bp, Artificial Sequence
  primer
ccagtccggtgtactcagacctaataac

SEQ ID NO: 1666 - DNA, 28 bp, Artificial Sequence
  primer
cgagacactgcatgagcgtagtcttatt

SEQ ID NO: 1667 - DNA, 28 bp, Artificial Sequence
  primer
gacggcttgtatacttctctacggtctg

SEQ ID NO: 1668 - DNA, 28 bp, Artificial Sequence
  primer
ttagctggatggaagccatattccgtag

SEQ ID NO: 1669 - DNA, 28 bp, Artificial Sequence
  primer
cagcctacacttgattactcaacaactc

SEQ ID NO: 1670 - DNA, 28 bp, Artificial Sequence
  primer
gtacgtagtgtcacgcgcctacgttcgt

SEQ ID NO: 1671 - DNA, 28 bp, Artificial Sequence
  primer
ctacaacttctcaatcatgcctctgttg

SEQ ID NO: 1672 - DNA, 28 bp, Artificial Sequence
  primer
cgaggacagaattcgacataaggagaga

SEQ ID NO: 1673 - DNA, 28 bp, Artificial Sequence
  primer
gccgaacgacacagtgagttgataggta

SEQ ID NO: 1674 - DNA, 28 bp, Artificial Sequence
  primer
gaacactatatgctgtcgctgtctgagg

SEQ ID NO: 1675 - DNA, 28 bp, Artificial Sequence
  primer
gttaagttcttcggcggtcatgctcatt

SEQ ID NO: 1676 - DNA, 28 bp, Artificial Sequence
  primer
ttgcttacagatcgcgtatccatagtat

SEQ ID NO: 1677 - DNA, 28 bp, Artificial Sequence
  primer
gaggaccacctctgcgaagttcactgtg

SEQ ID NO: 1678 - DNA, 28 bp, Artificial Sequence
  primer
aatcctagcatatcgagaacgacactga

SEQ ID NO: 1679 - DNA, 28 bp, Artificial Sequence
  primer
tgaatactatagccatagtcgacttccg

SEQ ID NO: 1680 - DNA, 28 bp, Artificial Sequence
  primer
gacatccacgaagctggtaatcggaacc

SEQ ID NO: 1681 - DNA, 28 bp, Artificial Sequence
  primer
ttagccgtcttagaagtgtctgaccggc

SEQ ID NO: 1682 - DNA, 28 bp, Artificial Sequence
  primer
ctattctgccgtaattgattccttcgtt

SEQ ID NO: 1683 - DNA, 28 bp, Artificial Sequence
  primer
acgcctctggtcgaaggtagattagctc

SEQ ID NO: 1684 - DNA, 28 bp, Artificial Sequence
  primer
cagcctattgatcgtaagtagatggtcc

SEQ ID NO: 1685 - DNA, 28 bp, Artificial Sequence
  primer
ttaagtgaggtggacaaccatcaacttc

SEQ ID NO: 1686 - DNA, 28 bp, Artificial Sequence
  primer
aaggccttgcggctaagtagtattcatc

SEQ ID NO: 1687 - DNA, 28 bp, Artificial Sequence
  primer
ttgtgatactaattcttctcaagagtca

SEQ ID NO: 1688 - DNA, 28 bp, Artificial Sequence
  primer
gcattaggtgacgaccttagtccatcac

SEQ ID NO: 1689 - DNA, 28 bp, Artificial Sequence
  primer
gcggatggacgtatacagtgagtcgtgc

SEQ ID NO: 1690 - DNA, 28 bp, Artificial Sequence
  primer
gaacatgccagcctcaactaggctaaga

SEQ ID NO: 1691 - DNA, 28 bp, Artificial Sequence
  primer
tccgtcattagagtatgagtgactacta

SEQ ID NO: 1692 - DNA, 28 bp, Artificial Sequence
  primer
aacacttagtaaccagttcggactggac

SEQ ID NO: 1693 - DNA, 28 bp, Artificial Sequence
  primer
cgctaactattgcgtatattcgcggctt

SEQ ID NO: 1694 - DNA, 28 bp, Artificial Sequence
  primer
gccatctacgatcttcggcttatcctag

SEQ ID NO: 1695 - DNA, 28 bp, Artificial Sequence
  primer
cctgagaatgttgactaagatcttgtga

SEQ ID NO: 1696 - DNA, 28 bp, Artificial Sequence
  primer
tcggttagtctaatcatcacgcaacgga

SEQ ID NO: 1697 - DNA, 28 bp, Artificial Sequence
  primer
attatctattgaagcagtgacagcgatc

SEQ ID NO: 1698 - DNA, 28 bp, Artificial Sequence
  primer
gaggagaatcacggaacacggtcacatg

SEQ ID NO: 1699 - DNA, 28 bp, Artificial Sequence
  primer
gctgcaagcattatgaccatggcatctg

SEQ ID NO: 1700 - DNA, 28 bp, Artificial Sequence
  primer
gaacaacctataacgacgttgtggacaa

SEQ ID NO: 1701 - DNA, 28 bp, Artificial Sequence
  primer
ttaatcatcgatagacgacatggaatca

SEQ ID NO: 1702 - DNA, 28 bp, Artificial Sequence
  primer
tcgagtgtaagcacactacgatctggaa

SEQ ID NO: 1703 - DNA, 28 bp, Artificial Sequence
  primer
gctacgcacagtctctgcacagctacac

SEQ ID NO: 1704 - DNA, 28 bp, Artificial Sequence
  primer
cctgtatgtacgttctggctaatacctt

SEQ ID NO: 1705 - DNA, 28 bp, Artificial Sequence
  primer
tgaagcaccggtacatggtgtatccgga

SEQ ID NO: 1706 - DNA, 28 bp, Artificial Sequence
  primer
tgctggaacctaactcggtgatgacgat

SEQ ID NO: 1707 - DNA, 28 bp, Artificial Sequence
  primer
cgctatcttactgccaagttctcatata

SEQ ID NO: 1708 - DNA, 28 bp, Artificial Sequence
  primer
aacgcgcgcgtatcggcaataatctcaa

SEQ ID NO: 1709 - DNA, 28 bp, Artificial Sequence
  primer
ccattaggatgaccatcgactattagag

SEQ ID NO: 1710 - DNA, 28 bp, Artificial Sequence
  primer
tactgctagactgcgtgcattcatggcg

SEQ ID NO: 1711 - DNA, 28 bp, Artificial Sequence
  primer
cattgcgcgctccacgaactctattgtc

SEQ ID NO: 1712 - DNA, 28 bp, Artificial Sequence
  primer
gacgcgcctagaactgtatagctctacg

SEQ ID NO: 1713 - DNA, 28 bp, Artificial Sequence
  primer
cattgcaacttgtcggtgatggcaatcc

SEQ ID NO: 1714 - DNA, 28 bp, Artificial Sequence
  primer
ttaatgcacatgcagtacggcaccacag

SEQ ID NO: 1715 - DNA, 28 bp, Artificial Sequence
  primer
agcggtacgtggacgagtggtaattaat

SEQ ID NO: 1716 - DNA, 28 bp, Artificial Sequence
  primer
gacgtattgctatgcattggaagatgct

SEQ ID NO: 1717 - DNA, 28 bp, Artificial Sequence
  primer
aacacttcgaccattgcgcctcaatggt

SEQ ID NO: 1718 - DNA, 28 bp, Artificial Sequence
  primer
cggtacgctctagcggtcataagatgca

SEQ ID NO: 1719 - DNA, 28 bp, Artificial Sequence
  primer
cctgaataacagccgcgcctaattagat

SEQ ID NO: 1720 - DNA, 28 bp, Artificial Sequence
  primer
aagcgtctaatgtgccttaagtcacatg

SEQ ID NO: 1721 - DNA, 28 bp, Artificial Sequence
  primer
gctctccaagaaccagaagtaagcatcg

SEQ ID NO: 1722 - DNA, 28 bp, Artificial Sequence
  primer
gaggagagttgtccgagtggtgtgatgt

SEQ ID NO: 1723 - DNA, 28 bp, Artificial Sequence
  primer
taacgagtggtgcgtctaagcaattgag

SEQ ID NO: 1724 - DNA, 28 bp, Artificial Sequence
  primer
ccaacagtatgctgacataactatgata

SEQ ID NO: 1725 - DNA, 28 bp, Artificial Sequence
  primer
gatccttgccacgcctatgagatatcgc

SEQ ID NO: 1726 - DNA, 28 bp, Artificial Sequence
  primer
aacgcgctaccgtccttgtgcatagagg

SEQ ID NO: 1727 - DNA, 28 bp, Artificial Sequence
  primer
ctacatgtgccttatagtacagaggaac

SEQ ID NO: 1728 - DNA, 28 bp, Artificial Sequence
  primer
cagcctcgtagttagcgtgattcatgcg

SEQ ID NO: 1729 - DNA, 29 bp, Artificial Sequence
  primer
ctcctcgccgattgaagtgcgtagaacta

SEQ ID NO: 1730 - DNA, 29 bp, Artificial Sequence
  primer
cagcaggcctcaataggataagccaacta

SEQ ID NO: 1731 - DNA, 29 bp, Artificial Sequence
  primer
gaccatcaatctcgaagactacgctctgt

SEQ ID NO: 1732 - DNA, 29 bp, Artificial Sequence
  primer
ggttgctccgtctgttcagcacactgtta

SEQ ID NO: 1733 - DNA, 29 bp, Artificial Sequence
  primer
aatgtcgactggccattatcgccaagtgt

SEQ ID NO: 1734 - DNA, 29 bp, Artificial Sequence
  primer
gatagcttgccatgcgaatggatctccag

SEQ ID NO: 1735 - DNA, 29 bp, Artificial Sequence
  primer
ccagaccggagccaattggctgccaatat

SEQ ID NO: 1736 - DNA, 29 bp, Artificial Sequence
  primer
aacgtcgctccatacgttacctaatgcag

SEQ ID NO: 1737 - DNA, 29 bp, Artificial Sequence
  primer
gaatatgacgcgaacagtctattcggatc

SEQ ID NO: 1738 - DNA, 29 bp, Artificial Sequence
  primer
gacgagaatgtattaaggataagcaaggt

SEQ ID NO: 1739 - DNA, 29 bp, Artificial Sequence
  primer
aagtcgtatgaatcgctatcacatgagtc

SEQ ID NO: 1740 - DNA, 29 bp, Artificial Sequence
  primer
gtcgtggagactacaattctcctcacgtt

SEQ ID NO: 1741 - DNA, 29 bp, Artificial Sequence
  primer
gttgccaccgttacacgactatcgacagt

SEQ ID NO: 1742 - DNA, 29 bp, Artificial Sequence
  primer
aggataggctacgccttactctcctaagc

SEQ ID NO: 1743 - DNA, 29 bp, Artificial Sequence
  primer
taatcatcctgttcgcctcgaggttgtta

SEQ ID NO: 1744 - DNA, 29 bp, Artificial Sequence
  primer
gacaagcagtaataattactgagtggacg

SEQ ID NO: 1745 - DNA, 29 bp, Artificial Sequence
  primer
tacagcgttacgcaggtatatcaaggtag

SEQ ID NO: 1746 - DNA, 29 bp, Artificial Sequence
  primer
ctaacatcacttactattagcggtctcgt

SEQ ID NO: 1747 - DNA, 29 bp, Artificial Sequence
  primer
ccgcgcttcttgacacgttctccactagg

SEQ ID NO: 1748 - DNA, 29 bp, Artificial Sequence
  primer
caagtaacatgagatgctatcggtacatt

SEQ ID NO: 1749 - DNA, 29 bp, Artificial Sequence
  primer
cgaccactaggctgtgaccacgatacgct

SEQ ID NO: 1750 - DNA, 29 bp, Artificial Sequence
  primer
caggtcatgtgacgcagtcggcagtcaac

SEQ ID NO: 1751 - DNA, 29 bp, Artificial Sequence
  primer
actccatcgttagttcttccgccgtgctg

SEQ ID NO: 1752 - DNA, 29 bp, Artificial Sequence
  primer
ctcaccacgtatgcgtcactcggttacgt

SEQ ID NO: 1753 - DNA, 29 bp, Artificial Sequence
  primer
tgcctatgctatggaccttgcgcgactct

SEQ ID NO: 1754 - DNA, 29 bp, Artificial Sequence
  primer
aatgaaggtcaacgctctgtagttacgcg

SEQ ID NO: 1755 - DNA, 29 bp, Artificial Sequence
  primer
caccattgattcatggcttccatcactgc

SEQ ID NO: 1756 - DNA, 29 bp, Artificial Sequence
  primer
gacacgcaaggtaattcgagattgcagca

SEQ ID NO: 1757 - DNA, 29 bp, Artificial Sequence
  primer
caccgagaggaaggttcgatcgcttctcg

SEQ ID NO: 1758 - DNA, 29 bp, Artificial Sequence
  primer
cagttatcggattgtgatattcactcctg

SEQ ID NO: 1759 - DNA, 29 bp, Artificial Sequence
  primer
atactgtaacgcctcaacctatgctgact

SEQ ID NO: 1760 - DNA, 29 bp, Artificial Sequence
  primer
atctgtcttattctggcacactcagactt

SEQ ID NO: 1761 - DNA, 29 bp, Artificial Sequence
  primer
tccaaccggtgacgtgctcttgatccaac

SEQ ID NO: 1762 - DNA, 29 bp, Artificial Sequence
  primer
cacactcagttcggctatctctgcgatag

SEQ ID NO: 1763 - DNA, 29 bp, Artificial Sequence
  primer
agctgtaagtcaggtctacgactcgtact

SEQ ID NO: 1764 - DNA, 29 bp, Artificial Sequence
  primer
gtcggcggcacgcacagctaacattcgta

SEQ ID NO: 1765 - DNA, 29 bp, Artificial Sequence
  primer
atatggtagccagccacgtatactgaaca

SEQ ID NO: 1766 - DNA, 29 bp, Artificial Sequence
  primer
tggacaatccgactctaacacagaggtag

SEQ ID NO: 1767 - DNA, 29 bp, Artificial Sequence
  primer
tccgccgctgacagttcaatctatcaatt

SEQ ID NO: 1768 - DNA, 29 bp, Artificial Sequence
  primer
ggttccttagaatatgcacctatcagcga

SEQ ID NO: 1769 - DNA, 29 bp, Artificial Sequence
  primer
cggctgtacgacatggatcataagagtgt

SEQ ID NO: 1770 - DNA, 29 bp, Artificial Sequence
  primer
tgcagatgtacgctgtggccagtggagag

SEQ ID NO: 1771 - DNA, 29 bp, Artificial Sequence
  primer
cctactcacttaacaataatcggttcggt

SEQ ID NO: 1772 - DNA, 29 bp, Artificial Sequence
  primer
cgcttcctactgcctgtgccgcgacataa

SEQ ID NO: 1773 - DNA, 29 bp, Artificial Sequence
  primer
ctagaccgaccggttatgcgctattgttc

SEQ ID NO: 1774 - DNA, 29 bp, Artificial Sequence
  primer
ttgtgagcacgtctgcggcaagcctatgg

SEQ ID NO: 1775 - DNA, 29 bp, Artificial Sequence
  primer
tcatcggccggcgctgttgttgttaccat

SEQ ID NO: 1776 - DNA, 29 bp, Artificial Sequence
  primer
gcggttaggtgcagttaggaagactatca

SEQ ID NO: 1777 - DNA, 29 bp, Artificial Sequence
  primer
tatgcggtcgtgaggcgtagcattctaga

SEQ ID NO: 1778 - DNA, 29 bp, Artificial Sequence
  primer
ccatctattcgtcgaactctcagctcgta

SEQ ID NO: 1779 - DNA, 29 bp, Artificial Sequence
  primer
atcagatctactgatcgcggtagagtatc

SEQ ID NO: 1780 - DNA, 29 bp, Artificial Sequence
  primer
tacacataggcggcgcagccttctaatta

SEQ ID NO: 1781 - DNA, 29 bp, Artificial Sequence
  primer
ttaaccgtagttcttagcttacgccgctc

SEQ ID NO: 1782 - DNA, 29 bp, Artificial Sequence
  primer
actatagaggacatggcactcctcttcta

SEQ ID NO: 1783 - DNA, 29 bp, Artificial Sequence
  primer
cagttcgtattaagattgaatgtagcggt

SEQ ID NO: 1784 - DNA, 29 bp, Artificial Sequence
  primer
agttatcggtatccgcttatccgtacgta

SEQ ID NO: 1785 - DNA, 29 bp, Artificial Sequence
  primer
agcttattcatacactgcaccacagcaag

SEQ ID NO: 1786 - DNA, 29 bp, Artificial Sequence
  primer
ccgtcggctagtctatcctctaattagaa

SEQ ID NO: 1787 - DNA, 29 bp, Artificial Sequence
  primer
gtccgcttccatgcctgctgtacgaacac

SEQ ID NO: 1788 - DNA, 29 bp, Artificial Sequence
  primer
tctcttcctccttcattgttcgctagctc

SEQ ID NO: 1789 - DNA, 29 bp, Artificial Sequence
  primer
tctcttgagcggtcctcatacaggtctgc

SEQ ID NO: 1790 - DNA, 29 bp, Artificial Sequence
  primer
gaccaagtgtaggtgatatcaccggtact

SEQ ID NO: 1791 - DNA, 29 bp, Artificial Sequence
  primer
aagattgtgataggttggtagttaccaca

SEQ ID NO: 1792 - DNA, 29 bp, Artificial Sequence
  primer
tcgcctccgaagagtatagcatcggcaga

SEQ ID NO: 1793 - DNA, 29 bp, Artificial Sequence
  primer
gaggtagttatgagcatcgaggtcctgtt

SEQ ID NO: 1794 - DNA, 29 bp, Artificial Sequence
  primer
ggacgcaagatcgcaggtacttgtaagct

SEQ ID NO: 1795 - DNA, 29 bp, Artificial Sequence
  primer
actcgtacacgtcatcgtgcaggtctcag

SEQ ID NO: 1796 - DNA, 29 bp, Artificial Sequence
  primer
taatccgtcaggagtgagatggctcgaca

SEQ ID NO: 1797 - DNA, 29 bp, Artificial Sequence
  primer
aagatggttccgcgcattgactagcaagt

SEQ ID NO: 1798 - DNA, 29 bp, Artificial Sequence
  primer
tccgcgatctgcggatcttgaatgctcac

SEQ ID NO: 1799 - DNA, 29 bp, Artificial Sequence
  primer
ttcacgagagtcaactgctagtatcctag

SEQ ID NO: 1800 - DNA, 29 bp, Artificial Sequence
  primer
ttccaactggattcttccaactcctcgaa

SEQ ID NO: 1801 - DNA, 29 bp, Artificial Sequence
  primer
cactactactcaagttatacggtgttgac

SEQ ID NO: 1802 - DNA, 29 bp, Artificial Sequence
  primer
caactggattctcaggatgcgtctctagc

SEQ ID NO: 1803 - DNA, 29 bp, Artificial Sequence
  primer
tggactagagtggagcgattacgtaatat

SEQ ID NO: 1804 - DNA, 29 bp, Artificial Sequence
  primer
gaggtcattcaactggactcgccacggac

SEQ ID NO: 1805 - DNA, 29 bp, Artificial Sequence
  primer
caggtgtgtaacgctgcaatcacatgaat

SEQ ID NO: 1806 - DNA, 29 bp, Artificial Sequence
  primer
tatgctgaggtattagttctaactatgcg

SEQ ID NO: 1807 - DNA, 29 bp, Artificial Sequence
  primer
cgtctgagtcggataaggaaggttaccgc

SEQ ID NO: 1808 - DNA, 29 bp, Artificial Sequence
  primer
gtactatcgtcgcaggcactatctctgcc

SEQ ID NO: 1809 - DNA, 29 bp, Artificial Sequence
  primer
gcttcctccttgcaacttcattgcttcga

SEQ ID NO: 1810 - DNA, 29 bp, Artificial Sequence
  primer
tgtctacgaagtagaagacacgaataatg

SEQ ID NO: 1811 - DNA, 29 bp, Artificial Sequence
  primer
ccgtcatctaaggcagagtacatccgcga

SEQ ID NO: 1812 - DNA, 29 bp, Artificial Sequence
  primer
ccggaggcgtactaactgaccacaacacc

SEQ ID NO: 1813 - DNA, 29 bp, Artificial Sequence
  primer
aactcgtcgctgcctgaataggtcagagt

SEQ ID NO: 1814 - DNA, 29 bp, Artificial Sequence
  primer
ttataagattaatgtcggtcagtgtcgga

SEQ ID NO: 1815 - DNA, 29 bp, Artificial Sequence
  primer
cgtctcgatggatccacacgaacctgttg

SEQ ID NO: 1816 - DNA, 29 bp, Artificial Sequence
  primer
atgccatcatggtcgtcctatcttaaggc

SEQ ID NO: 1817 - DNA, 29 bp, Artificial Sequence
  primer
gcgcttcagcgattcgtcatgcaaggcac

SEQ ID NO: 1818 - DNA, 29 bp, Artificial Sequence
  primer
ccaagcgataccgaggtacggttaacgag

SEQ ID NO: 1819 - DNA, 29 bp, Artificial Sequence
  primer
atatgacagacaggtggacctaagcaagc

SEQ ID NO: 1820 - DNA, 29 bp, Artificial Sequence
  primer
cactacatcgtcaggcctggaagcctcag

SEQ ID NO: 1821 - DNA, 29 bp, Artificial Sequence
  primer
gccgtgtagacgaggacattatgtcgtat

SEQ ID NO: 1822 - DNA, 29 bp, Artificial Sequence
  primer
caacgtatatacacaccttgtgaagagaa

SEQ ID NO: 1823 - DNA, 29 bp, Artificial Sequence
  primer
tccaacgtaattccgccgtctgtcgagac

SEQ ID NO: 1824 - DNA, 29 bp, Artificial Sequence
  primer
aattcgtgcttcgatcaccgtagactcag

SEQ ID NO: 1825 - DNA, 30 bp, Artificial Sequence
  primer
actatattgtattcacgtccgacgactcgc

SEQ ID NO: 1826 - DNA, 30 bp, Artificial Sequence
  primer
gacgagcttgtggtacactatacctatgag

SEQ ID NO: 1827 - DNA, 30 bp, Artificial Sequence
  primer
tgattcaagcaccaggcatgcttaagctag

SEQ ID NO: 1828 - DNA, 30 bp, Artificial Sequence
  primer
cggtctcctataggaaggctcattctgacg

SEQ ID NO: 1829 - DNA, 30 bp, Artificial Sequence
  primer
agtcagtgtcgaatcaatcaaggcgtcctt

SEQ ID NO: 1830 - DNA, 30 bp, Artificial Sequence
  primer
cgaacgtaatggccatcacgcgctggccta

SEQ ID NO: 1831 - DNA, 30 bp, Artificial Sequence
  primer
cgaacctggaccacctggcattaccattac

SEQ ID NO: 1832 - DNA, 30 bp, Artificial Sequence
  primer
acattaggttcctgtaatgtcttatcaacg

SEQ ID NO: 1833 - DNA, 30 bp, Artificial Sequence
  primer
cgtctaatgcaccgtatcgtcttcgcgcat

SEQ ID NO: 1834 - DNA, 30 bp, Artificial Sequence
  primer
tctatgacttacaacggaatcttacttcgt

SEQ ID NO: 1835 - DNA, 30 bp, Artificial Sequence
  primer
gtaaccgatcggtaccgtctgctattgttc

SEQ ID NO: 1836 - DNA, 30 bp, Artificial Sequence
  primer
ggtgattgataagcaacacatattaggagg

SEQ ID NO: 1837 - DNA, 30 bp, Artificial Sequence
  primer
aattatcgacgctaataggcgagctgttca

SEQ ID NO: 1838 - DNA, 30 bp, Artificial Sequence
  primer
ggaggtacatgacgagtggacagacagacc

SEQ ID NO: 1839 - DNA, 30 bp, Artificial Sequence
  primer
ctctaatccgttatgcggtgatgtaatccg

SEQ ID NO: 1840 - DNA, 30 bp, Artificial Sequence
  primer
gcaagcacgcggcttggcgaacttctatgc

SEQ ID NO: 1841 - DNA, 30 bp, Artificial Sequence
  primer
tagatgtaggcctggtaggcagaggagtaa

SEQ ID NO: 1842 - DNA, 30 bp, Artificial Sequence
  primer
ccgagtggcgaccacacaggtacgcattaa

SEQ ID NO: 1843 - DNA, 30 bp, Artificial Sequence
  primer
gtcctggctcagattagtgcacttagttat

SEQ ID NO: 1844 - DNA, 30 bp, Artificial Sequence
  primer
gcggtacctacatgttatgactcagacgac

SEQ ID NO: 1845 - DNA, 30 bp, Artificial Sequence
  primer
tctctgccaatgctggtctcatcgaatcca

SEQ ID NO: 1846 - DNA, 30 bp, Artificial Sequence
  primer
tctctacacagctacatactatactgtaac

SEQ ID NO: 1847 - DNA, 30 bp, Artificial Sequence
  primer
tacgacggacgctggtggtgtaagagaagg

SEQ ID NO: 1848 - DNA, 30 bp, Artificial Sequence
  primer
gcctcgatatatctacgtatagttcaagtt

SEQ ID NO: 1849 - DNA, 30 bp, Artificial Sequence
  primer
ggctcctgcattcattgaaggtcggccttg

SEQ ID NO: 1850 - DNA, 30 bp, Artificial Sequence
  primer
cagttcggtgattcaagagaacaatggtgg

SEQ ID NO: 1851 - DNA, 30 bp, Artificial Sequence
  primer
tataacgaagccggctggaacggtaactca

SEQ ID NO: 1852 - DNA, 30 bp, Artificial Sequence
  primer
ctgtatcaattcaagtgacagtggcacgtc

SEQ ID NO: 1853 - DNA, 30 bp, Artificial Sequence
  primer
agcaattgcggttcataggcgtaattatat

SEQ ID NO: 1854 - DNA, 30 bp, Artificial Sequence
  primer
catatggacctggagatcaccgttcagtcc

SEQ ID NO: 1855 - DNA, 30 bp, Artificial Sequence
  primer
gaaggccgttggtctatctcttactggagc

SEQ ID NO: 1856 - DNA, 30 bp, Artificial Sequence
  primer
gtgcgttcatctagcctaagacgctgacct

SEQ ID NO: 1857 - DNA, 30 bp, Artificial Sequence
  primer
gagtaacttatatcctctctacgacatcga

SEQ ID NO: 1858 - DNA, 30 bp, Artificial Sequence
  primer
attctacgctgatgtctccgctgaacagga

SEQ ID NO: 1859 - DNA, 30 bp, Artificial Sequence
  primer
tcatcaacgttactcactagtaccacggct

SEQ ID NO: 1860 - DNA, 30 bp, Artificial Sequence
  primer
aaccattcttgaacgttgagaacctggtgg

SEQ ID NO: 1861 - DNA, 30 bp, Artificial Sequence
  primer
acgacacctccgcggaacatacctgattag

SEQ ID NO: 1862 - DNA, 30 bp, Artificial Sequence
  primer
gcgcacttattgaagtaatctcatggccaa

SEQ ID NO: 1863 - DNA, 30 bp, Artificial Sequence
  primer
gcgccaattcagccagttagcgtctccgtg

SEQ ID NO: 1864 - DNA, 30 bp, Artificial Sequence
  primer
agcaacaagtcgctgtatatcgactggccg

SEQ ID NO: 1865 - DNA, 30 bp, Artificial Sequence
  primer
ccttacaatagacctcgcggcgttcatgcc

SEQ ID NO: 1866 - DNA, 30 bp, Artificial Sequence
  primer
ggatccaacttcagcgaagcaccaacgtcg

SEQ ID NO: 1867 - DNA, 30 bp, Artificial Sequence
  primer
gcgccagttctcgtactctcgagaagcgac

SEQ ID NO: 1868 - DNA, 30 bp, Artificial Sequence
  primer
gagtgcggccaatctggaactcatgacgtt

SEQ ID NO: 1869 - DNA, 30 bp, Artificial Sequence
  primer
cctgagagtgattcgtgtctgcgaagatgc

SEQ ID NO: 1870 - DNA, 30 bp, Artificial Sequence
  primer
gtgactggttaaggcaatattggtcgaccg

SEQ ID NO: 1871 - DNA, 30 bp, Artificial Sequence
  primer
ctatcaagccttacaaggtcacgtccacta

SEQ ID NO: 1872 - DNA, 30 bp, Artificial Sequence
  primer
actgcgtccttgcgtcggaactccttgtgt

SEQ ID NO: 1873 - DNA, 30 bp, Artificial Sequence
  primer
tgcaactcagtggcggcgacaccaagagct

SEQ ID NO: 1874 - DNA, 30 bp, Artificial Sequence
  primer
ttcggttctactaggatctctatctgagct

SEQ ID NO: 1875 - DNA, 30 bp, Artificial Sequence
  primer
agctaatctattaagacagattagacagga

SEQ ID NO: 1876 - DNA, 30 bp, Artificial Sequence
  primer
ggaccgctcttaggttatgcacctgcgtat

SEQ ID NO: 1877 - DNA, 30 bp, Artificial Sequence
  primer
ctctaatactagtccacaggttagtacgaa

SEQ ID NO: 1878 - DNA, 30 bp, Artificial Sequence
  primer
atccatatatgctcgtcgtcagccagtgtt

SEQ ID NO: 1879 - DNA, 30 bp, Artificial Sequence
  primer
gctattactgtgttgatgtccacaggagaa

SEQ ID NO: 1880 - DNA, 30 bp, Artificial Sequence
  primer
gctacggcgcagatctagacaactggaagt

SEQ ID NO: 1881 - DNA, 30 bp, Artificial Sequence
  primer
gcctcttgtgttagccgaataccaatgacc

SEQ ID NO: 1882 - DNA, 30 bp, Artificial Sequence
  primer
tgaggacgataacattacctctcgagtcgc

SEQ ID NO: 1883 - DNA, 30 bp, Artificial Sequence
  primer
cgattaccaatccgacgacttcgcagcagc

SEQ ID NO: 1884 - DNA, 30 bp, Artificial Sequence
  primer
atgacacgagtccagtacatatgcgaagac

SEQ ID NO: 1885 - DNA, 30 bp, Artificial Sequence
  primer
gcgctcgcatgcactagtgtagactgacga

SEQ ID NO: 1886 - DNA, 30 bp, Artificial Sequence
  primer
gcacatctcagaattgatggtctatgtcgc

SEQ ID NO: 1887 - DNA, 30 bp, Artificial Sequence
  primer
ttcttcgacgccgcgtactaataggtcaat

SEQ ID NO: 1888 - DNA, 30 bp, Artificial Sequence
  primer
ggaagcgcctctaacaaccgatgcttgtgg

SEQ ID NO: 1889 - DNA, 30 bp, Artificial Sequence
  primer
ctctagacgcgtcgtgactccaatctgttg

SEQ ID NO: 1890 - DNA, 30 bp, Artificial Sequence
  primer
gtagttcgtcggagtgacctcgtactcact

SEQ ID NO: 1891 - DNA, 30 bp, Artificial Sequence
  primer
atgctgtcgagtgtccggcatagagcacac

SEQ ID NO: 1892 - DNA, 30 bp, Artificial Sequence
  primer
gcgcatcttgcagcgtcctgtagttctgaa

SEQ ID NO: 1893 - DNA, 30 bp, Artificial Sequence
  primer
gcgattgttgaggaaccacagcggcaccta

SEQ ID NO: 1894 - DNA, 30 bp, Artificial Sequence
  primer
cacgcgtactctgcttgctgtgtggtcggt

SEQ ID NO: 1895 - DNA, 30 bp, Artificial Sequence
  primer
catccaacgcaggacctagtagtcatgctt

SEQ ID NO: 1896 - DNA, 30 bp, Artificial Sequence
  primer
ttctagttgtgatgagaatcgctagcgtgc

SEQ ID NO: 1897 - DNA, 30 bp, Artificial Sequence
  primer
cattctgaatctggtctctctcgatcatcc

SEQ ID NO: 1898 - DNA, 30 bp, Artificial Sequence
  primer
attaatgtagaggatagttccgttctctcc

SEQ ID NO: 1899 - DNA, 30 bp, Artificial Sequence
  primer
gtatcgcgcttacgaatgaggtgtggcttc

SEQ ID NO: 1900 - DNA, 30 bp, Artificial Sequence
  primer
gctggtgagagagccagattatcggtggag

SEQ ID NO: 1901 - DNA, 30 bp, Artificial Sequence
  primer
ggcacgagcaggtagaactagaacctagat

SEQ ID NO: 1902 - DNA, 30 bp, Artificial Sequence
  primer
tgtattatctcgaagcggtgcgttagagtc

SEQ ID NO: 1903 - DNA, 30 bp, Artificial Sequence
  primer
cacgtgttctagctactaatggcgtcaatt

SEQ ID NO: 1904 - DNA, 30 bp, Artificial Sequence
  primer
cgcgctacattacttcctacaccatgcgta

SEQ ID NO: 1905 - DNA, 30 bp, Artificial Sequence
  primer
tgaggcaactagtgttcgcaagatgacgga

SEQ ID NO: 1906 - DNA, 30 bp, Artificial Sequence
  primer
ttattattgtctgtggaacgcacgccagtc

SEQ ID NO: 1907 - DNA, 30 bp, Artificial Sequence
  primer
gctatagtattatccatgaattccgtcggc

SEQ ID NO: 1908 - DNA, 30 bp, Artificial Sequence
  primer
gtatcaatagctcaattcgtcagagttgtg

SEQ ID NO: 1909 - DNA, 30 bp, Artificial Sequence
  primer
tagtccatgcgtggatatattgagagctga

SEQ ID NO: 1910 - DNA, 30 bp, Artificial Sequence
  primer
gcacagtacgacttataacaggtctagatc

SEQ ID NO: 1911 - DNA, 30 bp, Artificial Sequence
  primer
actcaatggtggcacgctcggcgcagcata

SEQ ID NO: 1912 - DNA, 30 bp, Artificial Sequence
  primer
gtagtaccactccgccttaggcagcttaag

SEQ ID NO: 1913 - DNA, 30 bp, Artificial Sequence
  primer
cgctcaactgatgcgtgcaaccaatgttat

SEQ ID NO: 1914 - DNA, 30 bp, Artificial Sequence
  primer
gcagcttgactgcctagacagcagttacag

SEQ ID NO: 1915 - DNA, 30 bp, Artificial Sequence
  primer
gcaacttcttagtacgaattcatcgtccaa

SEQ ID NO: 1916 - DNA, 30 bp, Artificial Sequence
  primer
atccgtatgctgcggcagtggaggtggctt

SEQ ID NO: 1917 - DNA, 30 bp, Artificial Sequence
  primer
tgcggatcaatccagttctgtgtactgtga

SEQ ID NO: 1918 - DNA, 30 bp, Artificial Sequence
  primer
ttatgattatcaccggcgtaacattccgaa

SEQ ID NO: 1919 - DNA, 30 bp, Artificial Sequence
  primer
gctacctagattcttcaactcatcgctacc

SEQ ID NO: 1920 - DNA, 30 bp, Artificial Sequence
  primer
cagtgttagaatggcggtgtgtagccgcta

SEQ ID NO: 1921 - DNA, 35 bp, Artificial Sequence
  primer
gcttatagactacagctgcgaggtataaggtcact

SEQ ID NO: 1922 - DNA, 35 bp, Artificial Sequence
  primer
cgctcagcaggatgctatcctaagttaatgtggtg

SEQ ID NO: 1923 - DNA, 35 bp, Artificial Sequence
  primer
gaactgagcggacatcagctaggcctacaatacat

SEQ ID NO: 1924 - DNA, 35 bp, Artificial Sequence
  primer
tcgtgaacttctgcgttggtctctaccaaggcggt

SEQ ID NO: 1925 - DNA, 35 bp, Artificial Sequence
  primer
taagtcaggtatcttatcagtggtacacggtacga

SEQ ID NO: 1926 - DNA, 35 bp, Artificial Sequence
  primer
taataatgttgcgcgtgaccgaggaggaatccact

SEQ ID NO: 1927 - DNA, 35 bp, Artificial Sequence
  primer
ctaggagttctcgtaagctggagtaccgtaacgtg

SEQ ID NO: 1928 - DNA, 35 bp, Artificial Sequence
  primer
ggactctcctcagaggatccttcttgcgcaggcat

SEQ ID NO: 1929 - DNA, 35 bp, Artificial Sequence
  primer
gctagaggcctgagtacaccttctcgcatcaggat

SEQ ID NO: 1930 - DNA, 35 bp, Artificial Sequence
  primer
atatcgcgagcactaacgtcgttgtcgttctagga

SEQ ID NO: 1931 - DNA, 35 bp, Artificial Sequence
  primer
agcggttactatacctggcggctgacgttgttagt

SEQ ID NO: 1932 - DNA, 35 bp, Artificial Sequence
  primer
gagctaggtagatctccaagtgtagctaagaagag

SEQ ID NO: 1933 - DNA, 35 bp, Artificial Sequence
  primer
ggagtcgctggtgacgtatgccgaggatgagcttc

SEQ ID NO: 1934 - DNA, 35 bp, Artificial Sequence
  primer
cgccgacctcctgttcacgaagccgcctgatgtaa

SEQ ID NO: 1935 - DNA, 35 bp, Artificial Sequence
  primer
agtaggcacttagttatcgattacgttagttagtc

SEQ ID NO: 1936 - DNA, 35 bp, Artificial Sequence
  primer
ggatgacgtctcagtctacctcgcagtgtcgtcta

SEQ ID NO: 1937 - DNA, 35 bp, Artificial Sequence
  primer
ctggttcgcgttagcaatactaaggcagtcaggag

SEQ ID NO: 1938 - DNA, 35 bp, Artificial Sequence
  primer
atatggtcatattggcctcttcgaacacagactgt

SEQ ID NO: 1939 - DNA, 35 bp, Artificial Sequence
  primer
tatcagaggatagcaggtctgagttgcaaggctaa

SEQ ID NO: 1940 - DNA, 35 bp, Artificial Sequence
  primer
ggtggtctgaccatagctgttcttctcacagagac

SEQ ID NO: 1941 - DNA, 35 bp, Artificial Sequence
  primer
gcaataccaacgagatgagtattcgttgaagctct

SEQ ID NO: 1942 - DNA, 35 bp, Artificial Sequence
  primer
ccaagtcgacgctgcatgaatgagcgctattcact

SEQ ID NO: 1943 - DNA, 35 bp, Artificial Sequence
  primer
ccattagatcgcttcgagacaattaggagacatga

SEQ ID NO: 1944 - DNA, 35 bp, Artificial Sequence
  primer
gatgactgtacctcctatcattgagtgtggaccaa

SEQ ID NO: 1945 - DNA, 35 bp, Artificial Sequence
  primer
atatctggatgaatagtggttaggtaagcaagtaa

SEQ ID NO: 1946 - DNA, 35 bp, Artificial Sequence
  primer
accgactatgttaattcgtgtctggatggcagaat

SEQ ID NO: 1947 - DNA, 35 bp, Artificial Sequence
  primer
gtggcagtcttgctagtatcttagaccatcaccaa

SEQ ID NO: 1948 - DNA, 35 bp, Artificial Sequence
  primer
cgctatcttagtcgagcacaatgtcttcgtatagg

SEQ ID NO: 1949 - DNA, 35 bp, Artificial Sequence
  primer
attagtacggcacgaaccggccattcatggcagct

SEQ ID NO: 1950 - DNA, 35 bp, Artificial Sequence
  primer
agtacgactatcaagactccagcgctctccttgga

SEQ ID NO: 1951 - DNA, 35 bp, Artificial Sequence
  primer
atgagcctcggagcgaacgttatcgatcaggctgt

SEQ ID NO: 1952 - DNA, 35 bp, Artificial Sequence
  primer
ttgcgtgcagtagcaccgatacacagcgcttgtat

SEQ ID NO: 1953 - DNA, 35 bp, Artificial Sequence
  primer
aacggctgcatcacctacactatactcaacatcta

SEQ ID NO: 1954 - DNA, 35 bp, Artificial Sequence
  primer
gtcgctatgcgagaagtggcgtggaatgctatggt

SEQ ID NO: 1955 - DNA, 35 bp, Artificial Sequence
  primer
catggatacctactgacttgacttctagaggaccg

SEQ ID NO: 1956 - DNA, 35 bp, Artificial Sequence
  primer
gagtgacgcagacaccgtaacgtcgaatcttctag

SEQ ID NO: 1957 - DNA, 35 bp, Artificial Sequence
  primer
agtaccgtctgtgtgaatattgttcctacgttaca

SEQ ID NO: 1958 - DNA, 35 bp, Artificial Sequence
  primer
ggctaatcgatagtgacgagttctgcacgcctgaa

SEQ ID NO: 1959 - DNA, 35 bp, Artificial Sequence
  primer
ggcgagcgctcgtggttctgagtcgctgttagatg

SEQ ID NO: 1960 - DNA, 35 bp, Artificial Sequence
  primer
tatctccagcgttataagctactggagccgctcgg

SEQ ID NO: 1961 - DNA, 35 bp, Artificial Sequence
  primer
ccttctgcgcaagtcaaggattcgcttagatggac

SEQ ID NO: 1962 - DNA, 35 bp, Artificial Sequence
  primer
gttgctgacagccgttgcgtacttgccttaagaac

SEQ ID NO: 1963 - DNA, 35 bp, Artificial Sequence
  primer
gtggcctaatcactcgcgcttcataggccgatagg

SEQ ID NO: 1964 - DNA, 35 bp, Artificial Sequence
  primer
tgcatctagcctacatcggaccttgttatggtaat

SEQ ID NO: 1965 - DNA, 35 bp, Artificial Sequence
  primer
ggacagctactggacaccaccgaactggtagtgtc

SEQ ID NO: 1966 - DNA, 35 bp, Artificial Sequence
  primer
aactggcgatggacggccgctcttccgctacatag

SEQ ID NO: 1967 - DNA, 35 bp, Artificial Sequence
  primer
ggagcagttagctatggagcaggccgataacctga

SEQ ID NO: 1968 - DNA, 35 bp, Artificial Sequence
  primer
actctacggtgcacctcagccttcatgcaataggc

SEQ ID NO: 1969 - DNA, 35 bp, Artificial Sequence
  primer
cttgtagcacaatacattactctccacgtgatagc

SEQ ID NO: 1970 - DNA, 35 bp, Artificial Sequence
  primer
ggacgctatcgataccgttattcctactctgtcgg

SEQ ID NO: 1971 - DNA, 35 bp, Artificial Sequence
  primer
ggatgatcgtcaacgatcaactgacagttagtcga

SEQ ID NO: 1972 - DNA, 35 bp, Artificial Sequence
  primer
tgacagtagcaatgtctcacgtctgcacaacggaa

SEQ ID NO: 1973 - DNA, 35 bp, Artificial Sequence
  primer
gtcgcaggacctcacggatagtagtgcgaggtcta

SEQ ID NO: 1974 - DNA, 35 bp, Artificial Sequence
  primer
atatcggcggacgcaatgacagttgttggctgatg

SEQ ID NO: 1975 - DNA, 35 bp, Artificial Sequence
  primer
aagcaccaaggaggtatgttccatcgaggcgctcg

SEQ ID NO: 1976 - DNA, 35 bp, Artificial Sequence
  primer
gaccgcaccttatagctatatcctggtctagtact

SEQ ID NO: 1977 - DNA, 35 bp, Artificial Sequence
  primer
tctcagaggaaggttgagcgtctgaccaggttggc

SEQ ID NO: 1978 - DNA, 35 bp, Artificial Sequence
  primer
tggacctagagacctagctcgtctcttcgcgatcg

SEQ ID NO: 1979 - DNA, 35 bp, Artificial Sequence
  primer
cggagtggttccacgcgacctcgcaactaatcctt

SEQ ID NO: 1980 - DNA, 35 bp, Artificial Sequence
  primer
ggagccgcgcgcagactgaccttgcttgatctact

SEQ ID NO: 1981 - DNA, 35 bp, Artificial Sequence
  primer
actctaagtatatgcgcagttagtatactgaacca

SEQ ID NO: 1982 - DNA, 35 bp, Artificial Sequence
  primer
gagcattgcttcgcttcgatgtctattctgatcag

SEQ ID NO: 1983 - DNA, 35 bp, Artificial Sequence
  primer
gcttgtattgccactcgagtaggtcgtggcagtag

SEQ ID NO: 1984 - DNA, 35 bp, Artificial Sequence
  primer
atctggacattgcattcggtgtgtatacagaaggc

SEQ ID NO: 1985 - DNA, 35 bp, Artificial Sequence
  primer
ggttgcgatcagcttgatagcaggtcatatcctca

SEQ ID NO: 1986 - DNA, 35 bp, Artificial Sequence
  primer
gcaggtactaacctgagatgcgtagctaacacagg

SEQ ID NO: 1987 - DNA, 35 bp, Artificial Sequence
  primer
atctgcaaggacgtaacgtcctcggaaggtgaggt

SEQ ID NO: 1988 - DNA, 35 bp, Artificial Sequence
  primer
ataatcttacgagcctccagtgaataatgcaagca

SEQ ID NO: 1989 - DNA, 35 bp, Artificial Sequence
  primer
caatctccgcacagtcttgttcaggtacagactta

SEQ ID NO: 1990 - DNA, 35 bp, Artificial Sequence
  primer
atgtgcgcaattcagcgtaagtgcctattcataat

SEQ ID NO: 1991 - DNA, 35 bp, Artificial Sequence
  primer
tcggacgcacacatcctgttgtcgagaagaggaag

SEQ ID NO: 1992 - DNA, 35 bp, Artificial Sequence
  primer
tcggaagcatcacatgagcatcaggagttcattgc

SEQ ID NO: 1993 - DNA, 35 bp, Artificial Sequence
  primer
atctggttgtggacttctatacagtaccagagtgg

SEQ ID NO: 1994 - DNA, 35 bp, Artificial Sequence
  primer
cgtctgaatatagttagctagtagtgtaatccagg

SEQ ID NO: 1995 - DNA, 35 bp, Artificial Sequence
  primer
taatatctgatccgacctattatctaggactactc

SEQ ID NO: 1996 - DNA, 35 bp, Artificial Sequence
  primer
tatgcggccgtccgtacctcgtctgcttcagttgg

SEQ ID NO: 1997 - DNA, 35 bp, Artificial Sequence
  primer
tggctcaagttccatattgccaagacgacctggag

SEQ ID NO: 1998 - DNA, 35 bp, Artificial Sequence
  primer
gcagttctgctaggcggtccgaggcaattgaagag

SEQ ID NO: 1999 - DNA, 35 bp, Artificial Sequence
  primer
catggcacagacgaagtatgcaccacgctcattaa

SEQ ID NO: 2000 - DNA, 35 bp, Artificial Sequence
  primer
ggagcgtactacgaccattcaaccgaatatgttac

SEQ ID NO: 2001 - DNA, 35 bp, Artificial Sequence
  primer
gcgtagatctcgcgacagagacaaggtgcgaatgg

SEQ ID NO: 2002 - DNA, 35 bp, Artificial Sequence
  primer
tggactgaggttctccggtctatactcctgtagga

SEQ ID NO: 2003 - DNA, 35 bp, Artificial Sequence
  primer
tggctatagcaacggcttcttgtgatcgcattgca

SEQ ID NO: 2004 - DNA, 35 bp, Artificial Sequence
  primer
ggcgaagaatcatgcgagacggagtagacggacgt

SEQ ID NO: 2005 - DNA, 35 bp, Artificial Sequence
  primer
gagcattgcgagttgcacacgtgatatcagactgt

SEQ ID NO: 2006 - DNA, 35 bp, Artificial Sequence
  primer
ctgttgacctatgccagaatcaatacctcagatta

SEQ ID NO: 2007 - DNA, 35 bp, Artificial Sequence
  primer
gttaacaagtagatgccaagatacaacgagagacc

SEQ ID NO: 2008 - DNA, 35 bp, Artificial Sequence
  primer
gagcaagattatagttaggaagatagttaactcgc

SEQ ID NO: 2009 - DNA, 35 bp, Artificial Sequence
  primer
tccggagtcgagcatatgtgaccaactctcaacgc

SEQ ID NO: 2010 - DNA, 35 bp, Artificial Sequence
  primer
ggagctgcgatgccgttaccgacgtcatcttcaag

SEQ ID NO: 2011 - DNA, 35 bp, Artificial Sequence
  primer
gctctatcttacacattggcgtactggactcgcga

SEQ ID NO: 2012 - DNA, 35 bp, Artificial Sequence
  primer
ttctacatattcatcgcctaccgagttgcgcgaag

SEQ ID NO: 2013 - DNA, 35 bp, Artificial Sequence
  primer
tggacgtctgacctgtgtctacatcggtggtgcta

SEQ ID NO: 2014 - DNA, 35 bp, Artificial Sequence
  primer
ggcaggacagctccgtgttctactcgaaccgcact

SEQ ID NO: 2015 - DNA, 35 bp, Artificial Sequence
  primer
tgacaacctcatgtctccgaccgcaggcatacaat

SEQ ID NO: 2016 - DNA, 35 bp, Artificial Sequence
  primer
gcaggcctaacaagtggtcacgaggagtccttatt

SEQ ID NO: 2017 - DNA, 90 bp, Artificial Sequence
  marker sequence
cccatacacacaccatgaagcttgaactaattaacattctcaaactaattaacaagcatgcaagcatgtttttacacaatgacaatatat

SEQ ID NO: 2018 - DNA, 90 bp, Artificial Sequence
  marker sequence
atgggtgagggcgcagaggcaaagacatggaggtccggaagggtagaagctcacatcaagtcgagtatgttgaatgcaatcccatatata

SEQ ID NO: 2019 - DNA, 24 bp, Artificial Sequence
  primer
cccatacacacaccatgaagcttg

SEQ ID NO: 2020 - DNA, 24 bp, Artificial Sequence
  primer
ggtagaagctcacatcaagtcgag

SEQ ID NO: 2021 - DNA, 90 bp, Artificial Sequence
  marker sequence
aatcacagaacgaggtctggacgagaacagagctggacatctacacgcaccgcatggtagtagagcatgtactgcaaaagcttgaagcgc

SEQ ID NO: 2022 - DNA, 90 bp, Artificial Sequence
  marker sequence
gatgctgagggcgaagttgtcagccaagtcctcaatgtcataggcgagatcgcagtagttctgtaaccattccctgctaaactggtccat

SEQ ID NO: 2023 - DNA, 24 bp, Artificial Sequence
  primer
acgagaacagagctggacatctac

SEQ ID NO: 2024 - DNA, 24 bp, Artificial Sequence
  primer
tcaatgtcataggcgagatcgcag

SEQ ID NO: 2025 - DNA, 90 bp, Artificial Sequence
  marker sequence
agaccaacaagcagcaagtagtcagagaagtacaagagaaggagagcaagaaggatagtaagttgcaagcttaccgttacaaagatgata

SEQ ID NO: 2026 - DNA, 90 bp, Artificial Sequence
  marker sequence
ggaggagcacaactaggcgtttatcaagatgggtcatcgagctcttggtgtcttcaaccttcttgacatcaacttctccaatcttcgtct

SEQ ID NO: 2027 - DNA, 26 bp, Artificial Sequence
  primer
ggagagcaagaaggatagtaagttgc

SEQ ID NO: 2028 - DNA, 25 bp, Artificial Sequence
  primer
cgagctcttggtgtcttcaaccttc

SEQ ID NO: 2029 - DNA, 90 bp, Artificial Sequence
  marker sequence
tggggtagtcctgaagctctaggtatgcctcttcatctccctgcacctctggtgctagcacctcctgctcttcgggcacctctaccgggg

SEQ ID NO: 2030 - DNA, 90 bp, Artificial Sequence
  marker sequence
ggatactgatgtagctttcacccgggagtattccaaggtatcgattttccacggggaacgcgaagtgcactagttgaggtttagattgcc

SEQ ID NO: 2031 - DNA, 25 bp, Artificial Sequence
  primer
gaagctctaggtatgcctcttcatc

SEQ ID NO: 2032 - DNA, 25 bp, Artificial Sequence
  primer
gtgcactagttgaggtttagattgc

SEQ ID NO: 2033 - DNA, 90 bp, Artificial Sequence
  marker sequence
tcgggaaaacgaacgggcgaactacagatgtcagtacgaagtagtctatggcaggaaatacgtagtccatacgtggtgccagcccaagcc

SEQ ID NO: 2034 - DNA, 90 bp, Artificial Sequence
  marker sequence
agcaggagggagaaaggaaacgtggcattcatcggctgtctgccattgccatgtgagacaaggaaatctacttcacccccatctatcgag

SEQ ID NO: 2035 - DNA, 24 bp, Artificial Sequence
  primer
gggcgaactacagatgtcagtacg

SEQ ID NO: 2036 - DNA, 24 bp, Artificial Sequence
  primer
ctgtctgccattgccatgtgagac

SEQ ID NO: 2037 - DNA, 90 bp, Artificial Sequence
  marker sequence
agacataagattaactatgaacaaattcacgggtccgattcctttgggatttgcagcttgcaagaaccttcaaatactcattatatcttc

SEQ ID NO: 2038 - DNA, 90 bp, Artificial Sequence
  marker sequence
ttaagttgcagaatttgatacgaagaacttgaagcatggtgaggttgccgagctcattggggatggttccagaaaggctattgtagctta

SEQ ID NO: 2039 - DNA, 24 bp, Artificial Sequence
  primer
gaacaaattcacgggtccgattcc

SEQ ID NO: 2040 - DNA, 24 bp, Artificial Sequence
  primer
cgaagaacttgaagcatggtgagg

SEQ ID NO: 2041 - DNA, 60 bp, Artificial Sequence
  probe sequence
cacacaccatgaagcttgaactaattaacattctcaaactaattaacaagcatgcaagca

SEQ ID NO: 2042 - DNA, 60 bp, Artificial Sequence
  probe sequence
caagtcctcaatgtcataggcgagatcgcagtagttctgtaaccattccctgctaaactg

SEQ ID NO: 2043 - DNA, 60 bp, Artificial Sequence
  probe sequence
gtttatcaagatgggtcatcgagctcttggtgtcttcaaccttcttgacatcaacttctc

SEQ ID NO: 2044 - DNA, 50 bp, Artificial Sequence
  probe sequence
ctgaagctctaggtatgcctcttcatctccctgcacctctggtgctagca

SEQ ID NO: 2045 - DNA, 54 bp, Artificial Sequence
  probe sequence
ctgtctgccattgccatgtgagacaaggaaatctacttcacccccatctatcga

SEQ ID NO: 2046 - DNA, 60 bp, Artificial Sequence
  probe sequence
taagattaactatgaacaaattcacgggtccgattcctttgggatttgcagcttgcaaga

(19) United States
(12) Patent Application Publication
(10) Pub. No.: US 2020/0216879 A1
(43) Pub. Date: Jul. 9, 2020

(54) METHOD FOR PRODUCING DNA PROBE AND METHOD FOR ANALYZING GENOMIC DNA USING THE DNA PROBE

(71) Applicant: TOYOTA JIDOSHA KABUSHIKI KAISHA, Toyota-shi, Aichi-ken (JP)

(72) Inventors: Hiroyuki ENOKI, Toyota-shi (JP); Yoshie TAKEUCHI, Toyota-shi (JP); Minoru INAMORI, Toyota-shi (JP)

(73) Assignee: TOYOTA JIDOSHA KABUSHIKI KAISHA, Toyota-shi, Aichi-ken (JP)

(21) Appl. No.: 16/314,274

(22) PCT Filed: Jun. 26, 2017
(86) PCT No.: PCT/JP2017/023343

§ 371 Date: Dec. 28, 2018

(30) Foreign Application Priority Data
Jun. 29, 2016 (JP) 2016-129080

(51) Int. Cl. C12Q 1/6811; C12N 15/10; C12Q 1/6876

(52) U.S. Cl. CPC C12Q 1/6811; C12Q 2600/158; C12Q 1/6876; C12N 15/1093

(57) ABSTRACT

This invention provides a DNA probe that is applicable to a DNA library prepared in a simple manner with excellent reproducibility. Such DNA probe is produced by a method comprising steps of performing a nucleic acid amplification reaction in a reaction solution containing genomic DNA and a random primer at a high concentration, so as to obtain a DNA fragments with the use of the genomic DNA as a template; determining the nucleotide sequence of the resulting DNA fragments; and, on the basis of the nucleotide sequence of the DNA fragments obtained in the step above, designing a DNA probe used for detecting a DNA fragment.

### TECHNICAL FIELD

The present invention relates to a method for producing a DNA library that can be used for analyzing a DNA marker or other purposes and a method for gene analysis using such DNA library.

### BACKGROUND ART

In general, genomic analysis is performed to conduct comprehensive analysis of genetic information contained in the genome, such as nucleotide sequence information. However, an analysis aimed at determination of the nucleotide sequence for whole genome is disadvantageous in terms of the number of processes and the cost. In cases of organisms with large genomic sizes, in addition, genomic analysis based on nucleotide sequence analysis has limitations because of genome complexity.

Patent Literature 1 discloses an amplified fragment length polymorphism (AFLP) marker technique wherein a sample-specific marker is incorporated into a restriction-enzyme-treated fragment that had been ligated to an adaptor and only a part of the sequence of the restriction-enzyme-treated fragment is to be determined. According to the technique disclosed in Patent Literature 1, the complexity of genomic DNA is reduced by treating genomic DNA with a restriction enzyme, the nucleotide sequence of a target part of the restriction-enzyme-treated fragment is determined, and the target restriction-enzyme-treated fragment is thus determined sufficiently. The technique disclosed in Patent Literature 1, however, requires processes such as treatment of genomic DNA with a restriction enzyme and ligation reaction with the use of an adaptor. Thus, it is difficult to achieve a cost reduction.

Meanwhile, Patent Literature 2 discloses as follows. That is, a DNA marker for identification that is highly correlated with the results of taste evaluation was found from among DNA bands obtained by amplifying DNAs extracted from a rice sample via PCR in the presence of adequate primers by the so-called RAPD (randomly amplified polymorphic DNA) technique. The method disclosed in Patent Literature 2 involves the use of a plurality of sequence-tagged sites (STSs, which are primers) identified by particular sequences. According to the method disclosed in Patent Literature 2, a DNA marker for identification amplified with the use of an STS primer is detected via electrophoresis. However, the RAPD technique disclosed in Patent Literature 2 yields significantly poor reproducibility of PCR amplification, and, accordingly, such technique cannot be generally adopted as a DNA marker technique.

Patent Literature 3 discloses a method for producing a genomic library wherein PCR is carried out with the use of a single type of primer designed on the basis of a sequence that appears relatively frequently in the target genome, the entire genomic region is substantially uniformly amplified, and a genomic library can be thus produced. While Patent Literature 3 describes that a genomic library can be produced by conducting PCR with the use of a random primer containing a random sequence, it does not describe any actual procedures or results of experimentation. Accordingly, the method described in Patent Literature 3 is deduced to require nucleotide sequence information of the genome so as to identify the genome appearing frequency, which would increase the number of procedures and the cost. According to the method described in Patent Literature 3, in addition, the entire genome is to be amplified, and complexity of genomic DNA cannot be reduced, disadvantageously.

Patent Literature 4 discloses a high-throughput technique associated with markers that involves reduction in genome complexity by restriction enzyme treatment in combination with an array technique. According to the technique associated with markers disclosed in Patent Literature 4, genomic DNA is digested with restriction enzymes, an adaptor is ligated to the resulting genomic DNA fragment, a DNA fragment is amplified with the use of a primer hybridizing to the adaptor, and a DNA probe used for detection of such DNA fragment is then designed on the basis of the nucleotide sequence of the amplified DNA fragment.

In addition, Non-Patent Literature 1 discloses the development of high-density linkage map containing several thousands of DNA markers for sugarcane and wheat by making use of the technique disclosed in Patent Literature 4. Also, Non-Patent Literature 2 discloses the development of a high-density linkage map containing several thousands of DNA markers for buck wheat by making use of the technique disclosed in Patent Literature 4.

Further, Patent Literature 5 discloses a method involving the use of a random primer as a sample to be reacted with an array on which a probe is immobilized. However, Patent Literature 5 does not discloses a method in which a random primers is used to obtain an amplified fragment and the resulting amplified fragment is used to construct a DNA library.

### CITATION LIST

### Patent Literature

PTL 1: JP Patent No. 5389638
PTL 2: JP 2003-79375 A
PTL 3: JP Patent No. 3972106
PTL 4: JP Patent No. 5799484
PTL 5: JP 2014-204730 A

### Non Patent Literature

NPL 1: DNA Research 21, 555-567, 2014
NPL 2: Breeding Science 64: 291-299, 2014

### SUMMARY OF INVENTION

### Technical Problem

A technique for genome information analysis, such as genetic linkage analysis conducted with the use of a DNA marker, is desired to produce a DNA library in a more convenient and highly reproducible manner. In addition, such technique is desired to produce a DNA probe capable of detecting a DNA fragment contained in a DNA library with high accuracy. As described above, a wide variety of techniques for producing a DNA library and a DNA probe are known. To date, however, there have been no techniques known to be sufficient in terms of convenience and/or reproducibility. Under the above circumstances, it is an object of the present invention to provide a method for producing a DNA probe that is applicable to a DNA library produced by a method with more convenience and higher reproducibility, and it is another object to provide a method for analyzing genomic DNA with the use of such DNA probe.

### Solution to Problem

The present inventors have conducted concentrated studies in order to attain the above objects. As a result, they discovered that a DNA library could be produced with high reproducibility by conducting PCR with the use of a random primer while designating the concentration of such random primer within a designated range in a reaction solution and that a DNA probe could be easily designed on the basis of the nucleotide sequences of the DNA library to be produced. This has led to the completion of the present invention.

The present invention includes the following.

(1) A method for producing a DNA probe comprising steps of: conducting a nucleic acid amplification reaction in a reaction solution containing genomic DNA and a random primer at a high concentration using genomic DNA as a template to obtain DNA fragments; determining the nucleotide sequences of the obtained DNA fragments; and designing a DNA probe used for detecting a DNA fragment obtained in the above step on the basis of the nucleotide sequences of such DNA fragments.

(2) The method for producing a DNA probe according to (1), wherein DNA fragments are obtained from a plurality of different genomic DNAs with the use of the random primers and, on the basis of the nucleotide sequences of the DNA fragments, the DNA probe containing regions different between such genomic DNAs is designed.

(3) The method for producing a DNA probe according to (1), wherein the nucleotide sequence of the DNA fragment is compared with a known nucleotide sequence and the DNA probe containing a region different from that of the known nucleotide sequence is designed.

(4) The method for producing a DNA probe according to (1), wherein the reaction solution contains a random primer at a concentration of 4 to 200 microM.

(5) The method for producing a DNA probe according to (1), wherein the reaction solution contains a random primer at a concentration of 4 to 100 microM.

(6) The method for producing a DNA probe according to (1), wherein the random primers each contain 9 to 30 nucleotides.

(7) The method for producing a DNA probe according to (1), wherein the DNA fragments contain 100 to 500 nucleotides.

(8) A method for analyzing genomic DNA comprising steps of: bringing the DNA probe produced by the method for producing a DNA probe according to any of (1) to

(7) into contact with a DNA fragment derived from genomic DNA subjected to analysis; and detecting hybridization occurring between the DNA probe and the DNA fragment.

(9) The method for analyzing genomic DNA according to (8), which further comprises a step of conducting a nucleic acid amplification reaction with the use of the genomic DNA subjected to analysis and the random primer to obtain the DNA fragment.

(10) The method for analyzing genomic DNA according to (8), wherein the DNA fragment derived from genomic DNA is a DNA marker and the presence or absence of the DNA marker is detected with the use of the DNA probe.

(11) An apparatus for DNA analysis comprising the DNA probe produced by the method for producing a DNA probe according to any of (1) to (7) and a support comprising the DNA probe immobilized thereon.

(12) The apparatus for DNA analysis according to (11), wherein the support is a substrate or bead.

### Advantageous Effects of Invention

In the method for producing a DNA probe according to the present invention, a nucleotide sequence of a DNA probe is designed based on the nucleotide sequence of DNA fragments produced by the method of nucleic acid amplification using a random primer at a high concentration. According to the method of nucleic acid amplification using a random primer at a high concentration, DNA fragments can be amplified with excellent reproducibility. According to the present invention, therefore, a DNA probe applicable to a DNA fragment that can be obtained while achieving excellent reproducibility can be produced in a simple manner.

According to the method for producing a DNA probe according to the present invention, also, a DNA probe applicable to a DNA fragment can be produced while achieving excellent reproducibility, and the resulting DNA probe can be used for genetic analysis, such as genetic linkage analysis, involving the use of a DNA fragment of interest as a DNA marker.

The method for analyzing genomic DNA with the use of a DNA probe according to the present invention involves the use of a DNA probe applicable to a DNA fragment produced in a simple manner with excellent reproducibility. Accordingly, genomic DNA can be analyzed in a cost-effective manner with high accuracy.

### BRIEF DESCRIPTION OF DRAWINGS

FIG. 1 shows a flow chart demonstrating a method for producing a DNA library and a method for genetic analysis with the use of the DNA library.

FIG. 2 shows a characteristic diagram demonstrating a correlation between an amplified fragment length and a fluorescence unit (FU) in which the amplified fragment length is determined based on an electrophoretic pattern of the DNA library amplified via PCR using DNA of the sugarcane variety NiF8 as a template under general conditions.

FIG. 3 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template at an annealing temperature of 45 degrees C.

FIG. 4 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template at an annealing temperature of 40 degrees C.

FIG. 5 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template at an annealing temperature of 37 degrees C.

FIG. 6 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and 2.5 units of an enzyme.

FIG. 7 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and 12.5 units of an enzyme.

FIG. 8 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and MgCl₂ at the concentration doubled from the original level.

FIG. 9 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and MgCl₂ at the concentration tripled from the original level.

FIG. 10 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and MgCl₂ at the concentration quadrupled concentration.

FIG. 11 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 8 bases.

FIG. 12 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 9 bases.

FIG. 13 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 11 bases.

FIG. 14 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 12 bases.

FIG. 15 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 14 bases.

FIG. 16 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 16 bases.

FIG. 17 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 18 bases.

FIG. 18 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer comprising 20 bases.

FIG. 19 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 2 microM.

FIG. 20 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 4 micron

FIG. 21 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 6 microM.

FIG. 22 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 6 microM.

FIG. 23 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 8 microM.

FIG. 24 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 8 microM.

FIG. 25 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 10 microM.

FIG. 26 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 10 microM.

FIG. 27 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 20 microM.

FIG. 28 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 20 microM.

FIG. 29 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 40 microM.

FIG. 30 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 40 microM.

FIG. 31 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 60 microM.

FIG. 32 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 60 microM.

FIG. 33 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 100 microM.

FIG. 34 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 100 microM.

FIG. 35 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 200 microM.

FIG. 36 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 200 microM.

FIG. 37 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 300 microM.

FIG. 38 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 300 microM.

FIG. 39 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 400 microM.

FIG. 40 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 400 microM.

FIG. 41 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer at a concentration of 500 microM.

FIG. 42 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer at a concentration of 500 microM.

FIG. 43 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 600 microM.

FIG. 44 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 700 microM.

FIG. 45 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 800 microM.

FIG. 46 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 900 microM.

FIG. 47 shows a characteristic diagram demonstrating a correlation between an amplified fragment length and a fluorescence unit (FU) in which the amplified fragment length is determined based on an electrophoretic pattern of the DNA library amplified using DNA of the sugarcane variety NiF8 as a template and a random primer at a concentration of 1000 microM.

FIG. 48 shows a characteristic diagram demonstrating the results of MiSeq analysis of a DNA library amplified using DNA of the sugarcane variety NiF8 as a template and a random primer.

FIG. 49 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the rice variety Nipponbare as a template and a random primer.

FIG. 50 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the rice variety Nipponbare as a template and a random primer.

FIG. 51 shows a characteristic diagram demonstrating the results of MiSeq analysis of a DNA library amplified using DNA of the rice variety Nipponbare as a template and a random primer.

FIG. 52 shows a characteristic diagram demonstrating positions of MiSeq read patterns in the genome information of the rice variety Nipponbare.

FIG. 53 shows a characteristic diagram demonstrating the frequency distribution of the number of mismatched nucleotides between the random primer and the rice genome.

FIG. 54 shows a characteristic diagram demonstrating the number of reads of the sugarcane varieties NiF8 and Ni9 and hybrid progeny lines thereof at the marker N80521152.

FIG. 55 shows a photograph demonstrating electrophoretic patterns of the sugarcane varieties NiF8 and Ni9 and hybrid progeny lines thereof at the PCR marker N80521152.

FIG. 56 shows a characteristic diagram demonstrating the number of reads of the sugarcane varieties NiF8 and Ni9 and hybrid progeny lines thereof at the marker N80997192.

FIG. 57 shows a photograph demonstrating electrophoretic patterns of the sugarcane varieties NiF8 and Ni9 and hybrid progeny lines thereof at the PCR marker N80997192.

FIG. 58 shows a characteristic diagram demonstrating the number of reads of the sugarcane varieties NiF8 and Ni9 and hybrid progeny lines thereof at the marker N80533142.

FIG. 59 shows a photograph demonstrating electrophoretic patterns of the sugarcane varieties NiF8 and Ni9 and hybrid progeny lines thereof at the PCR marker N80533142.

FIG. 60 shows a characteristic diagram demonstrating the number of reads of the sugarcane varieties NiF8 and Ni9 and hybrid progeny lines thereof at the marker N91552391.

FIG. 61 shows a photograph demonstrating electrophoretic patterns of the sugarcane varieties NiF8 and Ni9 and hybrid progeny lines thereof at the PCR marker N91552391.

FIG. 62 shows a characteristic diagram demonstrating the number of reads of the sugarcane varieties NiF8 and Ni9 and hybrid progeny lines thereof at the marker N91653962.

FIG. 63 shows a photograph demonstrating electrophoretic patterns of the sugarcane varieties NiF8 and Ni9 and hybrid progeny lines thereof at the PCR marker N91653962.

FIG. 64 shows a characteristic diagram demonstrating the number of reads of the sugarcane varieties NiF8 and Ni9 and hybrid progeny lines thereof at the marker N91124801.

FIG. 65 shows a photograph demonstrating electrophoretic patterns of the sugarcane varieties NiF8 and Ni9 and hybrid progeny lines thereof at the PCR marker N91124801.

FIG. 66 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 9 bases.

FIG. 67 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 9 bases.

FIG. 68 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 10 bases.

FIG. 69 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 10 bases.

FIG. 70 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 11 bases.

FIG. 71 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 11 bases.

FIG. 72 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 12 bases.

FIG. 73 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 12 bases.

FIG. 74 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 14 bases.

FIG. 75 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 14 bases.

FIG. 76 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 16 bases.

FIG. 77 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 16 bases.

FIG. 78 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 18 bases.

FIG. 79 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 18 bases.

FIG. 80 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer comprising 20 bases.

FIG. 81 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer comprising 20 bases.

FIG. 82 shows a characteristic diagram demonstrating the results of investigating the reproducibility of the DNA library amplified using DNA of the sugarcane variety NiF8 as a template and random primers each comprising 8 to 35 bases used at a concentration of 0.6 to 300 microM.

FIG. 83 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a single type of random primer.

FIG. 84 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a single type of random primer.

FIG. 85 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and 2 types of random primers.

FIG. 86 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and 2 types of random primers.

FIG. 87 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and 3 types of random primers.

FIG. 88 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and 3 types of random primers.

FIG. 89 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and 12 types of random primers.

FIG. 90 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and 12 types of random primers.

FIG. 91 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and 24 types of random primers.

FIG. 92 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and 24 types of random primers.

FIG. 93 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and 48 types of random primers.

FIG. 94 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and 48 types of random primers.

FIG. 95 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer B comprising 10 nucleotides.

FIG. 96 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer B comprising 10 nucleotides.

FIG. 97 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer C comprising 10 nucleotides.

FIG. 98 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer C comprising 10 nucleotides.

FIG. 99 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer D comprising 10 nucleotides.

FIG. 100 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer D comprising 10 nucleotides.

FIG. 101 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer E comprising 10 nucleotides.

FIG. 102 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer E comprising 10 nucleotides.

FIG. 103 shows a characteristic diagram demonstrating a correlation between an amplified fragment length and a fluorescence unit (FU) in which the amplified fragment length is determined based on an electrophoretic pattern (appeared for the first time) of the DNA library amplified using DNA of the sugarcane variety NiF8 as a template and a random primer F comprising 10 nucleotides.

FIG. 104 shows a characteristic diagram demonstrating a correlation between an amplified fragment length and a fluorescence unit (FU) in which the amplified fragment length is determined based on an electrophoretic pattern (appeared for the second time) of the DNA library amplified using DNA of the sugarcane variety NiF8 as a template and a random primer F comprising 10 nucleotides.

FIG. 105 shows a characteristic diagram demonstrating a correlation between an amplified fragment length and a fluorescence unit (FU) in which the amplified fragment length is determined based on an electrophoretic pattern (appeared for the first time) of the DNA library amplified using human genomic DNA as a template and a random primer A comprising 10 nucleotides.

FIG. 106 shows a characteristic diagram demonstrating a correlation between an amplified fragment length and a fluorescence unit (FU) in which the amplified fragment length is determined based on an electrophoretic pattern (appeared for the second time) of the DNA library amplified using human genomic DNA as a template and a random primer A comprising 10 nucleotides.

FIG. 107 shows a flow chart demonstrating a process for producing a DNA microarray with the application of the method for producing a DNA probe according to the present invention.

FIG. 108 shows a characteristic diagram demonstrating the results of assaying signals obtained from a DNA probe concerning the DNA library amplified using genomic DNAs of NiF8 and Ni9 as templates and a random primer at a high concentration.

FIG. 109 shows a characteristic diagram demonstrating the results of comparison of signals obtained through repeated measurements concerning the DNA library amplified using genomic DNA of Ni9 as a template and a random primer at a high concentration.

FIG. 110 shows a characteristic diagram demonstrating the results of assaying signal levels obtained from the DNA probe reacting with the marker N80521152.

FIG. 111 shows a characteristic diagram demonstrating the results of assaying signal levels obtained from the DNA probe reacting with the marker N80997192.

FIG. 112 shows a characteristic diagram demonstrating the results of assaying signal levels obtained from the DNA probe reacting with the marker N80533142.

FIG. 113 shows a characteristic diagram demonstrating the results of assaying signal levels obtained from the DNA probe reacting with the marker N91552391.

FIG. 114 shows a characteristic diagram demonstrating the results of assaying signal levels obtained from the DNA probe reacting with the marker N91653962.

FIG. 115 shows a characteristic diagram demonstrating the results of assaying signal levels obtained from the DNA probe reacting with the marker N91124801.

### DESCRIPTION OF EMBODIMENTS

Hereafter, the present invention is described in detail.

According to the method for producing a DNA probe of the present invention, a nucleic acid amplification reaction is carried out in a reaction solution, which is prepared to contain a primer having an arbitrary nucleotide sequence (hereafter, referred to as a “random primer”) at a high concentration, and a nucleotide sequence of a DNA probe used for detecting an amplified nucleic acid fragment (i.e., a DNA fragment) is designed based on the nucleotide sequence of such DNA fragment. By conducting a nucleic acid amplification reaction in a reaction solution containing a random primer at a high concentration, a DNA fragment of interest can be amplified with excellent reproducibility. Hereafter, the obtained DNA fragment is referred to as a “DNA library.”

When a reaction solution contains a random primer at a high concentration, such concentration is higher than the concentration of a primer used in a general nucleic acid amplification reaction. When producing a DNA library, specifically, a random primer is used at a higher concentration than a primer used in a general nucleic acid amplification reaction. As a template contained in a reaction solution, genomic DNA prepared from a target organism for which a DNA library is to be produced can be used. A target organism species is not particularly limited, and a target organism species can be, for example, an animal including a human, a plant, a microorganism, or a virus. That is, a DNA library can be produced from any organism species.

When producing a DNA library, the concentration of a random primer may be prescribed as described above. Thus, a nucleic acid fragment (or nucleic acid fragments) can be amplified with high reproducibility. The term “reproducibility” used herein refers to an extent of concordance among nucleic acid fragments amplified by a plurality of nucleic acid amplification reactions carried out with the use of the same template and the same random primer. That is, the term “high reproducibility (or the expression “reproducibility is high”)” refers to a high extent of concordance among nucleic acid fragments amplified by a plurality of nucleic acid amplification reactions carried out with the use of the same template and the same random primer.

The extent of reproducibility can be evaluated by, for example, conducting a plurality of nucleic acid amplification reactions with the use of the same template and the same random primer, calculating the Spearman's rank correlation coefficient for the data of the nucleotide sequences of the resulting amplified fragments, and evaluating the extent of reproducibility on the basis of such coefficient. The Spearman's rank correlation coefficient is generally represented by the symbol p (rho). When p (rho) is greater than 0.9, for example, the reproducibility of the amplification reaction of interest can be evaluated to be sufficient.

Random Primer

A sequence constituting a random primer that can be used for producing a DNA library is not particularly limited. For example, a random primer comprising nucleotides having 9 to 30 bases can be used. In particular, a random primer may be composed of any nucleotide sequence comprising 9 to 30 bases, a nucleotide type (i.e., a sequence type) is not particularly limited, and a random primer may be composed of 1 or more types of nucleotide sequences, preferably 1 to 10,000 types of nucleotide sequences, more preferably 1 to 1,000 types of nucleotide sequences, further preferably 1 to 100 types of nucleotide sequences, and most preferably 1 to 96 types of nucleotide sequences. With the use of nucleotides (or a group of nucleotides) within the range mentioned above for a random primer, an amplified nucleic acid fragment can be obtained with higher reproducibility. When a random primer comprises a plurality of nucleotide sequences, it is not necessary that all nucleotide sequences comprise the same number of bases (9 to 30 nucleotides). A random primer may comprise a plurality of nucleotide sequences composed of a different number of bases.

When designing a plurality of types of nucleotide sequences for a random primer, 30% or more, preferably 50% or more, more preferably 70% or more, and further preferably 90% or more of the entire such sequences exhibit 70% or less, preferably 60% or less, more preferably 50% or less, and most preferably 40% or less identity. By designing a plurality of types of nucleotides for a random primer exhibiting the identity within such range, an amplified fragment can be obtained over the entire genomic DNA of the target organism species. Thus, uniformity of the amplified fragment can be enhanced.

A nucleotide sequence constituting a random primer is preferably designed to have a G-C content of 5% to 95%, more preferably 10% to 90%, further preferably 15% to 80%, and most preferably 20% to 70%. With the use of an aggregate of nucleotides having the G-C content within the aforementioned range as a random primer, amplified nucleic acid fragments can be obtained with higher reproducibility. G-C content is the percentage of guanine and cytosine contained in the whole nucleotide chain.

In particular, a nucleotide sequence used as a random primer is preferably designed to comprise continuous bases accounting for 80% or less, more preferably 70% or less, further preferably 60% or less, and most preferably 50% or less of the full-length sequence. Alternatively, the number of continuous bases in a nucleotide sequence used as a random primer is preferably 8 or less, more preferably 7 or less, further preferably 6 or less, and most preferably 5 or less. With the use of an aggregate of nucleotides comprising the number of continuous bases within the aforementioned range as a random primer, amplified nucleic acid fragments can be obtained with higher reproducibility.

In addition, it is preferable that a nucleotide sequence used as a random primer be designed to not comprise a complementary region of 6 or more, more preferably 5 or more, and further preferably 4 or more bases in a molecule. Thus, double strand formation occurring in a molecule can be prevented, and amplified nucleic acid fragments can be obtained with higher reproducibility.

When a plurality of types of nucleotide sequences are designed as random primers, in particular, it is preferable that a plurality of nucleotide sequences be designed to not comprise complementary regions of 6 or more, more preferably 5 or more, and further preferably 4 or more bases among a plurality of types of nucleotide sequences. Thus, double strand formation occurring between nucleotide sequences can be prevented, and amplified nucleic acid fragments can be obtained with higher reproducibility.

When a plurality of nucleotide sequences are designed as random primers, in addition, it is preferable that such sequences be designed to not comprise complementary regions of 6 or more, more preferably 5 or more, and further preferably 5 or more bases at the 3′ terminus. Thus, double strand formation occurring between nucleotide sequences can be prevented, and amplified nucleic acid fragments can be obtained with higher reproducibility.

The terms “complementary regions” and “complementary sequences” refer to, for example, regions and sequences exhibiting 80% to 100% identity to each other (e.g., regions and sequences each comprising 5 bases in which 4 or 5 bases are complementary to each other) or regions and sequences exhibiting 90% to 100% identity to each other (e.g., regions and sequences each comprising 5 bases in which 5 bases are complementary to each other).

Further, a nucleotide sequence used as a random primer is preferably designed to have a Tm value suitable for thermal cycling conditions (in particular, an annealing temperature) of a nucleic acid amplification reaction. A Tm value can be calculated by a conventional method, such as the nearest neighbor base pair approach, the Wallace method, and the GC % method, although a method of calculation is not particularly limited thereto. Specifically, a nucleotide sequence used as a random primer is preferably designed to have a Tm value of 10 to 85 degrees C., more preferably 12 to 75 degrees C. further preferably 14 to 70 degrees C., and most preferably 16 to 65 degrees C. By designing a random primer to have a Tm value within the aforementioned range, amplified nucleic acid fragments can be obtained with higher reproducibility under given thermal cycling conditions (in particular, at a given annealing temperature) of the nucleic acid amplification reaction.

When a plurality of nucleotide sequences are designed as random primers, in addition, a variation for Tm among a plurality of nucleotide sequences is preferably 50 degrees C. or less, more preferably 45 degrees C. or less, further preferably 40 degrees C. or less, and most preferably 35 degrees C. or less. By designing random primers while adjusting a variation for Tm among a plurality of nucleotide sequences within the range mentioned above, amplified nucleic acid fragments can be obtained with higher reproducibility under given thermal cycling conditions (in particular, at a given annealing temperature) of the nucleic acid amplification reaction.

Nucleic Acid Amplification Reaction

When producing a DNA library, many DNA fragments are obtained via the nucleic acid amplification reaction carried out with the use of random primers and genomic DNA as a template described above. At the time of the nucleic acid amplification reaction, in particular, the concentration of random primes in a reaction solution is prescribed higher than the concentration of primers in a conventional nucleic acid amplification reaction. Thus, many DNA fragments can be obtained with the use of genomic DNA as a template while achieving high reproducibility. Such many DNA fragments can be used for a DNA library that can be used for genotyping and other purposes.

A nucleic acid amplification reaction is aimed at synthesis of amplified fragments in a reaction solution containing genomic DNA as a template, the random primers, DNA polymerase, deoxynucleoside triphosphates as a substrate (i.e., dNTP, which is a mixture of dATP, dCTP, dTTP, and dGTP), and a buffer under the given thermal cycling conditions. It is necessary that a nucleic acid amplification reaction be carried out in a reaction solution containing Mg²⁺ at a given concentration. In the reaction solution of the composition described above, the buffer contains MgCl₂. When the buffer does not contain MgCl₂, the reaction solution of the composition described above further contains MgCl₂.

In a nucleic acid amplification reaction, in particular, it is preferable that the concentration of random primers be adequately determined in accordance with the base lengths of the random primers. When a plurality of types of nucleotide sequences having different numbers of bases are used as random primers, the number of bases constituting the random primers may be the average of such plurality of nucleotide sequences (the average may be a simple average or the weight average taking the amount of nucleotides into account).

Specifically, a nucleic acid amplification reaction is carried out with the use of a random primer comprising 9 to 30 bases at a concentration of 4 to 200 microM, and preferably at 4 to 100 microM. Under such conditions, many amplified fragments, and, in particular, many amplified fragments comprising 100 to 500 bases, can be obtained via a nucleic acid amplification reaction while achieving high reproducibility.

When a random primer comprises 9 to 10 bases, more specifically, the concentration of such random primer is preferably 40 to 60 microM. When a random primer comprises 10 to 14 bases, it is preferable that the concentration of such random primer satisfy the conditions defined by an inequation: y>3E+08x^{-6.974} and be 100 microM or less, provided that the base length of the random primer is represented by “y” and the concentration of the random primer is represented by “x.” When a random primer comprises 14 to 18 bases, the concentration of such random primer

is preferably 4 to 100 microM. When a random primer comprises 18 to 28 bases, it is preferable that the concentration of such random primer be 4 microM or more and satisfy the conditions defined by an inequation: y<8E+08x^{-5.533}. When a random primer comprises 28 to 29 bases, the concentration of such random primer is preferably 6 to 10 microM. By designating the random primer concentration in accordance with the number of bases constituting the random primer as described above, many amplified fragments can be obtained with more certainty while achieving high reproducibility.

As described in the examples below, the inequations: y>3E+08x^{-6.974} and y<8E+08x^{-5.533} are developed to be able to represent the concentration of a random primer at which many DNA fragments comprising 100 to 500 bases can be obtained with high reproducibility as a result of thorough inspection of the correlation between random primer length and random primer concentration.

While the amount of genomic DNA serving as a template in a nucleic acid amplification reaction is not particularly limited, it is preferably 0.1 to 1000 ng, more preferably 1 to 500 ng, further preferably 5 to 200 ng, and most preferably 10 to 100 ng, when the amount of the reaction solution is 50 microliters. By designating the amount of genomic DNA as a template within such range, many amplified fragments can be obtained without inhibiting the amplification reaction from a random primer, while achieving high reproducibility.

Genomic DNA can be prepared in accordance with a conventional technique without particular limitation. With the use of a commercialized kit, also, genomic DNA can be easily prepared from a target organism species. Genomic DNA extracted from an organism in accordance with a conventional technique or with the use of a commercialized kit may be used without further processing, genomic DNA extracted from an organism and then purified may be used, or genomic DNA subjected to restriction enzyme treatment or ultrasonic treatment may be used.

DNA polymerase used in a nucleic acid amplification reaction is not particularly limited, and an enzyme having DNA polymerase activity under thermal cycling conditions for a nucleic acid amplification reaction can be used. Specifically, heat-stable DNA polymerase used for a general nucleic acid amplification reaction can be used. Examples of DNA polymerases include thermophilic bacteria-derived DNA polymerase, such as Taq DNA polymerase, and hyperthermophilic archaea-derived DNA polymerase, such as KOD DNA polymerase and Pfu DNA polymerase. In a nucleic acid amplification reaction, it is particularly preferable that Pfu DNA polymerase be used as DNA polymerase in combination with the random primer described above. With the use of such DNA polymerase, many amplified fragments can be obtained with more certainty while achieving high reproducibility.

In a nucleic acid amplification reaction, the concentration of deoxynucleoside triphosphate as a substrate (i.e., dNTP, which is a mixture of dATP, dCTP, dTTP, and dGTP) is not particularly limited, and it can be 5 microM to 0.6 mM, preferably 10 microM to 0.4 mM, and more preferably 20 microM to 0.2 mM. By designating the concentration of dNTP serving as a substrate within such range, errors caused by incorrect incorporation by DNA polymerase can be prevented, and many amplified fragments can be obtained while achieving high reproducibility.

A buffer used in a nucleic acid amplification reaction is not particularly limited. For example, a solution comprising MgCl₂ as described above, Tris-HCl (pH 8.3), and KCl can be used. The concentration of Mg²⁺ is not particularly limited. For example, it can be 0.1 to 4.0 mM, preferably 0.2 to 3.0 mM, more preferably 0.3 to 2.0 mM, and further preferably 0.5 to 1.5 mM. By designating the concentration of Mg²⁺ in the reaction solution within such range, many amplified fragments can be obtained while achieving high reproducibility.

Thermal cycling conditions of a nucleic acid amplification reaction are not particularly limited, and a common thermal cycle can be adopted. A specific example of a thermal cycle comprises a first step of thermal denaturation in which genomic DNA as a template is dissociated into single strands, a cycle comprising thermal denaturation, annealing, and extension repeated a plurality of times (e.g., 20 to 40 times), a step of extension for a given period of lime according to need, and the final step of storage.

Thermal denaturation can be performed at, for example, 93 to 99 degrees C., preferably 95 to 98 degrees C., and more preferably 97 to 98 degrees C. Annealing can be performed at, for example, 30 to 70 degrees C., preferably 35 to 68 degrees C., and more preferably 37 to 65 degrees C., although it varies depending on a Tm value of the random primer. Extension can be performed at, for example, 70 to 76 degrees C., preferably 71 to 75 degrees C., and more preferably 72 to 74 degrees C. Storage can be performed at, for example, 4 degrees C.

The first step of thermal denaturation can be performed within the temperature range described above for a period of, for example, 5 seconds to 10 minutes, preferably 10 seconds to 5 minutes, and more preferably 30 seconds to 2 minutes. In the cycle comprising “thermal denaturation, annealing, and extension,” thermal denaturation can be carried out within the temperature range described above for a period of, for example, 2 seconds to 5 minutes, preferably 5 seconds to 2 minutes, and more preferably 10 seconds to 1 minute. In the cycle comprising “thermal denaturation, annealing, and extension,” annealing can be carried out within the temperature range described above for a period of, for example, 1 second to 3 minutes, preferably 3 seconds to 2 minutes, and more preferably 5 seconds to 1 minute. In the cycle comprising “thermal denaturation, annealing, and extension,” extension can be carried out within the temperature range described above for a period of, for example, 1 second to 3 minutes, preferably 3 seconds to 2 minutes, and more preferably 5 seconds to 1 minute.

When producing a DNA library, amplified fragments may be obtained by a nucleic acid amplification reaction that employs a hot start method. The hot start method is intended to prevent mis-priming or non-specific amplification caused by primer-dimer formation prior the cycle comprising “thermal denaturation, annealing, and extension.” The hot start method involves the use of an enzyme in which DNA polymerase activity has been suppressed by binding an anti-DNA polymerase antibody thereto or chemical modification thereof. Thus, DNA polymerase activity can be suppressed and a non-specific reaction prior to the thermal cycle can be prevented. According to the hot start method, a temperature is set high in the first thermal cycle, DNA polymerase activity is thus recovered, and the subsequent nucleic acid amplification reaction is then allowed to proceed.

As described above, many amplified fragments can be obtained with the use of genomic DNA as a template and a random primer by conducting a nucleic acid amplification reaction with the use of a random primer comprising 9 to 30 bases and prescribing the concentration thereof to 4 to 200 microM in a reaction solution. With the use of the random primer comprising 9 to 30 bases by prescribing the concentration thereof to 4 to 200 microM in a reaction solution, a nucleic acid amplification reaction can be performed with very high reproducibility. According to the nucleic acid amplification reaction, specifically, many amplified fragments can be obtained while achieving very high reproducibility. Accordingly, such many amplified fragments can be used for a DNA library in genetic analysis targeting genomic DNA.

By performing a nucleic acid amplification reaction with the use of the random primer comprising 9 to 30 bases and prescribing the concentration thereof in a reaction solution to 4 to 200 microM, in particular, many amplified fragments comprising about 100 to 500 bases can be obtained with the use of genomic DNA as a template. Such many amplified fragments comprising about 100 to 500 bases are suitable for mass analysis of nucleotide sequences with the use of, for example, a next-generation sequencer, and highly accurate sequence information can thus be obtained. According to the present invention, accordingly, a DNA library, including DNA fragments comprising about 100 to 500 bases, can be produced.

By performing a nucleic acid amplification reaction with the use of the random primer comprising 9 to 30 bases and prescribing the concentration thereof to 4 to 200 microM in a reaction solution, in particular, the entire genomic DNA can be uniformly amplified. In other words, amplified DNA fragments are not obtained from a particular region of genomic DNA by the nucleic acid amplification reaction with the use of such random primer, but amplified fragments are obtained from the entire genome. According to the present invention, specifically, a DNA library can be produced uniformly across the entire genome.

DNA Probe

In the present invention, the term “DNA probe” refers to a DNA fragment that has a nucleotide sequence complementary to the target DNA fragment and is able to hybridize to such DNA fragment. A DNA probe that is applicable to a so-called oligonucleotide microarray is particularly preferable. An oligonucleotide microarray is a microarray in which oligonucleotides comprising nucleotide sequences of interest are synthesized on a support and the synthesized oligonucleotides are used as DNA probes. The synthesized oligonucleotides serving as DNA probes comprise, for example, 20 to 100 bases, preferably 30 to 90 bases, and more preferably 50 to 60 bases.

The DNA probes designed in accordance with the present invention may be applied to a microarray comprising the synthesized oligonucleotides with the base length described above immobilized on a support, as with the case of the so-called Stanford-type microarray. Specifically, the DNA probes designed in accordance with the present invention can be applied to any microarrays according to conventional techniques. Thus, the DNA probes designed in accordance with the present invention can be applied to a microarray comprising a flat substrate, such as a glass or silicone substrate, as a support and a bead array comprising a microbead support.

According to the method for producing a DNA probe of the present invention, a nucleotide sequence of a DNA probe is designed to detect a DNA fragment (a. DNA library) on the basis of the nucleotide sequence of the DNA fragment. Specifically, the nucleotide sequence of the DNA fragment (the DNA library) produced in the manner described above is first determined, and a nucleotide sequence of a DNA probe is designed based on the determined nucleotide sequence. A method for determining a nucleotide sequence of a DNA fragment is not particularly limited. For example, a DNA sequencer in accordance with the Sanger method or a next-generation sequencer can be used. While a next-generation sequencer is not particularly limited, such sequencer is also referred to as a second-generation sequencer, and such sequencer is an apparatus for nucleotide sequencing that is capable of simultaneous determination of nucleotide sequences of several tens of millions of DNA fragments. A sequencing principle of the next-generation sequencer is not particularly limited. For example, sequencing can be carried out in accordance with the method in which target DNA is amplified on flow cells and sequencing is carried out while conducting synthesis with the use of bridge PCR method and sequencing-by-synthesis method, or in accordance with emulsion PCR method and the method of Pyrosequencing in which sequencing is carried out by assaying the amount of pyrophosphoric acids released at the time of and DNA synthesis. More specific examples of next-generation sequencers include MiniSeq, MiSeq, NextSeq, HiSeq, and HiSeq X Series (IIlumina) and Roche 454 GS FLX sequencers (Roche).

Subsequently, a DNA probe is designed to comprise, for example, a nucleotide sequence complementary to the nucleotide sequence of the DNA fragment (the DNA library) described above. More specifically, a region or a plurality of regions of the base lengths shorter than those of the DNA fragment (the DNA library) and covering at least a part of the DNA fragment (the DNA library) is/are identified, and the identified one or more regions are designed as probes for detecting the DNA fragment (the DNA library).

When a plurality of regions are designed for a particular DNA fragment, such DNA fragment is to be detected with the use of a plurality of DNA probes. A region may be designed for a particular DNA fragment, and two or more regions may be designed for another DNA fragment. Specifically, a different number of regions; that is, DNA probes, may be designed for each DNA fragment. When a plurality of DNA probes are to be designed for a DNA fragment, parts of such plurality of DNA probes may overlap with each other, or such plurality of DNA probes may be designed with intervals comprising several bases.

The number of bases constituting a DNA probe to be designed in the manner described above is not particularly limited. Such DNA probe can comprise 20 to 100 bases, preferably 30 to 90 bases, more preferably 40 to 80 bases, and most preferably 50 to 60 bases.

It is particularly preferable that a plurality of regions be designed, in such a manner that the entire region of a genomic DNA fragment, the nucleotide sequence of which had been determined, would be covered with a plurality of regions. In such a case, a plurality of probes can react with a genomic DNA fragment obtained from genomic DNA derived from a particular organism species via restriction enzyme treatment, and such genomic DNA fragment can be detected with the use of such plurality of probes.

A Tm value of a DNA probe is not particularly limited, and it can be 60 to 95 degrees C., preferably 70 to 90 degrees C., more preferably 75 to 85 degrees C., and most preferably 78 to 82 degrees C.

When preparing DNA fragments from genomic DNAs with the use of random primers as described above, DNA fragments are obtained from a plurality of different genomic DNAs, and nucleotide sequences of these DNA fragments with different origins can be determined independently from each other. By comparing the determined nucleotide sequences, regions having different nucleotide sequences among the genomic DNAs can be identified. According to the method for producing a DNA probe of the present invention. DNA probes can be designed to comprise regions having different nucleotide sequences among the genomic DNAs thus identified. Specifically, a DNA probe may be designed to comprise a region of a particular genomic DNA that is different from another genomic DNA, and another DNA probe may be designed to comprise a region of the other genomic DNA that is different from the aforementioned particular genomic DNA. With the use of a pair of DNA probes thus designed, a specific type of genomic DNA to be analyzed can be identified.

The nucleotide sequence of the DNA fragment amplified from genomic DNA with the use of a random primer may be compared with a known nucleotide sequence, and a DNA probe may be designed to comprise a region different from such known nucleotide sequence. A known nucleotide sequence can be obtained from a variety of conventional databases. While any databases can be used without particular limitation, the DDBJ database provided by the DNA Data Bank of Japan, the EMBL database provided by the European Bioinformatics Institute, the Genbank database provided by the National Center for Biotechnology Information, the KEGG database provided by the Kyoto Encyclopedia of Genes and Genomes, or a combined database comprising such various databases can be adequately used.

Apparatus for DNA Analysis

The apparatus for DNA analysis according to the present invention comprises the DNA probes designed in the manner described above immobilized on a support. An apparatus for DNA analysis comprising DNA probes immobilized on a support is occasionally referred to as a “DNA microarray.” Specifically, the apparatus for DNA analysis according to the present invention is not limited to a so-called DNA chip comprising DNA probes immobilized on a support (i.e., a DNA microarray in a narrow sense), and apparatuses composed to be capable of utilization of DNA probes designed in the manner described above on a support are within the scope of the present invention.

For example, a DNA microarray comprising DNA probes designed in the manner described above can be produced in accordance with a conventional technique. A DNA microarray can be produced by, for example, synthesizing an oligonucleotide comprising a nucleotide sequence of the DNA probe designed in the manner described above on a support based on such nucleotide sequence. A method for oligonucleotide synthesis is not particularly limited, and any conventional technique can be employed. For example, oligonucleotide synthesis can be performed on a support by photolithography in combination with chemical synthesis via light application. Alternatively, an oligonucleotide comprising a linker molecule having a high affinity with a support surface added to its terminus may be separately synthesized on the basis of the nucleotide sequence of the DNA probe designed in the manner described above, and the resulting oligonucleotide may then be immobilized on a support surface at a particular position. A DNA microarray can also be produced by spotting the DNA probe designed in the manner described above on a support with the use of a pin-type arrayer or a nozzle-type arrayer.

The DNA microarray thus produced (i.e., the apparatus for DNA analysis) comprises a DNA probe comprising a nucleotide sequence complementary to a DNA fragment amplified from genomic DNA derived from a particular type of organism with the use of a random primer at a high concentration. Specifically, the DNA microarray thus produced is intended to detect a DNA fragment amplified from genomic DNA with the use of a random primer at a high concentration with the use of a DNA probe.

A DNA microarray may be any of a microarray using a flat substrate made of glass or silicone as a support, a bead array comprising a microbead support, and a three-dimensional microarray comprising a probe immobilized on an inner wall of a hollow fiber.

Method of Genomic DNA Analysis

With the use of the DNA probe produced in the manner described above, analysis of genomic DNA, such as genotyping, can be performed. The DNA probe described above is equivalent to the DNA library produced with the use of a random primer at a high concentration. Such DNA library has very high reproducibility, the size of which is suitable for a next-generation sequencer, and it is uniform across the entire genome. Accordingly, the DNA library can be used as a DNA marker (it is also referred to as a genetic marker or a gene marker). The term “DNA marker” refers to a region in the genome serving as a marker associated with genetic traits. A DNA marker can be used for, for example, breeding comprising a step of selection with the use of genotype identification, linkage maps, gene mapping, or a marker, back crossing using a marker, quantitative trait locus mapping, bulked segregant analysis, variety identification, or discontinuous imbalance mapping.

Specifically, a DNA marker can be detected with the use of the DNA probe produced in the manner described above, and breeding comprising a step of selection with the use of genotype identification, linkage maps, gene mapping, and a marker, back crossing with the use of a marker, quantitative trait locus mapping, bulked segregant analysis, variety identification, or discontinuous imbalance mapping can be carried out.

More specifically, an example of a method for genomic DNA analysis involving the use of the DNA probe comprises bringing the DNA probe produced in the manner described above into contact with a DNA fragment derived from genomic DNA of the target of analysis. Such DNA fragment may be prepared with the use of the random primer that was used for producing the DNA library. Alternatively, a pair of primers that specifically amplify the DNA marker of interest may be designed on the basis of the nucleotide sequence of interest, and a DNA fragment may be prepared via a nucleic acid amplification reaction with the use of the pair of designed primers.

Subsequently, hybridization occurring between the DNA probe and the DNA fragment is detected in accordance with a conventional technique. For example, a label is added to the amplified DNA fragment, and hybridization of interest can be thus detected on the basis of the label. Any conventional substance may be used as a label. Examples of labels that can be used include a fluorescent molecule, a pigment molecule, and a radioactive molecule. A labeled nucleotide may be used in the step of DNA fragment amplification.

When a DNA microarray comprising a DNA probe is used, for example, a labeled DNA fragment is brought into contact with the DNA microarray under given conditions, and a DNA probe immobilized on the DNA microarray is allowed to hybridize to a labeled genomic DNA fragment. In this case, a probe hybridizes to a part of the DNA fragment, and it is preferable that hybridization be carried out under highly stringent conditions, so that hybridization does not occur in the presence of mismatch of a base, but it occurs only when the bases completely match. Under such highly stringent conditions, a slight change in single nucleotide polymorphism can be detected.

The stringency conditions can be adjusted in terms of reaction temperatures and salt concentrations. At a higher temperature, specifically, higher stringency conditions can be achieved. At a lower salt concentration, higher stringency conditions can be achieved. When a probe comprising 50 to 75 bases is used, for example, higher stringency conditions can be achieved by conducting hybridization at 40 to 44 degrees C. with 0.21 SDS and 6×SSC.

Hybridization occurring between a DNA probe and a labeled DNA fragment can be detected based on a label. After the hybridization reaction between the labeled DNA fragment and the DNA probe, specifically, an unreacted DNA fragment or the like is washed, and a label of the DNA fragment that had specifically hybridized to the DNA probe is then observed. When a label is a fluorescent substance, for example, the fluorescent wavelength is detected. When a label is a pigment molecule, the pigment wavelength is detected. More specifically, an apparatus used for general DNA microarray analysis, such as a fluorescence detector or image analyzer, can be used.

In particular, DNA fragments amplified using genomic DNA as a template and a random primer at a high concentration can be detected with the use of such DNA probe. When a DNA probe comprising regions that are different among a plurality of different genomic DNAs is used, the genomic DNA as the target of analysis can be analyzed in accordance with the DNA probe to which a DNA fragment derived from the genomic DNA as the target of analysis had hybridized. For example, a DNA probe reacting with a DNA marker comprising differences in nucleotide sequences among genomic DNAs of relative species may be used, so that the species of the genomic DNA as the target of analysis can be identified.

### EXAMPLES

Hereafter, the present invention is described in greater detail with reference to the following examples, although the technical scope of the present invention is not limited to these examples.

### Example 1

1. Flow chart

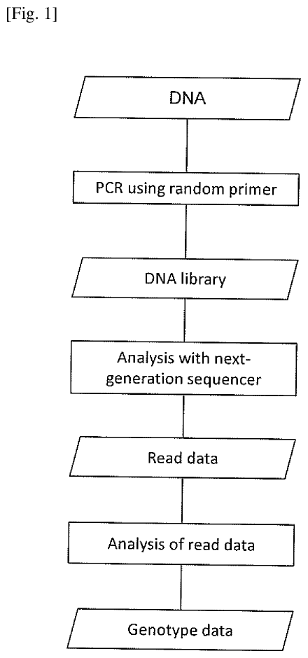

In this example, a DNA library was prepared via PCR using genomic DNAs extracted from various types of organism species as templates and various sets of random primers in accordance with the flow chart shown in FIG. 1. With the use of the prepared DNA library, also, sequence analysis was performed with the use of a so-called next-generation sequencer, and the genotype was analyzed based on the read data.

2. Materials

In this example, genomic DNAs were extracted from the sugarcane varieties NiF8 and Ni9, 22 hybrid progeny lines thereof, and the rice variety Nipponbare using the DNeasy Plant Mini kit (QIAGEN), and the extracted genomic DNAs were purified. The purified genomic DNAs were used as NiF8-derived genomic DNA, Ni9-derived genomic DNA, 22 hybrid sugarcane progeny-derived genomic DNAs, and Nipponbare-derived genomic DNA, respectively. In Example 1, human genomic DNA was purchased from TakaraBio and used as human-derived genomic DNA.

3. Method

3.1 Correlation Between PCR Condition and DNA Fragment Size

3.1.1 Random Primer Designing

In order to design random primers, GC content was set between 20% and 70%, and the number of continuous bases was adjusted to 5 or fewer. Sequence length was set at 16 levels (i.e., 8, 9, 10, 11, 12, 14, 16, 18, 20, 22, 24, 26, 28, 29, 30, and 35). For each sequence length, 96 types of nucleotide sequences were designed, and 96 sets of random primers were prepared. Concerning 10-base primers, 6 sets of random primers each comprising 96 types of random primers were designed (these 6 sets are referred to as 10-base primer A to 10-base primer F, respectively). In this example, specifically, 21 different sets of random primers were prepared.

Tables 1 to 21 show nucleotide sequences of random primers contained in such 21 different sets of random primers.

TABLE 1
Table 1 List of random primers
(10-base primers A)

| | | SEQ |
|---|---|---|
| | Primer | ID |
| No | sequence | NO: |
| 1 | AGACGTCGTT | 1 |
| 2 | GAGGCGATAT | 2 |
| 3 | GTGCGAACGT | 3 |
| 4 | TTATACTGCC | 4 |
| 5 | CAAGTTCGCA | 5 |
| 6 | ACAAGGTAGT | 6 |
| 7 | ACACAGCGAC | 7 |
| 8 | TTACCGATGT | 8 |
| 9 | CACAGAGTCG | 9 |
| 10 | TTCAGCGCGT | 10 |
| 11 | AGGACCGTGA | 11 |
| 12 | GTCTGTTCGC | 12 |
| 13 | ACCTGTCCAC | 13 |
| 14 | CCGCAATGAC | 14 |
| 15 | CTGCCGATCA | 15 |
| 16 | TACACGGAGC | 16 |
| 17 | CCGCATTCAT | 17 |
| 18 | GACTCTAGAC | 18 |
| 19 | GGAGAACTTA | 19 |
| 20 | TCCGGTATGC | 20 |
| 21 | GGTCAGGAGT | 21 |
| 22 | ACATTGGCAG | 22 |
| 23 | CGTAGACTGC | 23 |
| 24 | AGACTGTACT | 24 |
| 25 | TAGACGCAGT | 25 |
| 26 | CCGATAATCT | 26 |
| 27 | GAGAGCTAGT | 27 |
| 28 | GTACCGCGTT | 28 |
| 29 | GACTTGCGCA | 29 |
| 30 | CGTGATTGCG | 30 |
| 31 | ATCGTCTCTG | 31 |
| 32 | CGTAGCTACG | 32 |
| 33 | GCCGAATAGT | 33 |
| 34 | GTACCTAGGC | 34 |
| 35 | GCTTACATGA | 35 |
| 36 | TCCACGTAGT | 36 |
| 37 | AGAGGCCATC | 37 |
| 38 | CGGTGATGCT | 38 |
| 39 | CACTGTGCTT | 39 |
| 40 | CATGATGGCT | 40 |
| 41 | GCCACACATG | 41 |
| 42 | CACACACTGT | 42 |
| 43 | CAGAATCATA | 43 |
| 44 | ATCGTCTACG | 44 |
| 45 | CGAGCAATAC | 45 |
| 46 | ACAAGCGCAC | 46 |
| 47 | GCTTAGATGT | 47 |
| 48 | TGCATTCTGG | 48 |
| 49 | TGTCGGACCA | 49 |
| 50 | AGGCACTCGT | 50 |
| 51 | CTGCATGTGA | 51 |
| 52 | ACCACGCCTA | 52 |
| 53 | GAGGTCGTAC | 53 |
| 54 | AATACTCTGT | 54 |
| 55 | TGCCAACTGA | 55 |
| 56 | CCTGTTCGGT | 56 |
| 57 | GTAGAGAGTT | 57 |
| 58 | TACAGCGTAA | 58 |
| 59 | TGACGTGATG | 59 |
| 60 | AGACGTCGGT | 60 |
| 61 | CGCTAGGTTC | 61 |
| 62 | GCCTTATAGC | 62 |
| 63 | CCTTCGATCT | 63 |
| 64 | AGGCAACGTG | 64 |
| 65 | TGAGCGGTGT | 65 |
| 66 | GTGTCGAACG | 66 |
| 67 | CGATGTTGCG | 67 |
| 68 | AACAAGACAC | 68 |
| 69 | GATGCTGGTT | 69 |
| 70 | ACCGGTAGTC | 70 |
| 71 | GTGACTAGCA | 71 |
| 72 | AGCCTATATT | 72 |
| 73 | TCGTGAGCTT | 73 |
| 74 | ACACTATGGC | 74 |
| 75 | GACTCTGTCG | 75 |
| 76 | TCGATGATGC | 76 |
| 77 | CTTGGACACT | 77 |
| 78 | GGCTGATCGT | 78 |
| 79 | ACTCACAGGC | 79 |
| 80 | ATGTGCGTAC | 80 |
| 81 | CACCATCGAT | 81 |
| 82 | AGCCATTAAC | 82 |
| 83 | AATCGACTGT | 83 |
| 84 | AATACTAGCG | 84 |
| 85 | TCGTCACTGA | 85 |
| 86 | CAGGCTCTTA | 86 |
| 87 | GGTCGGTGAT | 87 |
| 88 | CATTAGGCGT | 88 |
| 89 | ACTCGCGAGT | 89 |
| 90 | TTCCGAATAA | 90 |
| 91 | TGAGCATCGT | 91 |
| 92 | GCCACGTAAC | 92 |
| 93 | GAACTACATG | 93 |
| 94 | TCGTGAGGAC | 94 |
| 95 | GCGGCCTTAA | 95 |
| 96 | GCTAAGGACC | 96 |

TABLE 2
Table 2 List of random primers
(10-base primers B)

| | | SEQ |
|---|---|---|
| | Primer | ID |
| No | sequence | NO |
| 1 | ATAGCCATTA | 97 |
| 2 | CAGTAATCAT | 98 |
| 3 | ACTCCTTAAT | 99 |
| 4 | TCGAACATTA | 100 |
| 5 | ATTATGAGGT | 101 |
| 6 | AATCTTAGAG | 102 |
| 7 | TTAGATGATG | 103 |
| 8 | TACATATCTG | 104 |
| 9 | TCCTTAATCA | 105 |
| 10 | GTTGAGATTA | 106 |
| 11 | TGTTAACGTA | 107 |
| 12 | CATACAGTAA | 108 |
| 13 | CTTATACGAA | 109 |
| 14 | AGATCTATGT | 110 |
| 15 | AAGACTTAGT | 111 |
| 16 | TGCGCAATAA | 112 |
| 17 | TTGGCCATAT | 113 |
| 18 | TATTACGAGG | 114 |
| 19 | TTATGATCGC | 115 |
| 20 | AACTTAGGAG | 116 |
| 21 | TCACAATCGT | 117 |
| 22 | GAGTATATGG | 118 |
| 23 | ATCAGGACAA | 119 |
| 24 | GTACTGATAG | 120 |
| 25 | CTTATACTCG | 121 |
| 26 | TAACGGACTA | 122 |
| 27 | GCGTTGTATA | 123 |
| 28 | CTTAAGTGCT | 124 |
| 29 | ATACGACTGT | 125 |
| 30 | ACTGTTATCG | 126 |
| 31 | AATCTTGACG | 127 |
| 32 | ACATCACCTT | 128 |
| 33 | GGTATAGTAC | 129 |
| 34 | CTAATCCACA | 130 |
| 35 | GCACCTTATT | 131 |
| 36 | ATTGACGGTA | 132 |
| 37 | GACATATGGT | 133 |
| 38 | GATAGTCGTA | 134 |
| 39 | GAATTATCGC | 135 |
| 40 | CTTAGGTGAT | 136 |
| 41 | CATACTACTG | 137 |
| 42 | TAACGCGAAT | 138 |
| 43 | CAAGTTACGA | 139 |
| 44 | AATCTCAAGG | 140 |
| 45 | GCAATCATCA | 141 |
| 46 | TGTAACGTTC | 142 |
| 47 | TATCGTTGGT | 143 |
| 48 | CGCTTAAGAT | 144 |
| 49 | TTAGAACTGG | 145 |
| 50 | GTCATAACGT | 146 |
| 51 | AGAGCAGTAT | 147 |
| 52 | CAACATCACT | 148 |
| 53 | CAGAAGCTTA | 149 |
| 54 | AACTAACGTG | 150 |
| 55 | TTATACCGCT | 151 |
| 56 | GAATTCGAGA | 152 |
| 57 | TTACGTAACC | 153 |
| 58 | GCATGGTTAA | 154 |
| 59 | GCACCTAATT | 155 |
| 60 | TGTAGGTTGT | 156 |
| 61 | CCATCTGGAA | 157 |
| 62 | TTCGCGTTGA | 158 |
| 63 | AACCGAGGTT | 159 |
| 64 | GTACGCTGTT | 160 |
| 65 | AGTATCCTGG | 161 |
| 66 | GGTTGTACAG | 162 |
| 67 | ACGTACACCA | 163 |
| 68 | TGTCGAGCAA | 164 |
| 69 | GTCGTGTTAC | 165 |
| 70 | GTGCAATAGG | 166 |
| 71 | ACTCGATGCT | 167 |
| 72 | GAATCGCGTA | 168 |
| 73 | CGGTCATTGT | 169 |
| 74 | ATCAGGCGAT | 170 |
| 75 | GTAAGATGCG | 171 |
| 76 | GGTCTCTTGA | 172 |
| 77 | TCCTCGCTAA | 173 |
| 78 | CTGCGTGATA | 174 |
| 79 | CATACTCGTC | 175 |
| 80 | ATCTGAGCTC | 176 |
| 81 | ACGGATAGTG | 177 |
| 82 | ACTGCAATGC | 178 |
| 83 | TAACGACGTG | 179 |
| 84 | TAGACTGTCG | 180 |
| 85 | CAGCACTTCA | 181 |
| 86 | AACATTCGCC | 182 |
| 87 | ACTAGTGCGT | 183 |
| 88 | ACGCTGTTCT | 184 |
| 89 | CGTCGAATGC | 185 |
| 90 | CTCTGACGGT | 186 |
| 91 | GTCGCCATGT | 187 |
| 92 | GGTCCACGTT | 188 |
| 93 | CGAGCGACTT | 189 |
| 94 | TTGACGCGTG | 190 |
| 95 | CTGAGAGCCT | 191 |
| 96 | CGCGCTAACT | 192 |

TABLE 3
Table 3 List of random primers
(10-base primers C)

| | | SEQ |
|---|---|---|
| | Primer | ID |
| No | sequence | NO |
| 1 | GGTCGTCAAG | 193 |
| 2 | AGGTTGACCA | 194 |
| 3 | TAACGGCAAC | 195 |
| 4 | GAGGCTGGAT | 196 |
| 5 | GTGCACACCT | 197 |
| 6 | TGAGGACCAG | 198 |
| 7 | TACTTGCGAG | 199 |
| 8 | AACTGTGAGA | 200 |
| 9 | CTCCATCAAC | 201 |
| 10 | CGGACTGTTA | 202 |
| 11 | TAGGACAGTC | 203 |
| 12 | AGAGGACACA | 204 |
| 13 | ACATTCGCGG | 205 |
| 14 | GCTTACTGCA | 206 |
| 15 | CAATACGTAA | 207 |
| 16 | AGACTTGCGC | 208 |
| 17 | GAGCGGTGTT | 209 |
| 18 | CGTGAGAGGT | 210 |
| 19 | AATCCGTCAG | 211 |
| 20 | ATACGTACCG | 212 |
| 21 | AACTGATTCC | 213 |
| 22 | CTGAGCGTAC | 214 |
| 23 | GTCGGATTCG | 215 |
| 24 | GCCGACCATA | 216 |
| 25 | GCAGAACTAA | 217 |
| 26 | CTAACGACCG | 218 |
| 27 | GCTGGACCAT | 219 |
| 28 | GACGCGGTTA | 220 |
| 29 | AGTGGTGAGC | 221 |
| 30 | CAGGCAGTCA | 222 |
| 31 | TCTGACGTCA | 223 |
| 32 | TACATGACGT | 224 |
| 33 | TGAGGCAACC | 225 |
| 34 | CAACTGCAGT | 226 |
| 35 | CGGAGATACG | 227 |
| 36 | CTTCGCAAGT | 228 |
| 37 | CTGGCATACG | 229 |
| 38 | TAACGTTCGC | 230 |
| 39 | CCGGCGTTAA | 231 |
| 40 | ACAAGACGCC | 232 |
| 41 | CCATTAGACT | 233 |
| 42 | GTCTGTGACA | 234 |
| 43 | GGCATTGGAC | 235 |
| 44 | TCTTCGCACG | 236 |
| 45 | TAGCCTGTGC | 237 |
| 46 | CACTGACCTA | 238 |
| 47 | CCGCACGATT | 239 |
| 48 | ATAGCACACG | 240 |
| 49 | GCACGTCATA | 241 |
| 50 | AAGCCGTTGG | 242 |
| 51 | CGGACCGTTA | 243 |
| 52 | TACACAGCGT | 244 |
| 53 | CGGACTTCAG | 245 |
| 54 | TAGAACGTCA | 246 |
| 55 | GGCATTGGAG | 247 |
| 56 | GGCACTCGTT | 248 |
| 57 | GTACCGTTAA | 249 |
| 58 | AATACGTGTC | 250 |
| 59 | CCATTGACGT | 251 |
| 60 | CGTGAATCGC | 252 |
| 61 | ATCAACGCGG | 253 |
| 62 | CGCCAAGGTA | 254 |
| 63 | AGAAGACGCC | 255 |
| 64 | CCGCATAGTC | 256 |
| 65 | CTTATATGTG | 257 |
| 66 | GGTCTCATCG | 258 |
| 67 | CCACCATGTC | 259 |
| 68 | ACGAATGTGT | 260 |
| 69 | GGTAGTAACA | 261 |
| 70 | GCCACTTAAT | 262 |
| 71 | ATATTGCGCC | 263 |
| 72 | GACCAATAGT | 264 |
| 73 | AACAACACGG | 265 |
| 74 | ATAGCCGATG | 266 |
| 75 | CGAGAGCATA | 267 |
| 76 | CGAGACATGA | 268 |
| 77 | CGCCAAGTTA | 269 |
| 78 | TTATAATCGC | 270 |
| 79 | TAGAAGTGCA | 271 |
| 80 | GGAGGCATGT | 272 |
| 81 | GCCACTTCGA | 273 |
| 82 | TCCACGGTAC | 274 |
| 83 | CAACTATGCA | 275 |
| 84 | CAAGGAGGAC | 276 |
| 85 | GAGGTACCTA | 277 |
| 86 | GAGCGCATAA | 278 |
| 87 | TCGTCACGTG | 279 |
| 88 | AACTGTGACA | 280 |
| 89 | TCCACGTGAG | 281 |
| 90 | ACACTGCTCT | 282 |
| 91 | TACGGTGAGC | 283 |
| 92 | CGGACTAAGT | 284 |
| 93 | AAGCCACGTT | 285 |
| 94 | CAATTACTCG | 286 |
| 95 | TCTGGCCATA | 287 |
| 96 | TCAGGCTAGT | 288 |

TABLE 4
Table 4 List of random primers
(10-base primers D)

| | | SEQ |
|---|---|---|
| | Primer | ID |
| No | sequence | NO |
| 1 | TTGACCCGGA | 289 |
| 2 | TTTTTATGGT | 290 |
| 3 | ATGTGGTGCG | 291 |
| 4 | AAGGCGCTAG | 292 |
| 5 | TCCAACTTTG | 293 |
| 6 | CCATCCCATC | 294 |
| 7 | CAATACGAGG | 295 |
| 8 | GAGTGTTACC | 296 |
| 9 | GCCTCCTGTA | 297 |
| 10 | CGAAGGTTGC | 298 |
| 11 | GAGGTGCTAT | 299 |
| 12 | TAGGATAATT | 300 |
| 13 | CGTTGTCCTC | 301 |
| 14 | TGAGACCAGC | 302 |
| 15 | TGCCCAAGCT | 303 |
| 16 | TACTGAATCG | 304 |
| 17 | TTACATAGTC | 305 |
| 18 | ACAAAGGAAA | 306 |
| 19 | CTCGCTTGGG | 307 |
| 20 | CCTTGCGTCA | 308 |
| 21 | TAATTCCGAA | 309 |
| 22 | GTGAGCTTGA | 310 |
| 23 | ATGCCGATTC | 311 |
| 24 | GCTTGGGCTT | 312 |
| 25 | ACAAAGCGCC | 313 |
| 26 | GAAAGCTCTA | 314 |
| 27 | TACCGACCGT | 315 |
| 28 | TCGAAGAGAC | 316 |
| 29 | GTCGCTTACG | 317 |
| 30 | GGGCTCTCGA | 318 |
| 31 | GCGCCCTTGT | 319 |
| 32 | GGCAATAGGC | 320 |
| 33 | CAAGTCAGGA | 321 |
| 34 | GGGTCGCAAT | 322 |
| 35 | CAGCAACCTA | 323 |
| 36 | TTCCCGCCAC | 324 |
| 37 | TGTGCATTTT | 325 |
| 38 | ATCAACGACG | 326 |
| 39 | GTGACGTCCA | 327 |
| 40 | CGATCTAGTC | 328 |
| 41 | TTACATCCTG | 329 |
| 42 | AGCCTTCAAT | 330 |
| 43 | TCCATCCGAT | 331 |
| 44 | GACTGGGTCT | 332 |
| 45 | TTCGGTGGAG | 333 |
| 46 | GACCAGCACA | 334 |
| 47 | CATTAACGGA | 335 |
| 48 | TTTTTCTTGA | 336 |
| 49 | CATTGCACTG | 337 |
| 50 | TGCGGCGATC | 338 |
| 51 | ATATTGCGGT | 339 |
| 52 | GACGTCGCTC | 340 |
| 53 | TCGCTTATCG | 341 |
| 54 | GCGCAGACAC | 342 |
| 55 | CATGTATTGT | 343 |
| 56 | TCTATAACCT | 344 |
| 57 | GTGGAGACAA | 345 |
| 58 | CGAAGATTAT | 346 |
| 59 | TAGCAACTGC | 347 |
| 60 | ATAATCGGTA | 348 |
| 61 | CAGGATGGGT | 349 |
| 62 | GACGATTCCC | 350 |
| 63 | CACGCCTTAC | 351 |
| 64 | AGTTGGTTCC | 352 |
| 65 | TCTTATCAGG | 353 |
| 66 | CGAGAAGTTC | 354 |
| 67 | GTGGTAGAAT | 355 |
| 68 | TAGGCTTGTG | 356 |
| 69 | ATGCGTTACG | 357 |
| 70 | ACTACCGAGG | 358 |
| 71 | CGAGTTGGTG | 359 |
| 72 | GGACGATCAA | 360 |
| 73 | AACAGTATGC | 361 |
| 74 | TTGGCTGATC | 362 |
| 75 | AGGATTGGAA | 363 |
| 76 | CATATGGAGA | 364 |
| 77 | CTGCAGGTTT | 365 |
| 78 | CTCTCTTTTT | 366 |
| 79 | AGTAGGGGTC | 367 |
| 80 | ACACCGCAAG | 368 |
| 81 | GAAGCGGGAG | 369 |
| 82 | GATACGGACT | 370 |
| 83 | TACGACGTGT | 371 |
| 84 | GTGCCTCCTT | 372 |
| 85 | GGTGACTGAT | 373 |
| 86 | ATATCTTACG | 374 |
| 87 | AATCATACGG | 375 |
| 88 | CTCTTGGGAC | 376 |
| 89 | GACGACAAAT | 377 |
| 90 | GTTGCGAGGT | 378 |
| 91 | AAACCGCACC | 379 |
| 92 | GCTAACACGT | 380 |
| 93 | ATCATGAGGG | 381 |
| 94 | GATTCACGTA | 382 |
| 95 | TCTCGAAAAG | 383 |
| 96 | CTCGTAACCA | 384 |

TABLE 5
Table 5 List of random primers
(10-base primers E)

| | | SEQ |
|---|---|---|
| | Primer | ID |
| No | sequence | NO |
| 1 | GTTACACACG | 385 |
| 2 | CGTGAAGGGT | 386 |
| 3 | ACGAGCATCT | 387 |
| 4 | ACGAGGGATT | 388 |
| 5 | GCAACGTCGG | 389 |
| 6 | CACGGCTAGG | 390 |
| 7 | CGTGACTCTC | 391 |
| 8 | TCTAGACGCA | 392 |
| 9 | CTGCGCACAT | 393 |
| 10 | ATGCTTGACA | 394 |
| 11 | TTTGTCGACA | 395 |
| 12 | ACGTGTCAGC | 396 |
| 13 | GAAAACATTA | 397 |
| 14 | ACATTAACGG | 398 |
| 15 | GTACAGGTCC | 399 |
| 16 | CTATGTGTAC | 400 |
| 17 | GCGTACATTA | 401 |
| 18 | GATTTGTGGC | 402 |
| 19 | TCGCGCGCTA | 403 |
| 20 | ACAAGGGCGA | 404 |
| 21 | AACGCGCGAT | 405 |
| 22 | CGTAAATGCG | 406 |
| 23 | TAGGCACTAC | 407 |
| 24 | GCGAGGATCG | 408 |
| 25 | CACGTTTACT | 409 |
| 26 | TACCACCACG | 410 |
| 27 | TTAACAGGAC | 411 |
| 28 | GCTGTATAAC | 412 |
| 29 | GTTGCTGGCA | 413 |
| 30 | AGTGTGGCCA | 414 |
| 31 | CTGCGGTTGT | 415 |
| 32 | TAGATCAGCG | 416 |
| 33 | TTCCGGTTAT | 417 |
| 34 | GATAAACTGT | 418 |
| 35 | TACAGTTGCC | 419 |
| 36 | CGATGGCGAA | 420 |
| 37 | CCGACGTCAG | 421 |
| 38 | TATGGTGCAA | 422 |
| 39 | GACGACAGTC | 423 |
| 40 | GTCACCGTCC | 424 |
| 41 | GGTTTTAACA | 425 |
| 42 | GAGGACAGTA | 426 |
| 43 | GTTACCTAAG | 427 |
| 44 | ATCACGTGTT | 428 |
| 45 | TAAGGCCTGG | 429 |
| 46 | TGTTCGTAGC | 430 |
| 47 | TGAGGACGTG | 431 |
| 48 | GTGCTGTGTA | 432 |
| 49 | GAGGGTACGC | 433 |
| 50 | CCGTGATTGT | 434 |
| 51 | AAAATCGCCT | 435 |
| 52 | CGATCGCAGT | 436 |
| 53 | ACGCAATAAG | 437 |
| 54 | AAGGTGCATC | 438 |
| 55 | CGCGTAGATA | 439 |
| 56 | CGAGCAGTGC | 440 |
| 57 | ATACGTGACG | 441 |
| 58 | AGATTGCGCG | 442 |
| 59 | ACGTGATGCC | 443 |
| 60 | GTACGCATCG | 444 |
| 61 | TCCCGACTTA | 445 |
| 62 | GTTTTTACAC | 446 |
| 63 | CCTGAGCGTG | 447 |
| 64 | CGGCATTGTA | 448 |
| 65 | TAGAGTGCGT | 449 |
| 66 | ATGGCCAGAC | 450 |
| 67 | CTTAGCATGC | 451 |
| 68 | ACAACACCTG | 452 |
| 69 | AGTGACTATC | 453 |
| 70 | CATGCTACAC | 454 |
| 71 | AAAGCGGGCG | 455 |
| 72 | AGATCGCCGT | 456 |
| 73 | CGTAGATATT | 457 |
| 74 | AATGGCAGAC | 458 |
| 75 | GTATAACGTG | 459 |
| 76 | ATGTGCGTCA | 460 |
| 77 | CCTGCCAACT | 461 |
| 78 | TTTATAACTC | 462 |
| 79 | ACGGTTACGC | 463 |
| 80 | TAGCCTCTTG | 464 |
| 81 | TCGCGAAGTT | 465 |
| 82 | GTCTACAACC | 466 |
| 83 | GTCTACTGCG | 467 |
| 84 | GTTGCGTCTC | 468 |
| 85 | GGGCCGCTAA | 469 |
| 86 | GTACGTCGGA | 470 |
| 87 | AGCGAGAGAC | 471 |
| 88 | TGGCTACGGT | 472 |
| 89 | AGGCATCACG | 473 |
| 90 | TAGCTCCTCG | 474 |
| 91 | GGCTAGTCAG | 475 |
| 92 | CTCACTTTAT | 476 |
| 93 | ACGGCCACGT | 477 |
| 94 | AGCGTATATC | 478 |
| 95 | GACACGTCTA | 479 |
| 96 | GCCAGCGTAC | 480 |

TABLE 6
Table 6 List of random primers
(10-base primers F)

| | | SEQ |
|---|---|---|
| | Primer | ID |
| No | sequence | NO |
| 1 | AACATTAGCG | 481 |
| 2 | AGTGTGCTAT | 482 |
| 3 | CACGAGCGTT | 483 |
| 4 | GTAACGCCTA | 484 |
| 5 | CACATAGTAC | 485 |
| 6 | CGCGATATCG | 486 |
| 7 | CGTTCTGTGC | 487 |
| 8 | CTGATCGCAT | 488 |
| 9 | TGGCGTGAGA | 489 |
| 10 | TTGCCAGGCT | 490 |
| 11 | GTTATACACA | 491 |
| 12 | AGTGCCAACT | 492 |
| 13 | TCACGTAGCA | 493 |
| 14 | TAATTCAGCG | 494 |
| 15 | AAGTATCGTC | 495 |
| 16 | CACAGTTACT | 496 |
| 17 | CCTTACCGTG | 497 |
| 18 | ACGGTGTCGT | 498 |
| 19 | CGCGTAAGAC | 499 |
| 20 | TTCGCACCAG | 500 |
| 21 | CACGAACAGA | 501 |
| 22 | GTTGGACATT | 502 |
| 23 | GGTGCTTAAG | 503 |
| 24 | TCGGTCTCGT | 504 |
| 25 | TCTAGTACGC | 505 |
| 26 | TTAGGCCGAG | 506 |
| 27 | CGTCAAGAGC | 507 |
| 28 | ACATGTCTAC | 508 |
| 29 | ATCGTTACGT | 509 |
| 30 | ACGGATCGTT | 510 |
| 31 | AATCTTGGCG | 511 |
| 32 | AGTATCTGGT | 512 |
| 33 | CAACCGACGT | 513 |
| 34 | TGGTAACGCG | 514 |
| 35 | GTGCAGACAT | 515 |
| 36 | GTCTAGTTGC | 516 |
| 37 | CAATTCGACG | 517 |
| 38 | CTTAGCACCT | 518 |
| 39 | TAATGTCGCA | 519 |
| 40 | CAATCGGTAC | 520 |
| 41 | AGCACGCATT | 521 |
| 42 | AGGTCCTCGT | 522 |
| 43 | TTGTGCCTGC | 523 |
| 44 | ACCGCCTGTA | 524 |
| 45 | GTACGTCAGG | 525 |
| 46 | GCACACAACT | 526 |
| 47 | TGAGCACTTA | 527 |
| 48 | GTGCCGCATA | 528 |
| 49 | ATGTTTTCGC | 529 |
| 50 | ACACTTAGGT | 530 |
| 51 | CGTGCCGTGA | 531 |
| 52 | TTACTAATCA | 532 |
| 53 | GTGGCAGGTA | 533 |
| 54 | GCGCGATATG | 534 |
| 55 | GAACGACGTT | 535 |
| 56 | ATCAGGAGTG | 536 |
| 57 | GCCAGTAAGT | 537 |
| 58 | GCAAGAAGCA | 538 |
| 59 | AACTCCGCCA | 539 |
| 60 | ACTTGAGCCT | 540 |
| 61 | CGTGATCGTG | 541 |
| 62 | AATTAGCGAA | 542 |
| 63 | ACTTCCTTAG | 543 |
| 64 | TGTGCTGATA | 544 |
| 65 | AGGCGCCTGA | 545 |
| 66 | CGTTTAGAGC | 546 |
| 67 | ACGCGTCTAA | 547 |
| 68 | GCGAATGTAC | 548 |
| 69 | CGTGATCCAA | 549 |
| 70 | CAACCAGATG | 550 |
| 71 | ACCATTAACC | 551 |
| 72 | CGATTCACGT | 552 |
| 73 | CTAGAACCTG | 553 |
| 74 | CCTAACGACA | 554 |
| 75 | GACGTGCATG | 555 |
| 76 | ATGTAACCTT | 556 |
| 77 | GATACAGTCG | 557 |
| 78 | CGTATGTCTC | 558 |
| 79 | AGATTATCGA | 559 |
| 80 | ATACTGGTAA | 560 |
| 81 | GTTGAGTAGC | 561 |
| 82 | ACCATTATCA | 562 |
| 83 | CACACTTCAG | 563 |
| 84 | GACTAGCGGT | 564 |
| 85 | AATTGTCGAG | 565 |
| 86 | CTAAGGACGT | 566 |
| 87 | ATTACGATGA | 567 |
| 88 | ATTGAAGACT | 568 |
| 89 | GCTTGTACGT | 569 |
| 90 | CCTACGTCAC | 570 |
| 91 | CACAACTTAG | 571 |
| 92 | GCGGTTCATC | 572 |
| 93 | GTACTCATCT | 573 |
| 94 | GTGCATCAGT | 574 |
| 95 | TCACATCCTA | 575 |
| 96 | CACGCGCTAT | 576 |

TABLE 7
Table 7 List of random primers
(8-base primers)

| | | SEQ |
|---|---|---|
| | Primer | ID |
| No | sequence | NO |
| 1 | CTATCTTG | 577 |
| 2 | AAGTGCGT | 578 |
| 3 | ACATGCGA | 579 |
| 4 | ACCAATGG | 580 |
| 5 | TGCGTTGA | 581 |
| 6 | GACATGTC | 582 |
| 7 | TTGTGCGT | 583 |
| 8 | ACATCGCA | 584 |
| 9 | GAAGACGA | 585 |
| 10 | TCGATAGA | 586 |
| 11 | TCTTGCAA | 587 |
| 12 | AGCAAGTT | 588 |
| 13 | TTCATGGA | 589 |
| 14 | TCAATTCG | 590 |
| 15 | CGGTATGT | 591 |
| 16 | ACCACTAC | 592 |
| 17 | TCGCTTAT | 593 |
| 18 | TCTCGACT | 594 |
| 19 | GAATCGGT | 595 |
| 20 | GTTACAAG | 596 |
| 21 | CTGTGTAG | 597 |
| 22 | TGGTAGAA | 598 |
| 23 | ATACTGCG | 599 |
| 24 | AACTCGTC | 600 |
| 25 | ATATGTGC | 601 |
| 26 | AAGTTGCG | 602 |
| 27 | GATCATGT | 603 |
| 28 | TTGTTGCT | 604 |
| 29 | CCTCTTAG | 605 |
| 30 | TCACAGCT | 606 |
| 31 | AGATTGAC | 607 |
| 32 | AGCCTGAT | 608 |
| 33 | CGTCAAGT | 609 |
| 34 | AAGTAGAC | 610 |
| 35 | TCAGACAA | 611 |
| 36 | TCCTTGAC | 612 |
| 37 | GTAGCTGT | 613 |
| 38 | CGTCGTAA | 614 |
| 39 | CCAATGGA | 615 |
| 40 | TTGAGAGA | 616 |
| 41 | ACAACACC | 617 |
| 42 | TCTAGTAC | 618 |
| 43 | GAGGAAGT | 619 |
| 44 | GCGTATTG | 620 |
| 45 | AAGTAGCT | 621 |
| 46 | TGAACCTT | 622 |
| 47 | TGTGTTAC | 623 |
| 48 | TAACCTGA | 624 |
| 49 | GCTATTCC | 625 |
| 50 | GTTAGATG | 626 |
| 51 | CAGGATAA | 627 |
| 52 | ACCGTAGT | 628 |
| 53 | CCGTGTAT | 629 |
| 54 | TCCACTCT | 630 |
| 55 | TAGCTCAT | 631 |
| 56 | CGCTAATA | 632 |
| 57 | TACCTCTG | 633 |
| 58 | TGCACTAC | 634 |
| 59 | CTTGGAAG | 635 |
| 60 | AATGCACG | 636 |
| 61 | CACTGTTA | 637 |
| 62 | TCGACTAG | 638 |
| 63 | CTAGGTTA | 639 |
| 64 | GCAGATGT | 640 |
| 65 | AGTTCAGA | 641 |
| 66 | CTCCATCA | 642 |
| 67 | TGGTTACG | 643 |
| 68 | ACGTAGCA | 644 |
| 69 | CTCTTCCA | 645 |
| 70 | CGTCAGAT | 646 |
| 71 | TGGATCAT | 647 |
| 72 | ATATCGAC | 648 |
| 73 | TTGTGGAG | 649 |
| 74 | TTAGAGCA | 650 |
| 75 | TAACTACC | 651 |
| 76 | CTATGAGG | 652 |
| 77 | CTTCTCAC | 653 |
| 78 | CGTTCTCT | 654 |
| 79 | GTCACTAT | 655 |
| 80 | TCGTTAGC | 656 |
| 81 | ATCGTGTA | 657 |
| 82 | GAGAGCAA | 658 |
| 83 | AGACGCAA | 659 |
| 84 | TCCAGTTA | 660 |
| 85 | AATGCCAC | 661 |
| 86 | ATCACGTG | 662 |
| 87 | ACTGTGCA | 663 |
| 88 | TCACTGCA | 664 |
| 89 | GCATCCAA | 665 |
| 90 | AGCACTAT | 666 |
| 91 | CGAAGGAT | 667 |
| 92 | CCTTGTGT | 668 |
| 93 | TGCGGATA | 669 |
| 94 | AGGAATGG | 670 |
| 95 | ATCGTAAC | 671 |
| 96 | GAATGTCT | 672 |

TABLE 8
Table 8 List of random primers
(9-base primers)

| | | SEQ |
|---|---|---|
| | Primer | ID |
| No | sequence | NO |
| 1 | TTGCTACAT | 673 |
| 2 | TAACGTATG | 674 |
| 3 | CAGTATGTA | 675 |
| 4 | TCAATAACG | 676 |
| 5 | CACACTTAT | 677 |
| 6 | GACTGTAAT | 678 |
| 7 | TATACACTG | 679 |
| 8 | ACTGCATTA | 680 |
| 9 | ACATTAAGC | 681 |
| 10 | CATATTACG | 682 |
| 11 | ATATCTACG | 683 |
| 12 | AGTAACTGT | 684 |
| 13 | ATGACGTTA | 685 |
| 14 | ATTATGCGA | 686 |
| 15 | AGTATACAC | 687 |
| 16 | TTAGCGTTA | 688 |
| 17 | TATGACACT | 689 |
| 18 | ATTAACGCT | 690 |
| 19 | TAGGACAAT | 691 |
| 20 | AAGACGTTA | 692 |
| 21 | TATAAGCGT | 693 |
| 22 | ATACCTGGC | 694 |
| 23 | CTCGAGATC | 695 |
| 24 | ATGGTGAGG | 696 |
| 25 | ATGTCGACG | 697 |
| 26 | GACGTCTGA | 698 |
| 27 | TACACTGCG | 699 |
| 28 | ATCGTCAGG | 700 |
| 29 | TGCACGTAC | 701 |
| 30 | GTCGTGCAT | 702 |
| 31 | GAGTGTTAC | 703 |
| 32 | AGACTGTAC | 704 |
| 33 | TGCGACTTA | 705 |
| 34 | TGTCCGTAA | 706 |
| 35 | GTAATCGAG | 707 |
| 36 | GTACCTTAG | 708 |
| 37 | ATCACGTGT | 709 |
| 38 | ACTTAGCGT | 710 |
| 39 | GTAATCGTG | 711 |
| 40 | ATGCCGTTA | 712 |
| 41 | ATAACGTGC | 713 |
| 42 | CTACGTTGT | 714 |
| 43 | TATGACGCA | 715 |
| 44 | CCGATAACA | 716 |
| 45 | ATGCGCATA | 717 |
| 46 | GATAAGCGT | 718 |
| 47 | ATATCTGCG | 719 |
| 48 | ACTTAGACG | 720 |
| 49 | ATCACCGTA | 721 |
| 50 | TAAGACACG | 722 |
| 51 | AATGCCGTA | 723 |
| 52 | AATCACGTG | 724 |
| 53 | TCGTTAGTC | 725 |
| 54 | CATCATGTC | 726 |
| 55 | TAAGACGGT | 727 |
| 56 | TGCATAGTG | 728 |
| 57 | GAGCGTTAT | 729 |
| 58 | TGCCTTACA | 730 |
| 59 | TTCGCGTTA | 731 |
| 60 | GTGTTAACG | 732 |
| 61 | GACACTGAA | 733 |
| 62 | CTGTTATCG | 734 |
| 63 | GGTCGTTAT | 735 |
| 64 | CGAGAGTAT | 736 |
| 65 | ATACAGTCC | 737 |
| 66 | AATTCACGC | 738 |
| 67 | TATGTGCAC | 739 |
| 68 | GATGACGTA | 740 |
| 69 | GATGCGATA | 741 |
| 70 | GAGCGATTA | 742 |
| 71 | TGTCACAGA | 743 |
| 72 | TACTAACCG | 744 |
| 73 | CATAACGAG | 745 |
| 74 | CGTATACCT | 746 |
| 75 | TATCACGTG | 747 |
| 76 | GAACGTTAC | 748 |
| 77 | GTCGTATAC | 749 |
| 78 | ATGTCGACA | 750 |
| 79 | ATACAGCAC | 751 |
| 80 | TACTTACGC | 752 |
| 81 | AACTACGGT | 753 |
| 82 | TAGAACGGT | 754 |
| 83 | GAATGTCAC | 755 |
| 84 | TGTACGTCT | 756 |
| 85 | AACATTGCG | 757 |
| 86 | TTGAACGCT | 758 |
| 87 | AATCAGGAC | 759 |
| 88 | ATTCGCACA | 760 |
| 89 | CCATGTACT | 761 |
| 90 | TGTCCTGTT | 762 |
| 91 | TAATTGCGC | 763 |
| 92 | GATAGTGTG | 764 |
| 93 | ATAGACGCA | 765 |
| 94 | TGTACCGTT | 766 |
| 95 | ATTGTCGCA | 767 |
| 96 | GTCACGTAA | 768 |

TABLE 9
Table 9 List of random primers
(11-base primers)

| | | SEQ |
|---|---|---|
| | Primer | ID |
| No | sequence | NO |
| 1 | TTACACTATGC | 769 |
| 2 | GCGATAGTCGT | 770 |
| 3 | CTATTCACAGT | 771 |
| 4 | AGAGTCACTGT | 772 |
| 5 | AGAGTCGAAGC | 773 |
| 6 | CTGAATATGTG | 774 |
| 7 | ACTCCACAGGA | 775 |
| 8 | ATCCTCGTAAG | 776 |
| 9 | TACCATCGCCT | 777 |
| 10 | AACGCCTATAA | 778 |
| 11 | CTGTCGAACTT | 779 |
| 12 | TCAGATGTCCG | 780 |
| 13 | CTGCTTATCGT | 781 |
| 14 | ACATTCGCACA | 782 |
| 15 | CCTTAATGCAT | 783 |
| 16 | GGCTAGCTACT | 784 |
| 17 | TTCCAGTTGGC | 785 |
| 18 | GAGTCACAAGG | 786 |
| 19 | CAGAAGGTTCA | 787 |
| 20 | TCAACGTGCAG | 788 |
| 21 | CAAGCTTACTA | 789 |
| 22 | AGAACTCGTTG | 790 |
| 23 | CCGATACAGAG | 791 |
| 24 | GTACGCTGATC | 792 |
| 25 | TCCTCAGTGAA | 793 |
| 26 | GAGCCAACATT | 794 |
| 27 | GAGATCGATGG | 795 |
| 28 | ATCGTCAGCTG | 796 |
| 29 | GAAGCACACGT | 797 |
| 30 | ATCACGCAACC | 798 |
| 31 | TCGAATAGTCG | 799 |
| 32 | TATTACCGTCT | 800 |
| 33 | CAGTCACGACA | 801 |
| 34 | TTACTCGACGT | 802 |
| 35 | GCAATGTTGAA | 803 |
| 36 | GACACGAGCAA | 804 |
| 37 | CGAGATTACAA | 805 |
| 38 | TACCGACTACA | 806 |
| 39 | ACCGTTGCCAT | 807 |
| 40 | ATGTAATCGCC | 808 |
| 41 | AAGCCTGATGT | 809 |
| 42 | AAGTAACGTGG | 810 |
| 43 | GTAGAGGTTGG | 811 |
| 44 | CTCTTGCCTCA | 812 |
| 45 | ATCGTGAAGTG | 813 |
| 46 | ACCAGCACTAT | 814 |
| 47 | CACCAGAATGT | 815 |
| 48 | GAGTGAACAAC | 816 |
| 49 | TAACGTTACGC | 817 |
| 50 | CTTGGATCTTG | 818 |
| 51 | GTTCCAACGTT | 819 |
| 52 | CAAGGACCGTA | 820 |
| 53 | GACTTCACGCA | 821 |
| 54 | CACACTACTGG | 822 |
| 55 | TCAGATGAATC | 823 |
| 56 | TATGGATCTGG | 824 |
| 57 | TCTTAGGTGTG | 825 |
| 58 | TGTCAGCGTCA | 826 |
| 59 | GTCTAGGACAG | 827 |
| 60 | GCCTCTTCATA | 828 |
| 61 | AGAAGTGTTAC | 829 |
| 62 | CATGAGGCTTG | 830 |
| 63 | TGGATTGCTCA | 831 |
| 64 | ATCTACCTAAG | 832 |
| 65 | ATGAGCAGTGA | 833 |
| 66 | CCAGGAGATAC | 834 |
| 67 | CCGTTATACTT | 835 |
| 68 | CTCAGTACAAG | 836 |
| 69 | GGTGATCGTAG | 837 |
| 70 | CGAACGAGACA | 838 |
| 71 | ACTACGAGCTT | 839 |
| 72 | TTGCCACAGCA | 840 |
| 73 | GTCAACTCTAC | 841 |
| 74 | TGGACTGTGTC | 842 |
| 75 | GGAATGGACTT | 843 |
| 76 | CGAGAACATAA | 844 |
| 77 | ACCTGGTCAGT | 845 |
| 78 | CGAACGACACA | 846 |
| 79 | AGTCTAGCCAT | 847 |
| 80 | AGGCCTAGATG | 848 |
| 81 | GGTGCGTTAGT | 849 |
| 82 | ATTGTGTCCGA | 850 |
| 83 | GCAGACATTAA | 851 |
| 84 | ATTGGCTCATG | 852 |
| 85 | GAGGTTACATG | 853 |
| 86 | CCTATAGGACC | 854 |
| 87 | TTAGACGGTCT | 855 |
| 88 | GATTGACGCAC | 856 |
| 89 | AAGACACCTCG | 857 |
| 90 | TCGAATAATCG | 858 |
| 91 | TCTATGTCGGA | 859 |
| 92 | TCGCATGAACC | 860 |
| 93 | TGTTATGTCTC | 861 |
| 94 | TGGATCCTACA | 862 |
| 95 | ATCGTTCAGCC | 863 |
| 96 | TACCGCAAGCA | 861 |

TABLE 10
Table 10 List of random primers
(12 base primers)

| | | SEQ |
|---|---|---|
| | Primer | ID |
| No | sequence | NO |
| 1 | GCTGTTGAACCG | 865 |
| 2 | ATACTCCGAGAT | 866 |
| 3 | CTTAAGGAGCGC | 867 |
| 4 | TATACTACAAGC | 868 |
| 5 | TAGTGGTCGTCA | 869 |
| 6 | GTGCTTCAGGAG | 870 |
| 7 | GACGCATACCTC | 871 |
| 8 | CCTACCTGTGGA | 872 |
| 9 | GCGGTCACATAT | 873 |
| 10 | CTGCATTCACGA | 874 |
| 11 | TGGATCCTTCAT | 875 |
| 12 | TTGTGCTGGACT | 876 |
| 13 | ATTGAGAGCTAT | 877 |
| 14 | TCGCTAATGTAG | 878 |
| 15 | CTACTGGCACAA | 879 |
| 16 | AGAGCCAGTCGT | 880 |
| 17 | AATACTGGCTAA | 881 |
| 18 | CTGCATGCATAA | 882 |
| 19 | TTGTCACAACTC | 883 |
| 20 | TGCTAACTCTCC | 884 |
| 21 | TCTCTAGTTCGG | 885 |
| 22 | TTACGTCCGCAA | 886 |
| 23 | GTGTTGCTACCA | 887 |
| 24 | CGCATGTATGCC | 888 |
| 25 | CCTGTTCTGATT | 889 |
| 26 | TAAGATGCTTGA | 890 |
| 27 | ATATATCTCAGC | 891 |
| 28 | TTCCTCGTGGTT | 892 |
| 29 | ATGTCGATCTAG | 893 |
| 30 | CATCCACTAATC | 894 |
| 31 | GCCTCTGGTAAC | 895 |
| 32 | AGTCAAGAGATT | 896 |
| 33 | ACTGAGGCGTTC | 897 |
| 34 | TAAGGCTGACAT | 898 |
| 35 | AGTTCGCATACA | 899 |
| 36 | GCAGAATTGCGA | 900 |
| 37 | GGTTATGAAGAA | 901 |
| 38 | AGAAGTCGCCTC | 902 |
| 39 | TTCGCGTTATTG | 903 |
| 40 | TACCTGGTCGGT | 904 |
| 41 | GGTTACCGAGGA | 905 |
| 42 | ACACACTTCTAG | 906 |
| 43 | GGAAGTGATTAA | 907 |
| 44 | TCCATCAGATAA | 908 |
| 45 | TGTCTGTATCAT | 909 |
| 46 | AATTGGCTATAG | 910 |
| 47 | ACGTCGGAAGGT | 911 |
| 48 | AGGCATCCGTTG | 912 |
| 49 | ACCGTCGCTTGA | 913 |
| 50 | TACCGTCAAGTG | 914 |
| 51 | CTCGATATAGTT | 915 |
| 52 | CGTCAACGTGGT | 916 |
| 53 | TAGTCAACGTAG | 917 |
| 54 | TGAGTAGGTCAG | 918 |
| 55 | CTTGGCATGTAC | 919 |
| 56 | TGCCGAGACTTC | 920 |
| 57 | CTAAGACTTAAG | 921 |
| 58 | TTCTCGTGTGCG | 922 |
| 59 | CACCTGCACGAT | 923 |
| 60 | ATTAAGCCTAAG | 924 |
| 61 | GGTGGAACCATG | 925 |
| 62 | ACTAACGCGACT | 926 |
| 63 | CAGTTGTGCTAT | 927 |
| 64 | ACGCTGTTAGCA | 928 |
| 65 | GTCAACGCTAAG | 929 |
| 66 | AGCTTAGGTATG | 930 |
| 67 | CGCAGGACGATT | 931 |
| 68 | AACCGGCTGTCT | 932 |
| 69 | GTTGCTCACGTG | 933 |
| 70 | GAATCTTCCGCG | 934 |
| 71 | AGAGCGTACACG | 935 |
| 72 | AAGGCTAATGTC | 936 |
| 73 | TCTATGTAGACG | 937 |
| 74 | AGACGGTCTAGT | 938 |
| 75 | TTGGTCACACGC | 939 |
| 76 | GTCGATATATGG | 940 |
| 77 | AACATGGATACG | 941 |
| 78 | TTCGCAGTTCCT | 942 |
| 79 | CGCATGTTGTGC | 943 |
| 80 | TGTTAAGTTGGA | 944 |
| 81 | CAAGTGTGATGA | 945 |
| 82 | CTGGTACCACGT | 946 |
| 83 | CGCTAGGATCAC | 947 |
| 84 | TGCTCATTACGG | 948 |
| 85 | TGCTCAGTAACA | 949 |
| 86 | ACGATCATAGCC | 950 |
| 87 | ACGATACGTGGA | 951 |
| 88 | GTTCGATGATGG | 952 |
| 89 | AAGAGCTGTGCC | 953 |
| 90 | GGTTGGATCAAC | 954 |
| 91 | GCGCGCTTATGA | 955 |
| 92 | CGTCGATCATCA | 956 |
| 93 | GAGACTGCACTC | 957 |
| 94 | GATAGATCGCAT | 958 |
| 95 | GGCCATCATCAG | 959 |
| 96 | GGTGTTCCACTG | 960 |

TABLE 11
Table 11 List of random primers
(14-base primers)

| | | SEQ |
|---|---|---|
| | Primer | ID |
| No | sequence | NO |
| 1 | AGCTATACAGAGGT | 961 |
| 2 | AGGCCGTTCTGTCT | 962 |
| 3 | CATTGGTCTGCTAT | 963 |
| 4 | CTACATACGCGCCA | 964 |
| 5 | GCTTAACGGCGCTT | 965 |
| 6 | TACGATACTCCACC | 966 |
| 7 | ACCGGCATAAGAAG | 967 |
| 8 | GGATGCTTCGATAA | 968 |
| 9 | GTGTACCTGAATGT | 969 |
| 10 | CGCGGATACACAGA | 970 |
| 11 | TTCCACGGCACTGT | 971 |
| 12 | TAGCCAGGCAACAA | 972 |
| 13 | AGCGTCAACACGTA | 973 |
| 14 | TAACGCTACTCGCG | 974 |
| 15 | TAGATAGACGATCT | 975 |
| 16 | ACTCTTGCAATGCT | 976 |
| 17 | ACTCGGTTAGGTCG | 977 |
| 18 | CATTATCTACGCAT | 978 |
| 19 | CACACCGGCGATTA | 979 |
| 20 | TACGCAGTACTGTG | 980 |
| 21 | CAAGCGCGTGAATG | 981 |
| 22 | GAATGGACTGACGA | 982 |
| 23 | CTAGCGCTGAAGTT | 983 |
| 24 | TGCGGCAGACCAAT | 984 |
| 25 | AAGGCATAGAGATT | 985 |
| 26 | TTCTCCTCGCCATG | 986 |
| 27 | TCATTGGTCGTGAA | 987 |
| 28 | ATTACGCTATACGA | 988 |
| 29 | ATGATCCTCCACGG | 989 |
| 30 | CGTCGTTAGTAATC | 990 |
| 31 | TGCACATAGTCTCA | 991 |
| 32 | GTCAAGGAGTCACG | 992 |
| 33 | GGTTGGAATCTTGC | 993 |
| 34 | CATCGGTGCACTCA | 994 |
| 35 | AATGCACTAGACGT | 995 |
| 36 | TACAGTCAGGCTCG | 996 |
| 37 | AGAGAAGCTTAGCC | 997 |
| 38 | CCATAGGATCGTAT | 998 |
| 39 | TTGTGCTACACCTG | 999 |
| 40 | CTCCAGTAATACTA | 1000 |
| 41 | TGATGCCGATGTGG | 1001 |
| 42 | GTCATACCGCTTAA | 1002 |
| 43 | ACGTTCTCTTGAGA | 1003 |
| 44 | CAGCCATATCGTGT | 1004 |
| 45 | TTGAACGTAGCAAT | 1005 |
| 46 | ACAATCGCGGTAAT | 1006 |
| 47 | GTTCCTGTAGATCC | 1007 |
| 48 | AGAGCCTTACGGCA | 1008 |
| 49 | AATATGGCGCCACC | 1009 |
| 50 | ACCATATAGGTTCG | 1010 |
| 51 | ATGCACCACAGCTG | 1011 |
| 52 | CTACTATTGAACAG | 1012 |
| 53 | TGCCATCACTCTAG | 1013 |
| 54 | GCGAACGAGAATCG | 1014 |
| 55 | GAATCAAGGAGACC | 1015 |
| 56 | CAACATCTATGCAG | 1016 |
| 57 | CAATCCGTCATGGA | 1017 |
| 58 | AGCTCTTAGCCATA | 1018 |
| 59 | AACAAGGCAACTGG | 1019 |
| 60 | GTCGTCGCTCCTAT | 1020 |
| 61 | GTCATCATTAGATG | 1021 |
| 62 | GCACTAAGTAGCAG | 1022 |
| 63 | ACCTTACCGGACCT | 1023 |
| 64 | GCTCAGGTATGTCA | 1024 |
| 65 | TGTCACGAGTTAGT | 1025 |
| 66 | CAGATGACTTACGT | 1026 |
| 67 | GAAGTAGCGATTGA | 1027 |
| 68 | GCAGGCAATCTGTA | 1028 |
| 69 | CCTTATACAACAAG | 1029 |
| 70 | CCTTAGATTGATTG | 1030 |
| 71 | AGCCACGAGTGATA | 1031 |
| 72 | GGATGACTCGTGAC | 1032 |
| 73 | CTTCGTTCGCCATT | 1033 |
| 74 | TCTTGCGTATTGAT | 1034 |
| 75 | CTTAACGTGGTGGC | 1035 |
| 76 | TGCTGTTACGGAAG | 1036 |
| 77 | CTGAATTAGTTCTC | 1037 |
| 78 | CCTCCAAGTACAGA | 1038 |
| 79 | CTGGTAATTCGCGG | 1039 |
| 80 | CGACTGCAATCTGG | 1040 |
| 81 | TGGATCGCGATTGG | 1041 |
| 82 | CGACTATTCCTGCG | 1042 |
| 83 | CAAGTAGGTCCGTC | 1043 |
| 84 | AGTAATCAGTGTTC | 1044 |
| 85 | TTATTCTCACTACG | 1045 |
| 86 | CATGTCTTCTTCGT | 1046 |
| 87 | AGGCACATACCATC | 1047 |
| 88 | AGGTTAGAGGATGT | 1048 |
| 89 | CAACTGGCAAGTGC | 1049 |
| 90 | CGCTCACATAGAGG | 1050 |
| 91 | GCAATGTCGAGATC | 1051 |
| 92 | GTTCTGTGGTGCTC | 1052 |
| 93 | AAGTGATCAGACTA | 1053 |
| 94 | ATTGAAGGATTCCA | 1054 |
| 95 | ACGCCATGCTACTA | 1055 |
| 96 | CTGAAGATGTCTGC | 1056 |

TABLE 12
Table 12 List of random primers
(16-base primers)

| | | SEQ |
|---|---|---|
| | Primer | ID |
| No | sequence | NO |
| 1 | GACAATCTCTGCCGAT | 1057 |
| 2 | GGTCCGCCTAATGTAA | 1058 |
| 3 | AGCCACAGGCAATTCC | 1059 |
| 4 | ATCTCAAGTTCTCAAC | 1060 |
| 5 | TGTAACGCATACGACG | 1061 |
| 6 | TATCTCGAATACCAGC | 1062 |
| 7 | ACCGCAACACAGGCAA | 1063 |
| 8 | GGCCAGTAACATGACT | 1064 |
| 9 | GTGAACAGTTAAGGTG | 1065 |
| 10 | CCAGGATCCGTATTGC | 1066 |
| 11 | GACCTAGCACTAGACC | 1067 |
| 12 | CGCCATCCTATTCACG | 1068 |
| 13 | AAGTGCAGTAATGGAA | 1069 |
| 14 | TCAACGCGTTCGTCTA | 1070 |
| 15 | AGCGGCCACTATCTAA | 1071 |
| 16 | CTCGGCGCCATATAGA | 1072 |
| 17 | CGATAACTTAGAAGAA | 1073 |
| 18 | CATAGGATGTGACGCC | 1074 |
| 19 | GGCTTGTCGTCGTATC | 1075 |
| 20 | CTTGTCTGAATATTAG | 1076 |
| 21 | ACAGTTCGAGTGTCGG | 1077 |
| 22 | CTCTAACCTGTGACGT | 1078 |
| 23 | CGCGCTAATTCAACAA | 1079 |
| 24 | ACTCACGAATGCGGCA | 1080 |
| 25 | AATCTTCGGCATTCAT | 1081 |
| 26 | AAGTATCAGGATCGCG | 1082 |
| 27 | AGTAACTCTGCAGACA | 1083 |
| 28 | GGATTGAACATTGTGC | 1084 |
| 29 | GTGATGCTCACGCATC | 1085 |
| 30 | CGTAGCGTAACGGATA | 1086 |
| 31 | TGCGATGCACCGTTAG | 1087 |
| 32 | CCAGTATGCTCTCAGG | 1088 |
| 33 | AATGACGTTGAAGCCT | 1089 |
| 34 | TCGATTCTATAGGAGT | 1090 |
| 35 | CGATAGGTTCAGCTAT | 1091 |
| 36 | CCATGTTGATAGAATA | 1092 |
| 37 | GAGCCACTTCTACAGG | 1093 |
| 38 | GCGAACTCTCGGTAAT | 1094 |
| 39 | GACCTGAGTAGCTGGT | 1095 |
| 40 | CGAGTCTATTAGCCTG | 1096 |
| 41 | GTAGTGCCATACACCT | 1097 |
| 42 | CCAGTGGTCTATAGCA | 1098 |
| 43 | GTCAGTGCGTTATTGC | 1099 |
| 44 | AGTGTCGGAGTGACGA | 1100 |
| 45 | AATCTCCGCTATAGTT | 1101 |
| 46 | CGAGTAGGTCTGACTT | 1102 |
| 47 | CTGTCGCTCTAATAAC | 1103 |
| 48 | GCTGTCAATATAACTG | 1104 |
| 49 | AGCTCAAGTTGAATCC | 1105 |
| 50 | AATTCATGCTCCTAAC | 1106 |
| 51 | CCAAGGTCTGGTGATA | 1107 |
| 52 | CTCCACGTATCTTGAA | 1108 |
| 52 | TAGCCGAACAACACTT | 1109 |
| 54 | AGTACACGACATATGC | 1110 |
| 55 | ACGTTCTAGACTCCTG | 1111 |
| 56 | CGACTCAAGCACTGCT | 1112 |
| 57 | TGAAGCTCACGATTAA | 1113 |
| 58 | TATCTAACGTATGGTA | 1114 |
| 59 | TATACCATGTTCCTTG | 1115 |
| 60 | TTCCTACGATGACTTC | 1116 |
| 61 | CTCTCCAATATGTGCC | 1117 |
| 62 | GAGTAGAGTCTTGCCA | 1118 |
| 63 | GCGAGATGTGGTCCTA | 1119 |
| 64 | AAGCTACACGGACCAC | 1120 |
| 65 | ATACAACTGGCAACCG | 1121 |
| 66 | CGGTAGATGCTATGCT | 1122 |
| 67 | TCTTGACCGGTCATCA | 1123 |
| 68 | AGATCGTGCATGCGAT | 1124 |
| 69 | TCCTCGAGACAGCCTT | 1125 |
| 70 | TAGCCGGTACCACTTA | 1126 |
| 71 | GTAAGGCAGCGTGCAA | 1127 |
| 72 | TAGTCTGCTCCTGGTC | 1128 |
| 73 | TGGATTATAGCAGCAG | 1129 |
| 74 | AAGAATGATCAGACAT | 1130 |
| 75 | CAGCGCTATATACCTC | 1131 |
| 76 | GAGTAGTACCTCCACC | 1132 |
| 77 | GACGTGATCCTCTAGA | 1133 |
| 78 | GTTCCGTTCACTACGA | 1134 |
| 79 | TGCAAGCACCAGGATG | 1135 |
| 80 | TTAGTTGGCGGCTGAG | 1136 |
| 81 | CAGATGCAGACATACG | 1137 |
| 82 | GACGCTTGATGATTAT | 1138 |
| 83 | TGGATCACGACTAGGA | 1139 |
| 84 | CTCGTCGGTATAACGC | 1140 |
| 85 | AAGCACGGATGCGATT | 1141 |
| 86 | AGATCTTCCGGTGAAC | 1142 |
| 87 | GGACAATAGCAACCTG | 1143 |
| 88 | GATAATCGGTTCCAAT | 1144 |
| 89 | CTCAAGCTACAGTTGT | 1145 |
| 90 | GTTGGCATGATGTAGA | 1146 |
| 91 | CAGCATGAGGTAAGTG | 1147 |
| 92 | GCCTCATCACACGTCA | 1148 |
| 93 | TCGATACTACACATCG | 1149 |
| 94 | TACACGAGGCTTGATC | 1150 |
| 95 | TTCTCGTGTCCGCATT | 1151 |
| 96 | GGTGAAGCAACAGCAT | 1152 |

TABLE 13
Table 13 List of random primers
(18-base primers)

| | | SEQ |
|---|---|---|
| | Primer | ID |
| No | sequence | NO |
| 1 | CGAACCGACTGTACAGTT | 1153 |
| 2 | CCGACTGCGGATAAGTTA | 1154 |
| 3 | CGACAGGTAGGTAAGCAG | 1155 |
| 4 | TGATACGTTGGTATACAG | 1156 |
| 5 | CTACTATAGAATACGTAG | 1157 |
| 6 | AGACTGTGGCAATGGCAT | 1158 |
| 7 | GGAAGACTGATACAACGA | 1159 |
| 8 | TATGCACATATAGCGCTT | 1160 |
| 9 | CATGGTAATCGACCGAGG | 1161 |
| 10 | GTCATTGCCGTCATTGCC | 1162 |
| 11 | CCTAAGAACTCCGAAGCT | 1163 |
| 12 | TCGCTCACCGTACTAGGA | 1164 |
| 13 | TATTACTGTCACAGCAGG | 1165 |
| 14 | TGAGACAGGCTACGAGTC | 1166 |
| 15 | AAGGTATGCGAACACGTT | 1167 |
| 16 | AACGGAGGAGTGAGCCAA | 1168 |
| 17 | CCACTATGGACATCATGG | 1169 |
| 18 | ATGGTGGTGGATAGCTCG | 1170 |
| 19 | TCACCGGTTACACATCGC | 1171 |
| 20 | AAGATACTGAGATATGGA | 1172 |
| 21 | GACCTGTTCTTGAACTAG | 1173 |
| 22 | AACTAGAGCTCTCGGTTA | 1174 |
| 23 | CTATGTTCTTACTCTCTT | 1175 |
| 24 | CAAGGCTATAAGCGGTTA | 1176 |
| 25 | GAAGCTAATTAACCGATA | 1177 |
| 26 | TTCACGTCTGCCAAGCAC | 1178 |
| 27 | ATGGTATAGATCGAGACA | 1179 |
| 28 | GTCACAGATTCACATCAT | 1180 |
| 29 | GTGCCTGTGAACTATCAG | 1181 |
| 30 | CAGCGTACAAGATAGTCG | 1182 |
| 31 | GCATGGCATGGTAGACCT | 1183 |
| 32 | GGTATGCTACTCTTCGCA | 1184 |
| 33 | ATGTTCAGTCACAAGCGA | 1185 |
| 34 | TAGGAAGTGTGTAATAGC | 1186 |
| 35 | AATCCATGTAGCTGTACG | 1187 |
| 36 | CCAGATTCACTGGCATAG | 1188 |
| 37 | TTGTCTCTACGTAATATC | 1189 |
| 38 | GTGGTGCTTGTGACAATT | 1190 |
| 39 | CAGCCTACTTGGCTGAGA | 1191 |
| 40 | TACTCAATGCATCTGTGT | 1192 |
| 41 | TGTAGAGAGACGAATATA | 1193 |
| 42 | GCCTACAACCATCCTACT | 1194 |
| 43 | GCGTGGCATTGAGATTCA | 1195 |
| 44 | GCATGCCAGCTAACTGAG | 1196 |
| 45 | GCGAGTAATCCGGTTGGA | 1197 |
| 46 | GCCTCTACCAGAACGTCA | 1198 |
| 47 | GTCAGCAGAAGACTGACC | 1199 |
| 48 | GATAACAGACGTAGCAGG | 1200 |
| 49 | CAGGAGATCGCATGTCGT | 1201 |
| 50 | CTGGAAGGAATGGAGCCA | 1202 |
| 51 | ATTGGTTCTCTACCACAA | 1203 |
| 52 | CTCATTGTTGACGGCTCA | 1204 |
| 53 | TTCAGGACTGTAGTTCAT | 1205 |
| 54 | AGACCGCACTAACTCAAG | 1206 |
| 55 | GGAATATTGTGCAGACCG | 1207 |
| 56 | CCTATTACTAATAGCTCA | 1208 |
| 57 | ATGGCATGAGTACTTCGG | 1209 |
| 58 | GACACGTATGCGTCTAGC | 1210 |
| 59 | GAAGGTACGGAATCTGTT | 1211 |
| 60 | TATAACGTCCGACACTGT | 1212 |
| 61 | GCTAATACATTACCGCCG | 1213 |
| 62 | GAAGCCAACACTCCTGAC | 1214 |
| 63 | CGAATAACGAGCTGTGAT | 1215 |
| 64 | GCCTACCGATCGCACTTA | 1216 |
| 65 | CTGAGGAGAATAGCCTGC | 1217 |
| 66 | CAGCATGGACAGTACTTC | 1218 |
| 67 | GGTATAGAGCCTTCCTTA | 1219 |
| 68 | CGCTCTGCATATATAGCA | 1220 |
| 69 | CGGCTCTACTATGCTCGT | 1221 |
| 70 | CCTAATGCGAAGCTCACC | 1222 |
| 71 | ACAACCGGTGAGGCAGTA | 1223 |
| 72 | TTGGTTCGAACCAACCGC | 1224 |
| 73 | ATACTAGGTTGAACTAAG | 1225 |
| 74 | GCGTTGAGAGTAACATAT | 1226 |
| 75 | AGTTGTATAATAAGCGTC | 1227 |
| 76 | GTATGATGCCGTCCAATT | 1228 |
| 77 | GGACTCTCTGAAGAGTCT | 1229 |
| 78 | GGACTCTCTTGACTTGAA | 1230 |
| 79 | GATAACAGTGCTTCGTCC | 1231 |
| 80 | GGCCATTATAGATGAACT | 1232 |
| 81 | ATAGAGAGCACAGAGCAG | 1233 |
| 82 | GTGTGAGTGTATCATAAC | 1234 |
| 83 | ATAACCTTAGTGCGCGTC | 1235 |
| 84 | CCGACTGATATGCATGGA | 1236 |
| 85 | GGATATCTGATCGCATCA | 1237 |
| 86 | CAGCATTAACGAGGCGAA | 1238 |
| 87 | GCGAGGCCTACATATTCG | 1239 |
| 88 | CGATAAGTGGTAAGGTCT | 1240 |
| 89 | AGATCCTGAGTCGAGCAA | 1241 |
| 90 | AAGATATAACGAGACCGA | 1242 |
| 91 | CCGACTGATTGAGAACGT | 1243 |
| 92 | TCGGCTTATATGACACGT | 1244 |
| 93 | AATAACGTACGCCGGAGG | 1245 |
| 94 | AACACAGCATTGCGCACG | 1246 |
| 95 | GTAGTCTGACAGCAACAA | 1247 |
| 96 | AGAATGACTTGAGCTGCT | 1248 |

TABLE 14
List of random primers (20-base primers)

| | | SEQ |
|---|---|---|
| | | ID |
| No | Primer sequence | NO |
| 1 | ACTGGTAGTAACGTCCACCT | 1249 |
| 2 | AGACTGGTTGTTATTCGCCT | 1250 |
| 3 | TATCATTGACAGCGAGCTCA | 1251 |
| 4 | TGGAGTCTGAAGAAGGACTC | 1252 |
| 5 | CATCTGGACTACGGCAACGA | 1253 |
| 6 | AACTGTCATAAGACAGACAA | 1254 |
| 7 | CCTCAACATGACATACACCG | 1255 |
| 8 | CAATACCGTTCGCGATTCTA | 1256 |
| 9 | GCGTCTACGTTGATTCGGCC | 1257 |
| 10 | TGAACAGAGGCACTTGCAGG | 1258 |
| 11 | CGACTAGAACCTACTACTGC | 1259 |
| 12 | GCACCGCACGTGGAGAGATA | 1260 |
| 13 | CTGAGAGACCGACTGATGCG | 1261 |
| 14 | TCGTCCTTCTACTTAATGAT | 1262 |
| 15 | CAAGCTATACCATCCGAATT | 1263 |
| 16 | CAATACGTATAGTCTTAGAT | 1264 |
| 17 | CCATCCACAGTGACCTATGT | 1265 |
| 18 | TATCCGTTGGAGAAGGTTCA | 1266 |
| 19 | CGCCTAGGTACCTGAGTACG | 1267 |
| 20 | CAGAGTGCTCGTGTTCGCGA | 1268 |
| 21 | CGCTTGGACATCCTTAAGAA | 1269 |
| 22 | GACCGCATGATTAGTCTTAC | 1270 |
| 23 | CTTGGCCGTAGTCACTCAGT | 1271 |
| 24 | GATAGCGATATTCAGTTCGC | 1272 |
| 25 | ATCCAACACTAAGACAACCA | 1273 |
| 26 | CCATTCTGTTGCGTGTCCTC | 1274 |
| 27 | ACATTCTGTACGCTTGCAGC | 1275 |
| 28 | TGCTGAACGCCAATCGCTTA | 1276 |
| 29 | TCCTCTACAAGAATATTGCG | 1277 |
| 30 | CGACCAACGCAGCCTGATTC | 1278 |
| 31 | ATTGCGAGCTTGAGTAGCGC | 1279 |
| 32 | AAGGTGCGAGCATAGGAATC | 1280 |
| 33 | CACTTAAGTGTGATATAGAT | 1281 |
| 34 | ATCGGTATGCTGACCTAGAC | 1282 |
| 35 | TACAATCTCGAATGCAGGAT | 1283 |
| 36 | CCATATGAAGCGCAGCCGTC | 1284 |
| 37 | CGTCTCGTGGACATTCGAGG | 1285 |
| 38 | CCGAGTACAGAAGCGTGGAA | 1286 |
| 39 | TTACGTGGTCGACAGGCAGT | 1287 |
| 40 | AGCTGCAATCTGCATGATTA | 1288 |
| 41 | ACCTGCCGAAGCAGCCTACA | 1289 |
| 42 | AACATGATAACCACATGGTT | 1290 |
| 43 | ATCCGACTGATTGAATTACC | 1291 |
| 44 | TCACGCTGACTCTTATCAGG | 1292 |
| 45 | GCGCGCTCGAAGTACAACAT | 1293 |
| 46 | ACAGCCAGATGCGTTGTTCC | 1294 |
| 47 | GGAGCTCTGACCTGCAAGAA | 1295 |
| 48 | AACATTAGCCTCAAGTAAGA | 1296 |
| 49 | TGTGATTATGCCGAATGAGG | 1297 |
| 50 | GAGTAATAATCCAATCAGTA | 1298 |
| 51 | CTCCTTGGCGACAGCTGAAC | 1299 |
| 52 | TTACGCACACATACACAGAC | 1300 |
| 53 | ACGCCGTATGGCGACTTAGG | 1301 |
| 54 | AGAACGACAATTACGATGGC | 1302 |
| 55 | TGCTAACGTACCACTGCCAC | 1303 |
| 56 | CATCCAGAATGTCTATCATA | 1304 |
| 57 | GGAGAACGCCTATAGCACTC | 1305 |
| 58 | ACCTCTTGTGACGGCCAGTC | 1306 |
| 59 | TGCCATAACTTGGCATAAGA | 1307 |
| 60 | ACAATTGTCTGACCACGCTC | 1308 |
| 61 | TCGTCACCTTCACAGAACGA | 1309 |
| 62 | AGCAGCAGATGATGATCCAA | 1310 |
| 63 | TCGTGCCTTGGATTCCAGGA | 1311 |
| 64 | TGTTATAGCCACGATACTAT | 1312 |
| 65 | AATCTCACCTGTACCTTCCG | 1313 |
| 66 | GAGTAGCGGAAGCGTTAGCG | 1314 |
| 67 | AATACTCCGGCGAGGTATAC | 1315 |
| 68 | TTCGCATCCTTGCACGAACA | 1316 |
| 69 | AACCGGCTAATACTACTGGC | 1317 |
| 70 | CTAGCATCTTAGACACCAGA | 1318 |
| 71 | TAGTTGCGTGATACAAGATA | 1319 |
| 72 | TCGTCTCGACACAGTTGGTC | 1320 |
| 73 | TCCGTTCGCGTGCGAACTGA | 1321 |
| 74 | TCTGACTCTGGTGTACAGTC | 1322 |
| 75 | ACAGCGCAATTATATCCTGT | 1323 |
| 76 | AGATCCGTACGTGAGACTAG | 1324 |
| 77 | TACATTGAAGCATCCGAACA | 1325 |
| 78 | CTCCTGAGAGATCAACGCCA | 1326 |
| 79 | TCACCTCGAATGAGTTCGTT | 1327 |
| 80 | TAGCGACTTAAGGTCCAAGC | 1328 |
| 81 | AGTACGTATTGCCGTGCAAG | 1329 |
| 82 | AGCCACGAACCGACGTCATA | 1330 |
| 83 | TGATGTGTACGCTACTACTA | 1331 |
| 84 | CCACTGTGTGCAGCAGACGA | 1332 |
| 85 | CTATTGTACAGCGAACGCTG | 1333 |
| 86 | CTCCGATATCGCACGGATCG | 1334 |
| 87 | AACTTATCGTCGGACGCATG | 1335 |
| 88 | TATCCTAATTCGTGCCGGTC | 1336 |
| 89 | ACAGCCTTCCTGTGTGGACT | 1337 |
| 90 | CCTCCGTGAGGATCGTACCA | 1338 |
| 91 | GCTCTAAGTAACAGAACTAA | 1339 |
| 92 | GACTTACCGCGCGTTCTGGT | 1340 |
| 93 | TCTGAGGATACACATGTGGA | 1341 |
| 94 | TGTAATCACACTGGTGTCGG | 1342 |
| 95 | CACTAGGCGGCAGACATACA | 1343 |
| 96 | CTAGAGCACAGTACCACGTT | 1344 |

TABLE 15
List of random primers (22-base primers)

| | | SEQ |
|---|---|---|
| | | ID |
| No | Primer sequence | NO |
| 1 | TTCAGAGGTCTACGCTTCCGGT | 1345 |
| 2 | AACACAGACTGCGTTATGCCAA | 1346 |
| 3 | TGCTGAGTTCTATACAGCAGTG | 1347 |
| 4 | ACCTATTATATGATAGCGTCAT | 1348 |
| 5 | ATCGTGAGCTACAGTGAATGCA | 1349 |
| 6 | CGTGATGTATCCGGCCTTGCAG | 1350 |
| 7 | TCTTCTGGTCCTAGAGTTGTGC | 1351 |
| 8 | TGATGTCGGCGGCGGATCAGAT | 1352 |
| 9 | TCGGCCTTAGCGTTCAGCATCC | 1353 |
| 10 | TTAAGTAGGTCAGCCACTGCAC | 1354 |
| 11 | CCAGGTGAGTTGATCTGACACC | 1355 |
| 12 | TATACTATTACTGTGTTCGATC | 1356 |
| 13 | CCGCAGTATGTCTAGTGTTGTC | 1357 |
| 14 | GTCTACCGCGTACGAAGCTCTC | 1358 |
| 15 | ATGCGAGTCCGTGGTCGATCCT | 1359 |
| 16 | TGGTAGATTGGTGTGAGAACTA | 1360 |
| 17 | AGGTTCGTCGATCAACTGCTAA | 1361 |
| 18 | ACGACAAGCATCCTGCGATATC | 1362 |
| 19 | TTGAATCACAGAGAGCGTGATT | 1363 |
| 20 | GTACTTAGTGCTTACGTCAGCT | 1364 |
| 21 | GATTATTAAGGCCAAGCTCATA | 1365 |
| 22 | GCATGCAGAGACGTACTCATCG | 1366 |
| 23 | TAGCGGATGGTGTCCTGGCACT | 1367 |
| 24 | TACGGCTGCCAACTTAATAACT | 1368 |
| 25 | CTCATATGACAACTTCTATAGT | 1369 |
| 26 | CAAGCAATAGTTGTCGGCCACC | 1370 |
| 27 | TTCAGCAATCCGTACTGCTAGA | 1371 |
| 28 | TGAGACGTTGCTGACATTCTCC | 1372 |
| 29 | GTTCCGATGAGTTAGATGTATA | 1373 |
| 30 | TTGACGCTTGGAGGAGTACAAG | 1374 |
| 31 | TTCATGTTACCTCCACATTGTG | 1375 |
| 32 | GAGCACGTGCCAGATTGCAACC | 1376 |
| 33 | GGTCGACAAGCACAAGCCTTCT | 1377 |
| 34 | TAGGCAGGTAAGATGACCGACT | 1378 |
| 35 | CGAGGCATGCCAAGTCGCCAAT | 1379 |
| 36 | AGTGTTGATAGGCGGATGAGAG | 1380 |
| 37 | TTCGGTCTAGACCTCTCACAAT | 1381 |
| 38 | GTGACGCTCATATCTTGCCACC | 1382 |
| 39 | GATGTAATTCTACGCGCGGACT | 1383 |
| 40 | GATGGCGATGTTGCATTACATG | 1384 |
| 41 | TATGCTCTGAATTAACGTAGAA | 1385 |
| 42 | AGGCAATATGGTGATCCGTAGC | 1386 |
| 43 | TGACAGCGATGCATACAGTAGT | 1387 |
| 44 | TTCTGCTAACGGTATCCAATAC | 1388 |
| 45 | GAGTCGTCCATACGATCTAGGA | 1389 |
| 46 | AGACGGACTCAACGCCAATTCC | 1390 |
| 47 | GTAGTGTTGAGCGGACCGAGCT | 1391 |
| 48 | AATATAACTAGATCATAGCCAG | 1392 |
| 49 | TCAATCGGAGAATACAGAACGT | 1393 |
| 50 | ATCTCCGTCGTCCGAACCAACA | 1394 |
| 51 | TAGGCGTTCAGCGGTATGCTTA | 1395 |
| 52 | TGCGTGCTATACAACCTATACG | 1396 |
| 53 | ATGGCCGGCATACATCTGTATG | 1397 |
| 54 | TGATGCTGACATAACACTGAAT | 1398 |
| 55 | ATCCAAGGTACCTGAACATCCT | 1399 |
| 56 | TAGTGACGACCAGGTGAGCCTC | 1400 |
| 57 | AGGAGGATCCGTCAAGTCGACC | 1401 |
| 58 | AGAGTATGCCAGATCGTGAGGC | 1402 |
| 59 | CCACTCACTAGGATGGCTGCGT | 1403 |
| 60 | TATCCAACCTGTTATAGCGATT | 1404 |
| 61 | TCTTGCAGTGAGTTGAGTCTGC | 1405 |
| 62 | CCACTGTTGTACATACACCTGG | 1406 |
| 63 | ATGCGCGTAGGCCACTAAGTCC | 1407 |
| 64 | ACAGCGGTCTACAACCGACTGC | 1408 |
| 65 | TCGCGCTCCAGACAATTGCAGC | 1409 |
| 66 | CCGGTAGACCAGGAGTGGTCAT | 1410 |
| 67 | ATCTCCTAACCTAGAGCCATCT | 1411 |
| 68 | CCACATCGAATCTAACAACTAC | 1412 |
| 69 | TAGTCTTATTGAATACGTCCTA | 1413 |
| 70 | TCCTTAAGCCTTGGAACTGGCG | 1414 |
| 71 | CCGTGATGGATTGACGTAGAGG | 1415 |
| 72 | GCCTGGATAACAGATGTCTTAG | 1416 |
| 73 | CTCGACCTATAATCTTCTGCCA | 1417 |
| 74 | AGCTACTTCTCCTTCCTAATCA | 1418 |
| 75 | ACACGCTATTGCCTTCCAGTTA | 1419 |
| 76 | AAGCCTGTGCATGCAATGAGAA | 1420 |
| 77 | TCGTTGGTTATAGCACAACTTC | 1421 |
| 78 | GCGATGCCTTCCAACATACCAA | 1422 |
| 79 | CCACCGTTAGCACGTGCTACGT | 1423 |
| 80 | GTTACCACAATGCCGCCATCAA | 1424 |
| 81 | GGTGCATTAAGAACGAACTACC | 1425 |
| 82 | TCCTTCCGGATAATGCCGATTC | 1426 |
| 83 | AACCGCAACTTCTAGCGGAAGA | 1427 |
| 84 | TCCTTAAGCAGTTGAACCTAGG | 1428 |
| 85 | TACTAAGTCAGATAAGATCAGA | 1429 |
| 86 | TTCGCCATAACTAGATGAATGC | 1430 |
| 87 | AAGAAGTTAGACGCGGTGGCTG | 1431 |
| 88 | GTATCTGATCGAAGAGCGGTGG | 1432 |
| 89 | TCAAGAGCTACGAAGTAAGTCC | 1433 |
| 90 | CGAGTACACAGCAGCATACCTA | 1434 |
| 91 | CTCGATAAGTTACTCTGCTAGA | 1435 |
| 92 | ATGGTGCTGGTTCTCCGTCTGT | 1436 |
| 93 | TCAAGCGGTCCAAGGCTGAGAC | 1437 |
| 94 | TGTCCTGCTCTGTTGCTACCGT | 1438 |
| 95 | AGTCATATCGCGTCACACGTTG | 1439 |
| 96 | GGTGAATAAGGACATGAGAAGC | 1440 |

TABLE 16
List of random primers (24-base primers)

| | | SEQ |
|---|---|---|
| | | ID |
| No | Primer sequence | NO |
| 1 | CCTGATCTTATCTAGTAGAGACTC | 1441 |
| 2 | TTCTGTGTAGGTGTGCCAATCACC | 1442 |
| 3 | GACTTCCAGATGCTTAAGACGACA | 1443 |
| 4 | GTCCTTCGACGGAGAACATCCGAG | 1444 |
| 5 | CTTGGTTAGTGTACCGTCAACGTC | 1445 |
| 6 | AAGCGGCATGTGCCTAATCGACGT | 1446 |
| 7 | CGACCGTCGTTACACGGAATCCGA | 1447 |
| 8 | TCGCAAGTGTGCCGTTCTGTTCAT | 1448 |
| 9 | CGTACTGAAGTTCGGAGTCGCCGT | 1449 |
| 10 | CCACTACAGAATGGTAGCAGATCA | 1450 |
| 11 | AGTAGGAGAGAGGCCTACACAACA | 1451 |
| 12 | AGCCAAGATACTCGTTCGGTATGG | 1452 |
| 13 | GTTCCGAGTACATTGAATCCTGGC | 1453 |
| 14 | AGGCGTACGAGTTATTGCCAGAGG | 1454 |
| 15 | GTGGCATCACACATATCTCAGCAT | 1455 |
| 16 | GAGACCGATATGTTGATGCCAGAA | 1456 |
| 17 | CAACTGTAGCCAGTCGATTGCTAT | 1457 |
| 18 | TATCAATGCAATGAGAGGATGCAG | 1458 |
| 19 | GTATGCTCGGCTCCAAGTACTGTT | 1459 |
| 20 | AGAGACTCTTATAGGCTTGACGGA | 1460 |
| 21 | ACTTAACAGATATGGATCATCGCC | 1461 |
| 22 | AATCAGAGCGAGTCTCGCTTCAGG | 1462 |
| 23 | ACCACCGAGGAACACGTGCGACAA | 1463 |
| 24 | TGGTACATGTCAACCGTAAGCCTG | 1464 |
| 25 | CGTGCCGCGGTGTTCTTGTATATG | 1465 |
| 26 | GACAAGCGCGCGTGAGACATATCA | 1466 |
| 27 | AGTGCACTCCGAACAAGAGTTAGT | 1467 |
| 28 | CCTCATTACCGCGTTAGGAGTCCG | 1468 |
| 29 | TGCTTATTGCTTAGTTGCTATCTC | 1469 |
| 30 | GCGTGATCCTGTTCTATTCGTTAG | 1470 |
| 31 | GGCCAGAACTATGACGAGTATAAG | 1471 |
| 32 | GATGGCGACTATCTAATTGCAATG | 1472 |
| 33 | TAGTAACCATACCTCTGTACAACT | 1473 |
| 34 | CGTGATCGCCAATACACATGTCGC | 1474 |
| 35 | TAATAACGGATCGATATGCACGCG | 1475 |
| 36 | ATCATCGCGCTAATACTATCTGAA | 1476 |
| 37 | CACGTCCGTGCAGGTCACTAGTAT | 1477 |
| 38 | AGGTCCAATGCCGAGCGATCAGAA | 1478 |
| 39 | CAGCATAACAACGAGCCAGGTCAG | 1479 |
| 40 | ATGGCGTCCAATACTCCGACCTAT | 1480 |
| 41 | AGGAACATCGTGAATAATGAAGAC | 1481 |
| 42 | TCTCGACGTTCATGTAATTAAGGA | 1482 |
| 43 | TCGCGGTTAACCTTACTTAGACGA | 1483 |
| 44 | ATCATATCTACGGCTCTGGCGCCG | 1484 |
| 45 | GCAGATGGAGACCAGAGGTACAGG | 1485 |
| 46 | AGACAGAAGATTACCACGTGCTAT | 1486 |
| 47 | CCACGGACAACATGCCGCTTAACT | 1487 |
| 48 | CTTGAAGTCTCAAGCTATGAGAGA | 1488 |
| 49 | ACAGCAGTCGTGCTTAGGTCACTG | 1489 |
| 50 | AGGTGTTAATGAACGTAGGTGAGA | 1490 |
| 51 | AGCCACTATGTTCAAGGCTGAGCC | 1491 |
| 52 | GCAGGCGGTGTCGTGTGACAATGA | 1492 |
| 53 | AGCCATTGCTACAGAGGTTACTTA | 1493 |
| 54 | ACAATCGAACCTACACTGAGTCCG | 1494 |
| 55 | CCGATCTCAATAGGTACCACGAAC | 1495 |
| 56 | GATACGTGGCGCTATGCTAATTAA | 1496 |
| 57 | AGAGAGATGGCACACATTGACGTC | 1497 |
| 58 | CTCAACTCATCCTTGTAGCCGATG | 1498 |
| 59 | GTGGAATAACGCGATACGACTCTT | 1499 |
| 60 | ATCTACCATGCGAATGCTCTCTAG | 1500 |
| 61 | ATACGCACGCCTGACACAAGGACC | 1501 |
| 62 | GTCCACTCTCAGTGTGTAGAGTCC | 1502 |
| 63 | AATATATCCAGATTCTCTGTGCAG | 1503 |
| 64 | CCTTCCGCCACATGTTCGACAAGG | 1504 |
| 65 | AGTGTGCCATCATCCGAGGAGCCA | 1505 |
| 66 | TCTATGCCGCTATGGCGTCGTGTA | 1506 |
| 67 | CGTAACCTAAGGTAATATGTCTGC | 1507 |
| 68 | TACTGACCGTATCAAGATTACTAA | 1508 |
| 69 | TCATCGGAGCGCCATACGGTACGT | 1509 |
| 70 | GCAAGAGGAATGAACGAAGTGATT | 1510 |
| 71 | GGCTGATTGACATCCTGACTTAGT | 1511 |
| 72 | AAGGCGCTAGATTGGATTAACGTA | 1512 |
| 73 | GCTAGCTAGAAGAATAGGATTCGT | 1513 |
| 74 | CAGGTGACGGCCTCTATAACTCAT | 1514 |
| 75 | CAGGTTACACATACCACTATCTTC | 1515 |
| 76 | TTGCTACGTACCGTCTTAATCCGT | 1516 |
| 77 | CTCAACATGTCTTGCAAGCTTCGA | 1517 |
| 78 | GGTGCGGTACGTAGAACCAGATCA | 1518 |
| 79 | AATGCTCTCCAAGATCCTGACCTA | 1519 |
| 80 | GCTTCGCAGGTCTGGATGATGGAG | 1520 |
| 81 | ACATTGACCAGACAGCACCTTGCG | 1521 |
| 82 | AGGTATCAATGTGCTTAATAGGCG | 1522 |
| 83 | TCCGGACACACGATTAGTAACGGA | 1523 |
| 84 | TACGAAGTACTACAGATCGGTCAG | 1524 |
| 85 | AATTGTCAGACGAATACTGCTGGA | 1525 |
| 86 | TGAATCATGAGCCAGAGGTTATGC | 1526 |
| 87 | CACAAGACACGTCATTAACATCAA | 1527 |
| 88 | GAATGACTACATTACTCCGCCAGG | 1528 |
| 89 | AGCCAGAGATACTGGAACTTGACT | 1529 |
| 90 | TATCAGACACATCACAATGGATAC | 1530 |
| 91 | CTAGGACACCGCTAGTCGGTTGAA | 1531 |
| 92 | GTATAACTGCGTGTCCTGGTGTAT | 1532 |
| 93 | ATGCAATACTAAGGTGGACCTCCG | 1533 |
| 94 | ATGCAGACGCTTGCGATAAGTCAT | 1534 |
| 95 | TTGCTCGATACACGTAGACCAGTG | 1535 |
| 96 | TACTGGAGGACGATTGTCTATCAT | 1536 |

TABLE 17
List of random primers (26-base primers)

| | | SEQ |
|---|---|---|
| | | ID |
| No | Primer sequence | NO |
| 1 | ACTAAGGCACGCTGATTCGAGCATTA | 1537 |
| 2 | CGGATTCTGGCACGTACAAGTAGCAG | 1538 |
| 3 | TTATGGCTCCAGATCTAGTCACCAGC | 1539 |
| 4 | CATACACTCCAGGCATGTATGATAGG | 1540 |
| 5 | AGTTGTAAGCCAACGAGTGTAGCGTA | 1541 |
| 6 | GTATCAGCTCCTTCCTCTGATTCCGG | 1542 |
| 7 | AACATACAGAATGTCTATGGTCAGCT | 1543 |
| 8 | GACTCATATTCATGTTCAGTATAGAG | 1544 |
| 9 | AGAGTGAACGAACGTGACCGACGCTC | 1545 |
| 10 | AATTGGCGTCCTTGCCACAACATCTT | 1546 |
| 11 | TCGTAGACGCCTCGTACATCCGAGAT | 1547 |
| 12 | CCGGCTCGTGAGGCGATAATCATATA | 1548 |
| 13 | AGTCCTGATCACGACCACGACTCACG | 1549 |
| 14 | GGCACTCAATCCTCCATGGAGAAGCT | 1550 |
| 15 | TCATCATTCCTCACGTTCACCGGTGA | 1551 |
| 16 | TCAACTCTGTGCTAACCGGTCGTACA | 1552 |
| 17 | TGTTCTTATGCATTAATGCCAGGCTT | 1553 |
| 18 | GATTCACGACCTCAACAGCATCACTC | 1554 |
| 19 | GGCGAGTTCGACCAGAATGCTGGACA | 1555 |
| 20 | TTCCGTATACAATGCGATTAAGATCT | 1556 |
| 21 | GAGTAATCCGTAACCGGCCAACGTTG | 1557 |
| 22 | CGCTTCCATCATGGTACGGTACGTAT | 1558 |
| 23 | CCGTCGTGGTGTGTTGACTGGTCAAC | 1559 |
| 24 | TATTCGCATCTCCGTATTAGTTGTAG | 1560 |
| 25 | TATTATTGTATTCTAGGCGGTGCAAC | 1561 |
| 26 | AGGCTGCCTACTTCCTCGTCATCTCG | 1562 |
| 27 | GTAACATACGGCTCATCGAATGCATC | 1563 |
| 28 | TTATGGCACGGATATTACCGTACGCC | 1564 |
| 29 | ATAGCACTTCCTCTAATGCTCTGCTG | 1565 |
| 30 | TCACAGGCAATAGCCTAATATTATAT | 1566 |
| 31 | GGCGGATGTTCGTTAATATTATAAGG | 1567 |
| 32 | TGCAATAGCCGTTGTCTCTGCCAGCG | 1568 |
| 33 | TACAGCGCGTTGGCGAGTACTGATAG | 1569 |
| 34 | TGCAGTTAGTACCTTCTCACGCCAAC | 1570 |
| 35 | CCATTGGCTACCTAGCAGACTCTACC | 1571 |
| 36 | AACAGTAGCTCGCGTCTTGCTCTCGT | 1572 |
| 37 | GCAGTCCATCAGCTCTCGCTTATAGA | 1573 |
| 38 | TATCTCTCTGTCGCCAGCTTGACCAA | 1574 |
| 39 | CAGACTGTTCAAGCTTGCTGTAGGAG | 1575 |
| 40 | TAACCGGAACTCGTTCAGCAACATTC | 1576 |
| 41 | TCAATTATGCATGTCGTCCGATCTCT | 1577 |
| 42 | TTGTCTAAGTCAACCTGTGGATAATC | 1578 |
| 43 | TCTAAGAGTGGTATGACCAGGAGTCC | 1579 |
| 44 | TCGTAGTACTACTGGAACAGGTAATC | 1580 |
| 45 | ATGTCAACATTCTAATCATCTCTCGG | 1581 |
| 46 | AGCGCGCAACTGTTACGGTGATCCGA | 1582 |
| 47 | GCGATAGAATAATGGTGTCACACACG | 1583 |
| 48 | AAGGCTGCGATGAGAGGCGTACATCG | 1584 |
| 49 | GGTTCATGGTCTCAGTCGTGATCGCG | 1585 |
| 50 | TAGTGACTCTATGTCACCTCGGAGCC | 1586 |
| 51 | ATGTGATAGCAATGGCACCTCTAGTC | 1587 |
| 52 | TCGCGAAGTGTAATGCATCATCCGCT | 1588 |
| 53 | ATGTGGCGACGATCCAAGTTCAACGC | 1589 |
| 54 | ACCTTGTATGAGTCGGAGTGTCCGGC | 1590 |
| 55 | ACCTCAAGAGAGTAGACAGTTGAGTT | 1591 |
| 56 | GGTGTAATCCTGTGTGCGAAGCTGGT | 1592 |
| 57 | ATAGCGGAACTGTACGACGCTCCAGT | 1593 |
| 58 | AAGCACGAGTCGACCATTAGCCTGGA | 1594 |
| 59 | ATTCCGGTAACATCAGAAGGTACAAT | 1595 |
| 60 | GTGCAACGGCAGTCCAGTATCCTGGT | 1596 |
| 61 | CCATCTTATACACGGTGACCGAAGAT | 1597 |
| 62 | GCACTTAATCAAGCTTGAGTGATGCT | 1598 |
| 63 | AGTATTACGTGAGTACGAAGATAGCA | 1599 |
| 64 | TTCTTAGGTTAAGTTCCTTCTGGACC | 1600 |
| 65 | GTCCTTGCTAGACACTGACCGTTGCT | 1601 |
| 66 | GCCGCTATGTGTGCTGCATCCTAAGC | 1602 |
| 67 | CCATCAATAACAGACTTATGTTGTGA | 1603 |
| 68 | CGCGTGTGCTTACAAGTGCTAACAAG | 1604 |
| 69 | CGATATGTGTTCGCAATAAGAGAGCC | 1605 |
| 70 | CGCGGATGTGAGCGGCTCAATTAGCA | 1606 |
| 71 | GCTGCATGACTATCGGATGGAGGCAT | 1607 |
| 72 | CTATGCCGTGTATGGTACGAGTGGCG | 1608 |
| 73 | CCGGCTGGAGTTCATTACGTAGGCTG | 1609 |
| 74 | TGTAGGCCTACTGAGCTAGTATTAGA | 1610 |
| 75 | CCGTCAAGTGACTATTCTTCTAATCT | 1611 |
| 76 | GGTCTTACGCCAGAGACTGCGCTTCT | 1612 |
| 77 | CGAAGTGTGATTATTAACTGTAATCT | 1613 |
| 78 | GCACGCGTGGCCGTAAGCATCGATTA | 1614 |
| 79 | ATCCTGCGTCGGAACGTACTATAGCT | 1615 |
| 80 | AGTATCATCATATCCATTCGCAGTAC | 1616 |
| 81 | AGTCCTGACGTTCATATATAGACTCC | 1617 |
| 82 | CTTGCAGTAATCTGAATCTGAAGGTT | 1618 |
| 83 | ATAACTTGGTTCCAGTAACGCATAGT | 1619 |
| 84 | GATAAGGATATGGCTGTAGCGAAGTG | 1620 |
| 85 | GTGGAGCGTTACAGACATGCTGAACA | 1621 |
| 86 | CGCTTCCGGCAGGCGTCATATAAGTC | 1622 |
| 87 | ATAACATTCTAACCTCTATAAGCCGA | 1623 |
| 88 | ACGATCTATGATCCATATGGACTTCC | 1624 |
| 89 | TGAAGCTCAGATATCATGCCTCGAGC | 1625 |
| 90 | AGACTTCACCGCAATAACTCGTAGAT | 1626 |
| 91 | AGACTAAGACATACGCCATCACCGCT | 1627 |
| 92 | TGTAGCGTGATGTATCGTAATTCTGT | 1628 |
| 93 | TGTGCTATTGGCACCTCACGCTGACC | 1629 |
| 94 | TGTAGATAAGTATCCAGCGACTCTCT | 1630 |
| 95 | AATTCGCCAATTGTGTGTAGGCGCAA | 1631 |
| 96 | CGATTATGAGTACTTGTAGACCAGCT | 1632 |

TABLE 18
List of random primers (28-base primers)

| | | SEQ |
|---|---|---|
| | | ID |
| No | Primer sequence | NO |
| 1 | TTGCAAGAACAACGTATCTCATATGAAC | 1633 |
| 2 | CACCGTGCTGTTATTACTTGGTATTCGG | 1634 |
| 3 | CACGTGTATTGTTGCACCAGAACGACAA | 1635 |
| 4 | ATGCACGTAATTACTTCCGGAGAAGACG | 1636 |
| 5 | TATGTTGTCTGATATGGTTCATGTGGCA | 1637 |
| 6 | AGCGCGACTAGTTGATGCCAACATTGTA | 1638 |
| 7 | ATAGGCAGGTCCAGGCTCGGAACAAGTC | 1639 |
| 8 | GCGGTAGTCGGTCAAGAACTAGAACCGT | 1640 |
| 9 | ACTATACACTCTAGCTATTAGGAAGCAT | 1641 |
| 10 | GATCATCTTGCTTCTCCTGTGGAGATAA | 1642 |
| 11 | CTACTACGAGTCCATAACTGATAGCCTC | 1643 |
| 12 | GCACAGACACCTGTCCTATCTAGCAGGA | 1644 |
| 13 | AAGCGAGGCGCGAAGGAGATGGAAGGAT | 1645 |
| 14 | CTGAAGACGCCAGTCTGGATAGGTGCCT | 1646 |
| 15 | GTAAGCTCTGTCCTTCGAGATTGATAAG | 1647 |
| 16 | GGTTAGAGAGATTATTGTGCGCATCCAT | 1648 |
| 17 | CCAGGAGGACCTATGATCTTGCCGCCAT | 1649 |
| 18 | ACTATTCGAGCTACTGTATGTGTATCCG | 1650 |
| 19 | GACATCGCGATACGTAACTCCGGAGTGT | 1651 |
| 20 | CCGCAATTCGTCTATATATTCTAGCATA | 1652 |
| 21 | CTACACTTGAGGTTGATGCTCAAGATCA | 1653 |
| 22 | CGATCAGTTCTAGTTCACCGCGGACAAT | 1654 |
| 23 | AAGAATGATGATTGGCCGCGAACCAAGC | 1655 |
| 24 | CACGACCGGAACTAGACTCCTACCAATT | 1656 |
| 25 | AGTTGCCTGTGAGTGAGGCTACTATCTC | 1657 |
| 26 | GATTCTTCCGATGATGATGCCAGTAGAA | 1658 |
| 27 | CGCTGAAGTGAACTATGCAAGCACCGCA | 1659 |
| 28 | ATTATCGTGATGGTGAGACTGAGCTCGT | 1660 |
| 29 | CGAGGCCACTCTGAGCCAGGTAAGTATC | 1661 |
| 30 | TGCCGAGGACAGCCGATCACATCTTCGT | 1662 |
| 31 | GTTGACATGAAGGTTATCGTCGATATTC | 1663 |
| 32 | GTGGTCCAGGTCAAGCTCTGATCGAATG | 1664 |
| 33 | CCAGTCCGGTGTACTCAGACCTAATAAC | 1665 |
| 34 | CGAGACACTGCATGAGCGTAGTCTTATT | 1666 |
| 35 | GACGGCTTGTATACTTCTCTACGGTCTG | 1667 |
| 36 | TTAGCTGGATGGAAGCCATATTCCGTAG | 1668 |
| 37 | CAGCCTACACTTGATTACTCAACAACTC | 1669 |
| 38 | GTACGTAGTGTCACGCGCCTACGTTCGT | 1670 |
| 39 | CTACAACTTCTCAATCATGCCTCTGTTG | 1671 |
| 40 | CGAGGACAGAATTCGACATAAGGAGAGA | 1672 |
| 41 | GCCGAACGACACAGTGAGTTGATAGGTA | 1673 |
| 42 | GAACACTATATGCTGTCGCTGTCTGAGG | 1674 |
| 43 | GTTAAGTTCTTCGGCGGTCATGCTCATT | 1675 |
| 44 | TTGCTTACAGATCGCGTATCCATAGTAT | 1676 |
| 45 | GAGGACCACCTCTGCGAAGTTCACTGTG | 1677 |
| 46 | AATCCTAGCATATCGAGAACGACACTGA | 1678 |
| 47 | TGAATACTATAGCCATAGTCGACTTCCG | 1679 |
| 48 | GACATCCACGAAGCTGGTAATCGGAACC | 1680 |
| 49 | TTAGCCGTCTTAGAAGTGTCTGACCGGC | 1681 |
| 50 | CTATTCTGCCGTAATTGATTCCTTCGTT | 1682 |
| 51 | ACGCCTCTGGTCGAAGGTAGATTAGCTC | 1683 |
| 52 | CAGCCTATTGATCGTAAGTAGATGGTCC | 1684 |
| 53 | TTAAGTGAGGTGGACAACCATCAACTTC | 1685 |
| 54 | AAGGCCTTGCGGCTAAGTAGTATTCATC | 1686 |
| 55 | TTGTGATACTAATTCTTCTCAAGAGTCA | 1687 |
| 56 | GCATTAGGTGACGACCTTAGTCCATCAC | 1688 |
| 57 | GCGGATGGACGTATACAGTGAGTCGTGC | 1689 |
| 58 | GAACATGCCAGCCTCAACTAGGCTAAGA | 1690 |
| 59 | TCCGTCATTAGAGTATGAGTGACTACTA | 1691 |
| 60 | AACACTTAGTAACCAGTTCGGACTGGAC | 1692 |
| 61 | CGCTAACTATTGCGTATATTCGCGGCTT | 1693 |
| 62 | GCCATCTACGATCTTCGGCTTATCCTAG | 1694 |
| 63 | CCTGAGAATGTTGACTAAGATCTTGTGA | 1695 |
| 64 | TCGGTTAGTCTAATCATCACGCAACGGA | 1696 |
| 65 | ATTATCTATTGAAGCAGTGACAGCGATC | 1697 |
| 66 | GAGGAGAATCACGGAACACGGTCACATG | 1698 |
| 67 | GCTGCAAGCATTATGACCATGGCATCTG | 1699 |
| 68 | GAACAACCTATAACGACGTTGTGGACAA | 1700 |
| 69 | TTAATCATCGATAGACGACATGGAATCA | 1701 |
| 70 | TCGAGTGTAAGCACACTACGATCTGGAA | 1702 |
| 71 | GCTACGCACAGTCTCTGCACAGCTACAC | 1703 |
| 72 | CCTGTATGTACGTTCTGGCTAATACCTT | 1704 |
| 73 | TGAAGCACCGGTACATGGTGTATCCGGA | 1705 |
| 74 | TGCTGGAACCTAACTCGGTGATGACGAT | 1706 |
| 75 | CGCTATCTTACTGCCAAGTTCTCATATA | 1707 |
| 76 | AACGCGCGCGTATCGGCAATAATCTCAA | 1708 |
| 77 | CCATTAGGATGACCATCGACTATTAGAG | 1709 |
| 78 | TACTGCTAGACTGCGTGCATTCATGGCG | 1710 |
| 79 | CATTGCGCGCTCCACGAACTCTATTGTC | 1711 |
| 80 | GACGCGCCTAGAACTGTATAGCTCTACG | 1712 |
| 81 | CATTGCAACTTGTCGGTGATGGCAATCC | 1713 |
| 82 | TTAATGCACATGCAGTACGGCACCACAG | 1714 |
| 83 | AGCGGTACGTGGACGAGTGGTAATTAAT | 1715 |
| 84 | GACGTATTGCTATGCATTGGAAGATGCT | 1716 |
| 85 | AACACTTCGACCATTGCGCCTCAATGGT | 1717 |
| 86 | CGGTACGCTCTAGCGGTCATAAGATGCA | 1718 |
| 87 | CCTGAATAACAGCCGCGCCTAATTAGAT | 1719 |
| 88 | AAGCGTCTAATGTGCCTTAAGTCACATG | 1720 |
| 89 | GCTCTCCAAGAACCAGAAGTAAGCATCG | 1721 |
| 90 | GAGGAGAGTTGTCCGAGTGGTGTGATGT | 1722 |
| 91 | TAACGAGTGGTGCGTCTAAGCAATTGAG | 1723 |
| 92 | CCAACAGTATGCTGACATAACTATGATA | 1724 |
| 93 | GATCCTTGCCACGCCTATGAGATATCGC | 1725 |
| 94 | AACGCGCTACCGTCCTTGTGCATAGAGG | 1726 |
| 95 | CTACATGTGCCTTATAGTACAGAGGAAC | 1727 |
| 96 | CAGCCTCGTAGTTAGCGTGATTCATGCG | 1728 |

TABLE 19
List of random primers (29-base primers)

| | | SEQ |
|---|---|---|
| | | ID |
| No | Primer sequence | NO |
| 1 | CTCCTCGCCGATTGAAGTGCGTAGAACTA | 1729 |
| 2 | CAGCAGGCCTCAATAGGATAAGCCAACTA | 1730 |
| 3 | GACCATCAATCTCGAAGACTACGCTCTGT | 1731 |
| 4 | GGTTGCTCCGTCTGTTCAGCACACTGTTA | 1732 |
| 5 | AATGTCGACTGGCCATTATCGCCAAGTGT | 1733 |
| 6 | GATAGCTTGCCATGCGAATGGATCTCCAG | 1734 |
| 7 | CCAGACCGGAGCCAATTGGCTGCCAATAT | 1735 |
| 8 | AACGTCGCTCCATACGTTACCTAATGCAG | 1736 |
| 9 | GAATATGACGCGAACAGTCTATTCGGATC | 1737 |
| 10 | GACGAGAATGTATTAAGGATAAGCAAGGT | 1738 |
| 11 | AAGTCGTATGAATCGCTATCACATGAGTC | 1739 |
| 12 | GTCGTGGAGACTACAATTCTCCTCACGTT | 1740 |
| 13 | GTTGCCACCGTTACACGACTATCGACAGT | 1741 |
| 14 | AGGATAGGCTACGCCTTACTCTCCTAAGC | 1742 |
| 15 | TAATCATCCTGTTCGCCTCGAGGTTGTTA | 1743 |
| 16 | GACAAGCAGTAATAATTACTGAGTGGACG | 1744 |
| 17 | TACAGCGTTACGCAGGTATATCAAGGTAG | 1745 |
| 18 | CTAACATCACTTACTATTAGCGGTCTCGT | 1746 |
| 19 | CCGCGCTTCTTGACACGTTCTCCACTAGG | 1747 |
| 20 | CAAGTAACATGAGATGCTATCGGTACATT | 1748 |
| 21 | CGACCACTAGGCTGTGACCACGATACGCT | 1749 |
| 22 | CAGGTCATGTGACGCAGTCGGCAGTCAAC | 1750 |
| 23 | ACTCCATCGTTAGTTCTTCCGCCGTGCTG | 1751 |
| 24 | CTCACCACGTATGCGTCACTCGGTTACGT | 1752 |
| 25 | TGCCTATGCTATGGACCTTGCGCGACTCT | 1753 |
| 26 | AATGAAGGTCAACGCTCTGTAGTTACGCG | 1754 |
| 27 | CACCATTGATTCATGGCTTCCATCACTGC | 1755 |
| 28 | GACACGCAAGGTAATTCGAGATTGCAGCA | 1756 |
| 29 | CACCGAGAGGAAGGTTCGATCGCTTCTCG | 1757 |
| 30 | CAGTTATCGGATTGTGATATTCACTCCTG | 1758 |
| 31 | ATACTGTAACGCCTCAACCTATGCTGACT | 1759 |
| 32 | ATCTGTCTTATTCTGGCACACTCAGACTT | 1760 |
| 33 | TCCAACCGGTGACGTGCTCTTGATCCAAC | 1761 |
| 34 | CACACTCAGTTCGGCTATCTCTGCGATAG | 1762 |
| 35 | AGCTGTAAGTCAGGTCTACGACTCGTACT | 1763 |
| 36 | GTCGGCGGCACGCACAGCTAACATTCGTA | 1764 |
| 37 | ATATGGTAGCCAGCCACGTATACTGAACA | 1765 |
| 38 | TGGACAATCCGACTCTAACACAGAGGTAG | 1766 |
| 39 | TCCGCCGCTGACAGTTCAATCTATCAATT | 1767 |
| 40 | GGTTCCTTAGAATATGCACCTATCAGCGA | 1768 |
| 41 | CGGCTGTACGACATGGATCATAAGAGTGT | 1769 |
| 42 | TGCAGATGTACGCTGTGGCCAGTGGAGAG | 1770 |
| 43 | CCTACTCACTTAACAATAATCGGTTCGGT | 1771 |
| 44 | CGCTTCCTACTGCCTGTGCCGCGACATAA | 1772 |
| 45 | CTAGACCGACCGGTTATGCGCTATTGTTC | 1773 |
| 46 | TTGTGAGCACGTCTGCGGCAAGCCTATGG | 1774 |
| 47 | TCATCGGCCGGCGCTGTTGTTGTTACCAT | 1775 |
| 48 | GCGGTTAGGTGCAGTTAGGAAGACTATCA | 1776 |
| 49 | TATGCGGTCGTGAGGCGTAGCATTCTAGA | 1777 |
| 50 | CCATCTATTCGTCGAACTCTCAGCTCGTA | 1778 |
| 51 | ATCAGATCTACTGATCGCGGTAGAGTATC | 1779 |
| 52 | TACACATAGGCGGCGCAGCCTTCTAATTA | 1780 |
| 53 | TTAACCGTAGTTCTTAGCTTACGCCGCTC | 1781 |
| 54 | ACTATAGAGGACATGGCACTCCTCTTCTA | 1782 |
| 55 | CAGTTCGTATTAAGATTGAATGTAGCGGT | 1783 |
| 56 | AGTTATCGGTATCCGCTTATCCGTACGTA | 1784 |
| 57 | AGCTTATTCATACACTGCACCACAGCAAG | 1785 |
| 58 | CCGTCGGCTAGTCTATCCTCTAATTAGAA | 1786 |
| 59 | GTCCGCTTCCATGCCTGCTGTACGAACAC | 1787 |
| 60 | TCTCTTCCTCCTTCATTGTTCGCTAGCTC | 1788 |
| 61 | TCTCTTGAGCGGTCCTCATACAGGTCTGC | 1789 |
| 62 | GACCAAGTGTAGGTGATATCACCGGTACT | 1790 |
| 63 | AAGATTGTGATAGGTTGGTAGTTACCACA | 1791 |
| 64 | TCGCCTCCGAAGAGTATAGCATCGGCAGA | 1792 |
| 65 | GAGGTAGTTATGAGCATCGAGGTCCTGTT | 1793 |
| 66 | GGACGCAAGATCGCAGGTACTTGTAAGCT | 1794 |
| 67 | ACTCGTACACGTCATCGTGCAGGTCTCAG | 1795 |
| 68 | TAATCCGTCAGGAGTGAGATGGCTCGACA | 1796 |
| 69 | AAGATGGTTCCGCGCATTGACTAGCAAGT | 1797 |
| 70 | TCCGCGATCTGCGGATCTTGAATGCTCAC | 1798 |
| 71 | TTCACGAGAGTCAACTGCTAGTATCCTAG | 1799 |
| 72 | TTCCAACTGGATTCTTCCAACTCCTCGAA | 1800 |
| 73 | CACTACTACTCAAGTTATACGGTGTTGAC | 1801 |
| 74 | CAACTGGATTCTCAGGATGCGTCTCTAGC | 1802 |
| 75 | TGGACTAGAGTGGAGCGATTACGTAATAT | 1803 |
| 76 | GAGGTCATTCAACTGGACTCGCCACGGAC | 1804 |
| 77 | CAGGTGTGTAACGCTGCAATCACATGAAT | 1805 |
| 78 | TATGCTGAGGTATTAGTTCTAACTATGCG | 1806 |
| 79 | CGTCTGAGTCGGATAAGGAAGGTTACCGC | 1807 |
| 80 | GTACTATCGTCGCAGGCACTATCTCTGCC | 1808 |
| 81 | GCTTCCTCCTTGCAACTTCATTGCTTCGA | 1809 |
| 82 | TGTCTACGAAGTAGAAGACACGAATAATG | 1810 |
| 83 | CCGTCATCTAAGGCAGAGTACATCCGCGA | 1811 |
| 84 | CCGGAGGCGTACTAACTGACCACAACACC | 1812 |
| 85 | AACTCGTCGCTGCCTGAATAGGTCAGAGT | 1813 |
| 86 | TTATAAGATTAATGTCGGTCAGTGTCGGA | 1814 |
| 87 | CGTCTCGATGGATCCACACGAACCTGTTG | 1815 |
| 88 | ATGCCATCATGGTCGTCCTATCTTAAGGC | 1816 |
| 89 | GCGCTTCAGCGATTCGTCATGCAAGGCAC | 1817 |
| 90 | CCAAGCGATACCGAGGTACGGTTAACGAG | 1818 |
| 91 | ATATGACAGACAGGTGGACCTAAGCAAGC | 1819 |
| 92 | CACTACATCGTCAGGCCTGGAAGCCTCAG | 1820 |
| 93 | GCCGTGTAGACGAGGACATTATGTCGTAT | 1821 |
| 94 | CAACGTATATACACACCTTGTGAAGAGAA | 1822 |
| 95 | TCCAACGTAATTCCGCCGTCTGTCGAGAC | 1823 |
| 96 | AATTCGTGCTTCGATCACCGTAGACTCAG | 1824 |

TABLE 20
List of random primers (30-base primers)

| | | SEQ |
|---|---|---|
| | | ID |
| No | Primer sequence | NO |
| 1 | ACTATATTGTATTCACGTCCGACGACTCGC | 1825 |
| 2 | GACGAGCTTGTGGTACACTATACCTATGAG | 1826 |
| 3 | TGATTCAAGCACCAGGCATGCTTAAGCTAG | 1827 |
| 4 | CGGTCTCCTATAGGAAGGCTCATTCTGACG | 1828 |
| 5 | AGTCAGTGTCGAATCAATCAAGGCGTCCTT | 1829 |
| 6 | CGAACGTAATGGCCATCACGCGCTGGCCTA | 1830 |
| 7 | CGAACCTGGACCACCTGGCATTACCATTAC | 1831 |
| 8 | ACATTAGGTTCCTGTAATGTCTTATCAACG | 1832 |
| 9 | CGTCTAATCCACCGTATCGTCTTCGCGCAT | 1833 |
| 10 | TCTATGACTTACAACGGAATCTTACTTCGT | 1834 |
| 11 | GTAACCGATCGGTACCGTCTGCTATTGTTC | 1835 |
| 12 | GGTGATTGATAAGCAACACATATTAGGAGG | 1836 |
| 13 | AATTATCGACGCTAATAGGCGAGCTGTTCA | 1837 |
| 14 | GGAGGTACATGACGAGTGGACAGACAGACC | 1838 |
| 15 | CTCTAATCCGTTATGCGGTGATGTAATCCG | 1839 |
| 16 | GCAAGCACGCGGCTTGGCGAACTTCTATGC | 1840 |
| 17 | TAGATGTAGGCCTGGTAGGCAGAGGAGTAA | 1841 |
| 18 | CCGAGTGGCGACCACACAGGTACGCATTAA | 1842 |
| 19 | GTCCTGGCTCAGATTAGTGCACTTAGTTAT | 1843 |
| 20 | GCGGTACCTACATGTTATGACTCAGACGAC | 1844 |
| 21 | TCTCTGCCAATGCTGGTCTCATCGAATCCA | 1845 |
| 22 | TCTCTACACAGCTACATACTATACTGTAAC | 1846 |
| 23 | TACGACGGACGCTGGTGGTGTAAGAGAAGG | 1847 |
| 24 | GCCTCGATATATCTACGTATAGTTCAAGTT | 1848 |
| 25 | GGCTCCTGCATTCATTGAAGGTCGGCCTTG | 1849 |
| 26 | CAGTTCGGTGATTCAAGAGAACAATGGTGG | 1850 |
| 27 | TATAACGAAGCCGGCTGGAACCGTAACTCA | 1851 |
| 28 | CTGTATCAATTCAAGTGACAGTGGCACGTC | 1852 |
| 29 | AGCAATTGCGGTTCATAGGCGTAATTATAT | 1853 |
| 30 | CATATGGACCTGGAGATCACCGTTCAGTCC | 1854 |
| 31 | GAAGGCCGTTGGTCTATCTCTTACTGGAGC | 1855 |
| 32 | GTGCGTTCATCTAGCCTAAGACGCTGACCT | 1856 |
| 33 | GAGTAACTTATATCCTCTCTACGACATCGA | 1857 |
| 34 | ATTCTACGCTGATGTCTCCGCTGAACAGGA | 1858 |
| 35 | TCATCAACGTTACTCACTAGTACCACGGCT | 1859 |
| 36 | AACCATTCTTGAACGTTGAGAACCTGGTGG | 1860 |
| 37 | ACGACACCTCCGCGGAACATACCTGATTAG | 1861 |
| 38 | GCGCACTTATTGAAGTAATCTCATGGCCAA | 1862 |
| 39 | GCGCCAATTCAGCCAGTTAGCGTCTCCGTG | 1863 |
| 40 | AGCAACAAGTCGCTGTATATCGACTGGCCG | 1864 |
| 41 | CCTTACAATAGACCTCGCGGCGTTCATGCC | 1865 |
| 42 | GGATCCAACTTCAGCGAAGCACCAACGTCG | 1866 |
| 43 | GCGCCAGTTCTCGTACTCTCGAGAAGCGAC | 1867 |
| 44 | GAGTGCGGCCAATCTGGAACTCATGACGTT | 1868 |
| 45 | CCTGAGAGTGATTCGTGTCTGCGAAGATGC | 1869 |
| 46 | GTGACTGGTTAAGGCAATATTGGTCGACCG | 1870 |
| 47 | CTATCAAGCCTTACAAGGTCACGTCCACTA | 1871 |
| 48 | ACTGCGTCCTTGCGTCGGAACTCCTTGTGT | 1872 |
| 49 | TGCAACTCAGTGGCGGCGACACCAAGAGCT | 1873 |
| 50 | TTCGGTTCTACTAGGATCTCTATCTGAGCT | 1874 |
| 51 | AGCTAATCTATTAAGACAGATTAGACAGGA | 1875 |
| 52 | GGACCGCTCTTAGGTTATGCACCTGCGTAT | 1876 |
| 53 | CTCTAATACTAGTCCACAGGTTAGTACGAA | 1877 |
| 54 | ATCCATATATGCTCGTCGTCAGCCAGTGTT | 1878 |
| 55 | GCTATTACTGTGTTGATGTCCACAGGAGAA | 1879 |
| 56 | GCTACGGCGCAGATCTAGACAACTGGAAGT | 1880 |
| 57 | GCCTCTTGTGTTAGCCGAATACCAATGACC | 1881 |
| 58 | TGAGGACGATAACATTACCTCTCGAGTCGC | 1882 |
| 59 | CGATTACCAATCCGACGACTTCGCAGCAGC | 1883 |
| 60 | ATGACACGAGTCCAGTACATATGCGAAGAC | 1884 |
| 61 | GCGCTCGCATGCACTAGTGTAGACTGACGA | 1885 |
| 62 | GCACATCTCAGAATTGATGGTCTATGTCGC | 1886 |
| 63 | TTCTTCGACGCCGCGTACTAATAGGTCAAT | 1887 |
| 64 | GGAAGCGCCTCTAACAACCGATGCTTGTGG | 1888 |
| 65 | CTCTAGACGCGTCGTGACTCCAATCTGTTG | 1889 |
| 66 | GTAGTTCGTCGGAGTGACCTCGTACTCACT | 1890 |
| 67 | ATGCTGTCGAGTGTCCGGCATAGAGCACAC | 1891 |
| 68 | GCGCATCTTGCAGCGTCCTGTAGTTCTGAA | 1892 |
| 69 | GCGATTGTTGAGGAACCACAGCGGCACCTA | 1893 |
| 70 | CACGCGTACTCTGCTTGCTGTGTGGTCGGT | 1894 |
| 71 | CATCCAACGCAGGACCTAGTAGTCATGCTT | 1895 |
| 72 | TTCTAGTTGTGATGAGAATCGCTAGCGTGC | 1896 |
| 73 | CATTCTGAATCTGGTCTCTCTCGATCATCC | 1897 |
| 74 | ATTAATGTAGAGGATAGTTCCGTTCTCTCC | 1898 |
| 75 | GTATCGCGCTTACGAATGAGGTGTGGCTTC | 1899 |
| 76 | GCTGGTGAGAGAGCCAGATTATCGGTGGAG | 1900 |
| 77 | GGCACGAGCAGGTAGAACTAGAACCTAGAT | 1901 |
| 78 | TGTATTATCTCGAAGCGGTGCGTTAGAGTC | 1902 |
| 79 | CACGTGTTCTAGCTACTAATGGCGTCAATT | 1903 |
| 80 | CGCGCTACATTACTTCCTACACCATGCGTA | 1904 |
| 81 | TGAGGCAACTAGTGTTCGCAAGATGACGGA | 1905 |
| 82 | TTATTATTGTCTGTGGAACGCACGCCAGTC | 1906 |
| 83 | GCTATAGTATTATCCATGAATTCCGTCGGC | 1907 |
| 84 | GTATCAATAGCTCAATTCGTCAGAGTTGTG | 1908 |
| 85 | TAGTCCATGCGTGGATATATTGAGAGCTGA | 1909 |
| 86 | GCACAGTACGACTTATAACAGGTCTAGATC | 1910 |
| 87 | ACTCAATGGTGGCACGCTCGGCGCAGCATA | 1911 |
| 88 | GTAGTACCACTCCGCCTTAGGCAGCTTAAG | 1912 |
| 89 | CGCTCAACTGATGCGTGCAACCAATGTTAT | 1913 |
| 90 | GCAGCTTGACTGCCTAGACAGCAGTTACAG | 1914 |
| 91 | GCAACTTCTTAGTACGAATTCATCGTCCAA | 1915 |
| 92 | ATCCGTATGCTGCGGCAGTGGAGGTGGCTT | 1916 |
| 93 | TGCGGATCAATCCAGTTCTGTGTACTGTGA | 1917 |
| 94 | TTATGATTATCACCGGCGTAACATTCCGAA | 1918 |
| 95 | GCTACCTAGATTCTTCAACTCATCGCTACC | 1919 |
| 96 | CAGTGTTAGAATGGCGGTGTGTAGCCGCTA | 1920 |

TABLE 21
List of random primers (35-base primers)

| | | SEQ |
|---|---|---|
| | | ID |
| No | Primer sequence | NO |
| 1 | GCTTATAGACTACAGCTGGGAGGTATAAGGTCACT | 1921 |
| 2 | CGCTCAGCAGGATGCTATCCTAAGTTAATGTGGTG | 1922 |
| 3 | GAACTGAGCGGACATCAGCTAGGCCTACAATACAT | 1923 |
| 4 | TCGTGAACTTCTGCGTTGGTCTCTACCAAGGCGGT | 1924 |
| 5 | TAAGTCAGGTATCTTATCAGTGGTACACGGTACGA | 1925 |
| 6 | TAATAATGTTGCGCGTGACCGAGGAGGAATCCACT | 1926 |
| 7 | CTAGGAGTTCTCGTAAGCTGGAGTACCGTAACGTG | 1927 |
| 8 | GGACTCTCCTCAGAGGATCCTTCTTGCGCAGGCAT | 1928 |
| 9 | GCTAGAGGCCTGAGTACACCTTCTCGCATCAGGAT | 1929 |
| 10 | ATATCGCGAGCACTAACGTCGTTGTCGTTCTAGGA | 1930 |
| 11 | AGCGGTTACTATACCTGGCGGCTGACGTTGTTAGT | 1931 |
| 12 | GAGCTAGGTAGATCTCCAAGTGTAGCTAAGAAGAG | 1932 |
| 13 | GGAGTCGCTGGTGACGTATGCCGAGGATGAGCTTC | 1933 |
| 14 | CGCCGACCTCCTGTTCACGAAGCCGCCTGATGTAA | 1934 |
| 15 | AGTAGGCACTTAGTTATCGATTACGTTAGTTAGTC | 1935 |
| 16 | GGATGACGTCTCAGTCTACCTCGCAGTGTCGTCTA | 1936 |
| 17 | CTGGTTCGCGTTAGCAATACTAAGGCAGTCAGGAG | 1937 |
| 18 | ATATGGTCATATTGGCCTCTTCGAACACAGACTGT | 1938 |
| 19 | TATCAGAGGATAGCAGGTCTGAGTTGCAAGGCTAA | 1939 |
| 20 | GGTGGTCTGACCATAGCTGTTCTTCTCACAGAGAC | 1940 |
| 21 | GCAATACCAACGAGATGAGTATTCGTTGAAGCTCT | 1941 |
| 22 | CCAAGTCGACGCTGCATGAATGAGCGCTATTCACT | 1942 |
| 23 | CCATTAGATCGCTTCGAGACAATTAGGAGACATGA | 1943 |
| 24 | GATGACTGTACCTCCTATCATTGAGTGTGGACCAA | 1944 |
| 25 | ATATCTGGATGAATAGTGGTTAGGTAAGCAAGTAA | 1945 |
| 26 | ACCGACTATGTTAATTCGTGTCTGGATGGCAGAAT | 1946 |
| 27 | GTGGCAGTCTTGCTAGTATCTTAGACCATCACCAA | 1947 |
| 28 | CGCTATCTTAGTCGAGCACAATGTCTTCGTATAGG | 1948 |
| 29 | ATTAGTACGGCACGAACCGGCCATTCATGGCAGCT | 1949 |
| 30 | AGTACGACTATCAAGACTCCAGCGCTCTCCTTGGA | 1950 |
| 31 | ATGAGCCTCGGAGCGAACGTTATCGATCAGGCTGT | 1951 |
| 32 | TTGCGTGCAGTAGCACCGATACACAGCGCTTGTAT | 1952 |
| 33 | AACGGCTGCATCACCTACACTATACTCAACATCTA | 1953 |
| 34 | GTCGCTATGCGAGAAGTGGCGTGGAATGCTATGGT | 1954 |
| 35 | CATGGATACCTACTGACTTGACTTCTAGAGGACCG | 1955 |
| 36 | GAGTGACGCAGACACCGTAACGTCGAATCTTCTAG | 1956 |
| 37 | AGTACCGTCTGTGTGAATATTGTTCCTACGTTACA | 1957 |
| 38 | GGCTAATCGATAGTGACGAGTTCTGCACGCCTGAA | 1958 |
| 39 | GGCGAGCGCTCGTGGTTCTGAGTCGCTGTTAGATG | 1959 |
| 40 | TATCTCCAGCGTTATAAGCTACTGGAGCCGCTCGG | 1960 |
| 41 | CCTTCTGCGCAAGTCAAGGATTCGCTTAGATGGAC | 1961 |
| 42 | GTTGCTGACAGCCGTTGCGTACTTGCCTTAAGAAC | 1962 |
| 43 | GTGGCCTAATCACTCGCGCTTCATAGGCCGATAGG | 1963 |
| 44 | TGCATCTAGCCTACATCGGACCTTGTTATGGTAAT | 1964 |
| 45 | GGACAGCTACTGGACACCACCGAACTGGTAGTGTC | 1965 |
| 46 | AACTGGCGATGGACGGCCGCTCTTCCGCTACATAG | 1966 |
| 47 | GGAGCAGTTAGCTATGGAGCAGGCCGATAACCTGA | 1967 |
| 48 | ACTCTACGGTGCACCTCAGCCTTCATGCAATAGGC | 1968 |
| 49 | CTTGTAGCACAATACATTACTCTCCACGTGATAGC | 1969 |
| 50 | GGACGCTATCGATACCGTTATTCCTACTCTGTCGG | 1970 |
| 51 | GGATGATCGTCAACGATCAACTGACAGTTAGTCGA | 1971 |
| 52 | TGACAGTAGCAATGTCTCACGTCTGCACAACGGAA | 1972 |
| 53 | GTCGCAGGACCTCACGGATAGTAGTGCGAGGTCTA | 1973 |
| 54 | ATATCGGCGGACGCAATGACAGTTGTTGGCTGATG | 1974 |
| 55 | AAGCACCAAGGAGGTATGTTCCATCGAGGCGCTCG | 1975 |
| 56 | GACCGCACCTTATAGCTATATCCTGGTCTAGTACT | 1976 |
| 57 | TCTCAGAGGAAGGTTGAGCGTCTGACCAGGTTGGC | 1977 |
| 58 | TGGACCTAGAGACCTAGCTCGTCTCTTCGCGATCG | 1978 |
| 59 | CGGAGTGGTTCCACGCGACCTCGCAACTAATCCTT | 1979 |
| 60 | GGAGCCGCGCGCAGACTGACCTTGCTTGATCTACT | 1980 |
| 61 | ACTCTAAGTATATGCGCAGTTAGTATACTGAACCA | 1981 |
| 62 | GAGCATTGCTTCGCTTCGATGTCTATTCTGATCAG | 1982 |
| 63 | GCTTGTATTGCCACTCGAGTAGGTCGTGGCAGTAG | 1983 |
| 64 | ATCTGGACATTGCATTCGGTGTGTATACAGAAGGC | 1984 |
| 65 | GGTTGCGATCAGCTTGATAGCAGGTCATATCCTCA | 1985 |
| 66 | GCAGGTACTAACCTGAGATGCGTAGCTAACACAGG | 1986 |
| 67 | ATCTGCAAGGACGTAACGTCCTCGGAAGGTGAGGT | 1987 |
| 68 | ATAATCTTACGAGCCTCCAGTGAATAATGCAAGCA | 1988 |
| 69 | CAATCTCCGCACAGTCTTGTTCAGGTACAGACTTA | 1989 |
| 70 | ATGTGCGCAATTCAGCGTAAGTGCCTATTCATAAT | 1990 |
| 71 | TCGGACGCACACATCCTGTTGTCGAGAAGAGGAAG | 1991 |
| 72 | TCGGAAGCATCACATGAGCATCAGGAGTTCATTGC | 1992 |
| 73 | ATCTGGTTGTGGACTTCTATACAGTACCAGAGTGG | 1993 |
| 74 | CGTCTGAATATAGTTAGCTAGTAGTGTAATCCAGG | 1994 |
| 75 | TAATATCTGATCCGACCTATTATCTAGGACTACTC | 1995 |
| 76 | TATGCGGCCGTCCGTACCTCGTCTGCTTCAGTTGG | 1996 |
| 77 | TGGCTCAAGTTCCATATTGCCAAGACGACCTGGAG | 1997 |
| 78 | GCAGTTCTGCTAGGCGGTCCGAGGCAATTGAAGAG | 1998 |
| 79 | CATGGCACAGACGAAGTATGCACCACGCTCATTAA | 1999 |
| 80 | GGAGCGTACTACGACCATTCAACCGAATATGTTAC | 2000 |
| 81 | GCGTAGATCTCGCGACAGAGACAAGGTGCGAATGG | 2001 |
| 82 | TGGACTGAGGTTCTCCGGTCTATACTCCTGTAGGA | 2002 |
| 83 | TGGCTATAGCAACGGCTTCTTGTGATCGCATTGCA | 2003 |
| 84 | GGCGAAGAATCATGCGAGACGGAGTAGACGGACGT | 2004 |
| 85 | GAGCATTGCGAGTTGCACACGTGATATCAGACTGT | 2005 |
| 86 | CTGTTGACCTATGCCAGAATCAATACCTCAGATTA | 2006 |
| 87 | GTTAACAAGTAGATGCCAAGATACAACGAGAGACC | 2007 |
| 88 | GAGCAAGATTATAGTTAGGAAGATAGTTAACTCGC | 2008 |
| 89 | TCCGGAGTCGAGCATATGTGACCAACTCTCAACGC | 2009 |
| 90 | GGAGCTGCGATGCCGTTACCGACGTCATCTTCAAG | 2010 |
| 91 | GCTCTATCTTACACATTGGCGTACTGGACTCGCGA | 2011 |
| 92 | TTCTACATATTCATCGCCTACCGAGTTGCGCGAAG | 2012 |
| 93 | TGGACGTCTGACCTGTGTCTACATCGGTGGTGCTA | 2013 |
| 94 | GGCAGGACAGCTCCGTGTTCTACTCGAACCGCACT | 2014 |
| 95 | TGACAACCTCATGTCTCCGACCGCAGGCATACAAT | 2015 |
| 96 | GCAGGCCTAACAAGTGGTCACGAGGAGTCCTTATT | 2016 |

3.1.2 Standard PCR

To the genomic DNA described in 2. above (15 ng, NiF8-derived genomic DNA), random primers (final concentration: 0.6 microM; 10-base primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 microliters. PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. In this example, numerous nucleic acid fragments obtained via PCR using random primers, including the standard PCR described above, are referred to as DNA libraries.

3.1.3 Purification of DNA Library and Electrophoresis

The DNA library obtained in 3.1.2 above was purified with the use of the MinElute PCR Purification Kit (QIAGEN) and subjected to electrophoresis with the use of the Agilent 2100 bioanalyzer (Agilent Technologies) to obtain a fluorescence unit (FU).

3.1.4 Examination of Annealing Temperature

To the genomic DNA described in 2. above (15 ng, NiF8-derived genomic DNA), random primers (final concentration: 0.6 microM; 10-base primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 microliters. PCR was carried out under thermal cycling conditions comprising 98 degrees C. for 2 minutes and 30 cycles of 98 degrees C. for 10 seconds, various annealing temperatures for 15 seconds, and 72 degrees C. for 20 seconds, followed by storage at 4 degrees C. In this example, annealing temperature of 37 degrees C., 40 degrees, and 45 degrees C. were examined. The DNA library obtained in this experiment was subjected to purification and electrophoresis in the same manner as in 3.1.3.

3.1.5 Examination of Enzyme Amount

To the genomic DNA described in 2. above (15 ng, NiF8-derived genomic DNA), random primers (final concentration: 0.6 microM; 10-base primer A), a 0.2 mM dNTP mixture, 1.0 mM MgCl₂, and 2.5 units or 125 units of DNA polymerase (PrimeSTAR, TAKARA) were added, and a reaction solution was prepared while adjusting the final reaction level to 50 microliters. PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. The DNA library obtained in this experiment was subjected to purification and electrophoresis in the same manner as in 3.1.3.

3.1.6 Examination of MgCl₂ Concentration

To the genomic DNA described in 2. above (15 ng, NiF8-derived genomic DNA), random primers (final concentration: 0.6 microM; 10-base primer A), a 0.2 mM dNTP mixture, MgCl₂ at a given concentration, and 1.25 units of DNA polymerase (PrimeSTAR, TAKARA) were added, and a reaction solution was prepared while adjusting the final reaction level to 50 microliters. PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. In this example, MgCl₂ concentrations, which are 2 times (2.0 mM). 3 times (3.0 mM), and 4 times (4.0 mM) greater than a common level, respectively, were examined. The DNA library obtained in this experiment was subjected to purification and electrophoresis in the same manner as in 3.1.3.

3.1.7 Examination of Base Length of Random Primer

To the genomic DNA described in 2. above (15 ng, NiF8-derived genomic DNA), random primers (final concentration: 0.6 microM), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 microliters. PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. In this example, the random primers comprising 8 bases (Table 7), 9 bases (Table 8), 11 bases (Table 9), 12 bases (Table 10), 14 bases (Table 11), 16 bases (Table 12), 18 bases (Table 13), and 20 bases (Table 14) were examined. The DNA library obtained in this experiment was subjected to purification and electrophoresis in the same manner as in 3.1.3.

3.1.8 Examination of Random Primer Concentration

To the genomic DNA described in 2. above (15 ng, NiF8-derived genomic DNA), random primers at a given concentration (10-base primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 microliters. PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. In this example, random primer concentrations of 2, 4, 6, 8, 10, 20, 40, 60, 100, 200, 300, 400, 500, 600, 700, 800, 900, and 1000 microM were examined. The DNA library obtained in this experiment was subjected to purification and electrophoresis in the same manner as in 3.1.3. In this experiment, the reproducibility of the repeated data was evaluated on the basis of the Spearman's rank correlation (rho>0.9).

3.2 Verification of Reproducibility Via MiSeq

3.2.1 Preparation of DNA Library

To the genomic DNA described in 2. above (15 ng, NiF8-derived genomic DNA), random primers (final concentration: 60 microM, 10-base primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 microliters. PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. The DNA library obtained in this experiment was subjected to purification and electrophoresis in the same manner as in 3.1.3.

3.2.2 Preparation of Sequence Library

From the DNA library obtained in 3.2.1, a sequence library for MiSeq analysis was prepared using the KAPA Library Preparation Kit (Roche).

3.2.3 MiSeq Analysis

With the use of the MiSeq Reagent Kit V2 500 Cycle (IIlumina), the sequence library for MiSeq analysis obtained in 3.2.2 was analyzed via 100 base paired-end sequencing.

3.2.4 Read Data Analysis

Random primer sequence information was deleted from the read data obtained in 3.2.3, and the read patterns were identified. The number of reads was counted for each read pattern, the number of reads of the repeated analyses, and the reproducibility was evaluated using the correlational coefficient.

3.3 Analysis of Rice Variety Nipponbare

3.3.1 Preparation of DNA Library

To the genomic DNA described in 2. above (15 ng, Nipponbare-derived genomic DNA), a random primer (final concentration: 60 microM, (10-base primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 microliters. PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. The DNA library obtained in this experiment was subjected to purification and electrophoresis in the same manner as in 3.1.3.

3.3.2 Preparation of Sequence Library, MiSeq Analysis, and Read Data Analysis

Preparation of a sequence library using the DNA library prepared from Nipponbare-derived genomic DNA, MiSeq analysis, and analysis of the read data were performed in accordance with the methods described in 3.2.2, 3.2.3, and 3.2.4, respectively.

3.3.3 Evaluation of Genomic Homogeneity

The read patterns obtained in 3.3.2 were mapped to the genomic information of Nipponbare (NC_008394 to NC_008405) using bowde2, and the genomic positions of the read patterns were identified.

3.3.4 Non-Specific Amplification

On the basis of the positional information of the read patterns identified in 3.3.3, the sequences of random primers were compared with the genome sequences to which such random primers would anneal, and the number of mismatches was determined.

3.4 Detection of Polymorphism and Identification of Genotype

3.4.1 Preparation of DNA Library

To the genomic DNA described in 2. above (15 ng, NiF8-derived genomic DNA, Ni9-derived genomic DNA, hybrid progeny-derived genomic DNA, or Nipponbare-derived genomic DNA), random primers (final concentration: 60 microM, 10-base primer A), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 microliters. PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. The DNA library obtained in this experiment was subjected to purification and electrophoresis in the same manner as in 3.1.3.

3.4.2 HiSeq Analysis

Analysis of the DNA libraries prepared in 3.4.1 was consigned to TakaraBio under conditions in which the number of samples was 16 per lane via 100 base paired-end sequencing, and the read data were obtained.

3.4.3 Read Data Analysis

Random primer sequence information was deleted from the read data obtained in 3.4.2, and the read patterns were identified. The number of reads was counted for each read pattern.

3.4.4 Detection of Polymorphism and Identification of Genotype

On the basis of the read patterns and the number of reads obtained as a results of analysis conducted in 3.4.3, polymorphisms peculiar to NiF8 and Ni9 were detected, and the read patterns thereof were designated as markers. On the basis of the number of reads, the genotypes of the 22 hybrid progeny lines were identified. The accuracy for genotype identification was evaluated on the basis of the reproducibility attained by the repeated data concerning the 22 hybrid progeny lines.

3.5 Experiment for Confirmation with PCR Marker

3.5.1 Primer Designing

Primers were designed for a total of 6 markers (i.e., 3 NiF8 markers and 3 Ni9 markers) among the markers identified in 3.4.4 based on the marker sequence information obtained via paired-end sequencing (Table 22).

TABLE 22
Marker sequence information and PCR marker primer information

| Geno- | Marker | | | | |
|---|---|---|---|---|---|
| type | name | Marker sequence 1* | Marker sequence 2* | Primer 1 | Primer 2 |
| NiF8 | N80521152 | CCCATACACACACCATGAA | ATGGGTGAGGGCGCAGAGGC | CCCATACA | GGTAGAAG |
| type | | GCTTGAACTAATTAACATT | AAAGACATGGAGGTCCGGAA | CACACCAT | CTCACATC |
| | | CTCAAACTAATTAACAAGC | GGGTAGAAGCTCACATCAAG | GAAGCTTG | AAGTCGAG |
| | | ATGCAAGCATGTTTTTACA | TCGAGTATGTTGAATGCAAT | (SEQ ID | (SEQ ID |
| | | CAATGACAATATAT | CCCATATATA | NO: 2019) | NO: 2020) |
| | | (SEQ ID NO: 2017) | (SEQ ID NO: 2018) | | |
| | N80997192 | AATCACAGAACGAGGTCTG | GATGCTGAGGGCGAAGTTGT | ACGAGAAC | TCAATGTC |
| | | GACGAGAACAGAGCTGGAC | CAGCCAAGTCCTCAATGTCA | AGAGCTGG | ATAGGCGA |
| | | ATCTACACGCACCGCATGG | TAGGCGAGATCGCAGTAGTT | ACATCTAC | GATCGCAG |
| | | TAGTAGAGCATGTACTGCA | CTGTAACCATTCCCTGCTAA | (SEQ ID | (SEQ ID |
| | | AAAGCTTGAAGCGC | ACTGGTCCAT | NO: 2023) | NO: 2024) |
| | | (SEQ ID NO: 2021) | (SEQ ID NO: 2022) | | |
| | N80533142 | AGACCAACAAGCAGCAAGT | GGAGGAGCACAACTAGGCGT | GGAGAGCAA | CGAGCTCTT |
| | | AGTCAGAGAAGTACAAGAG | TTATCAAGATGGGTCATCGA | GAAGGATAG | GGTGTCTTC |
| | | AAGGAGAGCAAGAAGGATA | GCTCTTGGTGTCTTCAACCT | TAAGTTGC | AACCTTC |
| | | GTAAGTTGCAAGCTTACCG | TCTTGACATCAACTTCTCCA | (SEQ ID | (SEQ ID |
| | | TTACAAAGATGATA | ATCTTCGTCT | NO: 2027) | NO: 2028) |
| | | (SEQ ID NO: 2025) | (SEQ ID NO: 2026) | | |
| Ni9 | N91552391 | TGGGGTAGTCCTGAAGCTC | GGATACTGATGTAGCTTTCA | GAAGCTCTA | GTGCACTAG |
| type | | TAGGTATGCCTCTTCATCT | CCCGGGAGTATTCCAAGGTA | GGTATGCCT | TTGAGGTTT |
| | | CCCTGCACCTCTGGTGCTA | TCGATTTTCCACGGGGAACG | CTTCATC | AGATTGC |
| | | GCACCTCCTGCTCTTCGGG | CGAAGTGCACTAGTTGAGGT | (SEQ ID | (SEQ ID |
| | | CACCTCTACCGGGG | TTAGATTGCC | NO: 2031) | NO: 2032) |
| | | (SEQ ID NO: 2029) | (SEQ ID NO: 2030) | | |
| | N91653962 | TCGGAAAACGAACGGGCGA | AGCAGGAGGGAGAAAGGAAA | GGGCGAACT | CTGTCTGCC |
| | | ACTACAGATGTCAGTACGA | CGTGGCATTCATCGGCTGTC | ACAGATGTC | ATTGCCATG |
| | | AGTAGTCTATGGCAGGAAA | TGCCATTGCCATGTGAGACA | AGTACG | TGAGAC |
| | | TACGTAGTCCATACGTGGT | AGGAAATCTACTTCACCCCC | (SEQ ID | (SEQ ID |
| | | GCCAGCCCAAGCC | ATCTATCGAG | NO: 2035) | NO: 2036) |
| | | (SEQ ID NO: 2033) | (SEQ ID NO: 2034) | | |
| | N91124801 | AGACATAAGATTAACTATG | TTAAGTTGCAGAATTTGATA | GAACAAATT | CGAAGAACT |
| | | AACAAATTCACGGGTCCGA | CGAAGAACTTGAAGCATGGT | CACGGGTCC | TGAAGCATG |
| | | TTCCTTTGGGATTTGCAGC | GAGGTTGCCGAGCTCATTGG | GATTCC | GTGAGG |
| | | TTGCAAGAACCTTCAAATA | GGATGGTTCCAGAAAGGCTA | (SEQ ID | (SEQ ID |
| | | CTCATTATATCTTC | TTGTAGCTTA | NO: 2039) | NO: 2040) |
| | | (SEQ ID NO: 2037) | (SEQ ID NO: 2038) | | |

*Marker sequence information by paired-end sequencing

3.5.2 PCR and Electrophoresis

With the use of the TaKaRa Multiplex PCR Assay Kit Ver.2 (TAKARA) and the genomic DNA described in 2. above (15 ng, NiF8-derived genomic DNA, Ni9-derived genomic DNA, or hybrid progeny-derived genomic DNA) as a template, 1.25 microliters of Multiplex PCR enzyme mix, 12.5 microliters of 2x Multiplex PCR buffer, and the 0.4 microM primer designed in 3.5.1 were added, and a reaction solution was prepared while adjusting the final reaction level to 25 microliters. PCR was carried out under thermal cycling conditions comprising 94 degrees C. for 1 minute, 30 cycles of 94 degrees C. for 30 seconds, 60 degrees C. for 30 seconds, and 72 degrees C. for 30 seconds, and retention at 72 degrees C. for 10 minutes, followed by storage at 4 degrees C. The amplified DNA fragment was subjected to electrophoresis with the use of TapeStation (Agilent Technologies).

3.5.3 Comparison of Genotype Data

On the basis of the results of electrophoresis obtained in 3.5.2, the genotype of the marker was identified on the basis of the presence or absence of a band, and the results were compared with the number of reads of the marker.

3.6 Correlation Between Random Primer Density and Length

3.6.1 Influence of Random Primer Length at High Concentration

To the genomic DNA described in 2. above (15 ng, NiF8-derived genomic DNA), random primers of a given length (final concentration: 10 microM), a 0.2 mM dNTP mixture, 1.0 mM MgCl₂, and 1.25 units of DNA polymerase (PrimeSTAR, TAKARA) were added, and a reaction solution was prepared while adjusting the final reaction level to 50 microliters. In this experiment, the random primer lengths of 9 bases (Table 8), 10 bases (Table 1, 10-base primer A), 11 bases (Table 9), 12 bases (Table 10), 14 bases (Table 11), 16 bases (Table 12), 18 bases (Table 13), and 20 bases (Table 14) were examined. In the reaction system using a random primer of 9 bases, PCR was carried out under thermal cycling conditions comprising 98 degrees C. for 2 minutes and 30 cycles of 98 degrees C. for 10 seconds, 37 degrees C. for 15 seconds, and 72 degrees C. for 20 seconds, followed by storage at 4 degrees C. In the reaction system using a random primer of 10 or more bases, PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. The DNA library obtained in this experiment was subjected to purification and electrophoresis in the same manner as in 3.1.3.

3.6.2 Correlation Between Random Primer Density and Length

To the genomic DNA described in 2. above (15 ng, NiF8-derived genomic DNA), random primers of a given length were added to a given concentration therein, a 0.2 mM dNTP mixture, 1.0 mM MgCl₂, and 1.25 units of DNA polymerase (PrimeSTAR, TAKARA) were added thereto, and a reaction solution was prepared while adjusting the final reaction level to 50 microliters. In this experiment, random primers comprising 8 to 35 bases shown in Tables 1 to 21 were examined, and the random primer concentration from 0.6 to 300 microM was examined

In the reaction system using random primers each comprising 8 bases and 9 bases, PCR was carried out under thermal cycling conditions comprising 98 degrees C. for 2 minutes and 30 cycles of 98 degrees C. for 10 seconds, 37 degrees C. for 15 seconds, and 72 degrees C. for 20 seconds, followed by storage at 4 degrees C. In the reaction system using a random primer of 10 or more bases, PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. The DNA library obtained in this experiment was subjected to purification and electrophoresis in the same manner as in 3.1.3. Also, the reproducibility of the repeated data was evaluated on the basis of the Spearman's rank correlation (rho>0.9).

3.7 Number of Random Primers

To the genomic DNA described in 2. above (15 ng, NiF8-derived genomic DNA), 1, 2, 3, 12, 24, or 48 types of random primers selected from the 96 types of random primers comprising 10 bases (10-base primer A) shown in Table 1 were added to the final concentration of 60 microM therein, a 0.2 mM dNTP mixture, 1.0 mM MgCl₂. and 1.25 units of DNA polymerase (PrimeSTAR, TAKARA) were added thereto, and a reaction solution was prepared while adjusting the final reaction level to 50 microliters. In this experiment, as the 1, 2, 3, 12, 24, or 48 types of random primers, random primers were selected successively from No. 1 shown in Table 1, and the selected primers were then examined PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. The DNA library obtained in this experiment was subjected to purification and electrophoresis in the same manner as in 3.1.3. Also, the reproducibility of the repeated data was evaluated on the basis of the Spearman's rank correlation (rho>0.9).

3.8 Random Primer Sequence

To the genomic DNA described in 2. above (15 ng, NiF8-derived genomic DNA), a set of primers selected from the 5 sets of random primers shown in Tables 2 to 6 was added to the final concentration of 60 microM therein, a 0.2 mM dNTP mixture, 1.0 mM MgCl₂, and 1.25 units of DNA polymerase (PrimeSTAR, TAKARA) were added thereto, and a reaction solution was prepared while adjusting the final reaction level to 50 microliters. PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. The DNA library obtained in this experiment was subjected to purification and electrophoresis in the same manner as in 3.1.3. Also, the reproducibility of the repeated data was evaluated on the basis of the Spearman's rank correlation (rho>0.9). 3.9 DNA library using human-derived genomic DNA

To the genomic DNA described in 2. above (15 ng, human-derived genomic DNA), a random primer (final concentration: 60 microM; 10-base primer A), a 0.2 mM dNTP mixture, 1.0 mM MgCl₁, and 1.25 units of DNA polymerase (PrimeSTAR, TAKARA) were added, and a reaction solution was prepared while adjusting the final reaction level to 50 microliters. PCR was carried out under thermal cycling conditions comprising 98 degrees C. for 2 minutes and 30 cycles of 98 degrees C. for 10 seconds, 50 degrees C. for 15 seconds, and 72 degrees C. for 20 seconds, followed by storage at 4 degrees C. The DNA library obtained in this experiment was subjected to purification and electrophoresis in the same manner as in 3.1.3. Also, the reproducibility of the repeated data was evaluated on the basis of the Spearman's rank correlation (rho>0.9).

4. Results and Examination

4.1 Correlation Between PCR Conditions and DNA Library Size

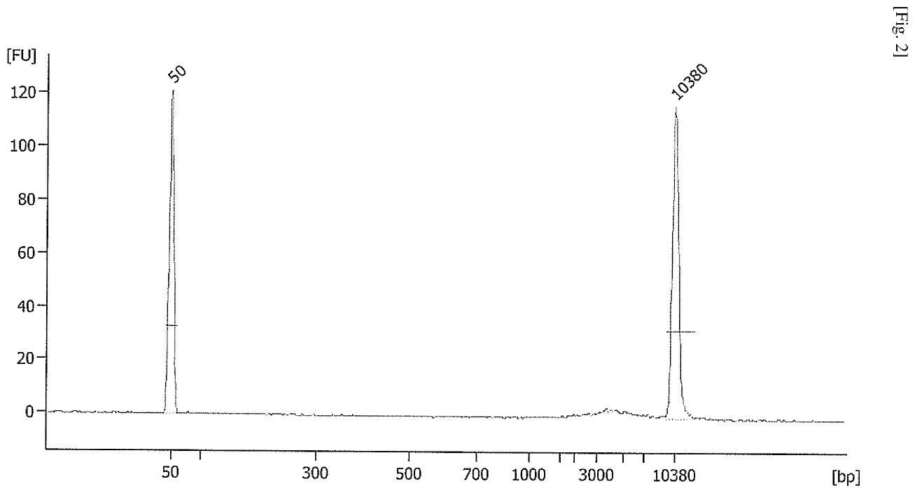

When PCR was conducted with the use of random primers in accordance with conventional PCR conditions (3.1.2 described above), the amplified DNA library size was as large as 2 kbp or more, but amplification of the DNA library of a target size (i.e., 100-bp to 500-bp) was not observed (FIG. 2). A DNA library of 100 bp to 500 bp could not be obtained because it was highly unlikely that a random primer would function as a primer in a region of 500 bp or smaller. In order to prepare a DNA library of the target size (i.e., 100 bp to 500 bp), it was considered necessary to induce non-specific amplification with high reproducibility.

The correlation between the annealing temperature (3.1.4 above), the enzyme amount (3.1.5 above), the MgCl₂ concentration (3.1.6 above), the primer length (3.1.7 above), and the primer concentration (3.18 above), which are considered to affect PCR specificity, and the DNA library size were examined.

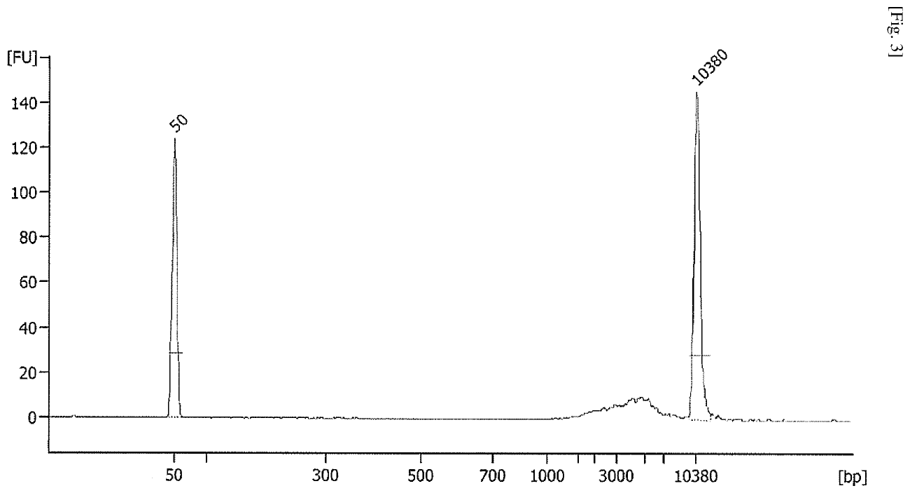

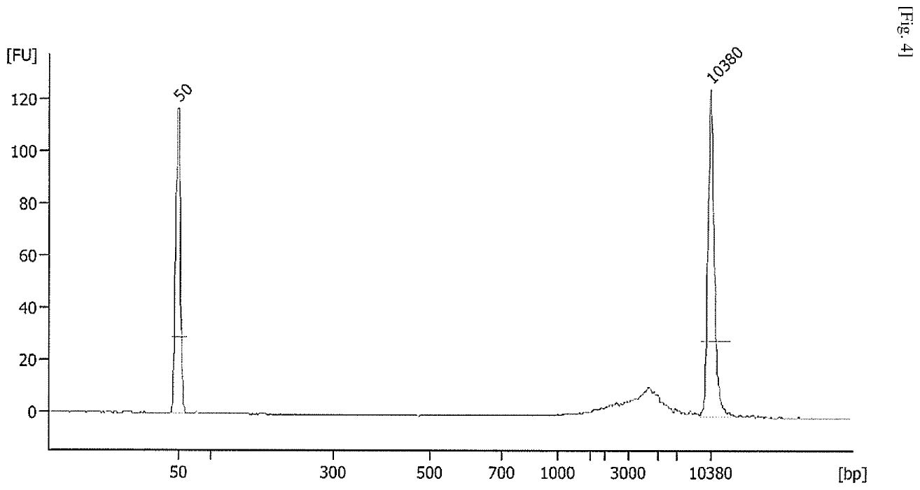

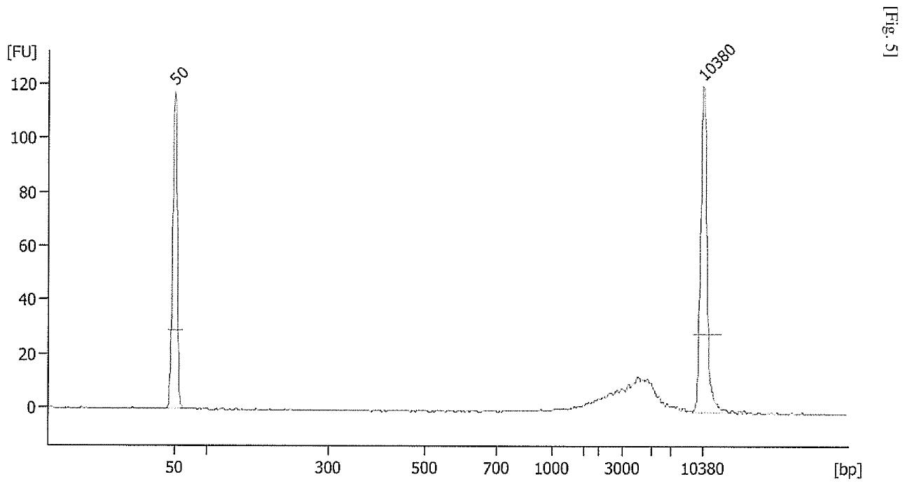

FIG. 3 shows the results of the experiment described in 3.1.4 attained at an annealing temperature of 45 degrees C. FIG. 4 shows the results attained at an annealing temperature of 40 degrees C., and FIG. 5 shows the results attained at an annealing temperature of 37 degrees C. By reducing the annealing temperature from 45 degrees C., 40 degrees C., to 37 degrees C., as shown in FIGS. 3 to 5, the amounts of high-molecular-weight DNA library amplified increased, although amplification of low-molecular-weight DNA library was not observed.

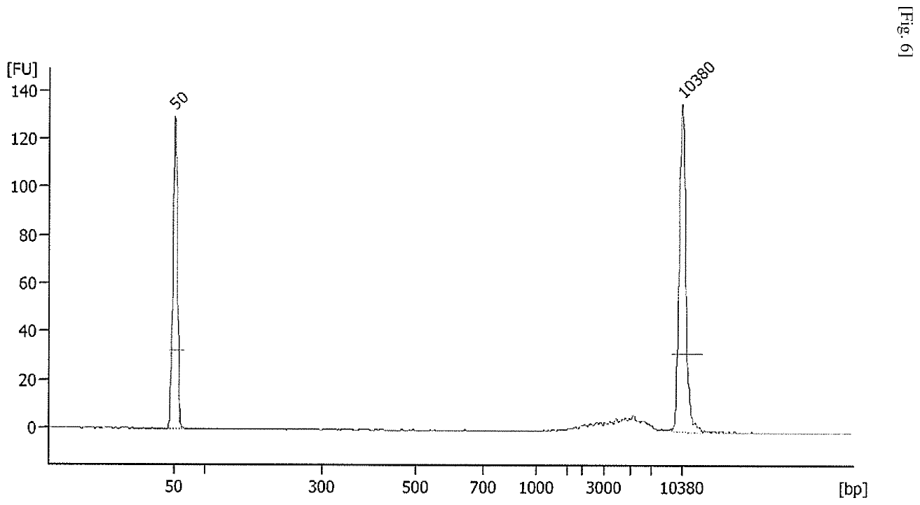

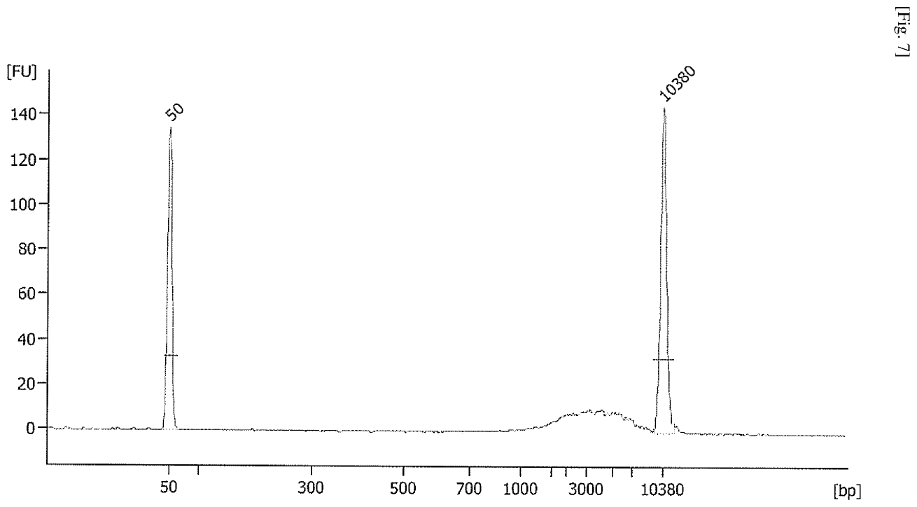

FIG. 6 shows the results of the experiment described in 3.1.5 attained when the enzyme amount is increased by 2 times, and FIG. 7 shows the results attained when the enzyme amount is increased by 10 times the original amount. By increasing the enzyme amount by 2 times or 10 times a common amount, as shown in FIGS. 6 and 7, the amounts of high-molecular-weight DNA library amplified increased, although amplification of low-molecular-weight DNA library was not observed.

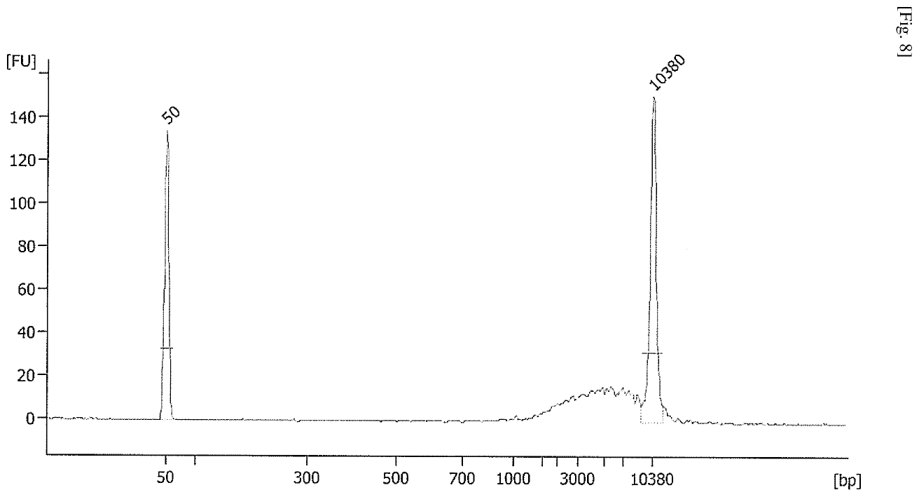

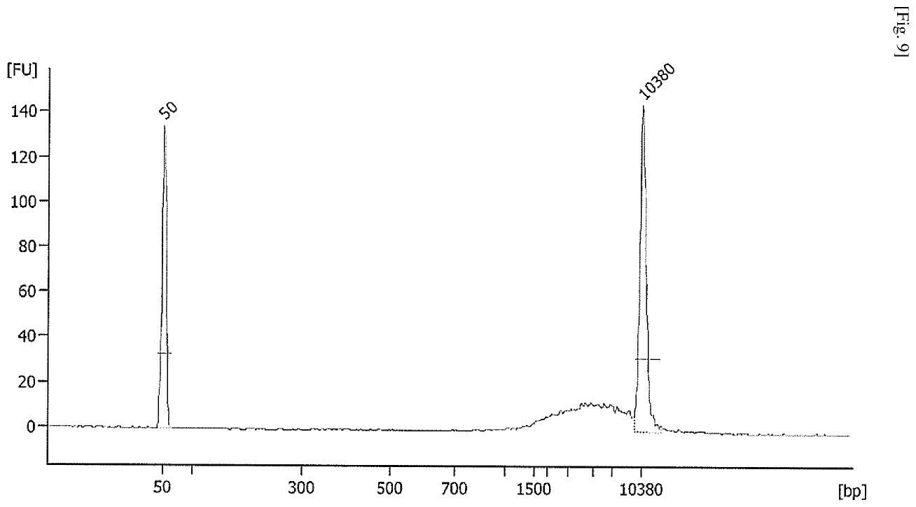

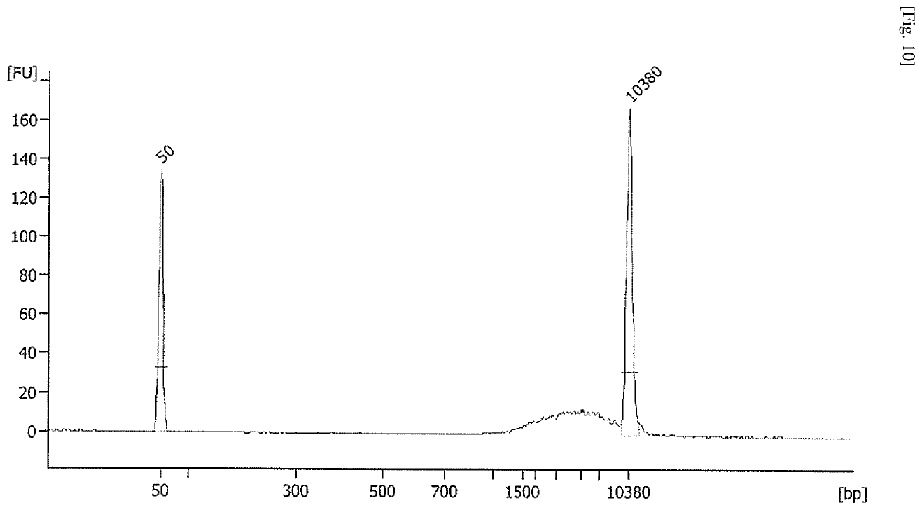

FIG. 8 shows the results of the experiment described in 3.1.6 attained when the MgCl₂ concentration is increased by 2 times a common amount, FIG. 9 shows the results attained when the MgCl₂ concentration is increased by 3 times, and FIG. 10 shows the results attained when the MgCl₂ concentration is increased by 4 times. By increasing the MgCl₂ concentration by 2 times, 3 times, and 4 times the common amount, as shown in FIGS. 8 to 10, the amounts of high-molecular-weight DNA library amplified varied, although amplification of a low-molecular-weight DNA library was not observed.

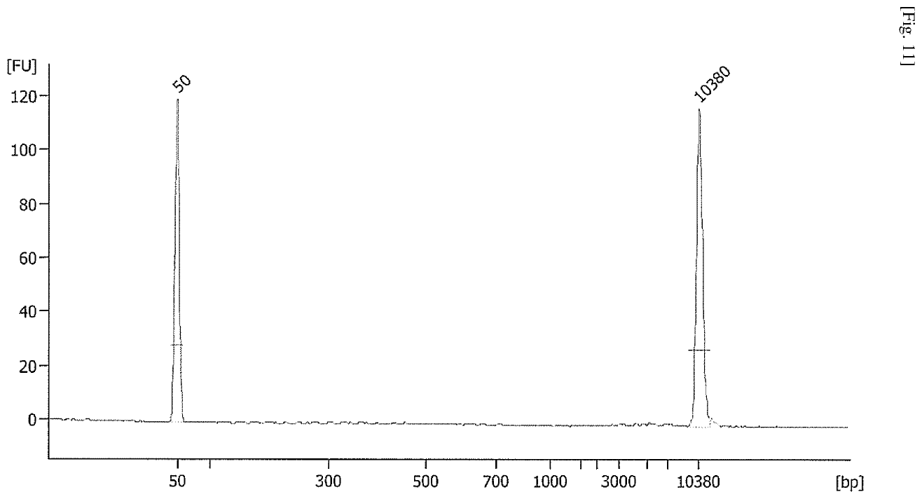

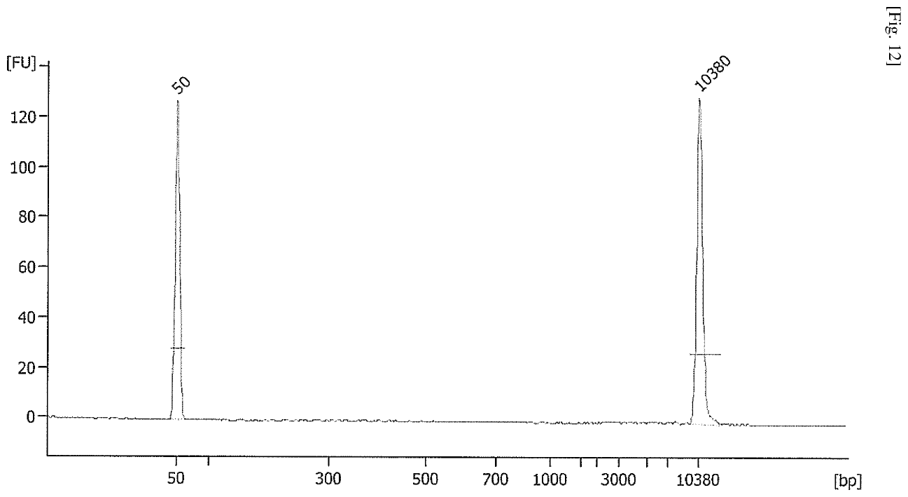

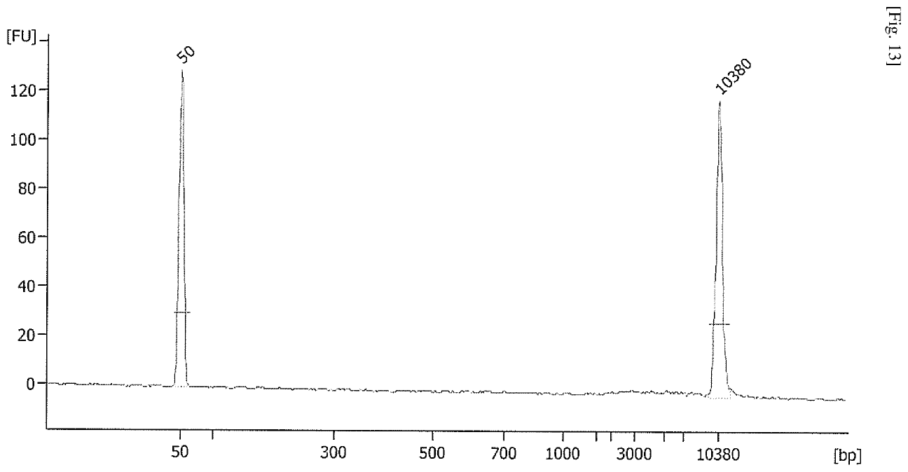

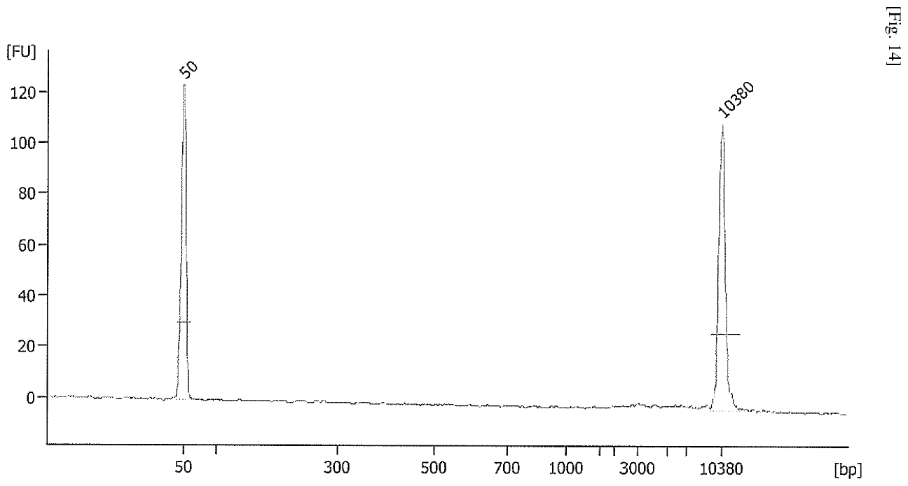

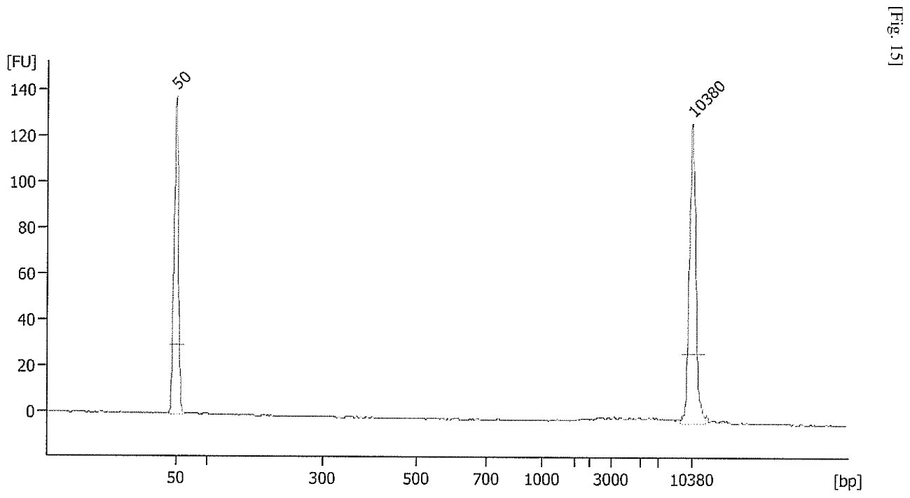

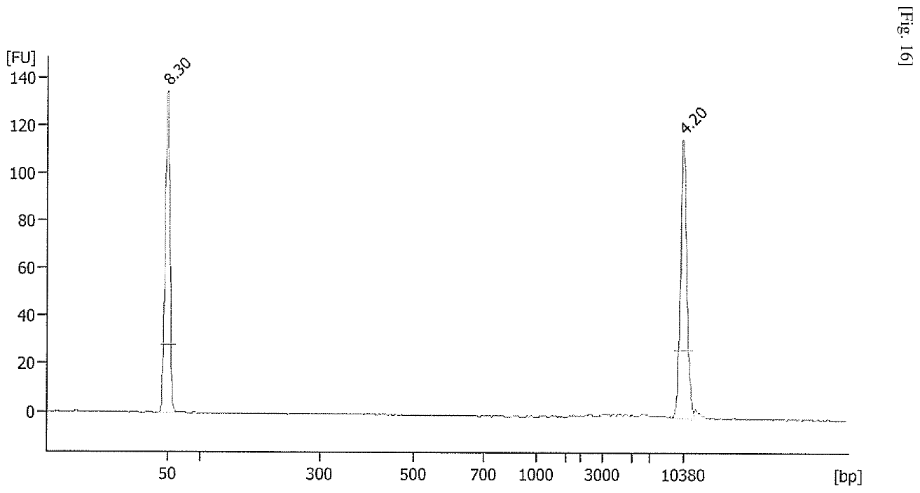

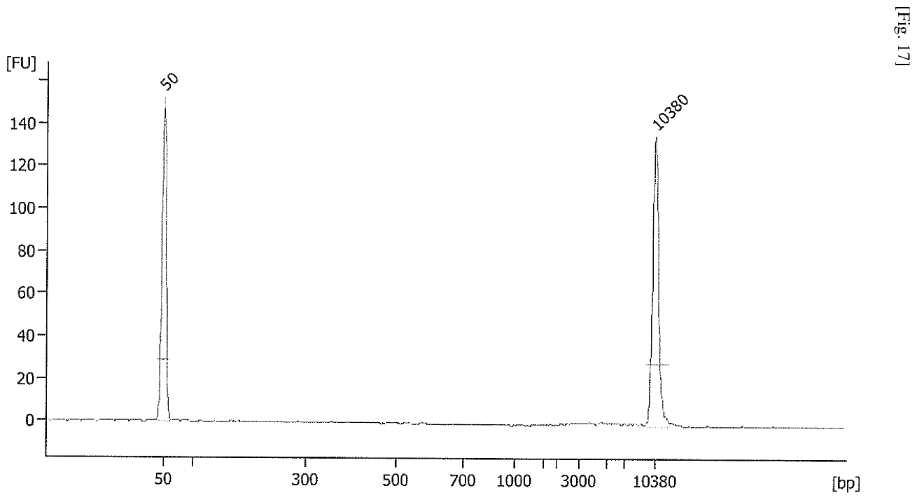

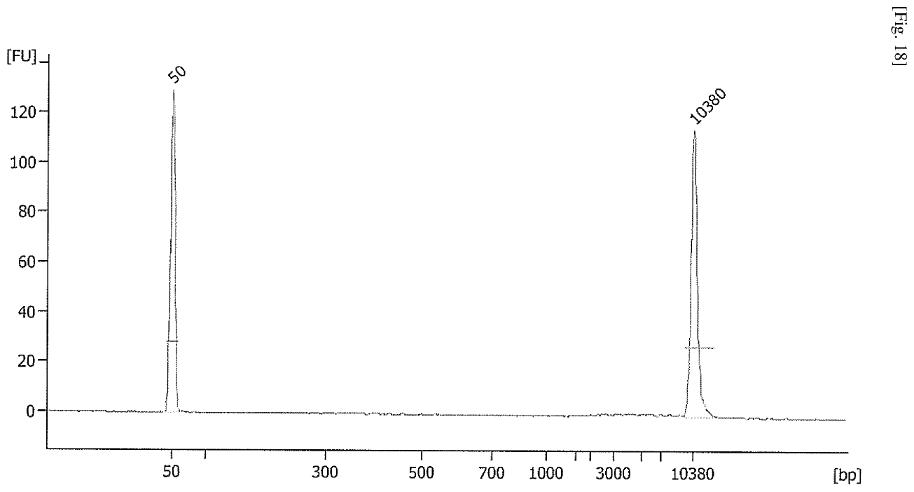

FIGS. 11 to 18 show the results of the experiment described in 3.1.7 attained at the random primer lengths of 8 bases, 9 bases, 11 bases, 12 bases, 14 bases, 16 bases, 18 bases, and 20 bases, respectively. Regardless of the length of a random primer, as shown in FIGS. 11 to 18, no significant change was observed in comparison with the results shown in FIG. 2 (a random primer comprising 10 bases).

The results of experiment described in 3.1.8 are summarized in Table 23.

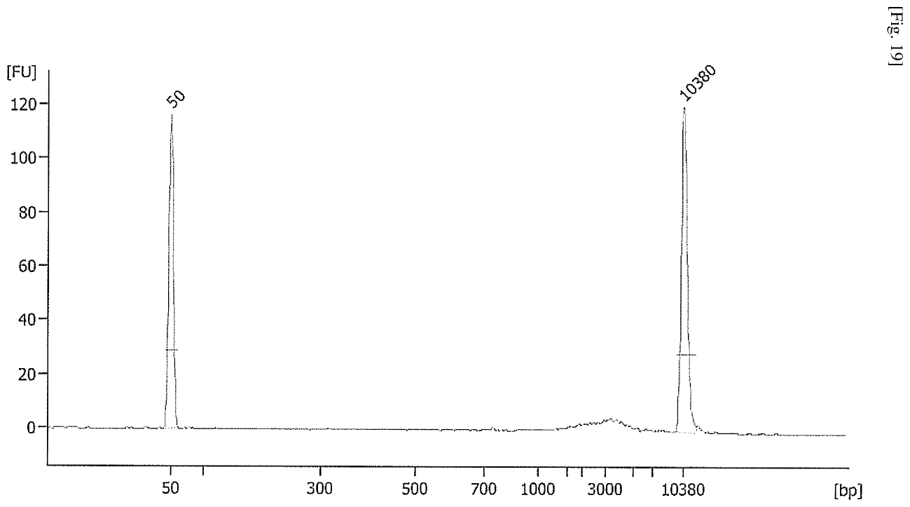

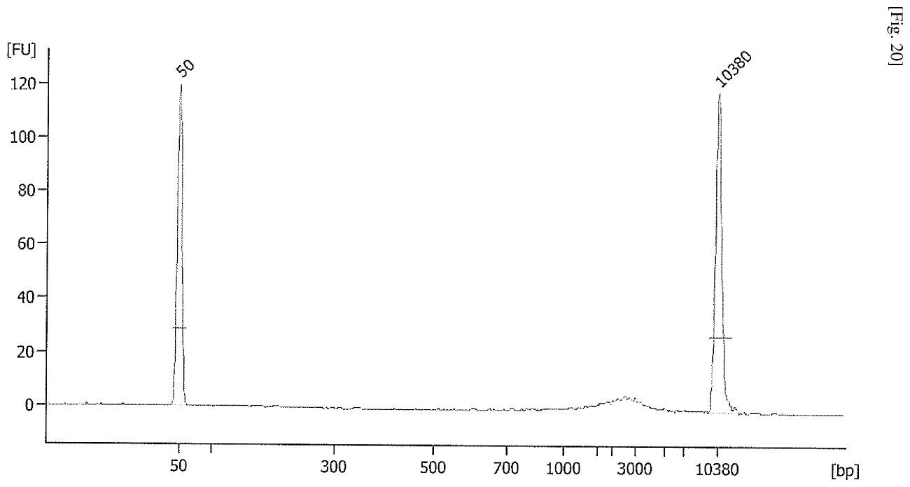

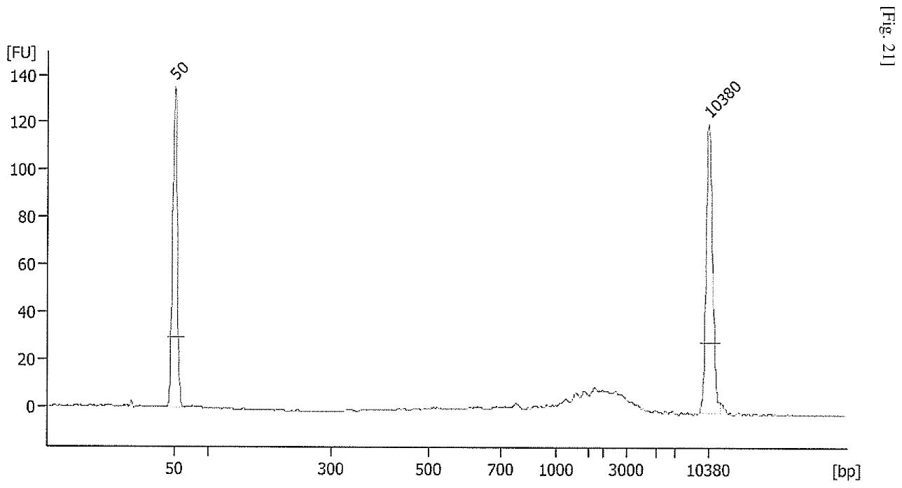

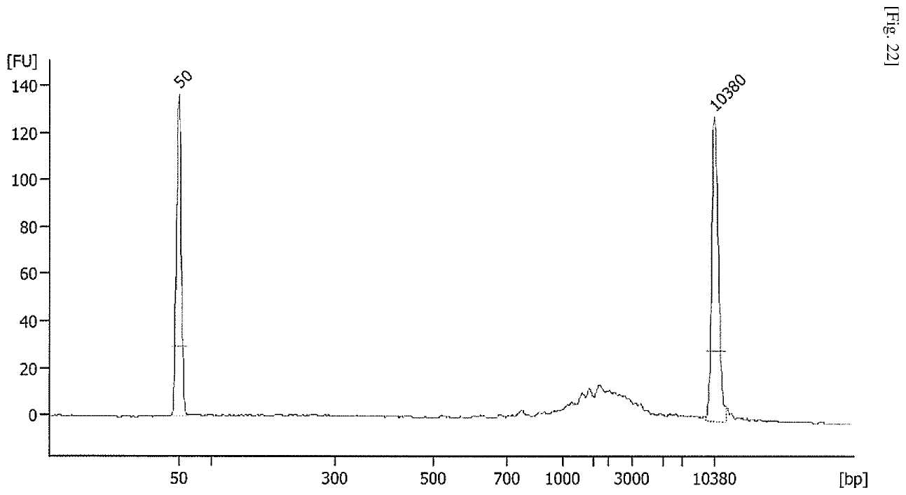

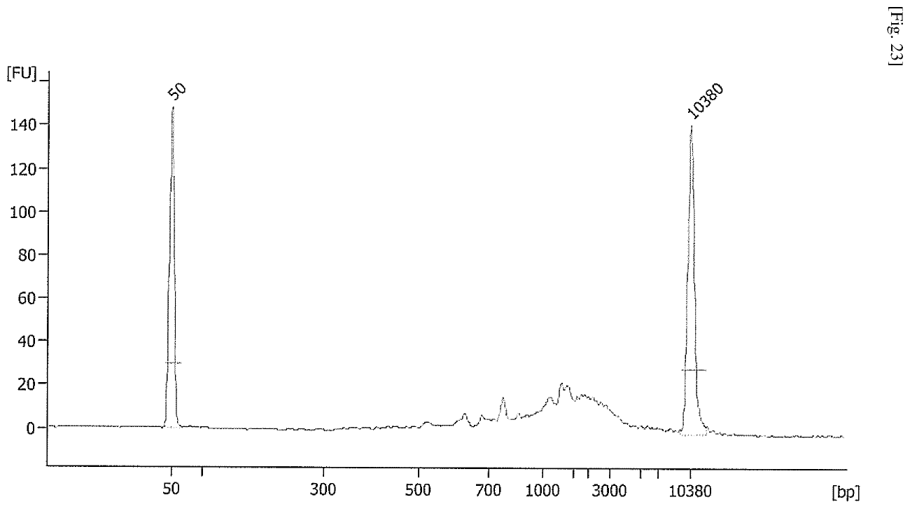

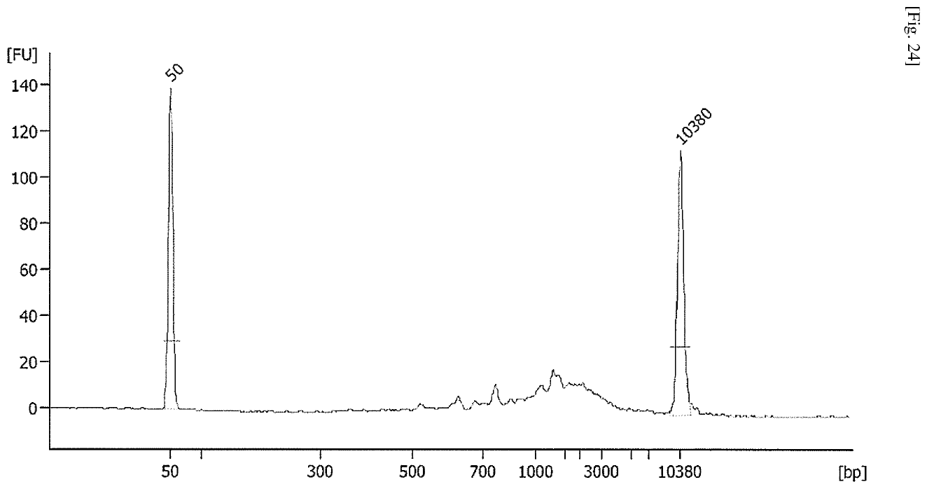

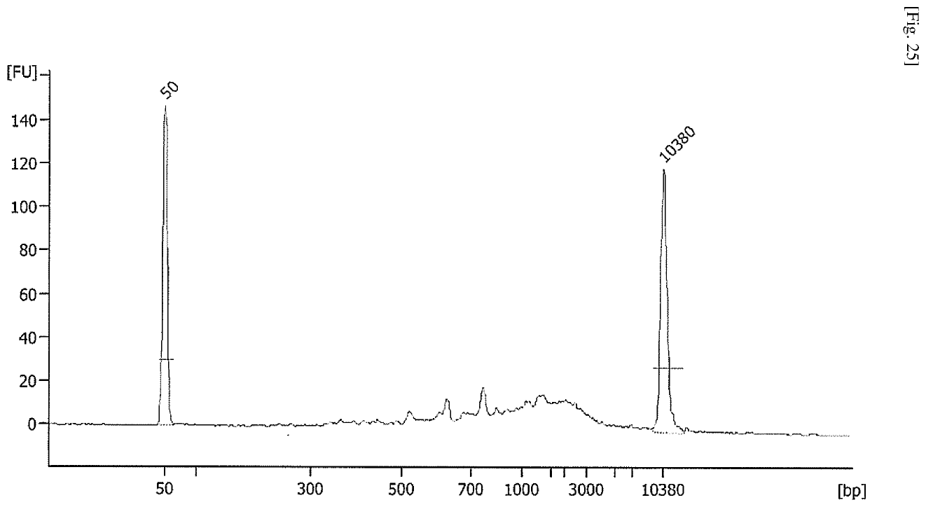

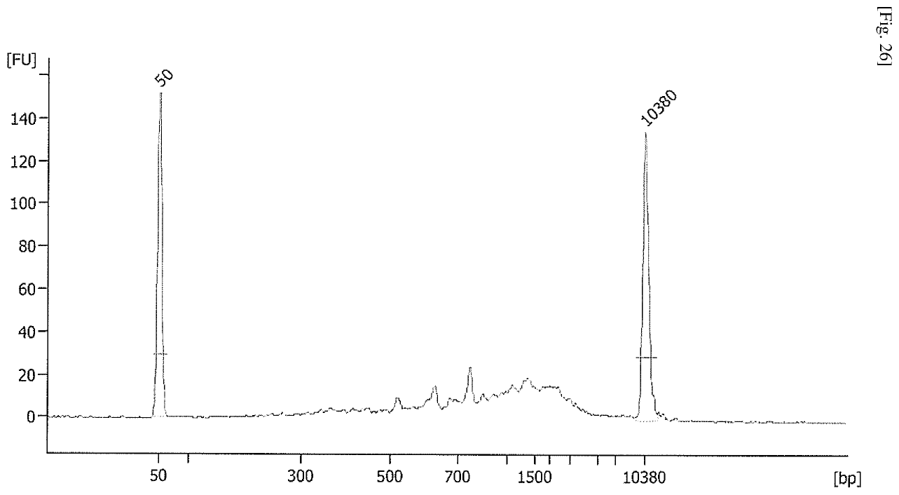

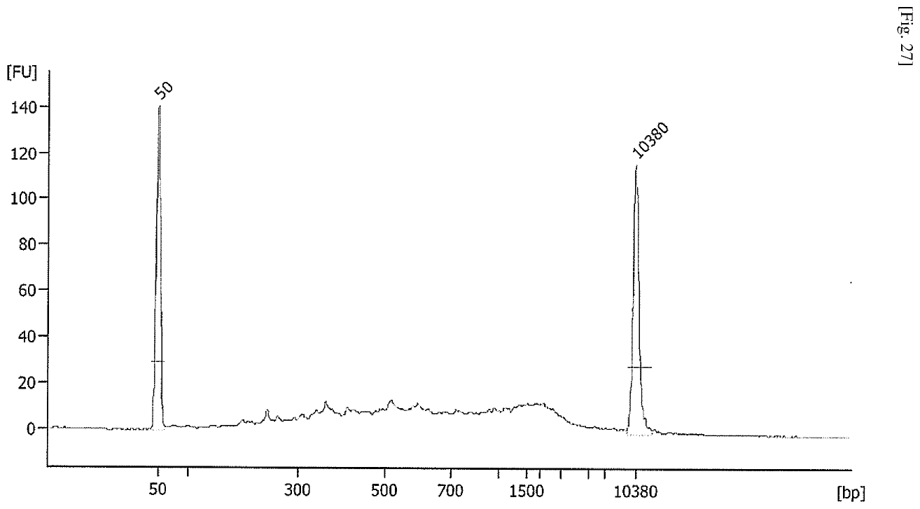

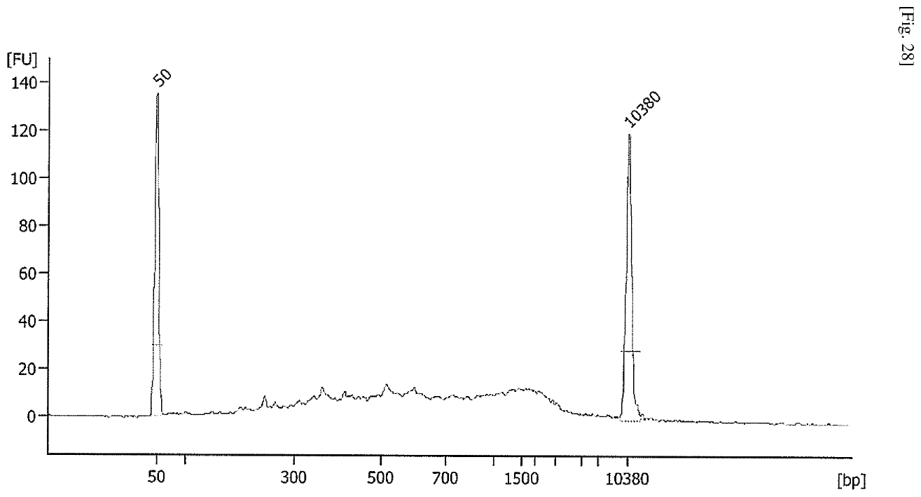

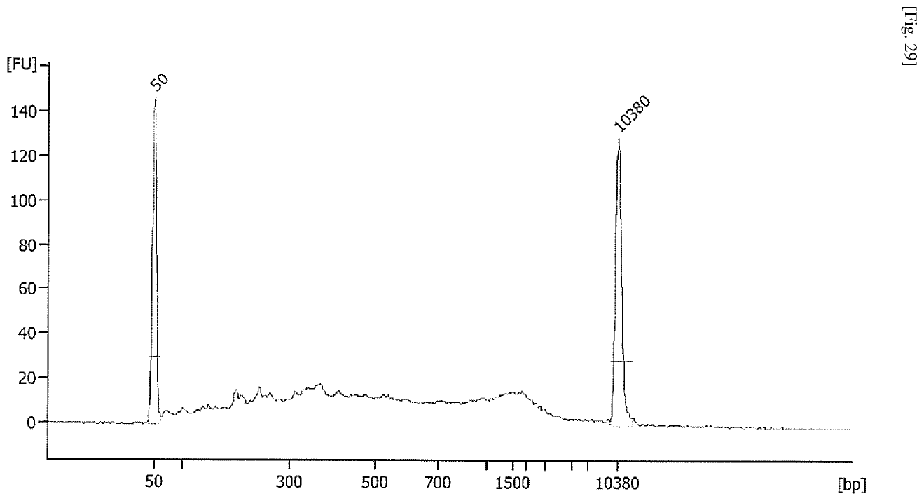

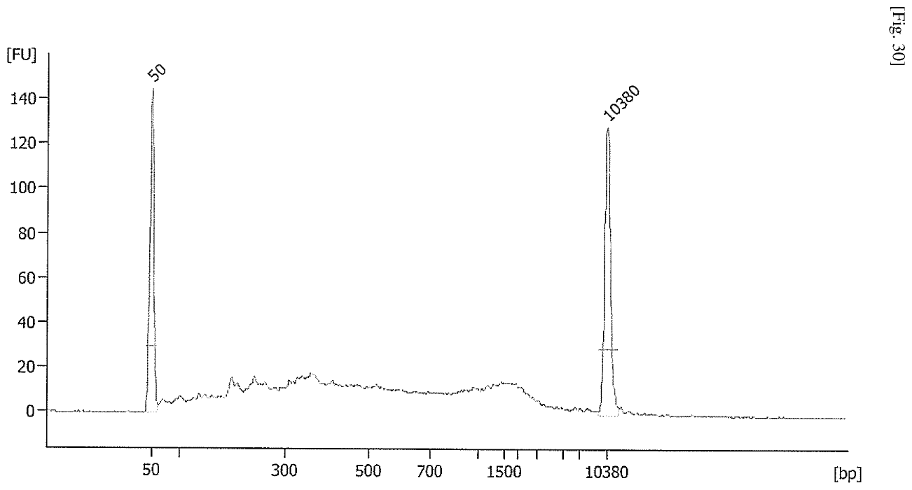

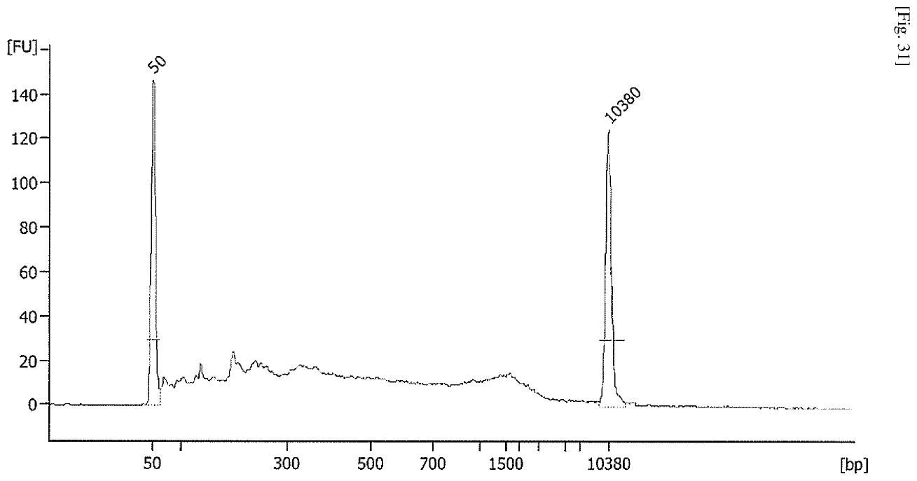

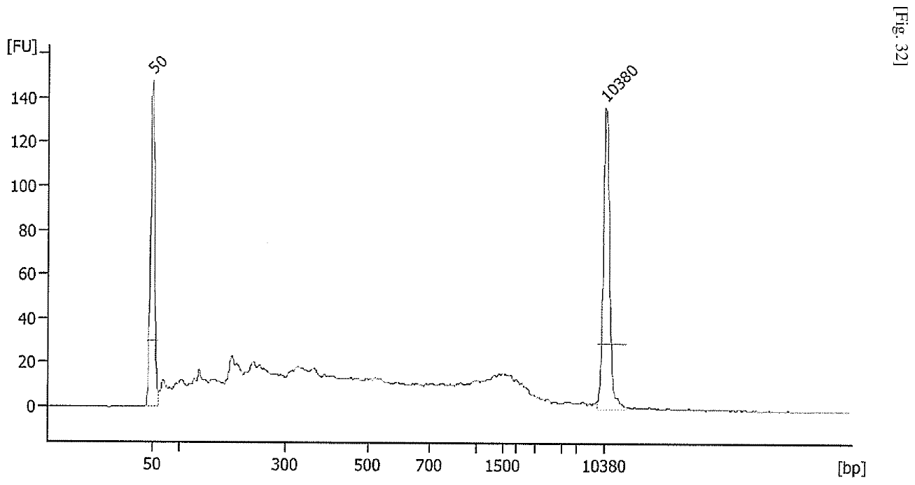

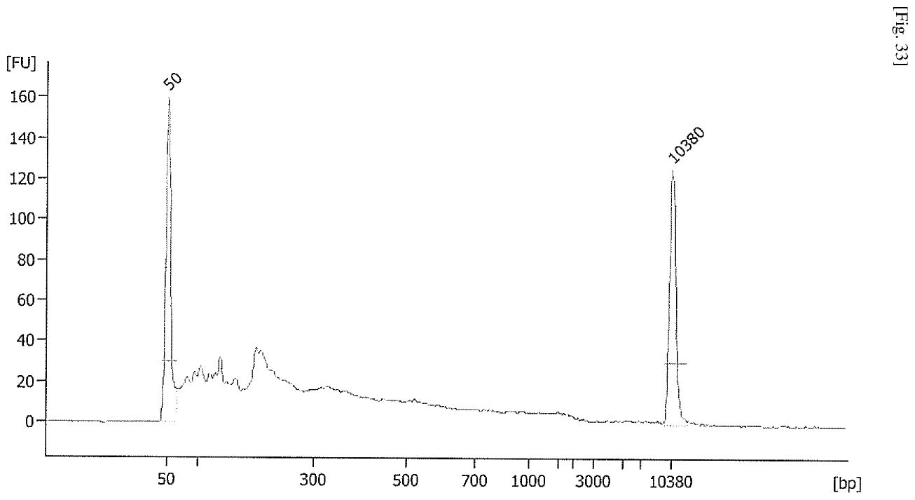

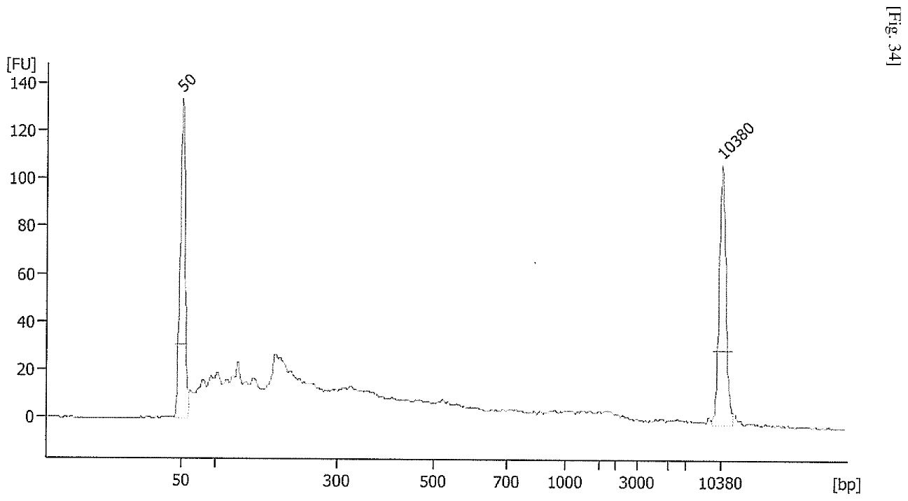

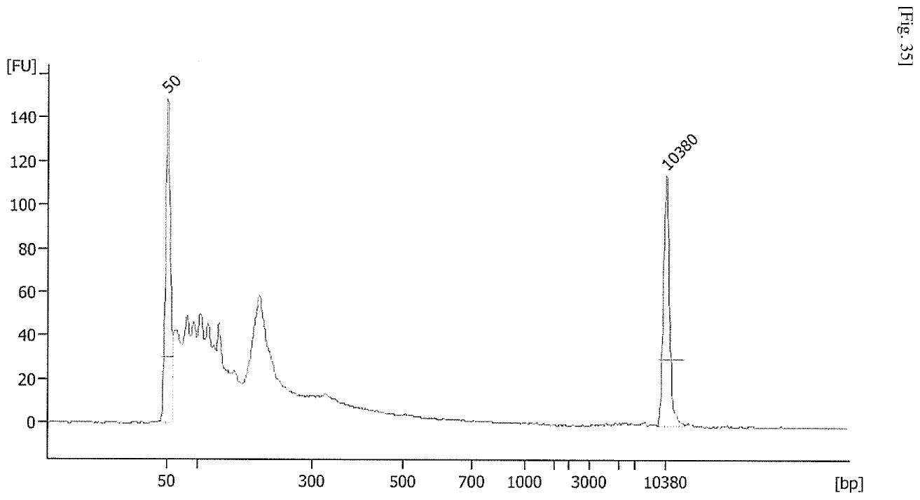

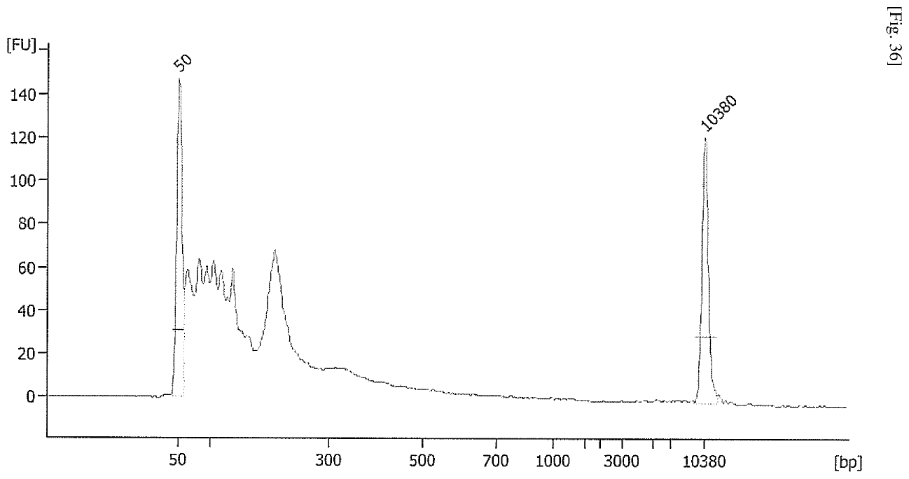

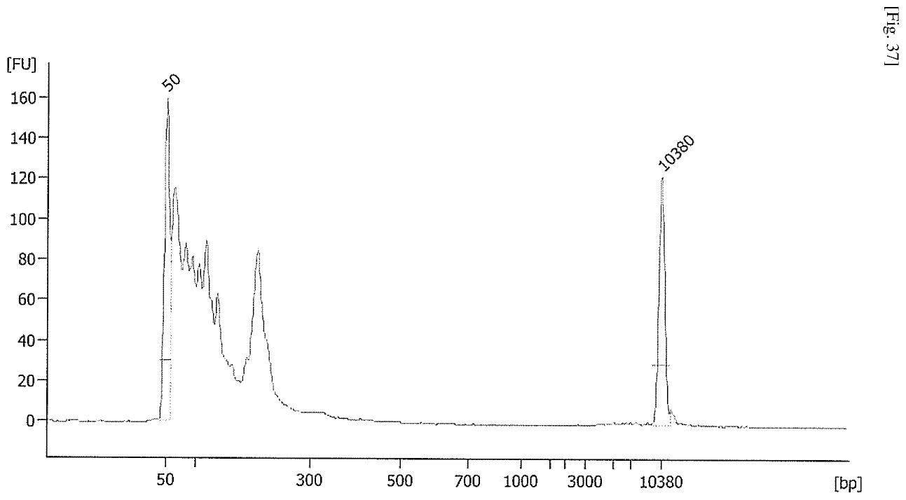

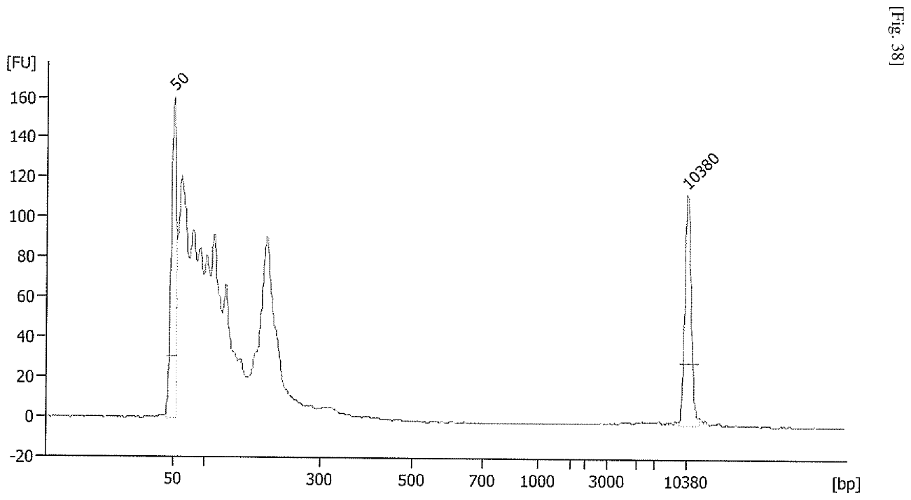

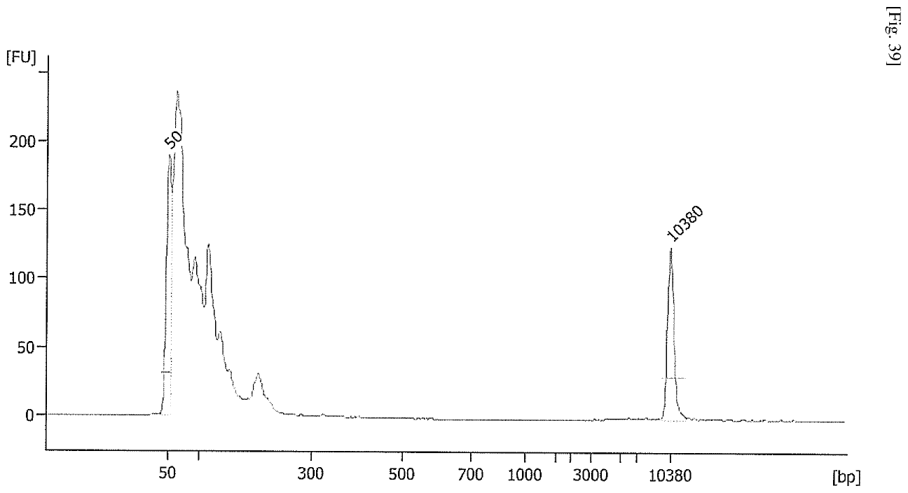

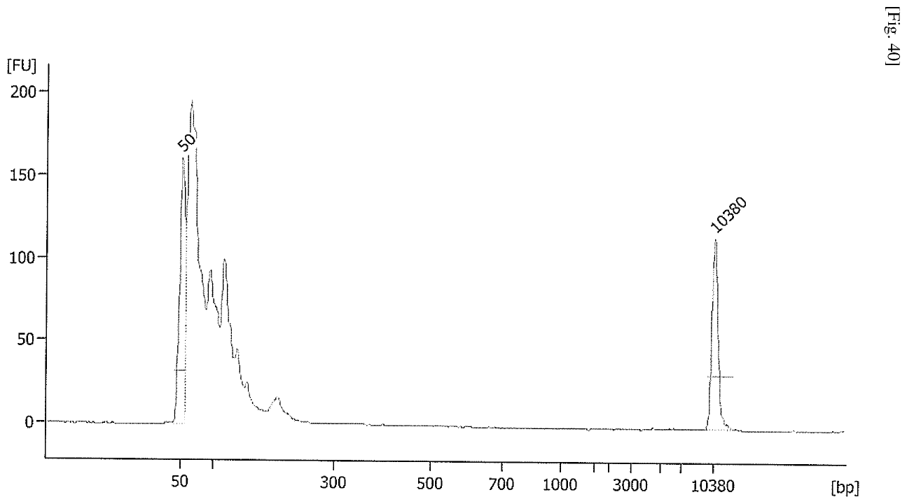

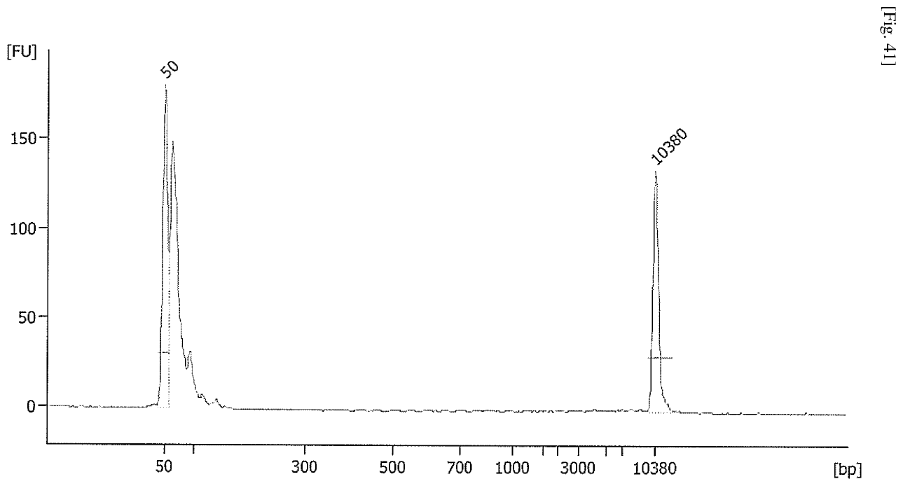

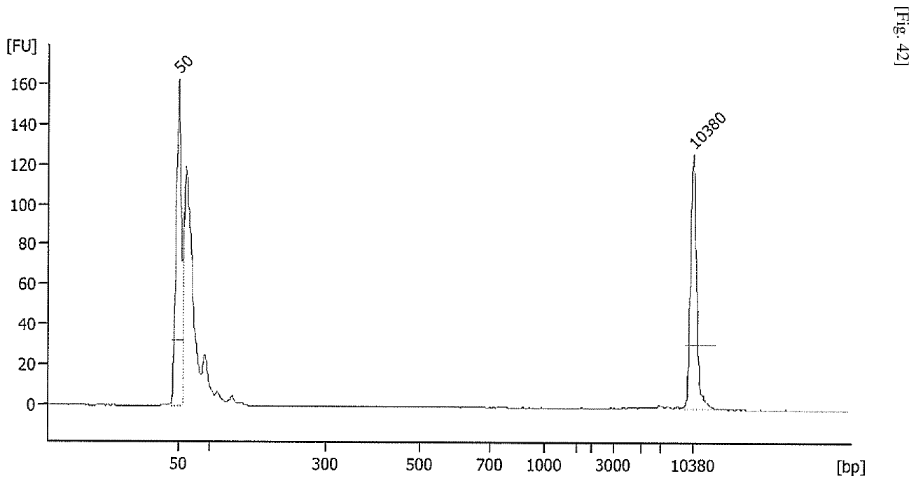

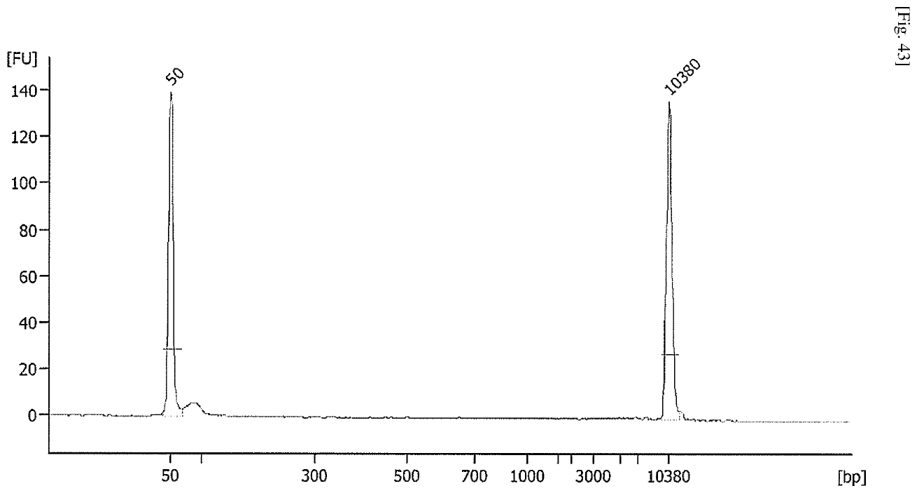

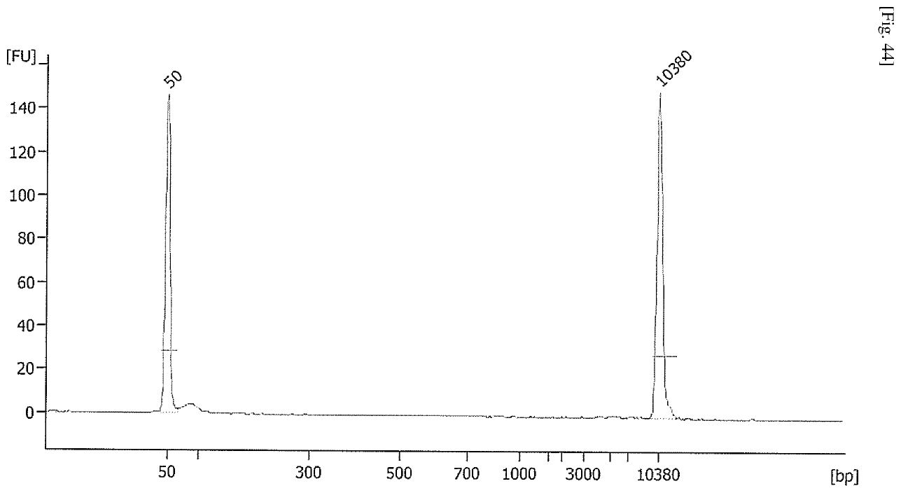

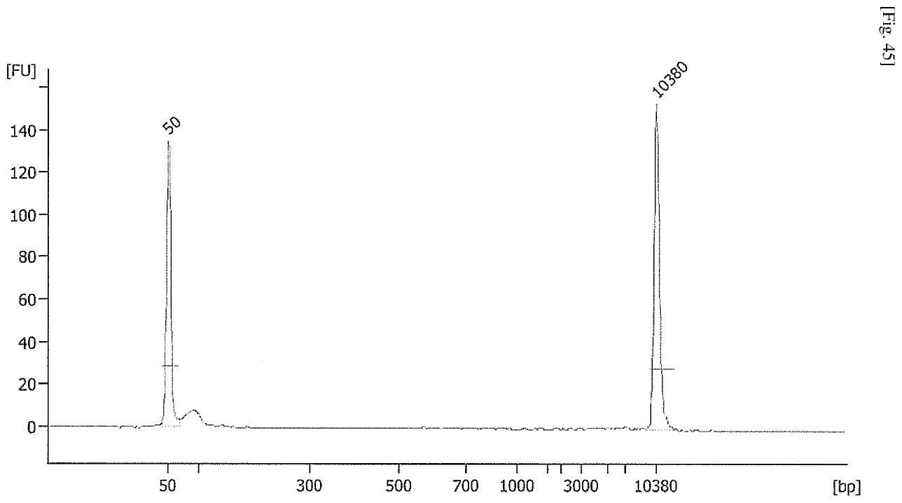

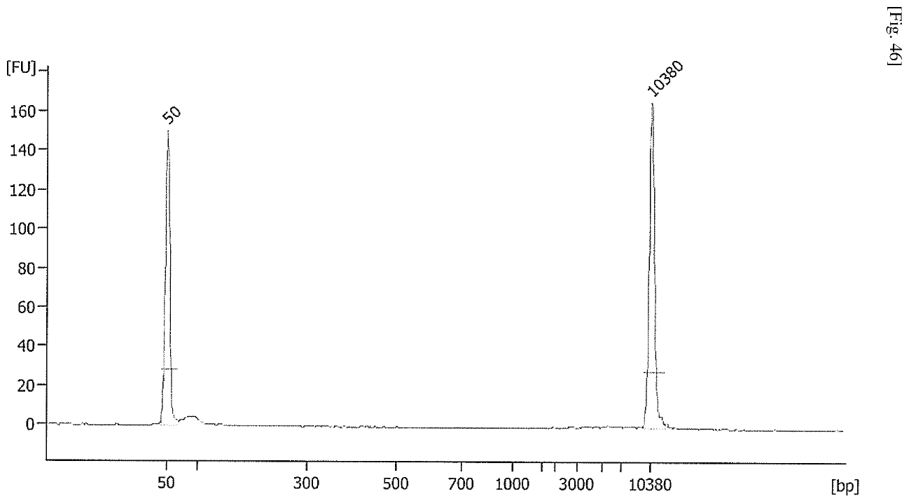

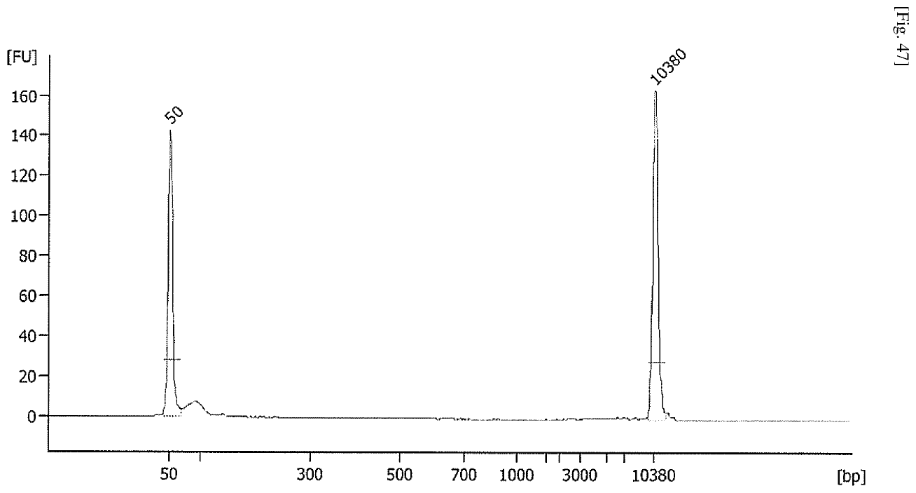

TABLE 23

| Concentration | | | Correlational |
|---|---|---|---|
| (μM) | Repeat | FIG. No. | coefficient (ρ) |
| 2 | — | FIG. 19 | — |
| 4 | — | FIG. 20 | — |
| 6 | First | FIG. 21 | 0.889 |
| | Second | FIG. 22 | |
| 8 | First | FIG. 23 | 0.961 |
| | Second | FIG. 24 | |
| 10 | First | FIG. 25 | 0.979 |
| | Second | FIG. 26 | |
| 20 | First | FIG. 27 | 0.950 |
| | Second | FIG. 28 | |
| 40 | First | FIG. 29 | 0.975 |
| | Second | FIG. 30 | |
| 60 | First | FIG. 31 | 0.959 |
| | Second | FIG. 32 | |
| 100 | First | FIG. 33 | 0.983 |
| | Second | FIG. 34 | |
| 200 | First | FIG. 35 | 0.991 |
| | Second | FIG. 36 | |
| 300 | First | FIG. 37 | 0.995 |
| | Second | FIG. 38 | |
| 400 | First | FIG. 39 | 0.988 |
| | Second | FIG. 40 | |
| 500 | First | FIG. 41 | 0.971 |
| | Second | FIG. 42 | |
| 600 | — | FIG. 43 | — |
| 700 | — | FIG. 44 | — |
| 800 | — | FIG. 45 | — |
| 900 | — | FIG. 46 | — |
| 1000 | — | FIG. 47 | — |

With the use of random primers comprising 10 bases, as shown in FIGS. 19 to 47, amplification was observed in a 1-kbp DNA fragment at the random primer concentration of 6 microM. As the concentration increased, the molecular weight of a DNA fragment decreased. Reproducibility at the random primer concentration of 6 to 500 microM was examined. As a result, a relatively low rho value of 0.889 was attained at the concentration of 6 microM, which is 10 times higher than the usual level. At the concentration of 8 microM, which is equivalent to 13.3 times higher than the usual level, and at 500 microM, which is 833.3 times higher than the usual level, a high rho value of 0.9 or more was attained. The results demonstrate that a DNA fragment of 1 kbp or smaller can be amplified while achieving high reproducibility by elevating the random primer concentration to a level significantly higher than the concentration employed under general PCR conditions. When the random primer concentration is excessively higher than 500 microliter, amplification of a DNA fragment of a desired size cannot be observed. In order to amplify a low-molecular-weight DNA fragment with excellent reproducibility, accordingly, it was found that the random primer concentration should fall within an optimal range, which is higher than the concentration employed in a general PCR procedure and equivalent to or lower than a given level.

4.2 Confirmation of Reproducibility Via MiSeq

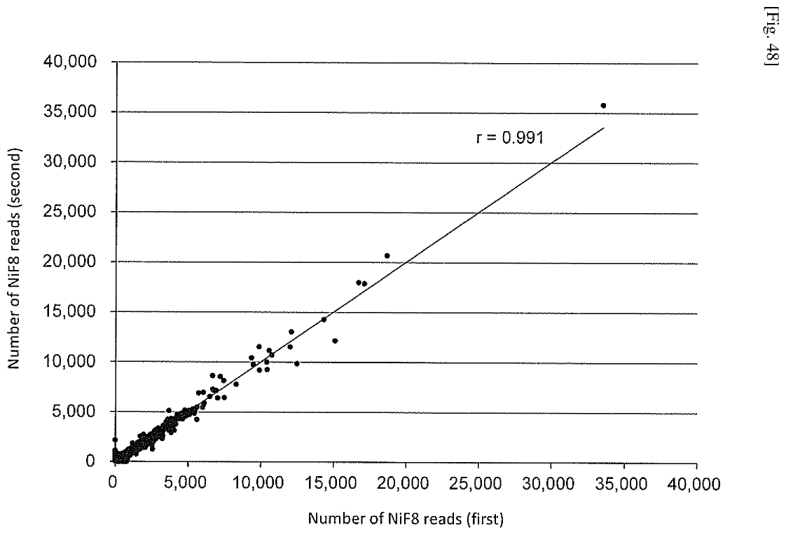

In order to confirm the reproducibility for DNA library production, as described in 3.2 above, the DNA library amplified with the use of the genomic DNA extracted from NiF8 as a template and random primers was analyzed with the use of a next-generation sequencer (MiSeq), and the results are shown in FIG. 48. As a result of 3.2.4 above, 47,484 read patterns were obtained. As a result of comparison of the number of reads obtained through repeated measurements, a high correlation (i.e., a correlational coefficient “r” of 0.991) was obtained, as with the results of electrophoresis. Accordingly, it was considered that a DNA library could be produced with satisfactory reproducibility with the use of random primers.

4.3 Analysis of Rice Variety Nipponbare

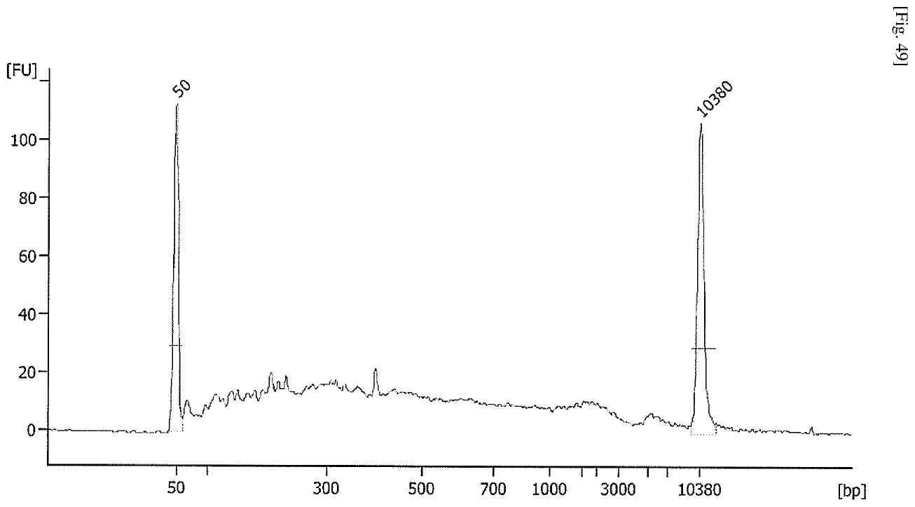

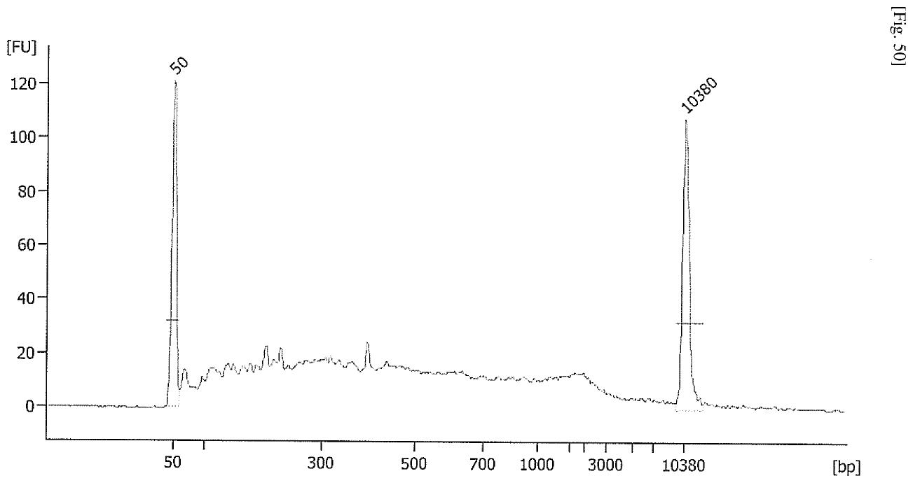

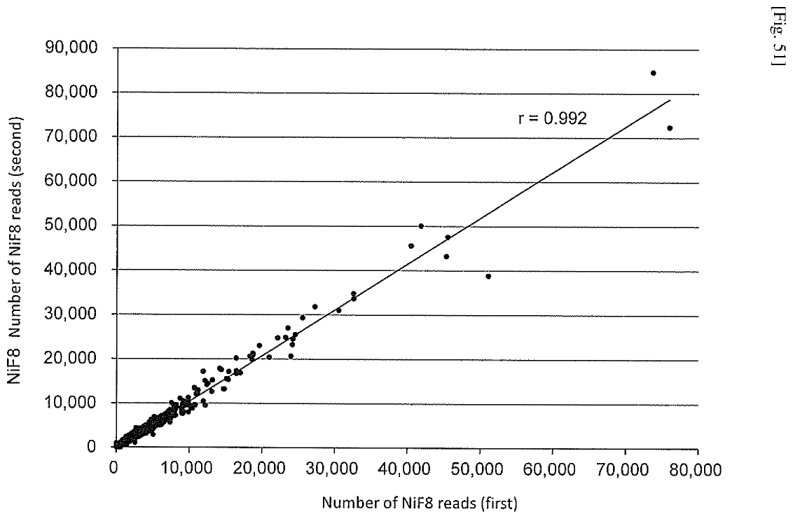

As described in 3.3 above, a DNA library was prepared with the use of genomic DNA extracted from the rice variety Nipponbare, the genomic information of which has been disclosed, as a template, and random primers and subjected to electrophoresis, and the results are shown in FIGS. 49 and 50. On the basis of the results shown in FIGS. 49 and 50, the rho value was found to be as high as 0.979. Also, FIG. 51 shows the results of analysis of the read data with the use of MiSeq. On the basis of the results shown in FIG. 51, the correlational coefficient “r” was found to be as high as 0.992. These results demonstrate that a DNA library of rice could be produced with very high reproducibility with the use of random primers.

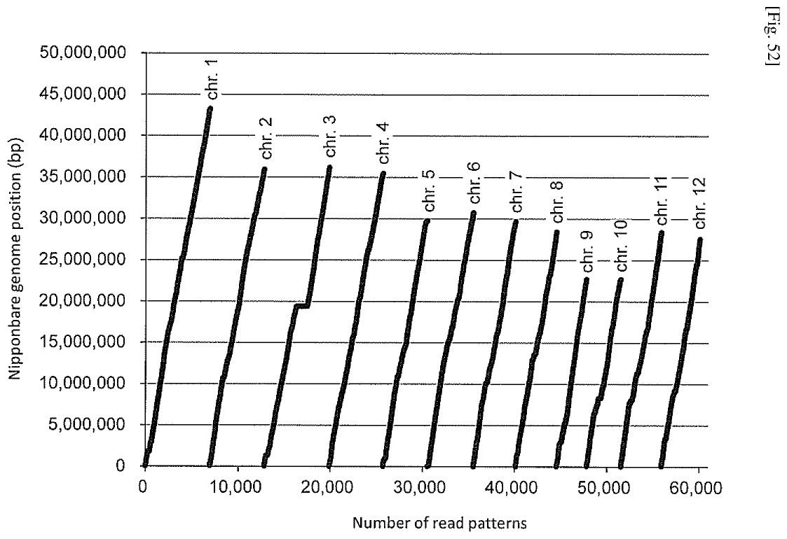

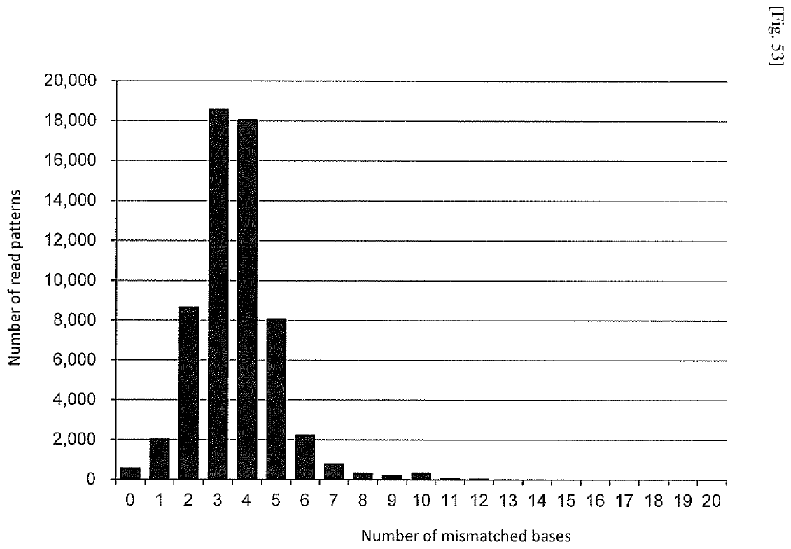

As described in 3.3.3, the obtained read pattern was mapped to the genomic information of Nipponbare. As a result, DNA fragments were found to be evenly amplified throughout the genome at intervals of 6.2 kbp (FIG. 52). As a result of comparison of the sequence and genome information of random primers, 3.6 mismatches were found on average, and one or more mismatches were observed in 99.0% of primer pairs (FIG. 53). The results demonstrate that a DNA library involving the use of random primers is produced with satisfactory reproducibility via non-specific amplification evenly throughout the genome.

4.4 Detection of Polymorphism and Genotype Identification of Sugarcane

As described in 3.4, DNA libraries of the sugarcane varieties NiF8 and Ni9 and 22 hybrid progeny lines were produced with the use of random primers, the resulting DNA libraries were analyzed with the next-generation sequencer (HiSeq), the polymorphisms of the parent varieties were detected, and the genotypes of the hybrid progenies were identified on the basis of the read data. Table 24 shows the results.

TABLE 24
Number of sugarcane NiF8 and Ni0 markers and accuracy for genotype identification

| | | F1_01 | | F1_02 | | Total | |
|---|---|---|---|---|---|---|---|
| | Number of markers | Consistency | Reproducibility | Consistency | Reproducibility | Consistency | Reproducibility |
| NiF8 type | 8,683 | 8,680 | 99.97% | 8,682 | 99.99% | 17,362 | 99.98% |
| Ni9 type | 11,655 | 11,650 | 99.96% | 11,651 | 99.97% | 23,301 | 99.96% |
| Total | 20,338 | 20,330 | 99.96% | 20,333 | 99.98% | 40,663 | 99.97% |

As shown in Table 24, 8,683 NiF8 markers and 11,655 Ni9 markers; that is, a total of 20,338 markers, were produced. In addition, reproducibility for genotype identification of hybrid progeny lines was as high as 99.97%. This indicates that the accuracy for genotype identification is very high. In particular, sugarcane is polyploid (8x+n), the number of chromosomes is as large as 100 to 130, and the genome size is as large as 10 Gbp, which is at least 3 times greater than that of humans. Accordingly, it is very difficult to identify the genotype throughout the genomic DNA. As described above, numerous markers can be produced with the use of random primers, and the sugarcane genotype can thus be identified with high accuracy.

4.5 Experiment for Confirmation with PCR Marker

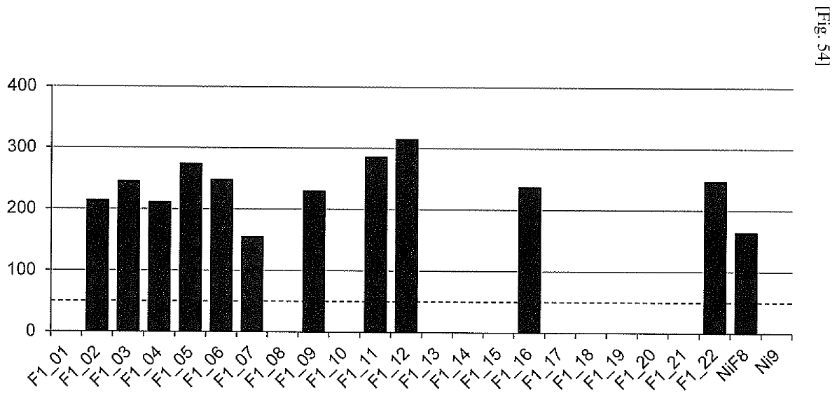

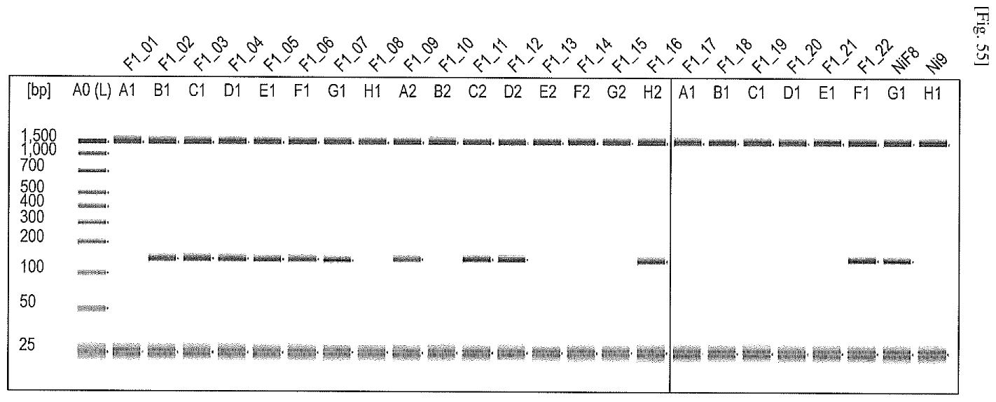

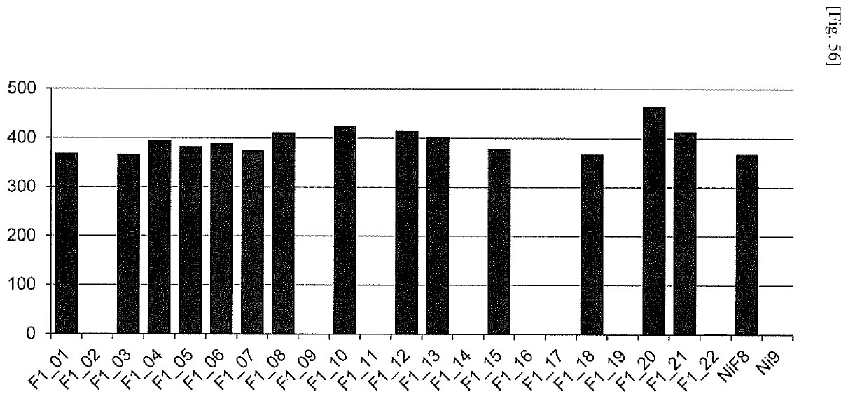

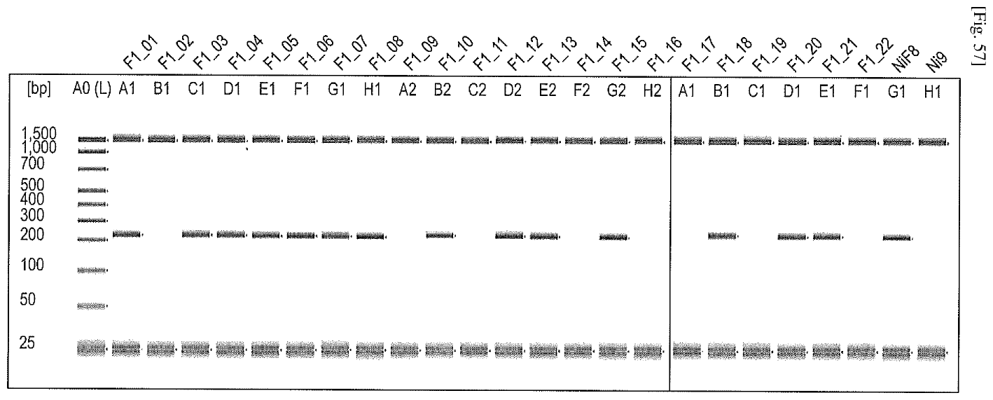

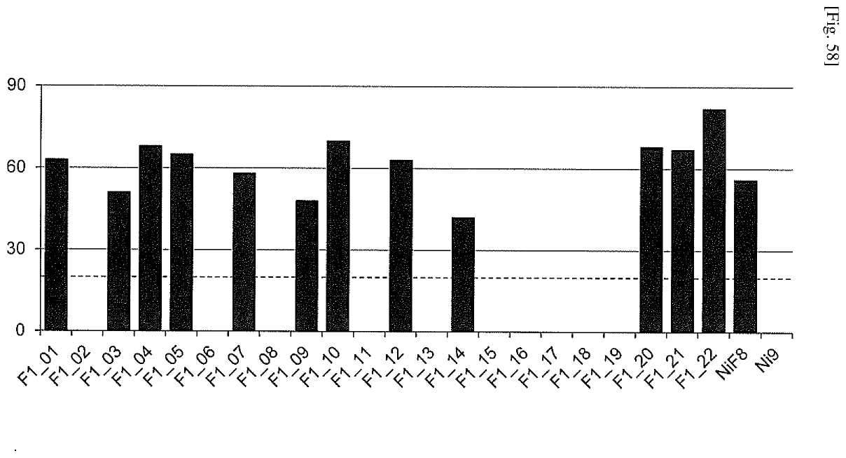

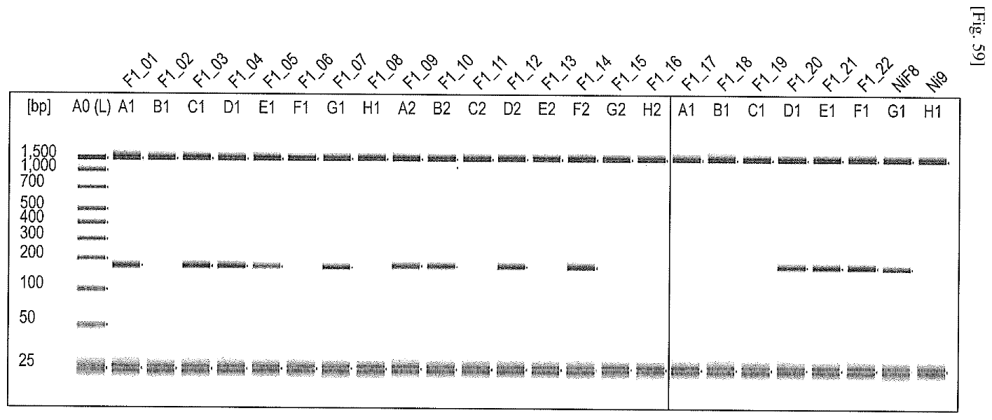

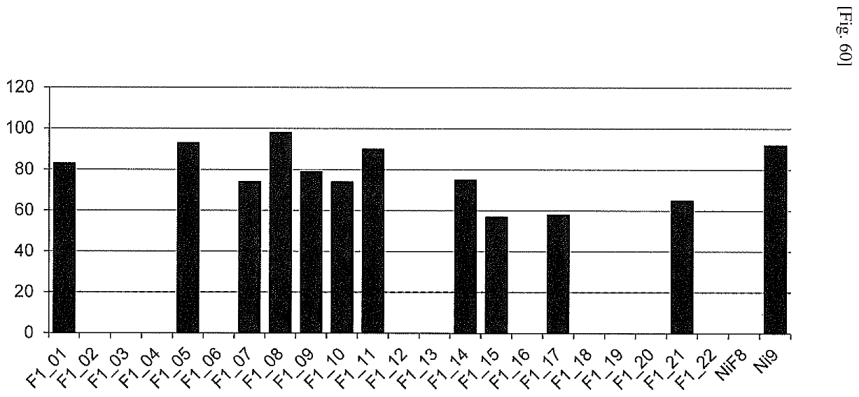

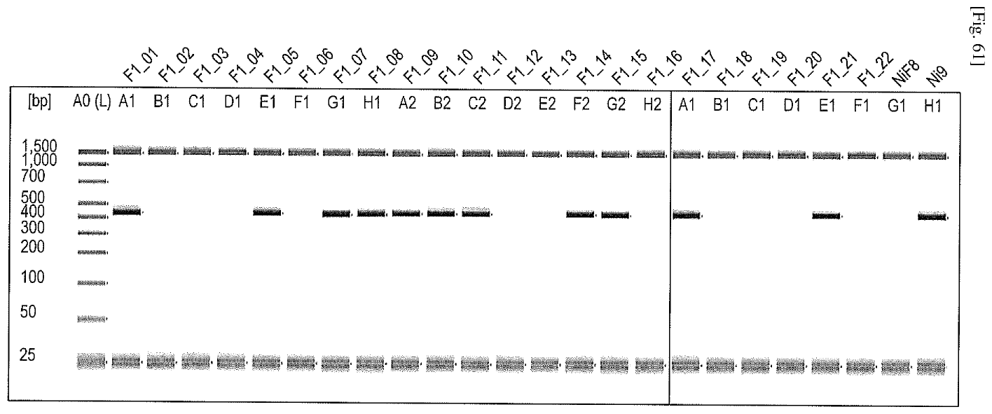

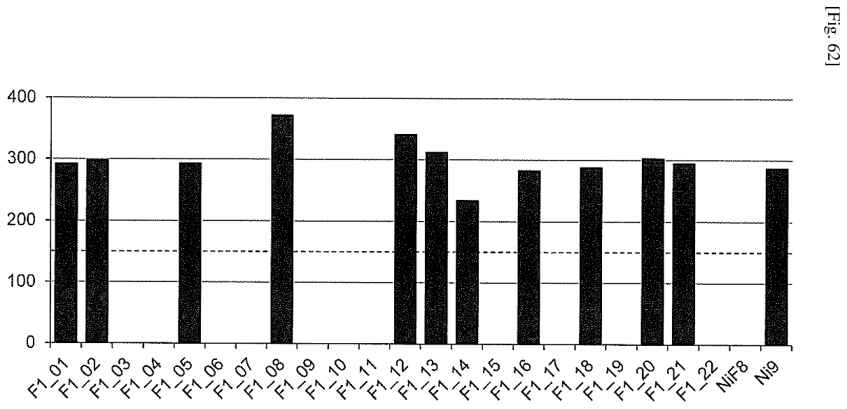

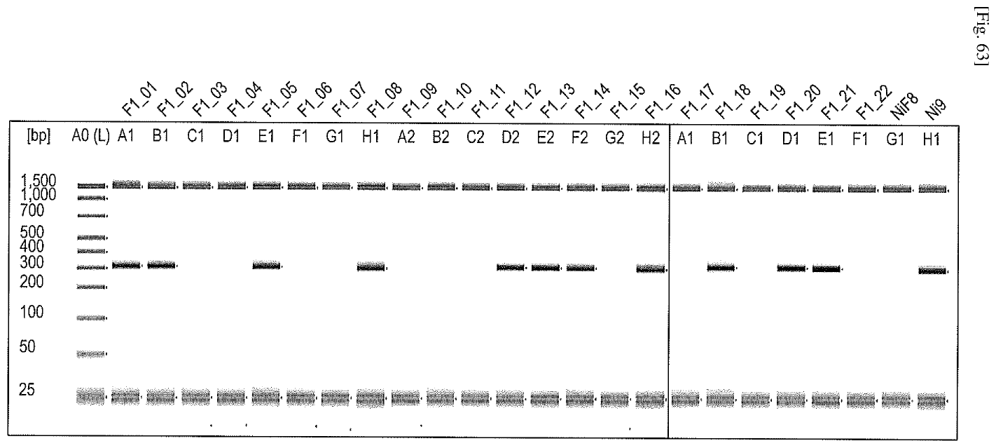

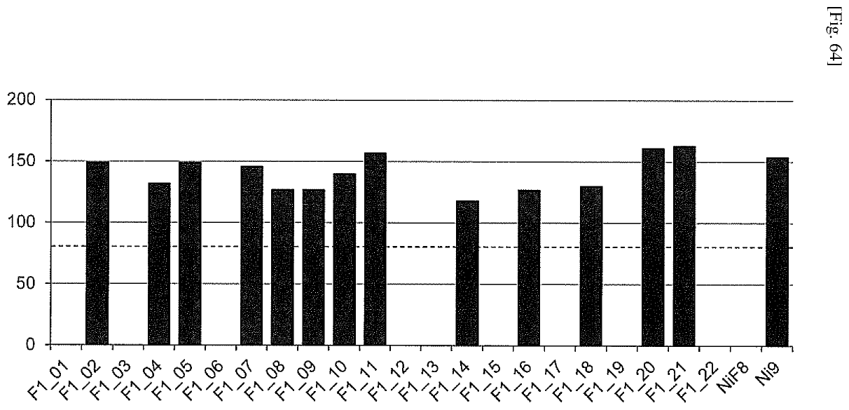

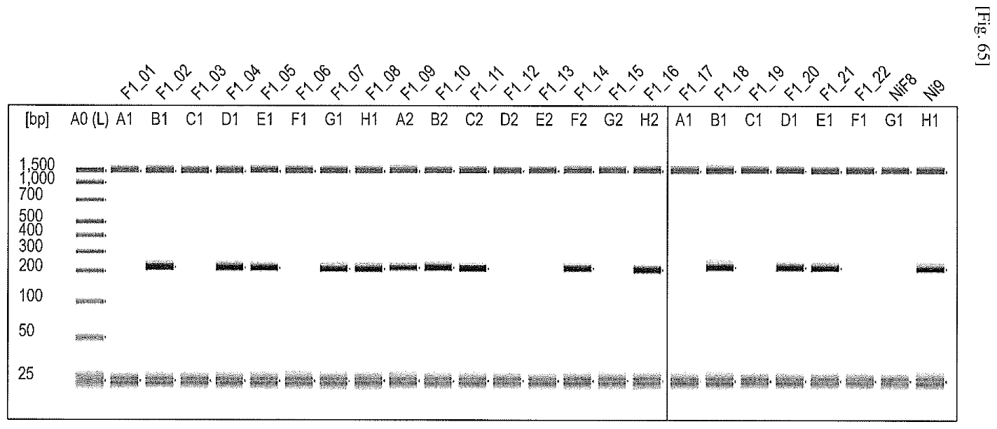

As described in 3.5 above, the sugarcane varieties NiF8 and Ni9 and 22 hybrid progeny lines were subjected to PCR with the use of the primers shown in Table 22, genotypes were identified via electrophoresis, and the results were compared with the number of reads. FIGS. 54 and 55 show the number of reads and the electrophoretic pattern of the NiF8 marker N80521152, respectively. FIGS. 56 and 57 show the number of reads and the electrophoretic pattern of the NiF8 marker N80997192, respectively. FIGS. 58 and 59 show the number of reads and the electrophoretic pattern of the NiF8 marker N80533142, respectively. FIGS. 60 and 61 show the number of reads and the electrophoretic pattern of the Ni9 marker N91552391, respectively. FIGS. 62 and 63 show the number of reads and the electrophoretic pattern of the Ni9 marker N91653962, respectively. FIGS. 64 and 65 show the number of reads and the electrophoretic pattern of the Ni9 marker N91124801, respectively.

As shown in FIGS. 54 to 65, the results for all the PCR markers designed in 3.5 above were consistent with the results of analysis with the use of a next-generation sequencer. It was thus considered that genotype identification with the use of a next-generation sequencer would be applicable as a marker technique.

4.6 Correlation Between Random Primer Density and Length

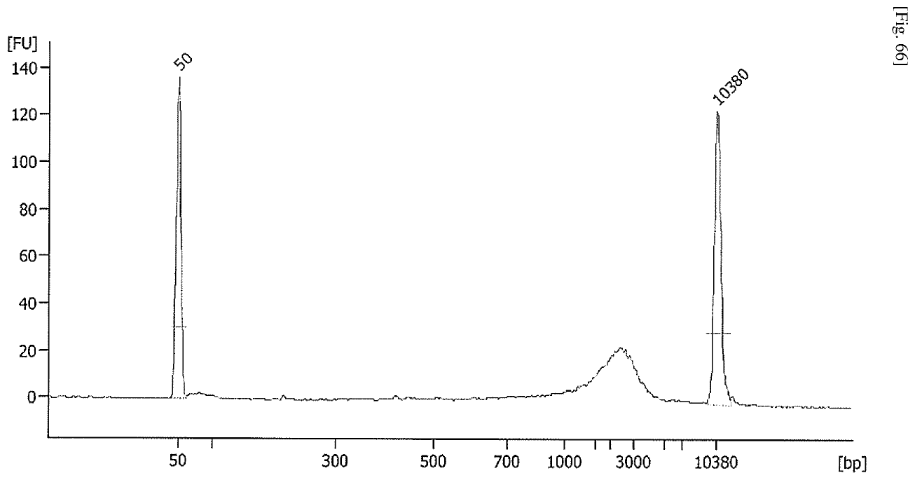

As described in 3.6.1, the results of DNA library production with the use of random primers comprising 9 bases (Table 8), 10 bases (Table 1, 10-base primer A), 11 bases (Table 9), 12 bases (Table 10), 14 bases (Table 11), 16 bases (Table 12), 18 bases (Table 13), and 20 bases (Table 14) are shown in FIGS. 66 to 81. The results are summarized in Table 25.

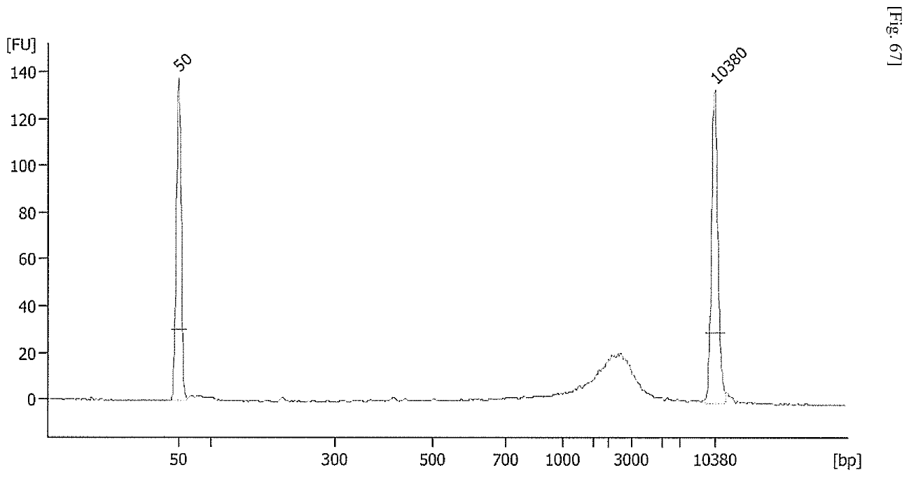

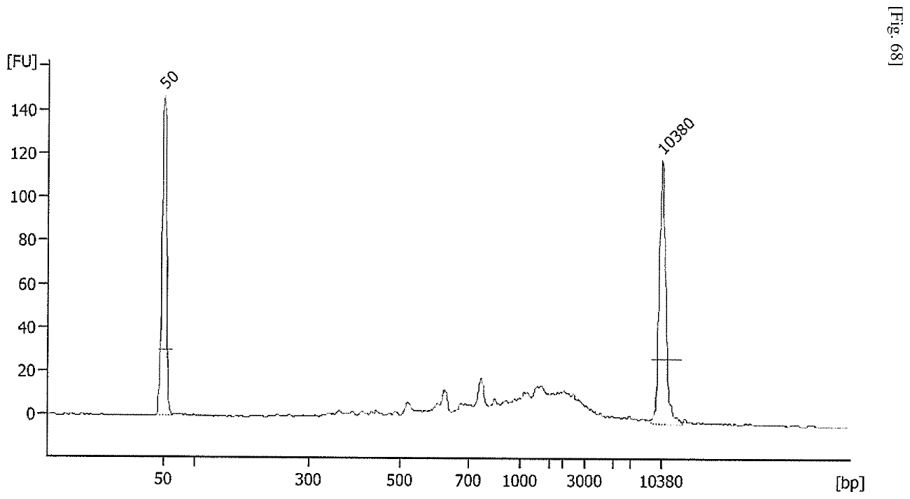

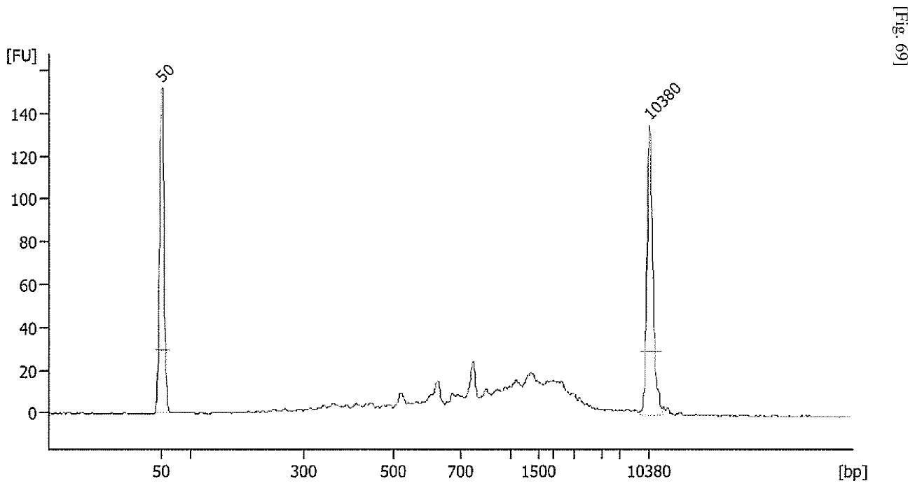

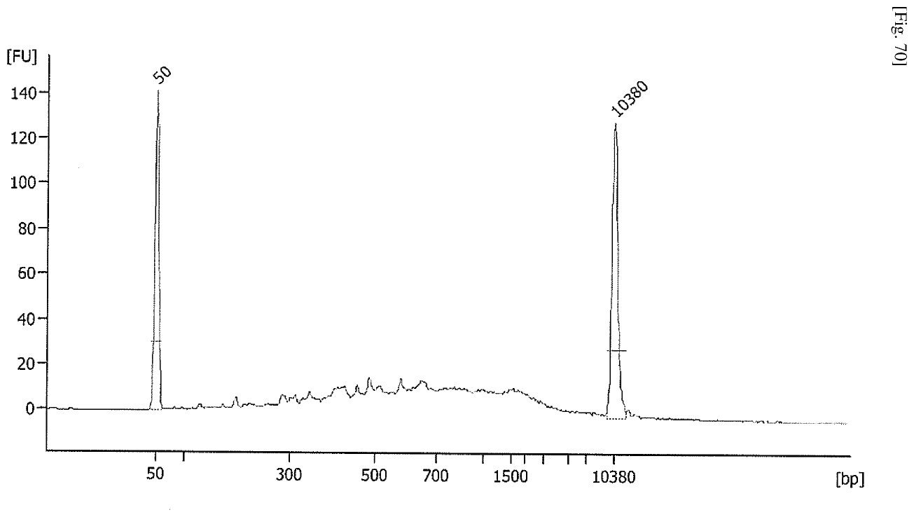

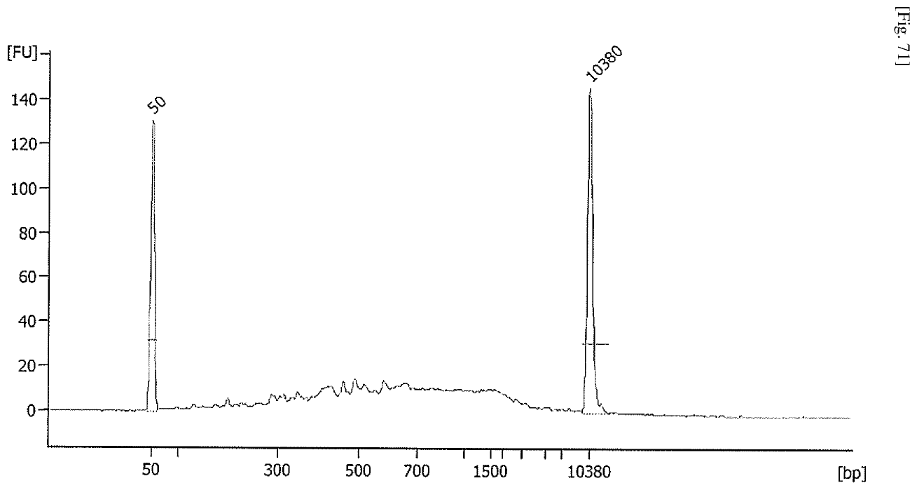

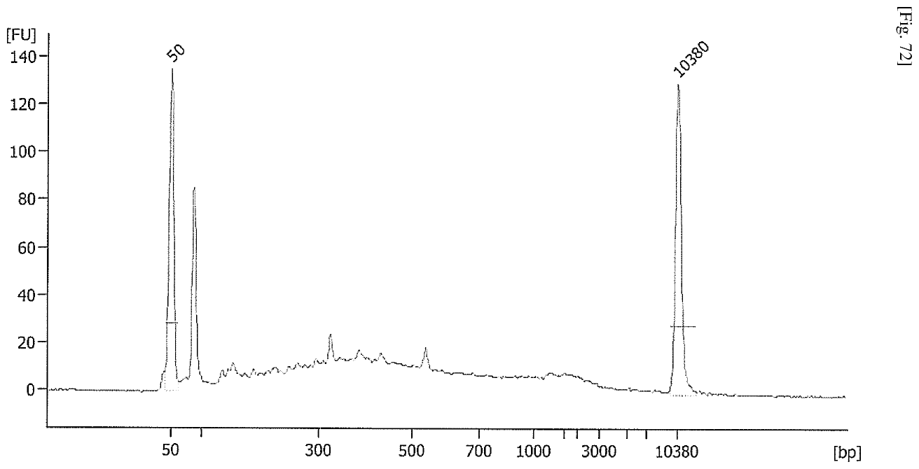

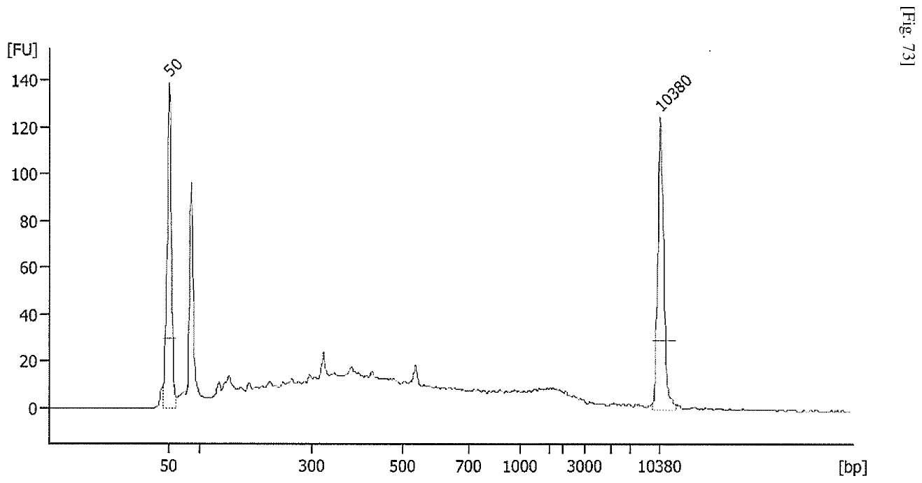

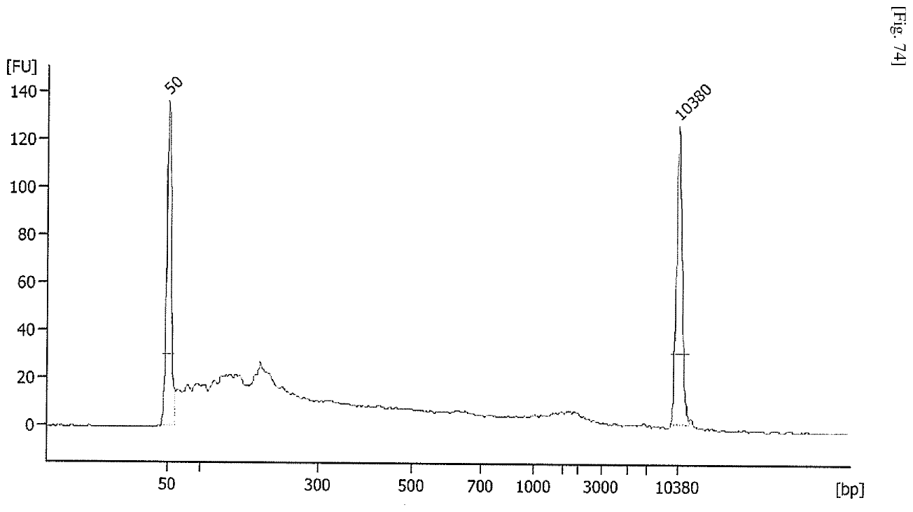

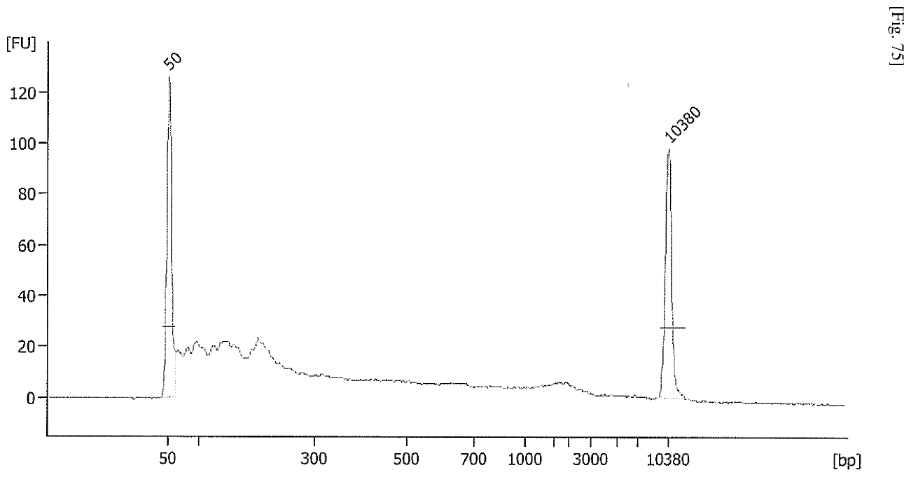

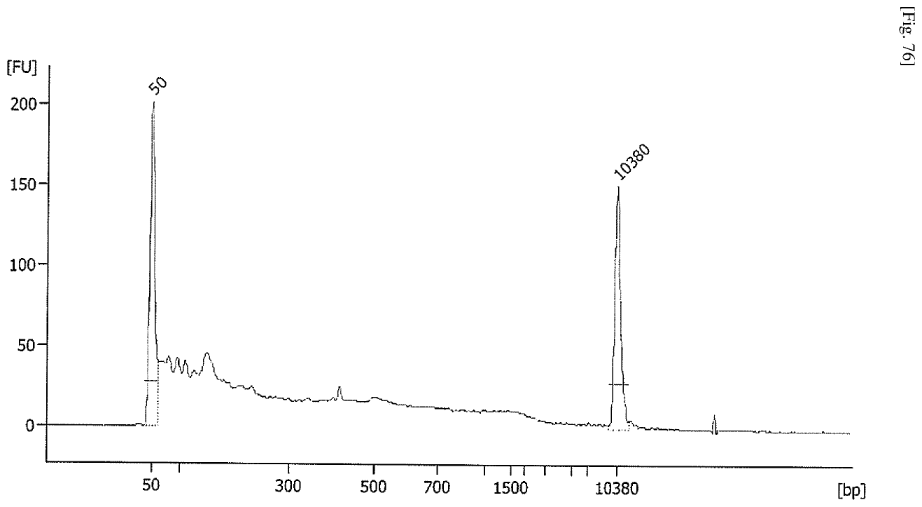

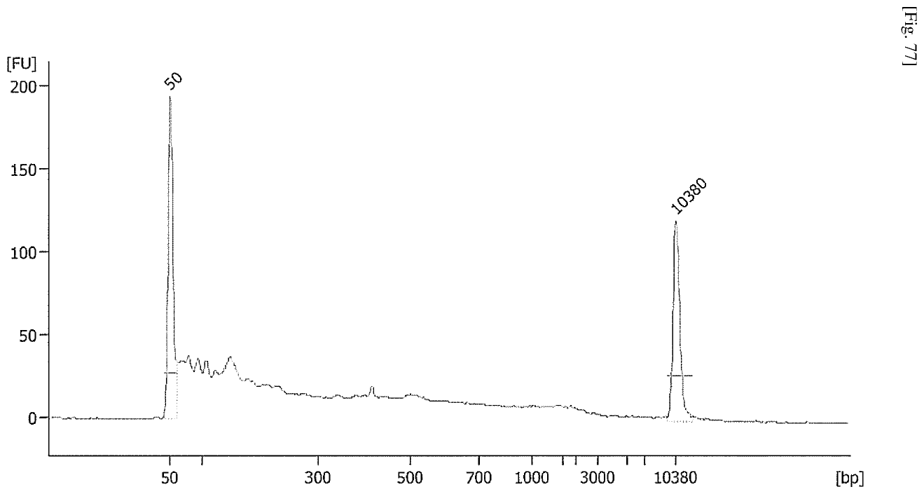

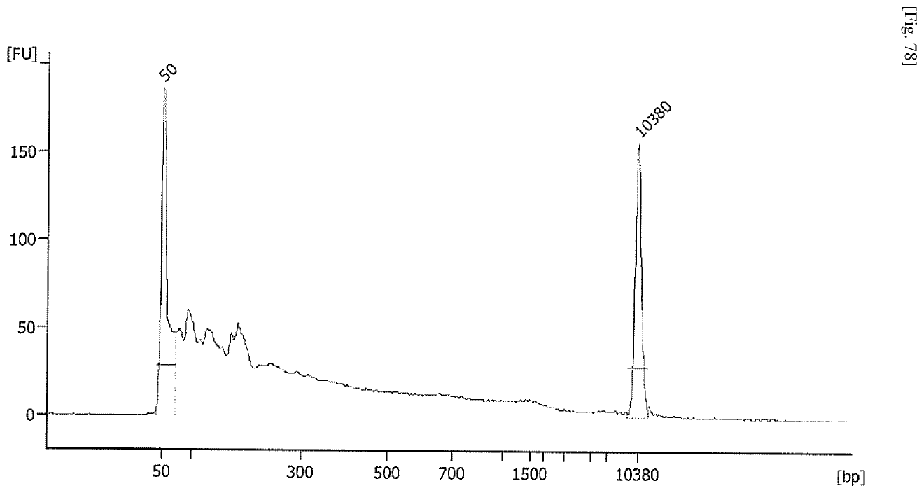

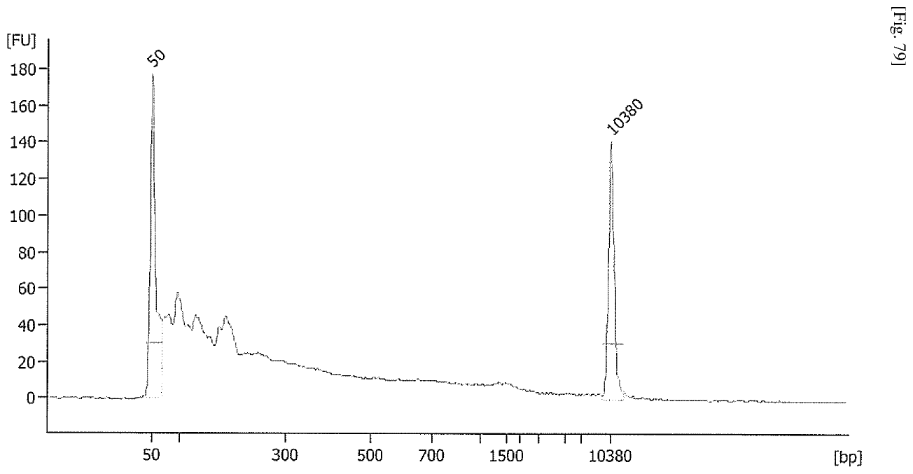

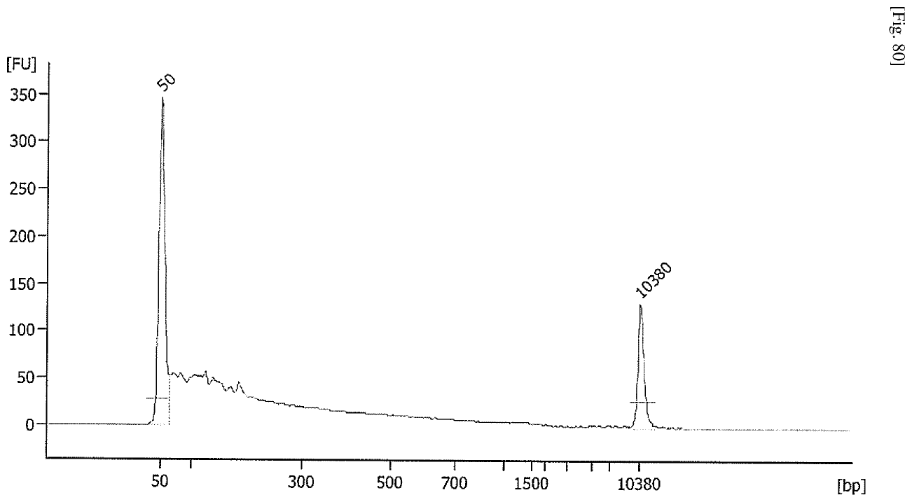

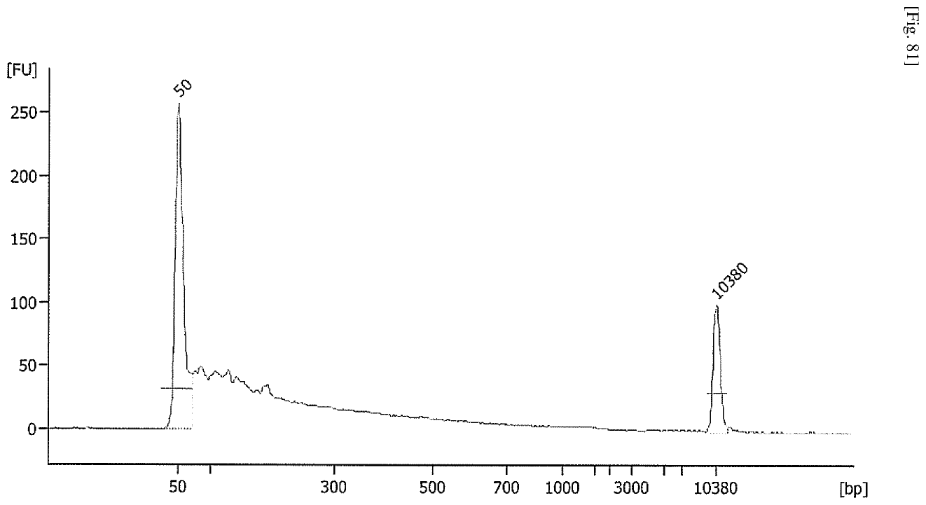

TABLE 25

| Random primer | | | Correlational |
|---|---|---|---|
| length | Repeat | FIG. No. | coefficient (ρ) |
| 9 | First | FIG. 66 | 0.981 |
| | Second | FIG. 67 | |
| 10 | First | FIG. 68 | 0.979 |
| | Second | FIG. 69 | |
| 11 | First | FIG. 70 | 0.914 |
| | Second | FIG. 71 | |
| 12 | First | FIG. 72 | 0.957 |
| | Second | FIG. 73 | |
| 14 | First | FIG. 74 | 0.984 |
| | Second | FIG. 75 | |
| 16 | First | FIG. 76 | 0.989 |
| | Second | FIG. 77 | |
| 18 | First | FIG. 78 | 0.995 |
| | Second | FIG. 79 | |
| 20 | First | FIG. 80 | 0.999 |
| | Second | FIG. 81 | |

When random primers were used at a high concentration of 10.0 microM, which is 13.3 times greater than the usual level, as shown in FIGS. 66 to 81, it was found that a low-molecular-weight DNA fragment could be amplified with the use of random primers comprising 9 to 20 bases while achieving very high reproducibility. As the base length of a random primer increased (12 bases or more, in particular), the molecular weight of the amplified fragment was likely to be decreased. When random primers comprising 9 bases were used, the amount of the DNA fragment amplified was increased by setting the annealing temperature at 37 degrees C.

In order to elucidate the correlation between the density and the length of random primers, as described in 3.6.2 above, PCR was carried out with the use of random primers comprising 8 to 35 bases at the concentration of 0.6 to 300 microM, so as to produce a DNA library. The results are shown in Table 26.

TABLE 26
Correlation between concentration, and length of random primer relative to DNA library

| | Concentration | | | | | | | | | | | | | | | | |
|---|---|---|---|---|---|---|---|---|---|---|---|---|---|---|---|---|---|
| Primer | relative to | Primer length | | | | | | | | | | | | | | | |
| μM | standard | 8 | 9 | 10 | 11 | 12 | 14 | 16 | 18 | 20 | 22 | 24 | 26 | 28 | 29 | 30 | 35 |
| 0.6 | standard | x | x | x | x | x | x | x | x | x | x | x | x | x | x | x | x |
| 2 | 3.3 x | x | x | x | x | x | x | x | x | x | x | x | x | x | x | x | x |
| 4 | 6.7 x | x | x | x | x | x | ∘ | ∘ | ∘ | ∘ | ∘ | ∘ | ∘ | ∘ | x | x | x |
| 6 | 10.0 x | x | x | x | x | x | ∘ | ∘ | ∘ | ∘ | ∘ | ∘ | ∘ | ∘ | ∘ | ∘ | x |
| 8 | 13.3 x | x | x | x | x | ∘ | ∘ | ∘ | ∘ | ∘ | ∘ | ∘ | ∘ | ∘ | ∘ | x | x |
| 10 | 16.7 x | x | x | x | x | ∘ | ∘ | ∘ | ∘ | ∘ | ∘ | ∘ | ∘ | ∘ | ∘ | x | x |
| 20 | 33.3 x | x | x | x | ∘ | ∘ | ∘ | ∘ | ∘ | ∘ | ∘ | ∘ | x | x | x | x | x |
| 40 | 66.7 x | x | ∘ | ∘ | ∘ | ∘ | ∘ | ∘ | ∘ | ∘ | x | x | x | x | x | x | x |
| 60 | 100.0 x | x | ∘ | ∘ | ∘ | ∘ | ∘ | ∘ | ∘ | ∘ | x | x | x | x | x | x | x |
| 100 | 166.7 x | — | x | ∘ | ∘ | ∘ | ∘ | ∘ | ∘ | x | — | — | — | — | — | — | — |
| 200 | 333.3 x | — | x | ∘ | ∘ | x | x | x | x | x | — | — | — | — | — | — | — |
| 300 | 500.0 x | — | x | x | x | x | x | x | x | x | — | — | — | — | — | — | — |

∘: DNA library covering 100 to 500 bases is amplified with good reproducibility (ρ > 0.9)
x: DNA library not covering 100 to 500 bases or reproducibility is poor (ρ ≤ 0.9)
—: Unperformed

As shown in Table 26, it was found that a low-molecular-weight (100 to 500 bases) DNA fragment could be amplified with high reproducibility with the use of random primers comprising 9 to 30 bases at 4.0 to 200 microM. In particular, it was confirmed that low-molecular-weight (100 to 500 bases) DNA fragments could be amplified assuredly with high reproducibility with the use of random primers comprising 9 to 30 bases at 4.0 to 100 microM.

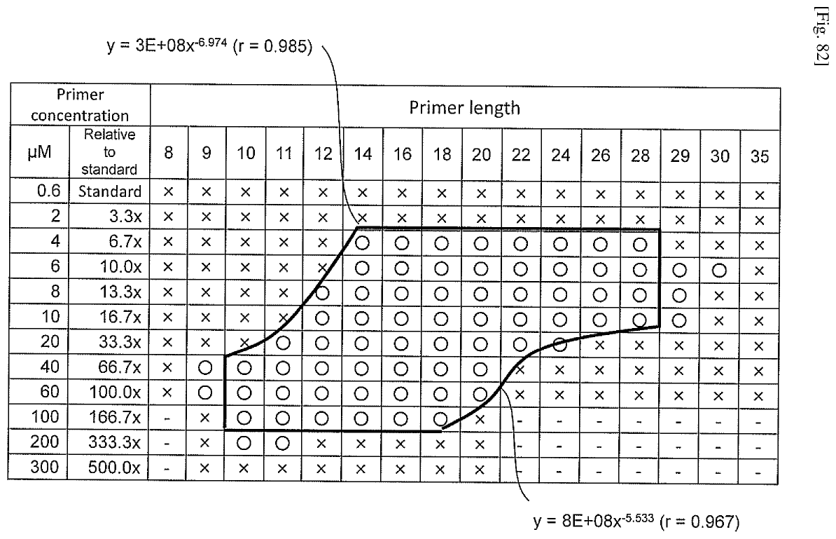

The results shown in Table 26 are examined in greater detail. As a result, the correlation between the length and the concentration of random primers is found to be preferably within a range surrounded by a frame as shown in FIG. 82. More specifically, the random primer concentration is preferably 40 to 60 microM when the random primers comprise 9 to 10 bases. It is preferable that a random primer concentration satisfy the condition represented by an inequation: y>3E+08x^{-6.974} provided that the base length of the random primer is represented by y and the random primer concentration is represented by x, and 100 microM or lower, when the random primer comprises 10 to 14 bases. The random primer concentration is preferably 4 to 100 mM when the random primer comprises 14 to 18 bases. When a random primer comprises 18 to 28 bases, the random primer concentration is preferably 4 microM or higher, and it satisfies the condition represented by an inequation: y<8E+08x^{-5.533}. When a random primer comprises 28 to 29 bases, the random primer concentration is preferably 4 to 10 microM. The inequations y>3E+08x^{-6.974} and y<8E+08x^{-5.533} are determined on the basis of the Microsoft Excel power approximation.

By prescribing the number of bases and the concentration of random primers within given ranges as described above, it was found that low-molecular-weight (100 to 500 bases) DNA fragments could be amplified with high reproducibility. For example, the accuracy of the data obtained via analysis of high-molecular-weight DNA fragments with the use of a next-generation sequencer is known to deteriorate to a significant extent. As described in this example, the number of bases and the concentration of random primers may be prescribed within given ranges, so that a DNA library with a molecular size suitable for analysis with a next-generation sequencer can be produced with satisfactory reproducibility, and such DNA library can be suitable for marker analysis with the use of a next-generation sequencer.

4.7 Number of Random Primers

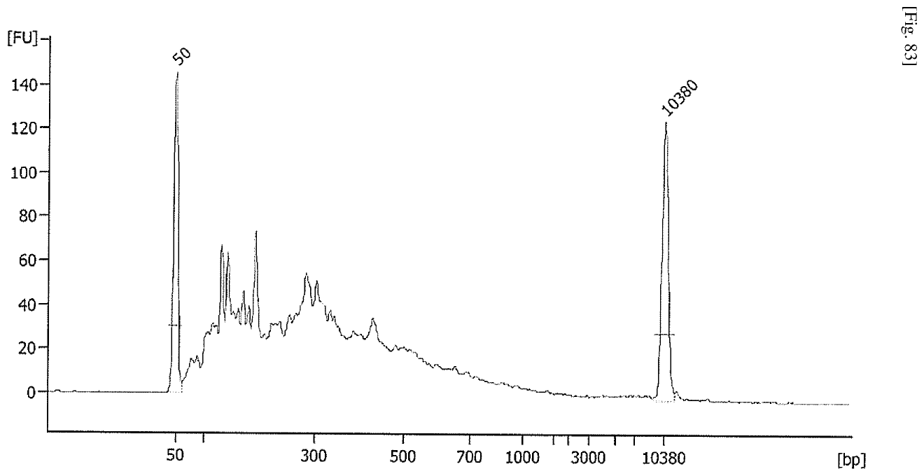

As described in 3.7 above, 1, 2, 3, 12, 24, or 48 types of random primers (concentration: 60 microM) were used to produce a DNA library, and the results are shown in FIGS. 83 to 94. The results are summarized in Table 27.

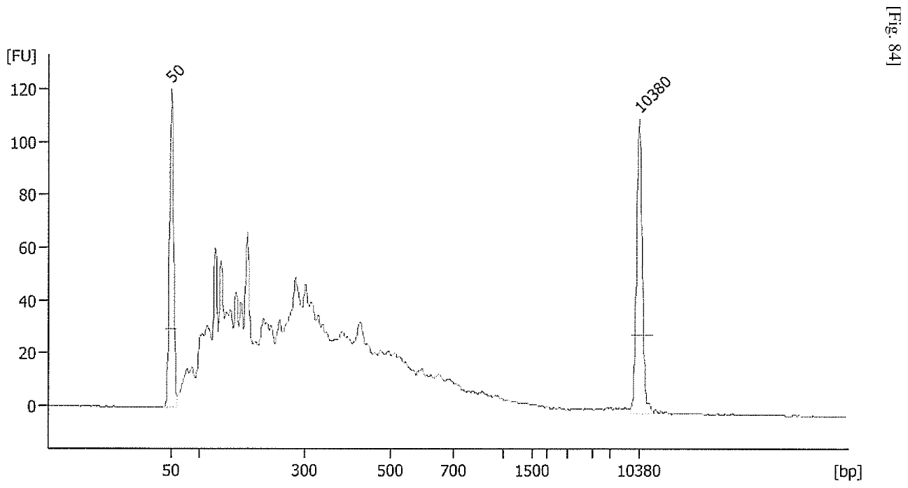

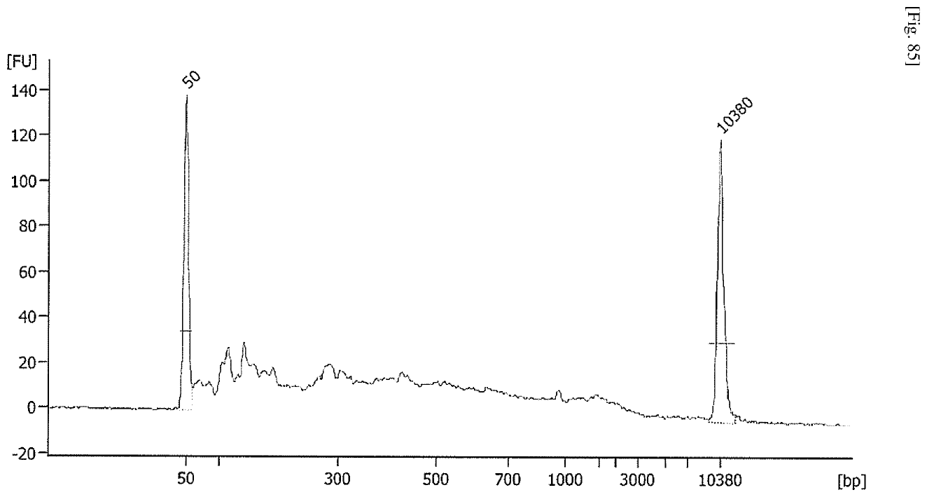

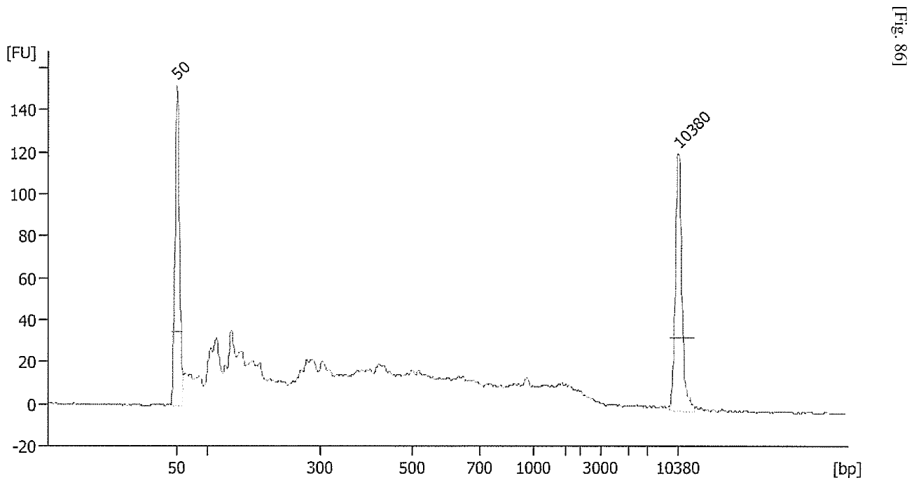

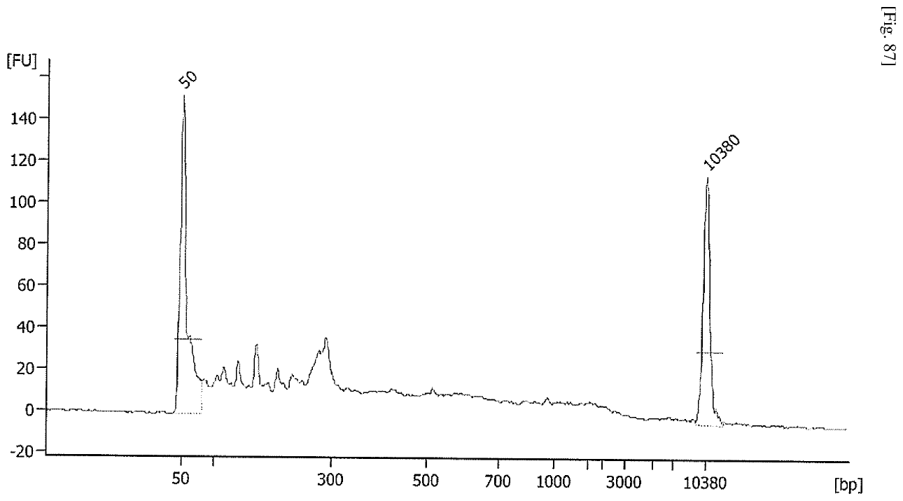

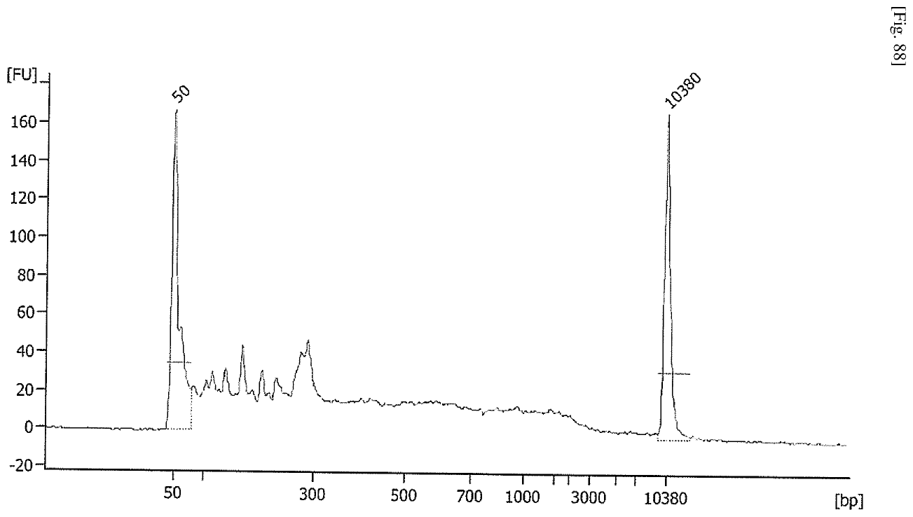

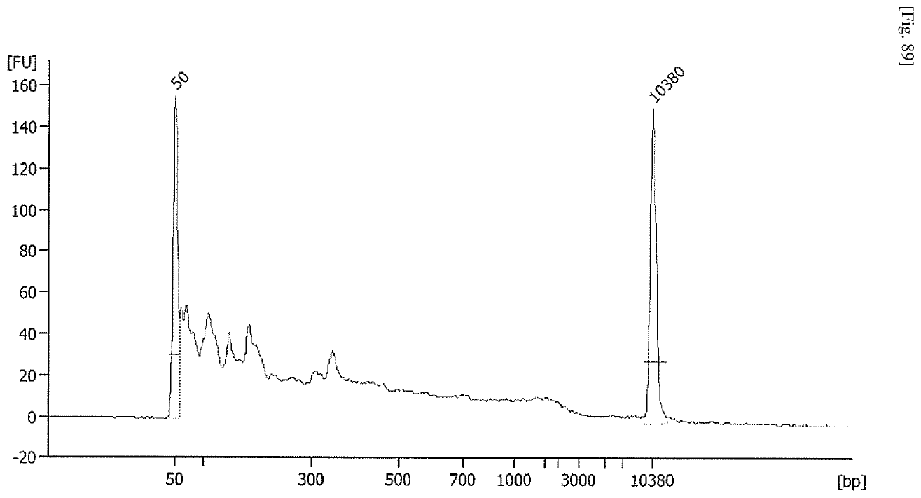

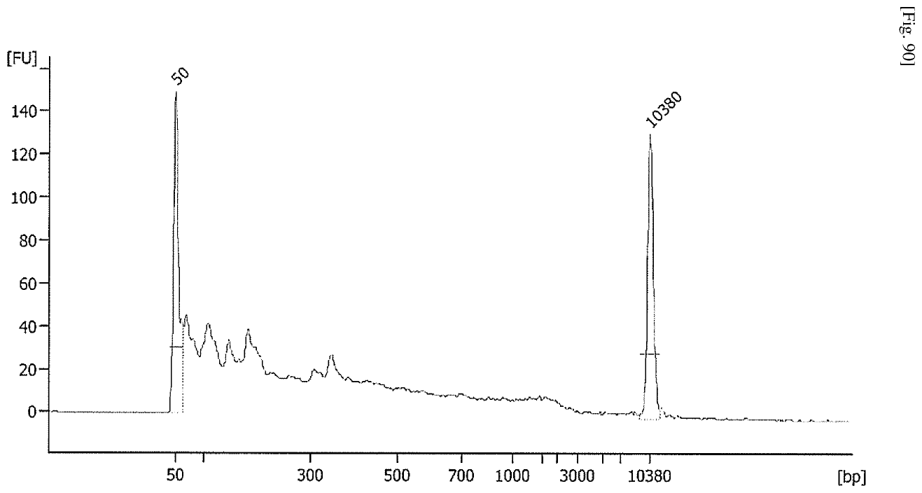

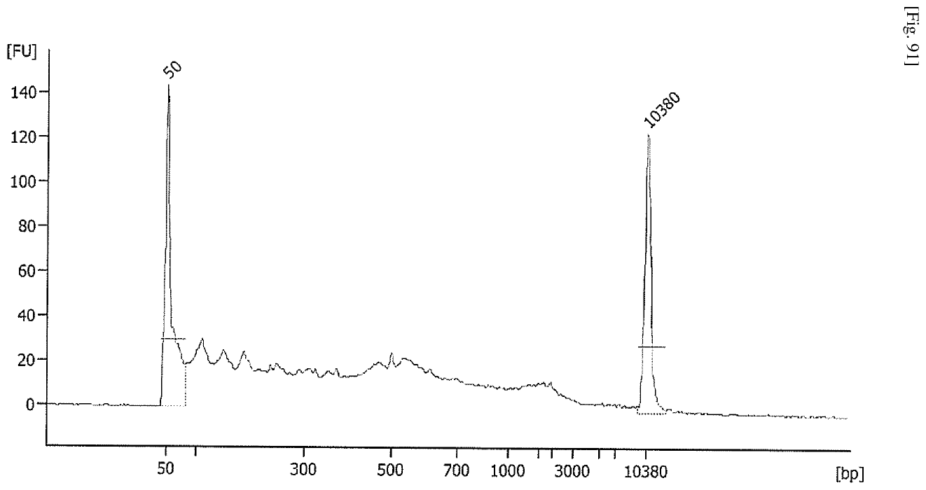

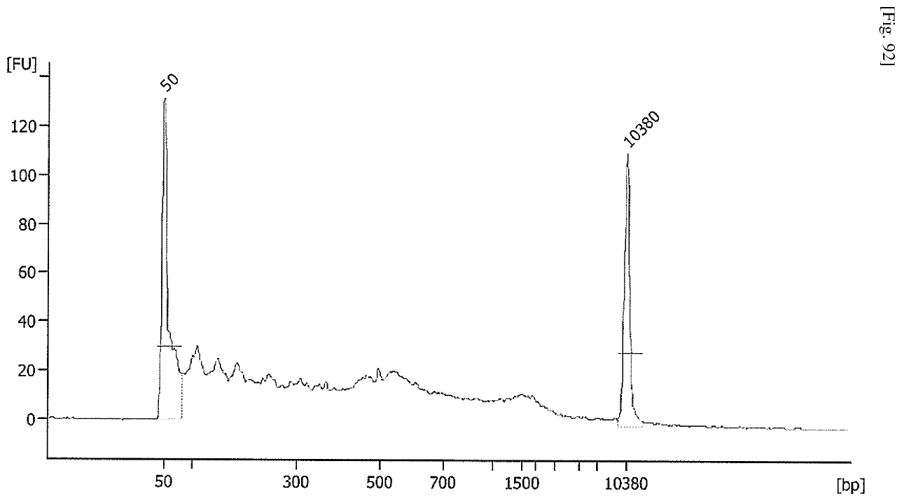

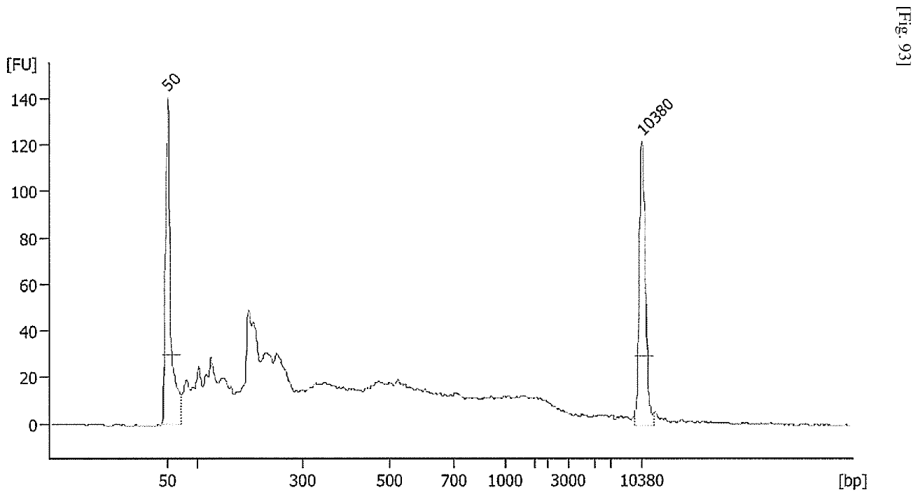

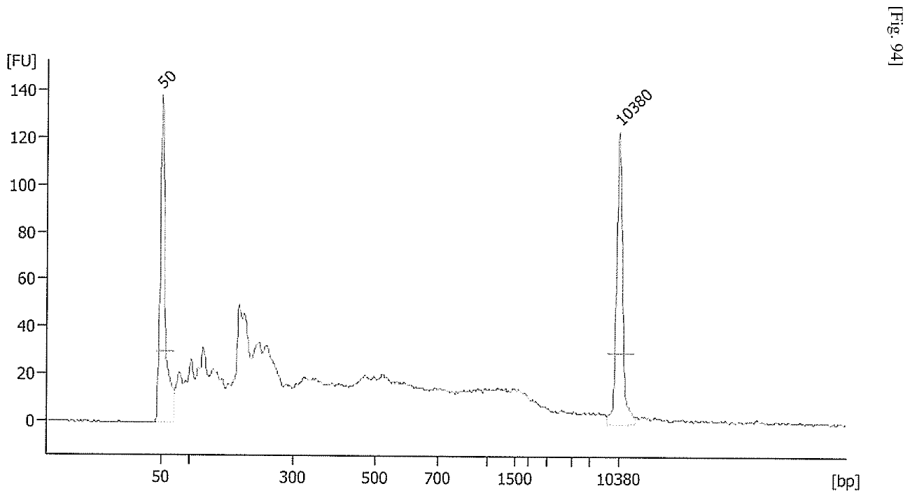

TABLE 27

| Number of random | | | Correlational |
|---|---|---|---|
| primers | Repeat | FIG. No. | coefficient (ρ) |
| 1 | First | FIG. 83 | 0.984 |
| | Second | FIG. 84 | |
| 2 | First | FIG. 85 | 0.968 |
| | Second | FIG. 86 | |
| 3 | First | FIG. 87 | 0.974 |
| | Second | FIG. 88 | |
| 12 | First | FIG. 89 | 0.993 |
| | Second | FIG. 90 | |
| 24 | First | FIG. 91 | 0.986 |
| | Second | FIG. 92 | |
| 48 | First | FIG. 93 | 0.978 |
| | Second | FIG. 94 | |

As shown in FIGS. 83 to 94, it was found that low-molecular-weight DNA fragments could be amplified with the use of any of 1, 2, 3, 12, 24, or 48 types of random primers while achieving very high reproducibility. As the number of types of random primers increases, in particular, a peak in the electrophoretic pattern lowers, and a deviation is likely to disappear.

4.8 Random Primer Sequence

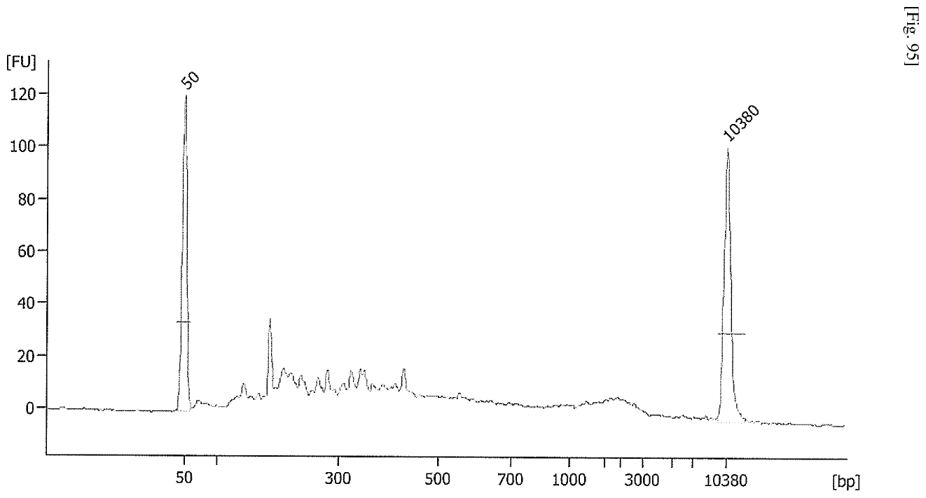

As described in 3.8 above, DNA libraries were produced with the use of sets of random primers shown in Tables 2 to 6 (i.e., 10-base primer B, 10-base primer C, 10-base primer D, 10-base primer E, and 10-base primer F), and the results are shown in FIGS. 95 to 104. The results are summarized in Table 28.

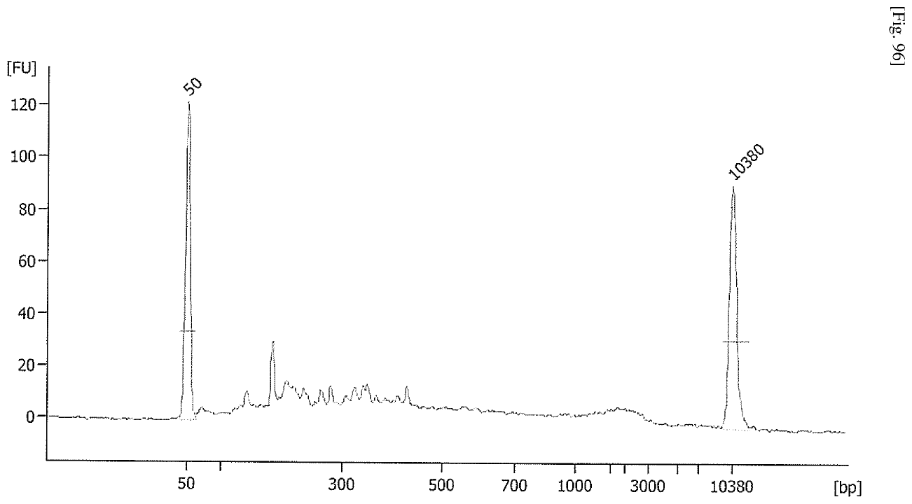

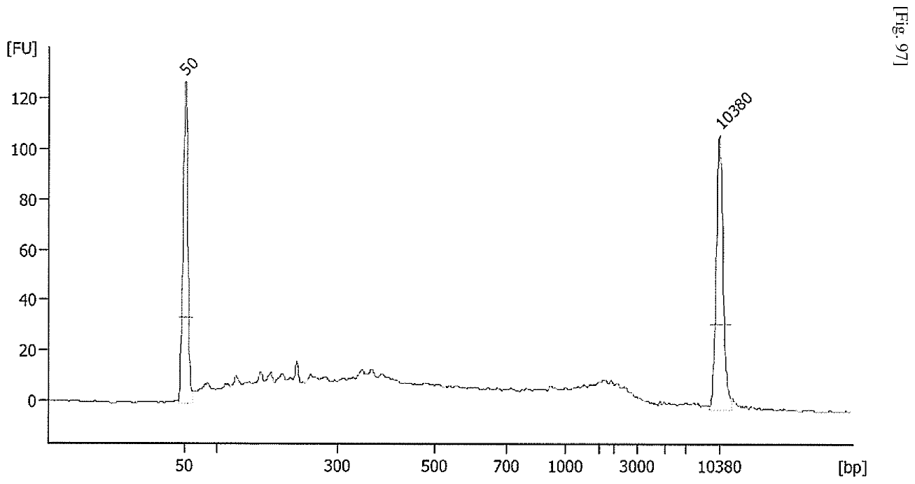

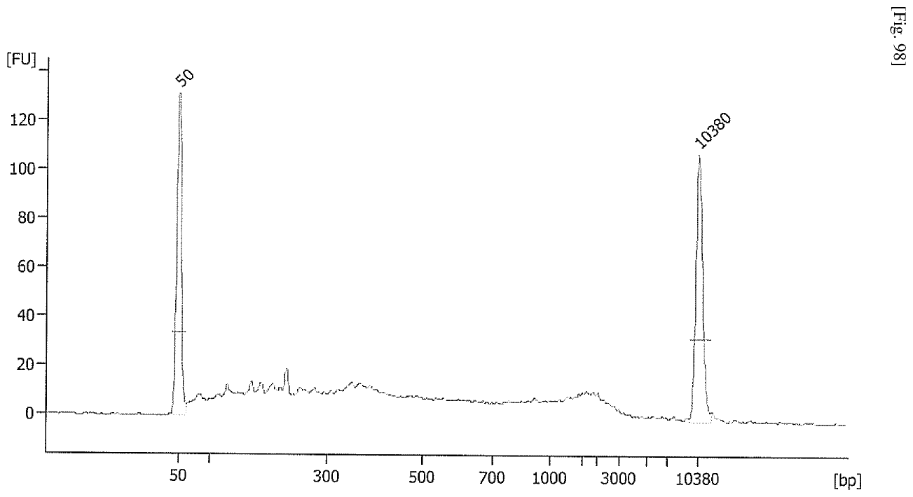

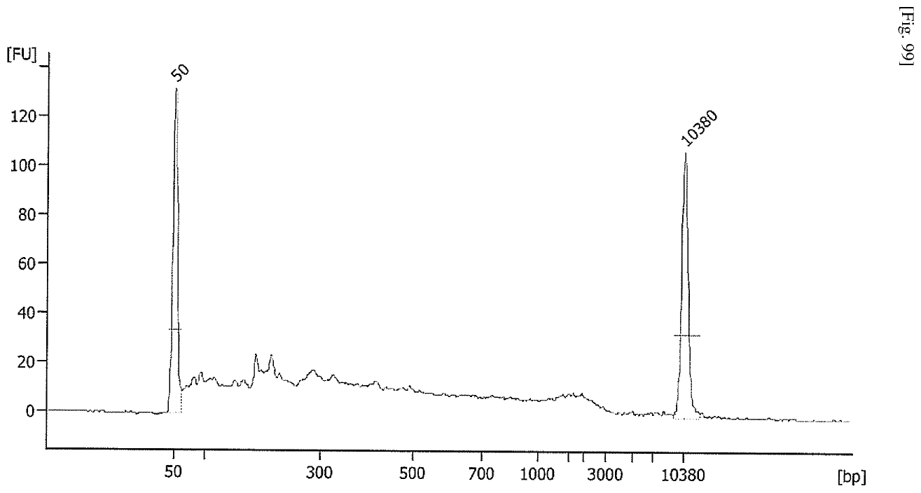

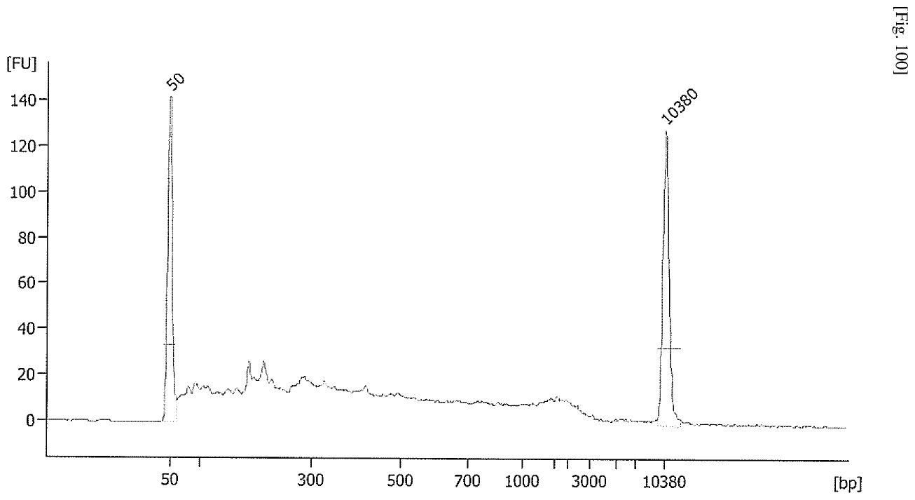

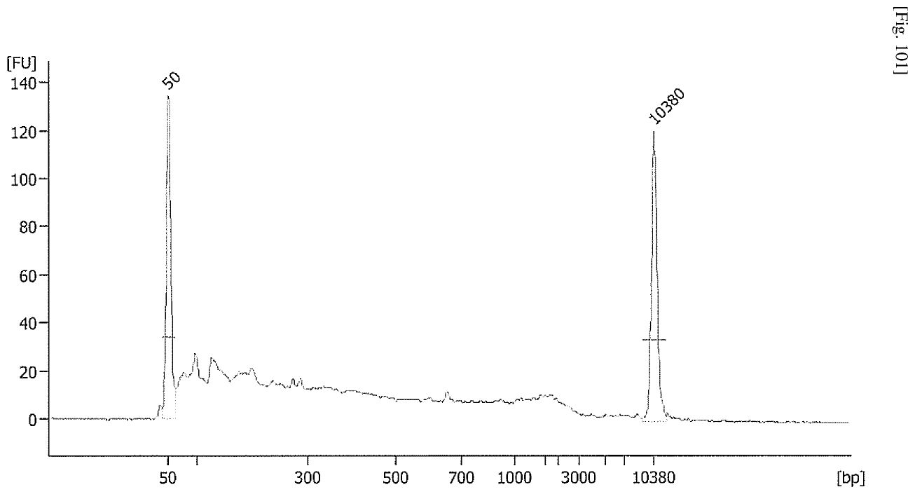

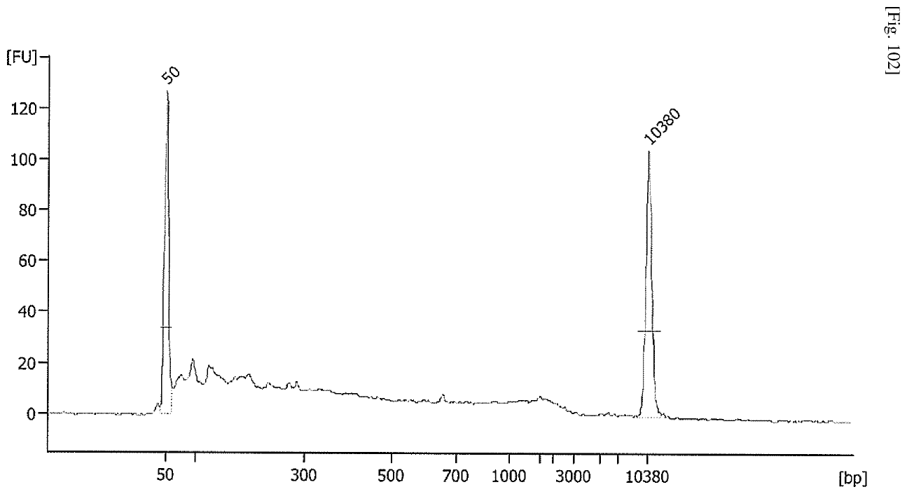

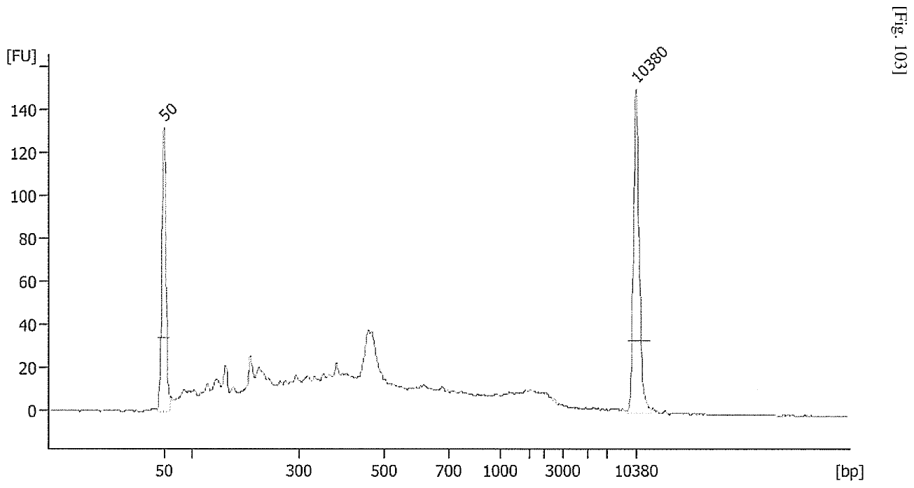

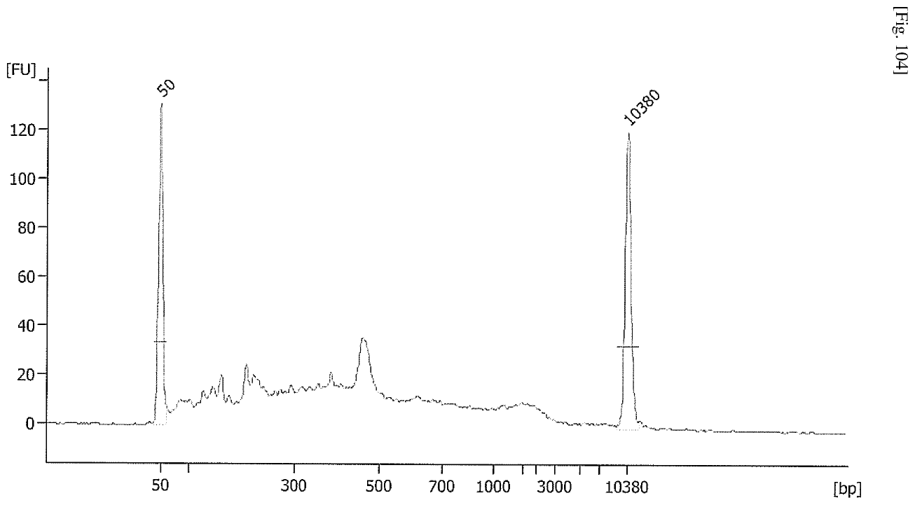

TABLE 28

| | | | Correlational |
|---|---|---|---|
| Set of random primers | Repeat | FIG. No. | coefficient (ρ) |
| 10-base primers B | First | FIG. 95 | 0.916 |
| | Second | FIG. 96 | |
| 10-base primers C | First | FIG. 97 | 0.965 |
| | Second | FIG. 98 | |
| 10-base primers D | First | FIG. 99 | 0.986 |
| | Second | FIG. 100 | |
| 10-base primers E | First | FIG. 101 | 0.983 |
| | Second | FIG. 102 | |
| 10-base primers F | First | FIG. 103 | 0.988 |
| | Second | FIG. 104 | |

As shown in FIGS. 95 to 104, it was found that low-molecular-weight DNA fragments could be amplified with the use of any sets of 10-base primer B, 10-base primer C, 10-base primer D, 10-base primer E, or 10-base primer F while achieving very high reproducibility.

4.9 Production of Human DNA Library

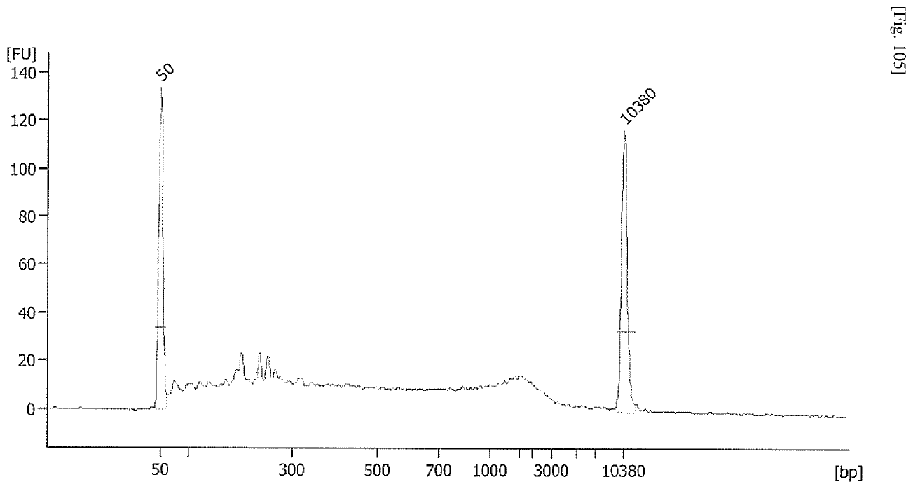

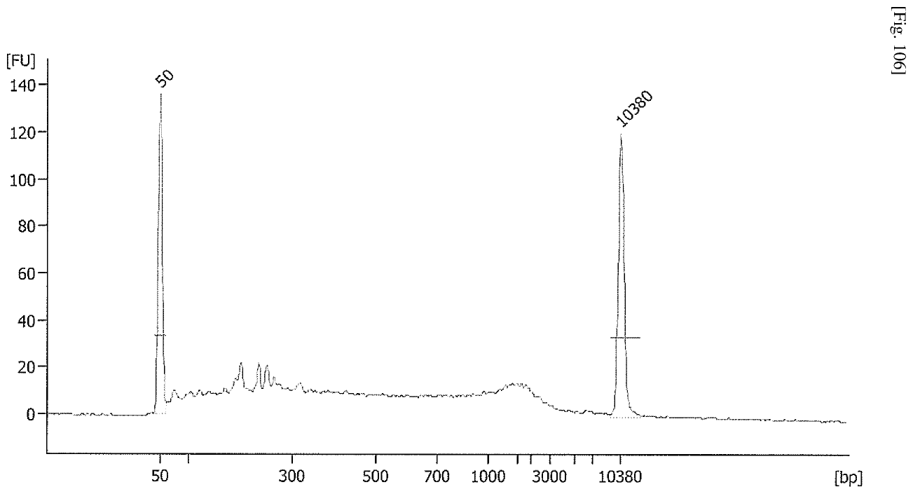

As described in 3.9 above, a DNA library was produced with the use of human-derived genomic DNA and random primers at a final concentration of 60 microM (10-base primer A), and the results are shown in FIGS. 105 and 106. FIG. 105 shows the results of the first repeated experiment, and FIG. 106 shows the results of the second repeated experiment. As shown in FIGS. 105 and 106, it was found that low-molecular-weight DNA fragments could be amplified while achieving very high reproducibility even if human-derived genomic DNA was used.

### Example 2

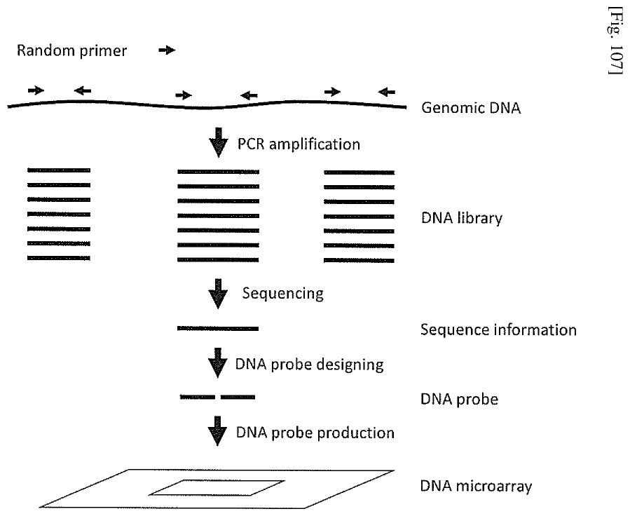

In Example 2, a DNA probe was designed in accordance with the step schematically shown in FIG. 107, and a DNA microarray comprising the designed DNA probe was produced. In this example, whether or not a DNA marker could be detected with the use of such DNA microarray was examined

In this example, a DNA library was produced in the same manner as described in 3.2.1 of Example 1, except that the random primers comprising 10 bases shown in Table 1 and 30 ng of genomic DNAs of the sugarcane varieties NiF8 and Ni9 were used. In this example, also, a sequence library was produced in the same manner as described in 3.2.2 of Example 1 and the sequence library was subjected to MiSeq analysis in the same manner as described in 3.2.3.

In this example, 306,176 types of DNA probes comprising 50 to 60 bases were designed on the basis of the sequence information of the DNA libraries of NiF8 and Ni9 obtained as a result of MiSeq analysis, so as to adjust a TM at around 80 degrees C. The sequences of the designed DNA probes were compared with the sequence information of NiF8 and Ni9, and 9,587 types of probes peculiar to NiF8, which are not found in the Ni9 DNA library, and 9,422 types of probes peculiar to Ni9, which are not found in the NiF8 DNA library, were selected. On the bases of a total of 19,002 types of the selected DNA probes, production of G3 CGH 8×60K Microarrays was consigned to Agilent Technologies, Inc.

With the use of the DNA microarrays thus produced, DNA libraries produced from NiF8, Ni9, and 22 hybrid progeny lines were subjected to detection.

DNA libraries of NiF8, Ni9, and 22 hybrid progeny lines were produced in the same manner as described in 3.2.1 of Example 1. Two DNA libraries were produced for Ni9 and for 2 hybrid progeny lines (i.e., F1_01 and F1_02), so as to obtain the repeated data. The DNA libraries were fluorescently labeled with the use of Cy3-Random Nonamers of the NimbleGen One-Color DNA Labeling Kit in accordance with the NimbleGen Arrays User's Guide.

With the use of the DNA microarrays and the fluorescently-labeled DNA libraries, subsequently, hybridization was carried out in accordance with the array-comparative genomic hybridization (array-CGH) method using the Agilent in-situ oligo-DNA microarray kit. Subsequently, signals on the DNA microarrays when a relevant DNA library was used were detected with the use of the SureScan scanner.

On the basis of the signals detected for NiF8, Ni9, and 22 hybrid progeny lines, 7,140 types of DNA probes exhibiting clear signal intensities were identified. DNA fragments corresponding to such DNA probes can be used as NiF8 markers and Ni9 markers. In this example, genotype data were obtained on the basis of signals obtained from DNA probes corresponding to the NiF8 markers and the Ni9 markers, genotype data obtained through repeated measurements of two hybrid progeny lines (F1_01 and F1_02) were compared, and the accuracy for genotype identification was evaluated on the basis of the data reproducibility.

In this example, genotype data were obtained with the use of PCR markers in order to compare such data with the results of the DNA microarray experiment described above. Specifically, the primers described in 3.5 of Example 1 (Table 22) were used for the 3 NiF8 markers and the 3 Ni9 markers described in 3.5 of Example 1 (i.e., a total of 6 markers). PCR and electrophoresis were performed in the manner as described in 3.5.2 of Example 1, and the results were compared with the signals obtained from the DNA microarray.

In this example, the DNA probes shown below were designed for the 6 markers shown in Table 22 (Table 29).

TABLE 29

| Marker | |
|---|---|
| name | DNA probe sequence |
| N80521152 | CACACACCATGAAGCTTGAACTAATTAACATTCTCAAA |
| | CTAATTAACAAGCATGCAAGCA (SEQ ID |
| | NO: 2041) |
| N80997192 | CAAGTCCTCAATGTCATAGGCGAGATCGCAGTAGTTCT |
| | GTAACCATTCCCTGCTAAACTG (SEQ ID |
| | NO: 2042) |
| N80533142 | GTTTATCAAGATGGGTCATCGAGCTCTTGGTGTCTTC |
| | AACCTTCTTGACATCAACTTCTC (SEQ ID |
| | NO: 2043) |
| N91552391 | CTGAAGCTCTAGGTATGCCTCTTCATCTCCCTGCACC |
| | TCTGGTGCTAGCA (SEQ ID NO: 2044) |
| N91653962 | CTGTCTGCCATTGCCATGTGAGACAAGGAAATCTACT |
| | TCACCCCCATCTATCGA (SEQ ID NO: 2045) |
| N91124801 | TAAGATTAACTATGAACAAATTCACGGGTCCGATTCC |
| | TTTGGGATTTGCAGCTTGCAAGA (SEQ ID |
| | NO: 2046) |

### Results and Examination

### DNA Microarray Analysis

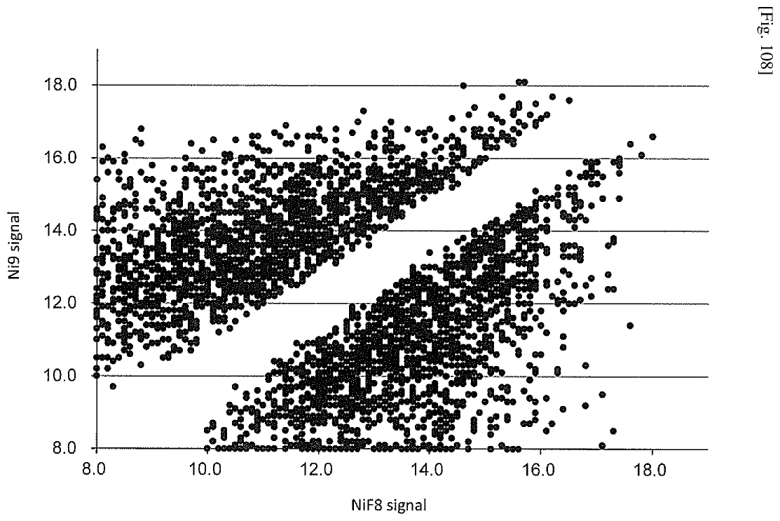

The sugarcane varieties NiF8 and Ni9 and 22 hybrid progeny lines were analyzed with the use of the DNA microarray produced in the manner described above. As a result, 3,570 markers exhibiting apparently different signals between parent varieties were identified as shown in Table 30 (FIG. 108).

TABLE 30

| | Number of | F1_01 | | F1_02 | | Total | |
|---|---|---|---|---|---|---|---|
| | markers | Consistency | Reproducibility | Consistency | Reproducibility | Consistency | Reproducibility |
| NiF8 | 1,695 | 1,695 | 100.00% | 1,695 | 100.00% | 3,390 | 100.00% |
| type | | | | | | | |
| Ni9 | 1,875 | 1,874 | 99.95% | 1,875 | 100.00% | 3,749 | 99.97% |
| type | | | | | | | |
| Total | 3,570 | 3,569 | 99.97% | 3,570 | 100.00% | 7,139 | 99.99% |

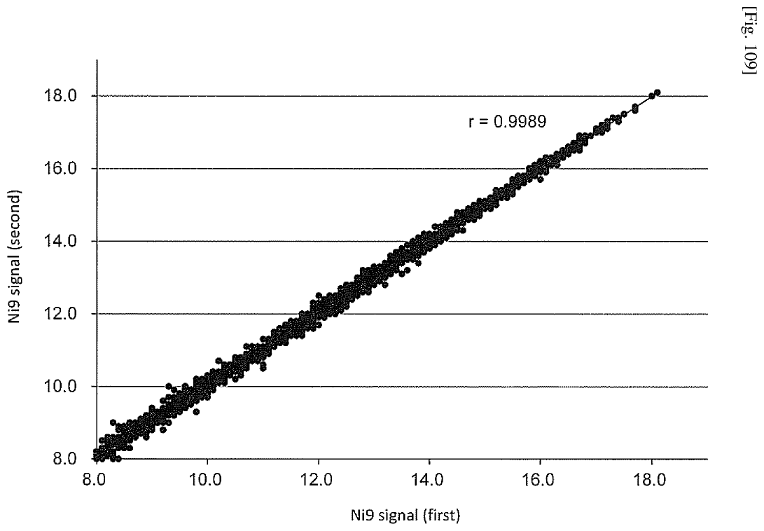

Concerning Ni9, signals obtained through repeated procedures were compared, and a high correlation was found therebetween as a consequence (FIG. 109: r=0.9989). On the basis of the results, the use of random primers at a high concentration was predicted to enable the production of a DNA library with excellent reproducibility and the use of a DNA probe was predicted to enable the detection of a DNA fragment contained in a DNA library (i.e., a marker).

As a result of DNA microarray analysis using the 22 hybrid progeny lines, a total of 78,540 genotype data were obtained, and no missing values were observed for any markers. In order to evaluate the accuracy for genotype identification, the data obtained through repeated analyses of F1_01 and those of F1_02 were compared. As a result, all the data concerning the NiF8 markers were consistent. Concerning the Ni9 marker, a result concerning F1_01 was different, although all the results concerning F1_02 were consistent. With respect to all the markers, 7,139 data out of 7,140 genotype data were consistent; that is, a very high degree of reproducibility was observed (i.e., the degree of consistency: 99.99%).

Experiment for Confirmation with the Use of PCR Marker

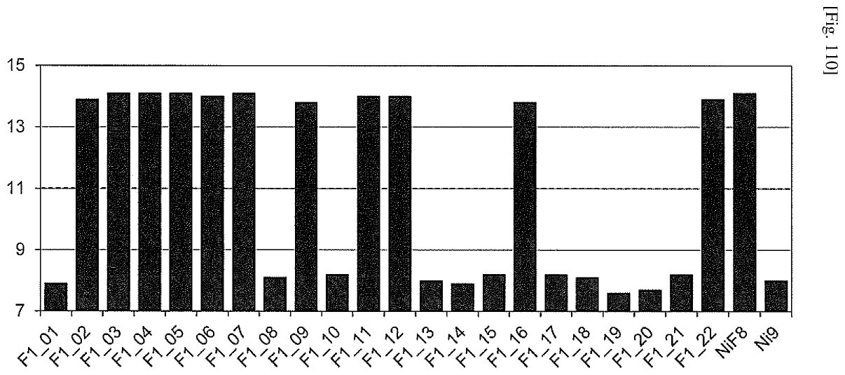

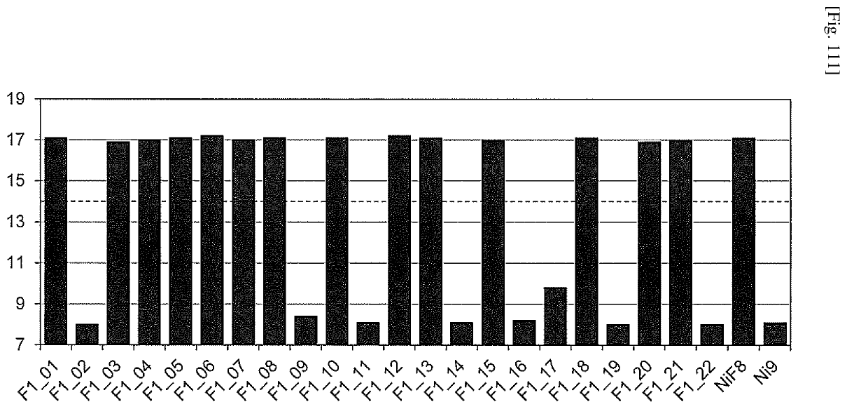

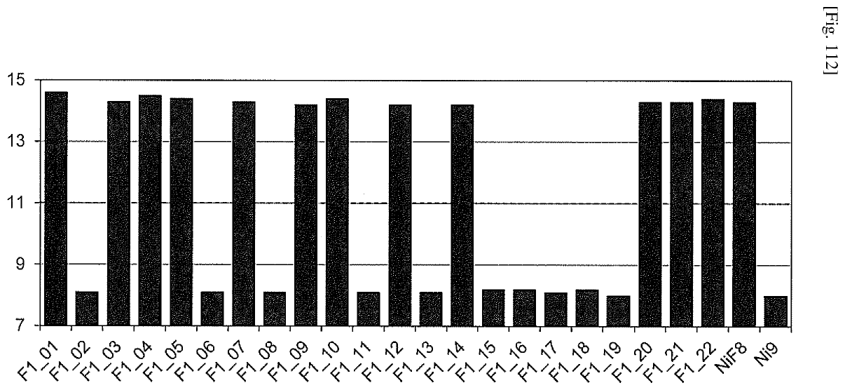

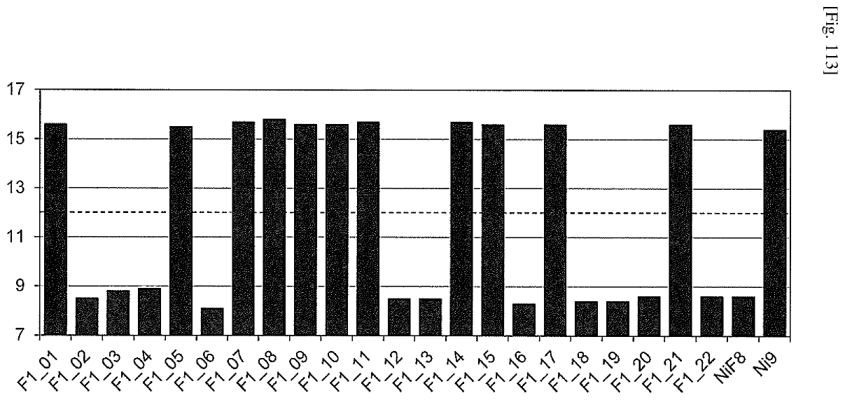

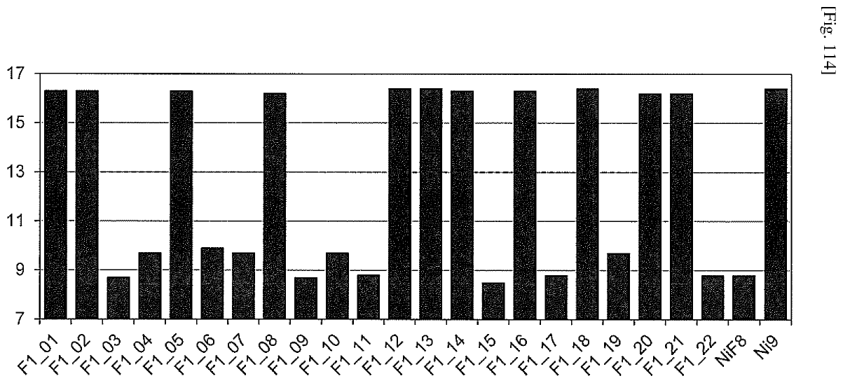

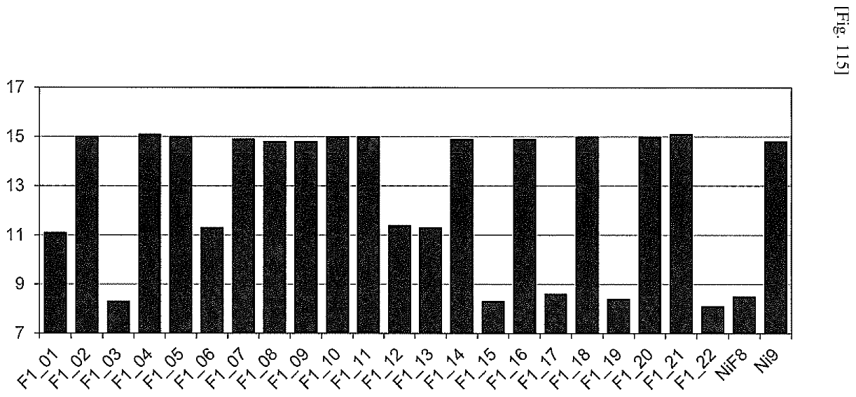

Concerning a total of 6 markers (i.e., 3 NiF8 markers and 3 Ni9 markers), primers designed on the basis of the paired-end marker sequence information were used to subject NiF8, Ni9, and 22 hybrid progeny lines to PCR, the genotypes thereof were identified via electrophoresis, and the results were compared with the signals obtained from the DNA microarray. FIG. 110 shows the results of measurement of signal levels obtained from the DNA probes corresponding to the marker (N80521152), FIG. 111 shows the results of measurement of signal levels obtained from the DNA probes reacting with the marker (N80997192), FIG. 112 shows the results of measurement of signal levels obtained from the DNA probes reacting with the marker (N80533142), FIG. 113 shows the results of measurement of signal levels obtained from the DNA probes reacting with the marker (N91552391), FIG. 114 shows the results of measurement of signal levels obtained from the DNA probes reacting with the marker (N91653962), and FIG. 115 shows the results of measurement of signal levels obtained from the DNA probes reacting with the marker (N91124801). FIG. 55 shows the electrophoretic pattern for the marker (N80521152), FIG. 57 shows the electrophoretic pattern for the marker (N80997192), FIG. 59 shows the electrophoretic pattern for the marker (N80533142), FIG. 61 shows the electrophoretic pattern for the marker (N91552391), FIG. 63 shows the electrophoretic pattern for the marker (N91653962), and FIG. 65 shows the electrophoretic pattern for the marker (N91124801). As a result of comparison of the results of electrophoretic patterns and the results of measurement of signal values obtained from DNA probes, the results for all markers are found to be consistent among all the markers. The results demonstrate that a DNA probe may be designed on the basis of the nucleotide sequence of the DNA fragment contained in the DNA library resulting from the use of a random primer at a high concentration, so that the DNA fragment can be detected with high accuracy.

1. A method for producing a DNA probe comprising steps of: conducting a nucleic acid amplification reaction in a reaction solution containing genomic DNA and a random primer at a high concentration using genomic DNA as a template to obtain DNA fragments; determining the nucleotide sequence of the obtained DNA fragments; and designing a DNA probe used for detecting a DNA fragment obtained in the above step on the basis of the nucleotide sequence of the DNA fragments.

2. The method for producing a DNA probe according to claim 1, wherein DNA fragments are obtained from a plurality of different genomic DNAs with the use of the random primers and, on the basis of the nucleotide sequence of the DNA fragments, the DNA probe containing regions different between the genomic DNAs is designed.

3. The method for producing a DNA probe according to claim 1, wherein the nucleotide sequence of the DNA fragment is compared with a known nucleotide sequence and the DNA probe containing a region different from that of the known nucleotide sequence is designed.

4. The method for producing a DNA probe according to claim 1, wherein the reaction solution contains a random primer at a concentration of 4 to 200 microM.

5. The method for producing a DNA probe according to claim 1, wherein the reaction solution contains a random primer at a concentration of 4 to 100 microM.

6. The method for producing a DNA probe according to claim 1, wherein the random primers each contain 9 to 30 nucleotides.

7. The method for producing a DNA probe according to claim 1, wherein the DNA fragment contains 100 to 500 nucleotides.

8. A method for analyzing genomic DNA comprising steps of: bringing the DNA probe produced by the method for producing a DNA probe according to claim 1 into contact with a DNA fragment derived from genomic DNA subjected to analysis; and detecting hybridization occurring between the DNA probe and the DNA fragment.

9. The method for analyzing genomic DNA according to claim 8, which further comprises a step of conducting a nucleic acid amplification reaction with the use of the genomic DNA subjected to analysis and the random primer to obtain the DNA fragments.

10. The method for analyzing genomic DNA according to claim 8, wherein the DNA fragment derived from genomic DNA is a DNA marker and the presence or absence of the DNA marker is detected with the use of the DNA probe.

11. An apparatus for DNA analysis comprising the DNA probe produced by the method for producing a DNA probe according to claim 1 and a support comprising the DNA probe immobilized thereon.

12. The apparatus for DNA analysis according to claim 11, wherein the support is a substrate or bead.
